(19) United States
(12) Patent Application Publication
(10) Pub. No.: US 2025/0145596 A1
(43) Pub. Date: May 8, 2025

(54) HETEROCYCLIC GLP-1 AGONISTS

(71) Applicant: Gasherbrum Bio, Inc., South San Francisco, CA (US)

(72) Inventors: Qinghua MENG, Shanghai (CN); Weiqiang XING, Shanghai (CN)

(21) Appl. No.: 18/837,146

(22) PCT Filed: Feb. 8, 2023
(86) PCT No.: PCT/CN2023/074938

§ 371 Date: Aug. 8, 2024

(30) Foreign Application Priority Data
Feb. 9, 2022 (WO) PCT/CN2022/075603

(51) Int. Cl. C07D 405/14; A61K 31/4545; A61K 31/496; A61K 31/497; A61K 31/501; A61K 31/506; A61K 45/06; C07D 401/14; C07D 409/14; C07D 413/14

(52) U.S. Cl. CPC C07D 405/14; A61K 31/4545; A61K 31/496; A61K 31/497; A61K 31/501; A61K 31/506; A61K 45/06; C07D 401/14; C07D 409/14; C07D 413/14

(57) ABSTRACT

Provided are GLP-1 agonists and pharmaceutical compositions comprising the same, as well as methods for treating a GLP-1 associated disease, disorder, or condition.

### CROSS REFERENCE TO RELATED APPLICATIONS

The application claims the benefit of International Patent Application Number PCT/CN2022/075603, filed on Feb. 9, 2022, which is incorporated herein by reference in its entirety.

### FIELD

This disclosure relates to GLP-1 agonists, pharmaceutical compositions, and methods of use thereof.

### BACKGROUND

Incretin metabolic hormones, including glucagon-like peptide-1 (GLP-1) and glucose-dependent insulinotropic polypeptide (GIP), are important in the regulation of glucose homeostasis. Medicaments targeting this family of intestinal peptides, such as GLP-1 agonists, have been shown to suppress glucagon production, decrease gastric motility, and increase satiety.

Diabetes mellitus refers to a group of metabolic disorders characterized by persistent hyperglycemia. The most common form, type 2 diabetes mellitus (T2DM) is an acquired condition that accounts for more than 90% of diabetes cases. Typical onset occurs in obese or otherwise sedentary adults and begins with insulin resistance. Though lifestyle changes can be useful in management of this disorder, patients with T2DM may be required to take antidiabetic medications, including dipeptidyl peptidase-4 inhibitors, SGLT2 inhibitors, and sulfonylureas, among others.

In healthy individuals, the incretin hormones glucose-dependent insulinotropic polypeptide (GIP) and glucagon-like peptide 1 (GLP-1) provide tandem modulation of insulin secretory response to glucose ingestion. While this incretin effect is significantly diminished (if at all present) in cases of T2DM, GLP-1 retains insulinotropic properties, even as endocrine pancreatic response to GIP is effectively halted. As such, incretin mimetics and other GLP-1-based therapies can help stimulate insulin production in T2DM patients.

### SUMMARY

The present application describes heterocyclic GLP-1 agonists, as well as pharmaceutical compositions comprising the compounds disclosed herein. Also provided are methods for treating GLP-1-associated diseases, disorders, and conditions.

In one aspect, provided is a compound of Table 1 or a pharmaceutically acceptable salt or solvate thereof.

This disclosure also provides pharmaceutical compositions comprising one or more compounds of Table 1, or a pharmaceutically acceptable salt or solvate thereof, and a pharmaceutically acceptable excipient.

Also provided herein are pharmaceutical compositions comprising a compound of Table 1, or a pharmaceutically acceptable salt, stereoisomer, mixture of stereoisomers, or prodrug thereof, and a pharmaceutically acceptable excipient.

Also provided herein are methods for treating type 2 diabetes mellitus in a patient in need thereof, the methods comprising administering to the patient a therapeutically effective amount of a compound of Table 1, or a pharmaceutically acceptable salt or solvate thereof, or a pharmaceutical composition thereof.

Also provided herein are methods for treating type 2 diabetes mellitus in a patient, the methods comprising administering to a patient identified or diagnosed as having type 2 diabetes mellitus a therapeutically effective amount of a compound of Table 1, or a pharmaceutically acceptable salt or solvate thereof, or a pharmaceutical composition thereof.

Also provided herein are methods for treating diabetes mellitus in a patient, the methods comprising determining that the patient has type 2 diabetes mellitus; and administering to the patient a therapeutically effective amount of a compound of Table 1, or a pharmaceutically acceptable salt or solvate thereof, or a pharmaceutical composition thereof. In some embodiments, the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof. In some embodiments, the level of HbA1c is greater than or about 6.5%. In some embodiments, the level of fasting plasma glucose is greater than or about 126 mg/dL. In some embodiments, the level of non-fasting plasma glucose is greater than or about 200 mg/dL.

In some embodiments, the methods further comprise obtaining a sample from the patient. In some embodiments, the sample is a body fluid sample. In some embodiments, the patient is about 40 to about 70 years old and is overweight or obese. In some embodiments, the patient has a body mass index (BMI) greater than or about 22 kg/m². In some embodiments, the patient has a BMI greater than or about 30 kg/m².

In some embodiments, the methods for the treatment of type 2 diabetes mellitus comprise a reduction in fasting plasma glucose levels. In some embodiments, the fasting plasma glucose levels are reduced to about or below 100 mg/dL.

In some embodiments, the methods for the treatment of type 2 diabetes mellitus comprise a reduction in HbA1c levels. In some embodiments, the HbA1c levels are reduced to about or below 5.7%.

In some embodiments, the methods for the treatment of type 2 diabetes mellitus comprise a reduction in glucagon levels.

In some embodiments, the methods for the treatment of type 2 diabetes mellitus comprise an increase in insulin levels.

In some embodiments, the methods for the treatment of type 2 diabetes mellitus comprise a decrease in BMI. In some embodiments, the BMI is decreased to about or below 25 kg/m².

In some embodiments, the compound of Table 1, or a pharmaceutically acceptable salt or solvate thereof, or a pharmaceutical composition thereof, is administered orally.

In some embodiments, the methods of treatment for type 2 diabetes mellitus further comprise administering an additional therapy or therapeutic agent to the patient. In some embodiments, the additional therapy or therapeutic agent is selected from the group consisting of an antidiabetic agent, an anti-obesity agent, a GLP-1 receptor agonist, an agent to treat non-alcoholic steatohepatitis (NASH), anti-emetic agent, gastric electrical stimulation, dietary monitoring, physical activity, or any combinations thereof. In some embodiments, the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof. In some embodiments, the biguanide is metformin. In some embodiments, the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof. In some embodiments, the GLP-1 receptor agonist is selected from the group consisting of liraglutide, exenatide, dulaglutide, albiglutide, taspoglutide, lixisenatide, semaglutide, or any combinations thereof. In some embodiments, the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASKI) inhibitor, or any combinations thereof. In some embodiments, the compound of Table 1, or a pharmaceutically acceptable salt or solvate thereof, or a pharmaceutical composition thereof, and the additional therapeutic agent are administered as separate dosages sequentially in any order.

Also provided herein are methods for modulating insulin levels in a patient in need of such modulating, the method comprising administering to the patient an effective amount of a compound of Table 1, or a pharmaceutically acceptable salt or solvate thereof, or a pharmaceutical composition thereof. In some embodiments, the modulation results in an increase of insulin levels.

Also provided herein are methods for modulating glucose levels in a patient in need of such modulating, the method comprising administering to the patient an effective amount of a compound of Table 1, or a pharmaceutically acceptable salt or solvate thereof, or a pharmaceutical composition thereof. In some embodiments, the modulation results in a decrease of glucose levels.

Also provided herein are methods for treating a GLP-1 associated disease, disorder, or condition, the method comprising administering to a patient in need thereof an effective amount of a compound of Table 1, or a pharmaceutically acceptable salt or solvate thereof, or a pharmaceutical composition thereof. In some embodiments, the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof. In some embodiments, the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof. In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

Before the present compounds and methods are described, it is to be understood that the disclosure is not limited to the methodologies, protocols, cell lines, assays, and reagents described, as these may vary. It is also to be understood that the terminology used herein is intended to describe embodiments of the present disclosure, and is in no way intended to limit the scope of the present disclosure as set forth in the appended claims.

### Definitions

It must be noted that as used herein, and in the appended claims, the singular forms “a,” “an,” and “the” include plural references unless the context clearly dictates otherwise.

Unless defined otherwise, all technical, and scientific terms used herein have the same meanings as commonly understood by one of ordinary skill in the art to which this disclosure pertains. Although any methods and materials similar or equivalent to those described herein can be used in the practice or testing of the present disclosure, exemplary methods, devices, and materials are now described. All publications cited herein are incorporated herein by reference in their entirety for the purpose of describing and disclosing the methodologies, reagents, and tools reported in the publications that might be used in connection with the disclosure.

Provided herein are heterocyclic GLP-1 agonists for use in the management of T2DM and other conditions where activation of GLP-1 activity is useful.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “halo” or “halogen” means —F (sometimes referred to herein as “fluoro” or “fluoros”), —Cl (sometimes referred to herein as “chloro” or “chloros”), —Br (sometimes referred to herein as “bromo” or “bromos”), and —I (sometimes referred to herein as “iodo” or “iodos”).

As used herein, the term “alkyl” refers to saturated linear or branched-chain monovalent hydrocarbon radicals, containing the indicated number of carbon atoms. For example, “(C₁₋₆)alkyl” refers to saturated linear or branched-chain monovalent hydrocarbon radicals of one to six carbon atoms. Non-limiting examples of alkyl include methyl, ethyl, 1-propyl, isopropyl, 1-butyl, isobutyl, sec-butyl, tert-butyl, 2-methyl-2-propyl, pentyl, neopentyl, and hexyl.

As used herein, the term “alkenyl” refers to a linear or branched mono-unsaturated hydrocarbon chain, containing the indicated number of carbon atoms. For example, “(C₂ s)alkenyl” refers a linear or branched mono unsaturated hydrocarbon chain of two to six carbon atoms. Non-limiting examples of alkenyl include ethenyl, propenyl, butenyl, or pentenyl.

As used herein, the term “alkynyl” refers to a linear or branched di-unsaturated hydrocarbon chain, containing the indicated number of carbon atoms. For example, “(C₂₋₆)alkynyl” refers to a linear or branched di-unsaturated hydrocarbon chain having two to six carbon atoms. Non-limiting examples of alkynyl include ethynyl, propynyl, butynyl, or pentynyl.

As used herein, the term “cycloalkyl” refers to a saturated or partially unsaturated cyclic hydrocarbon, containing the indicated number of carbon atoms. For example, “(C₃₋₆)cycloalkyl” refers to a saturated or partially unsaturated cyclic hydrocarbon having three to six ring carbon atoms. Non-limiting examples of cycloalkyl include cyclopropyl, cyclobutyl, cyclopentyl, and cyclohexyl. Cycloalkyl may be partially unsaturated. Non-limiting examples of partially unsaturated cycloalkyl include cyclohexenyl, cyclopentenyl, cycloheptenyl, cyclooctenyl, and the like. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

As used herein, the term “heterocyclyl” refers to a mono-, bi-, tri-, or polycyclic nonaromatic ring system containing indicated number of ring atoms (e.g., 3-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, the heteroatoms selected from O, N, S, or S(O)₁₋₂ (e.g., carbon atoms and 1-3, 1-6, or 1-9 heteroatoms of N, O, S, or S(O)₁₋₂ if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl groups may be partially unsaturated. Non-limiting examples of partially unsaturated heterocyclyl include dihydropyrrolyl, dihydropyridinyl, tetrahydropyridinyl, dihydrofuranyl, dihydropyranyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyl include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, the term “aryl” refers to a mono-, bi-, tri- or polycyclic hydrocarbon group containing the indicated numbers of carbon atoms, wherein at least one ring in the system is aromatic (e.g., C₆ monocyclic, C₁₀ bicyclic, or C₁₄ tricyclic aromatic ring system). Examples of aryl groups include phenyl, naphthyl, tetrahydronaphthyl, and the like.

As used herein, the term “heteroaryl” refers to a mono-, bi-, tri- or polycyclic group having indicated numbers of ring atoms (e.g., 5-6 ring atoms; e.g., 5, 6, 9, 10, or 14 ring atoms); wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others.

As used herein, the term “haloalkyl” refers to an alkyl radical as defined herein, wherein one or more (e.g., 1 to 5, or 1 to 3) hydrogen atoms is replaced with one or more (e.g., 1 to 5, or 1 to 3) halogen atoms. Non-limiting examples include fluoromethyl, difluoromethyl, trifluoromethyl, 2-fluoroethyl, 2,2-difluoroethyl, 2,2,2-trifluoroethyl, chloromethyl, dichloromethyl, chloroethyl, trichloroethyl, bromomethyl, and iodomethyl.

As used herein, the term “alkoxy” refers to an —O-alkyl radical, wherein the radical is on the oxygen atom. For example, “C₁₋₆ alkoxy” refers to an —O—(C₁₋₆ alkyl) radical, wherein the radical is on the oxygen atom. Examples of alkoxy include methoxy, ethoxy, propoxy, isopropoxy, butoxy and tert-butoxy. Accordingly, as used herein, the term “haloalkoxy” refers to an —O-haloalkyl radical, wherein the radical is on the oxygen atom.

As used herein, the term “amino” refers to amine of formula —N(R^{N1})₂, where each R^{N1} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, —NH₂, —NH(alkyl), —N(alkyl)₂, alkyl, alkenyl, alkynyl, haloalkyl, alkoxy, or haloalkoxy).

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

As used herein, when a ring is described as being “aromatic,” it means the ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thiazole, isoxazole, isothiazole, and the like. When a ring system comprising at least two rings is described as “aromatic,” it means the ring system comprises one or more aromatic ring(s). Accordingly, when a ring system comprising at least two rings is described as “non-aromatic,” none of the constituent rings of the ring system is aromatic.

As used herein, when a ring is described as being “partially unsaturated,” it means the ring has one or more additional degrees of unsaturation (in addition to the degree of unsaturation attributed to the ring itself, e.g., one or more double bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like. When a ring system comprising at least two rings is described as “partially unsaturated,” it means the ring system comprises one or more partially unsaturated ring(s), provided that none of the constituent rings of the ring system is aromatic.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term “GLP-1R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-1R molecules, isoforms, precursors, mutants, variants, derivatives, splice variants, alleles, different species, and active fragments thereof.

The term “GLP-1 associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1 RA” as used herein refers to an agonist of the glucagon-like peptide-1 (GLP-1) receptor. GLP-1 RAs enhance glucose-dependent insulin secretion; suppress inappropriately elevated glucagon levels, both in fasting and postprandial states; and slow gastric emptying. Karla et al., Glucagon-like peptide-1 receptor agonists in the treatment of type 2 diabetes: Past, present, and future, Indian J Endocrinol Metab. 2016 March-April; 20(2): 254-267. GLP-1 RAs have been shown to treat type 2 diabetes. Examples of GLP-1 RAs include, but are not limited to, albiglutide (TANZEUM®), dulaglutide (LY2189265, TRULICITY®), efpeglenatide, exenatide (BYETTA®, BYDUREON®, Exendin-4), liraglutide (VICTOZA®, NN2211), lixisenatide (LYXUMIA®), semaglutide (OZEMPIC®), tirzepatide, ZP2929, NNC0113-0987, BPI-3016, and TT401. See, also, for example, additional GLP-1 receptor agonists described in U.S. Pat. Nos. 10,370,426; 10,308,700; 10,259,823; 10,208,019; 9,920,106; 9,839,664; 8,129,343; 8,536,122; 7,919,598; 6,414,126; 6,628,343; and RE45313.

The term “pharmaceutically acceptable” as used herein indicates that the compound, or salt or composition thereof is compatible chemically and/or toxicologically with the other ingredients comprising a formulation and/or the patient being treated therewith.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, aluminum, ammonium, calcium, and magnesium salts. Salts derived from organic bases include, but are not limited to, salts of NH₃, or primary, secondary, tertiary amines, such as salts derived from a N-containing heterocycle, a N-containing heteroaryl, or derived from an amine of formula N(R^{N})₃ (e.g., HN⁺(R^{N})₃ or (alkyl)N⁺(R^{N})₃) where each R^{N} is independently hydrogen, alkyl, alkenyl, alkynyl, haloalkyl, cycloalkyl, heterocyclyl, aryl, or heteroaryl, wherein each is optionally substituted, such as by one or more (e.g., 1-5 or 1-3) substituents (e.g., halo, cyano, hydroxy, amino, alkyl, alkenyl, alkynyl, haloalkyl, alkoxy, or haloalkoxy). Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Table 1, or a pharmaceutically acceptable salt or solvate thereof) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21^{st} ed.; Lippincott Williams & Wilkins: Philadelphia, PA, 2005; Handbook of Pharmaceutical Excipients, 6^{th} ed.; Rowe et al., Eds.; The Pharmaceutical Press and the American Pharmaceutical Association: 2009; Handbook of Pharmaceutical Additives, 3^{rd} ed.; Ash and Ash Eds.; Gower Publishing Company: 2007; Pharmaceutical Preformulation and Formulation, 2^{nd} ed.; Gibson Ed.; CRC Press LLC: Boca Raton, FL, 2009.

The term “pharmaceutical composition” refers to a mixture of a compound of Table 1, or a pharmaceutically acceptable salt or solvate thereof as described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing,” as used herein, is the prevention of the onset, recurrence or spread, in whole or in part, of the disease or condition as described herein, or a symptom thereof.

The terms “subject,” “patient” or “individual,” as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the patient is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination of the disclosure.

The term “pharmaceutical combination,” as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulation,” as used herein, refers to a regulation or an adjustment (e.g., increase or decrease) and can include, for example agonism, partial agonism or antagonism.

It is understood that the substituents as defined herein are not intended to include impermissible substitution patterns (e.g., methyl substituted with 5 fluoro groups or a hydroxy group attached to an ethenylic or acetylenic carbon atom). Such impermissible substitution patterns are well known to the skilled artisan.

### Compounds

Provided herein is a compound selected from Table 1, or a pharmaceutically acceptable salt or solvate thereof:

TABLE 1

| Ex. | Structure |
|---|---|
| 1 | 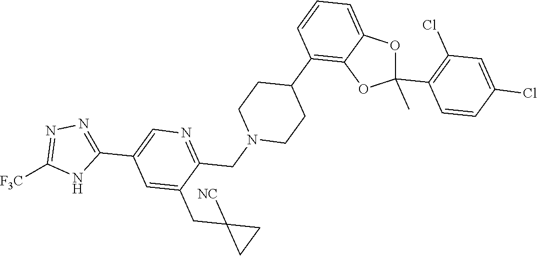 [SMILES: CC1(c2ccc(Cl)cc2Cl)Oc2cccc(C3CCN(Cc4ncc(-c5nnc(C(F)(F)F)[nH]5)cc4CC4(C#N)CC4)CC3)c2O1] |
| 2 | 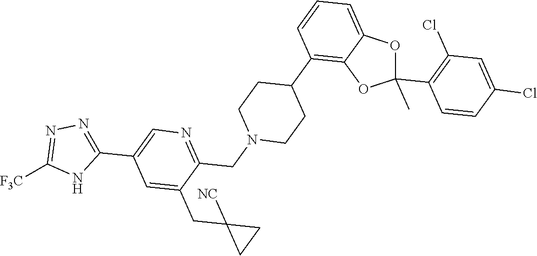 [SMILES: CC1(c2ccc(Cl)cc2Cl)Oc2cccc(C3CCN(Cc4ncc(-c5nnc(C(F)(F)F)[nH]5)cc4CC4(C#N)CC4)CC3)c2O1] |
| | enantiomer 1 |
| 3 | 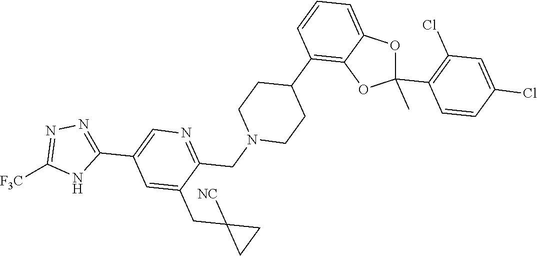 [SMILES: CC1(c2ccc(Cl)cc2Cl)Oc2cccc(C3CCN(Cc4ncc(-c5nnc(C(F)(F)F)[nH]5)cc4CC4(C#N)CC4)CC3)c2O1] |
| | enantiomer 2 |
| 4 | 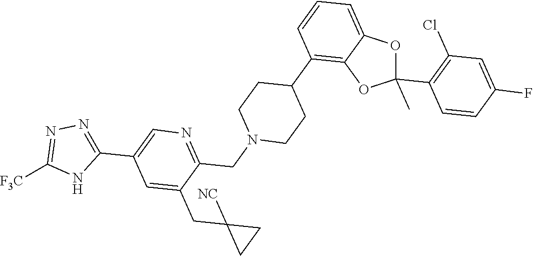 [SMILES: CC1(c2ccc(F)cc2Cl)Oc2cccc(C3CCN(Cc4ncc(-c5nnc(C(F)(F)F)[nH]5)cc4CC4(C#N)CC4)CC3)c2O1] |
| 5 | 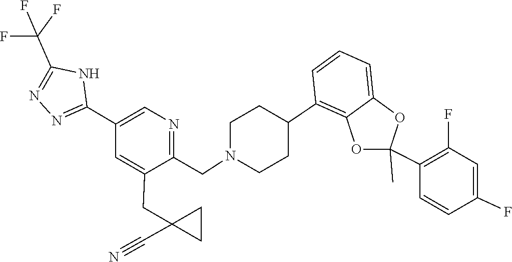 [SMILES: CC1(c2ccc(F)cc2F)Oc2cccc(C3CCN(Cc4ncc(-c5nnc(C(F)(F)F)[nH]5)cc4CC4(C#N)CC4)CC3)c2O1] |
| 6 | 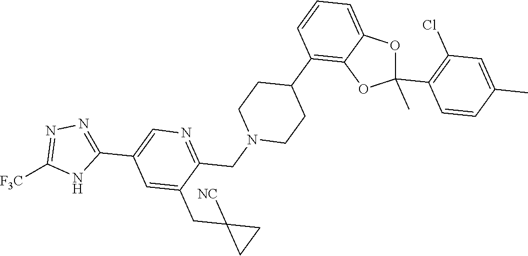 [SMILES: Cc1ccc(C2(C)Oc3cccc(C4CCN(Cc5ncc(-c6nnc(C(F)(F)F)[nH]6)cc5CC5(C#N)CC5)CC4)c3O2)c(Cl)c1] |
| 7 | 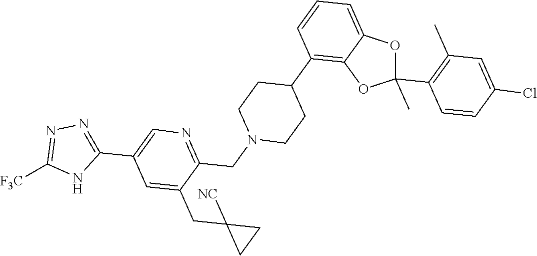 [SMILES: Cc1cc(Cl)ccc1C1(C)Oc2cccc(C3CCN(Cc4ncc(-c5nnc(C(F)(F)F)[nH]5)cc4CC4(C#N)CC4)CC3)c2O1] |
| 8 | 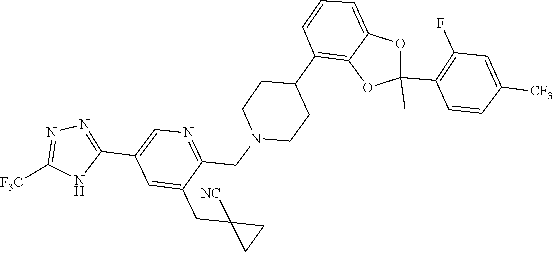 [SMILES: CC1(c2ccc(C(F)(F)F)cc2F)Oc2cccc(C3CCN(Cc4ncc(-c5nnc(C(F)(F)F)[nH]5)cc4CC4(C#N)CC4)CC3)c2O1] |
| 9 | 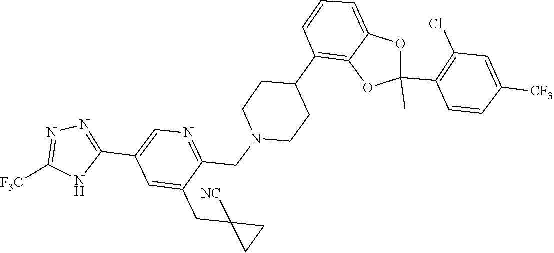 [SMILES: CC1(c2ccc(C(F)(F)F)cc2Cl)Oc2cccc(C3CCN(Cc4ncc(-c5nnc(C(F)(F)F)[nH]5)cc4CC4(C#N)CC4)CC3)c2O1] |
| 10 | 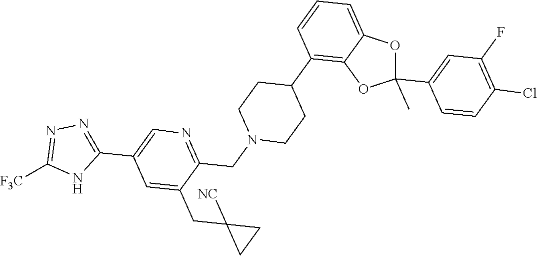 [SMILES: CC1(c2ccc(Cl)c(F)c2)Oc2cccc(C3CCN(Cc4ncc(-c5nnc(C(F)(F)F)[nH]5)cc4CC4(C#N)CC4)CC3)c2O1] |
| 11 | 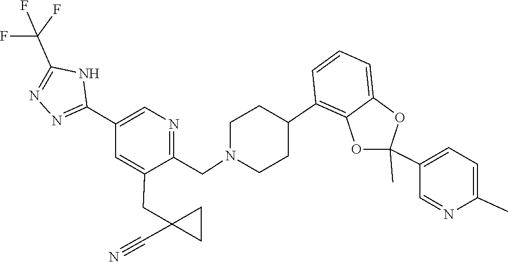 [SMILES: Cc1ccc(C2(C)Oc3cccc(C4CCN(Cc5ncc(-c6nnc(C(F)(F)F)[nH]6)cc5CC5(C#N)CC5)CC4)c3O2)cn1] |
| 12 | 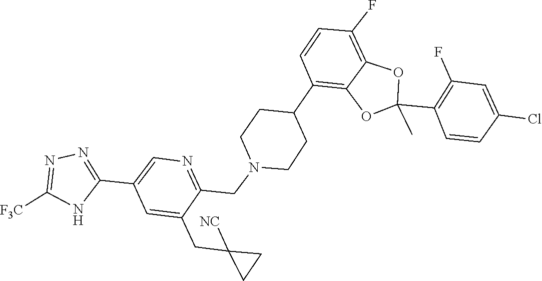 [SMILES: CC1(c2ccc(Cl)cc2F)Oc2c(F)ccc(C3CCN(Cc4ncc(-c5nnc(C(F)(F)F)[nH]5)cc4CC4(C#N)CC4)CC3)c2O1] |
| 13 | 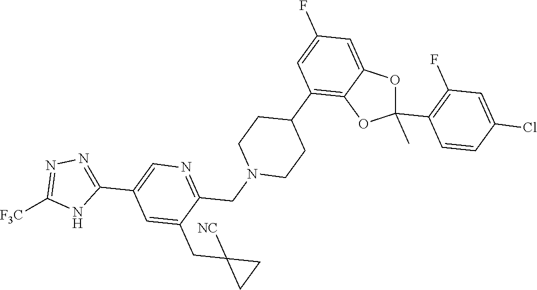 [SMILES: CC1(c2ccc(Cl)cc2F)Oc2cc(F)cc(C3CCN(Cc4ncc(-c5nnc(C(F)(F)F)[nH]5)cc4CC4(C#N)CC4)CC3)c2O1] |
| | enantiomer 1 |
| 14 | 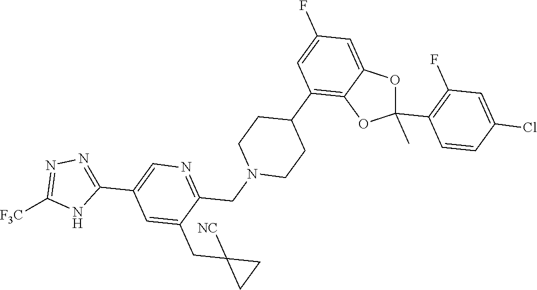 [SMILES: CC1(c2ccc(Cl)cc2F)Oc2cc(F)cc(C3CCN(Cc4ncc(-c5nnc(C(F)(F)F)[nH]5)cc4CC4(C#N)CC4)CC3)c2O1] |
| | enantiomer 2 |
| 15 | 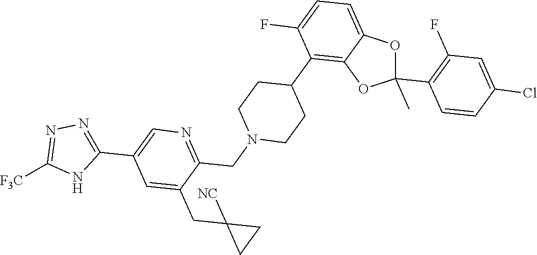 [SMILES: CC1(c2ccc(Cl)cc2F)Oc2ccc(F)c(C3CCN(Cc4ncc(-c5nnc(C(F)(F)F)[nH]5)cc4CC4(C#N)CC4)CC3)c2O1] |
| 16 | 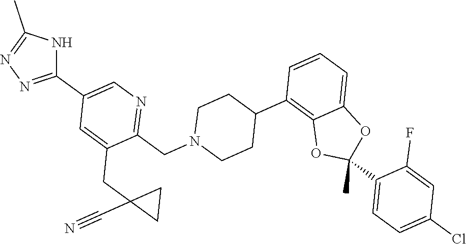 [SMILES: Cc1nnc(-c2cnc(CN3CCC(c4cccc5c4O[C@@](C)(c4ccc(Cl)cc4F)O5)CC3)c(CC3(C#N)CC3)c2)[nH]1] |
| 17 | 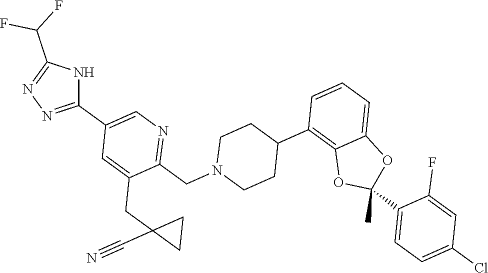 [SMILES: C[C@]1(c2ccc(Cl)cc2F)Oc2cccc(C3CCN(Cc4ncc(-c5nnc(C(F)F)[nH]5)cc4CC4(C#N)CC4)CC3)c2O1] |
| 18 | 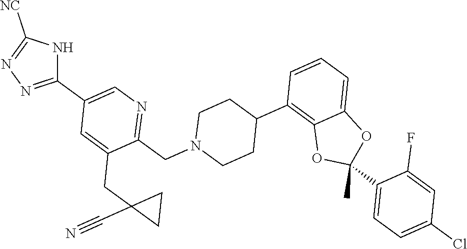 [SMILES: C[C@]1(c2ccc(Cl)cc2F)Oc2cccc(C3CCN(Cc4ncc(-c5nnc(C#N)[nH]5)cc4CC4(C#N)CC4)CC3)c2O1] |
| 19 | 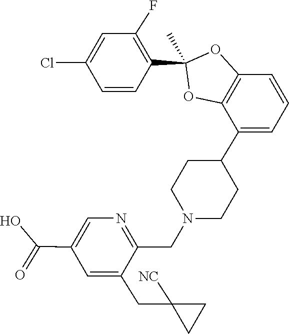 [SMILES: C[C@]1(c2ccc(Cl)cc2F)Oc2cccc(C3CCN(Cc4ncc(C(=O)O)cc4CC4(C#N)CC4)CC3)c2O1] |
| 20 | 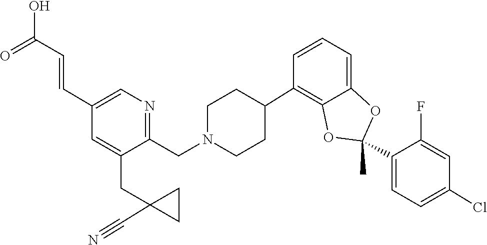 [SMILES: C[C@]1(c2ccc(Cl)cc2F)Oc2cccc(C3CCN(Cc4ncc(/C=C/C(=O)O)cc4CC4(C#N)CC4)CC3)c2O1] |
| 21 | 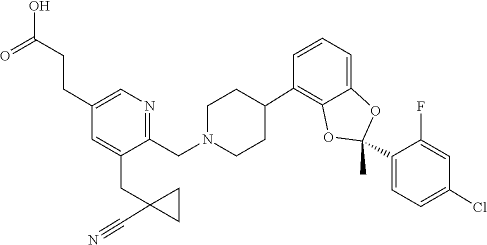 [SMILES: C[C@]1(c2ccc(Cl)cc2F)Oc2cccc(C3CCN(Cc4ncc(CCC(=O)O)cc4CC4(C#N)CC4)CC3)c2O1] |
| 22 | 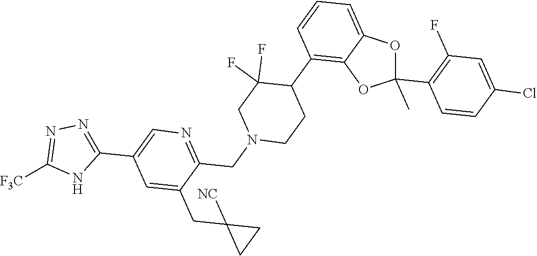 [SMILES: CC1(c2ccc(Cl)cc2F)Oc2cccc(C3CCN(Cc4ncc(-c5nnc(C(F)(F)F)[nH]5)cc4CC4(C#N)CC4)CC3(F)F)c2O1] |
| 23 | 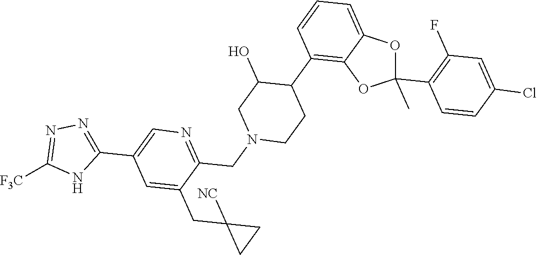 [SMILES: CC1(c2ccc(Cl)cc2F)Oc2cccc(C3CCN(Cc4ncc(-c5nnc(C(F)(F)F)[nH]5)cc4CC4(C#N)CC4)CC3O)c2O1] |
| | trans- diastereomer 1 |
| 24 | 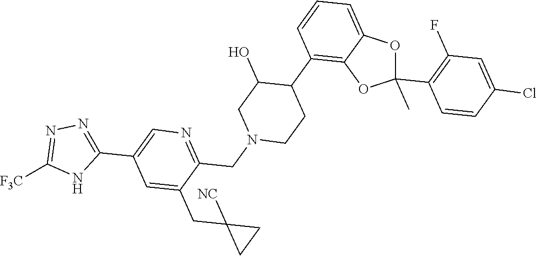 [SMILES: CC1(c2ccc(Cl)cc2F)Oc2cccc(C3CCN(Cc4ncc(-c5nnc(C(F)(F)F)[nH]5)cc4CC4(C#N)CC4)CC3O)c2O1] |
| | trans- diastereomer 2 |
| 25 | 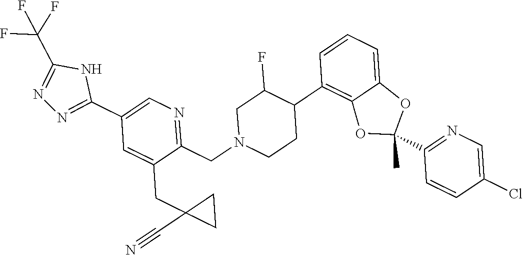 [SMILES: C[C@]1(c2ccc(Cl)cn2)Oc2cccc(C3CCN(Cc4ncc(-c5nnc(C(F)(F)F)[nH]5)cc4CC4(C#N)CC4)CC3F)c2O1] |
| | trans, diastereomer 1 |
| 26 | 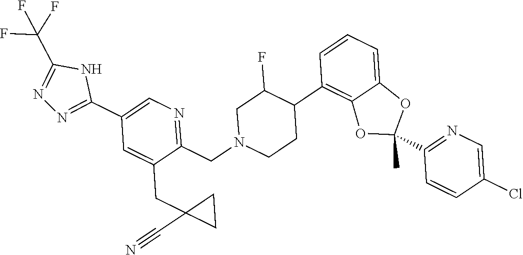 [SMILES: C[C@]1(c2ccc(Cl)cn2)Oc2cccc(C3CCN(Cc4ncc(-c5nnc(C(F)(F)F)[nH]5)cc4CC4(C#N)CC4)CC3F)c2O1] |
| | trans, diastereomer 2 |
| 27 | 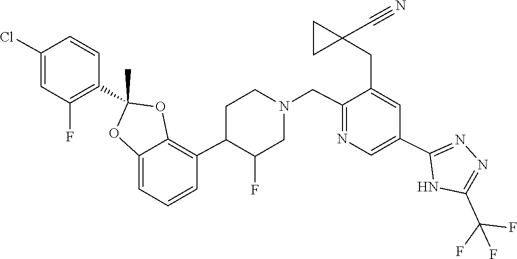 [SMILES: C[C@]1(c2ccc(Cl)cc2F)Oc2cccc(C3CCN(Cc4ncc(-c5nnc(C(F)(F)F)[nH]5)cc4CC4(C#N)CC4)CC3F)c2O1] |
| | trans, diastereomer 1 |
| 28 | 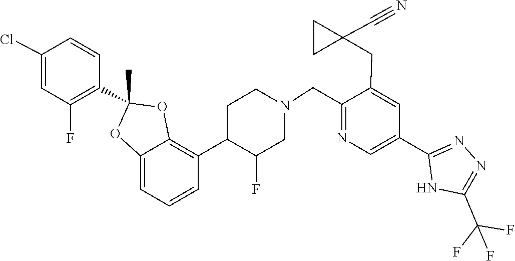 [SMILES: C[C@]1(c2ccc(Cl)cc2F)Oc2cccc(C3CCN(Cc4ncc(-c5nnc(C(F)(F)F)[nH]5)cc4CC4(C#N)CC4)CC3F)c2O1] |
| | trans, diastereomer 2 |
| 29 | 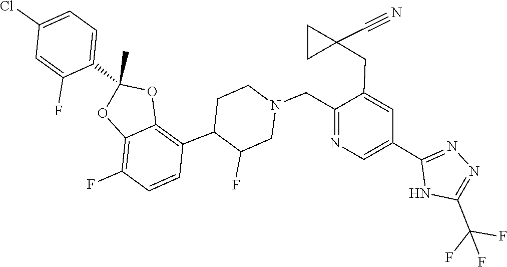 [SMILES: C[C@]1(c2ccc(Cl)cc2F)Oc2c(F)ccc(C3CCN(Cc4ncc(-c5nnc(C(F)(F)F)[nH]5)cc4CC4(C#N)CC4)CC3F)c2O1] |
| | trans, single diastereomer 1 |
| 30 | 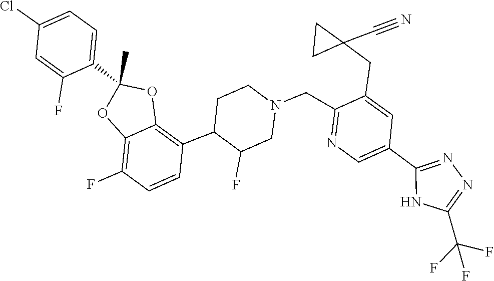 [SMILES: C[C@]1(c2ccc(Cl)cc2F)Oc2c(F)ccc(C3CCN(Cc4ncc(-c5nnc(C(F)(F)F)[nH]5)cc4CC4(C#N)CC4)CC3F)c2O1] |
| | trans, single diastereomer 2 |
| 31 | 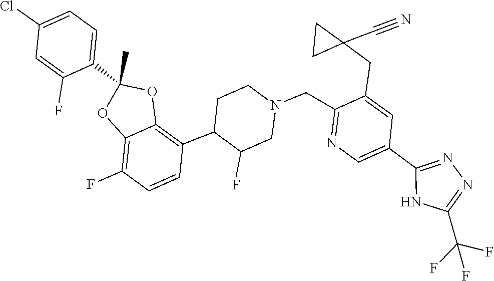 [SMILES: C[C@]1(c2ccc(Cl)cc2F)Oc2c(F)ccc(C3CCN(Cc4ncc(-c5nnc(C(F)(F)F)[nH]5)cc4CC4(C#N)CC4)CC3F)c2O1] |
| | trans, single diastereomer 2 |
| 32 | 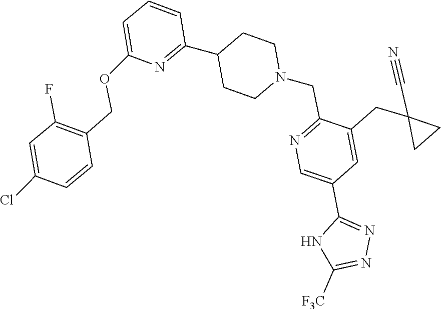 [SMILES: N#CC1(Cc2cc(-c3nnc(C(F)(F)F)[nH]3)cnc2CN2CCC(c3cccc(OCc4ccc(Cl)cc4F)n3)CC2)CC1] |
| 33 | 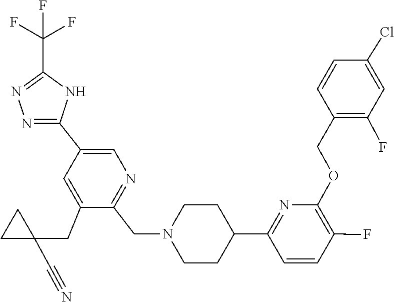 [SMILES: N#CC1(Cc2cc(-c3nnc(C(F)(F)F)[nH]3)cnc2CN2CCC(c3ccc(F)c(OCc4ccc(Cl)cc4F)n3)CC2)CC1] |
| 34 | 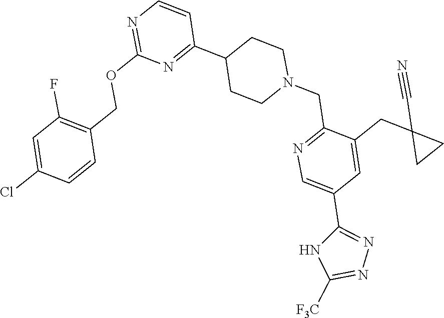 [SMILES: N#CC1(Cc2cc(-c3nnc(C(F)(F)F)[nH]3)cnc2CN2CCC(c3ccnc(OCc4ccc(Cl)cc4F)n3)CC2)CC1] |
| 35 | 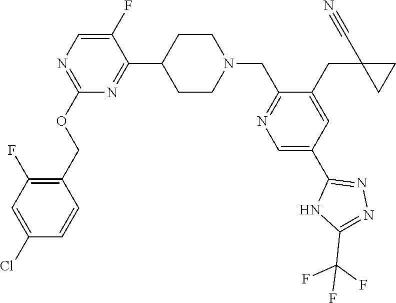 [SMILES: N#CC1(Cc2cc(-c3nnc(C(F)(F)F)[nH]3)cnc2CN2CCC(c3nc(OCc4ccc(Cl)cc4F)ncc3F)CC2)CC1] |
| 36 | 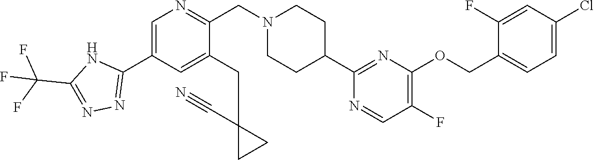 [SMILES: N#CC1(Cc2cc(-c3nnc(C(F)(F)F)[nH]3)cnc2CN2CCC(c3ncc(F)c(OCc4ccc(Cl)cc4F)n3)CC2)CC1] |
| 37 | 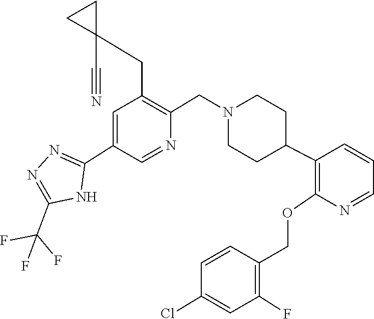 [SMILES: N#CC1(Cc2cc(-c3nnc(C(F)(F)F)[nH]3)cnc2CN2CCC(c3cccnc3OCc3ccc(Cl)cc3F)CC2)CC1] |
| 38 | 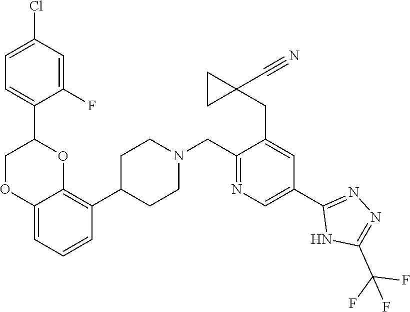 [SMILES: N#CC1(Cc2cc(-c3nnc(C(F)(F)F)[nH]3)cnc2CN2CCC(c3cccc4c3OC(c3ccc(Cl)cc3F)CO4)CC2)CC1] |
| 39 | 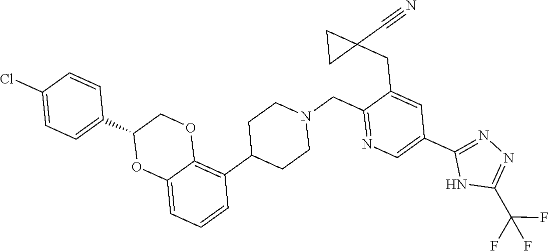 [SMILES: N#CC1(Cc2cc(-c3nnc(C(F)(F)F)[nH]3)cnc2CN2CCC(c3cccc4c3OC[C@@H](c3ccc(Cl)cc3)O4)CC2)CC1] |
| 40 | 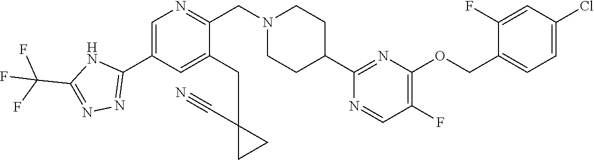 [SMILES: N#CC1(Cc2cc(-c3nnc(C(F)(F)F)[nH]3)cnc2CN2CCC(c3ncc(F)c(OCc4ccc(Cl)cc4F)n3)CC2)CC1] |
| 41 | 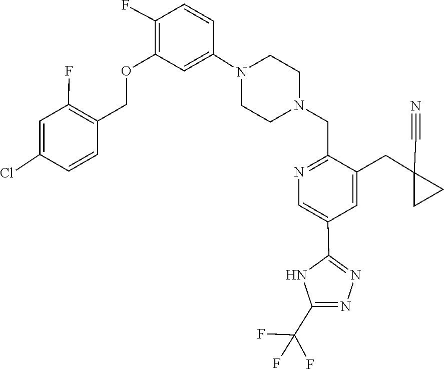 [SMILES: N#CC1(Cc2cc(-c3nnc(C(F)(F)F)[nH]3)cnc2CN2CCN(c3ccc(F)c(OCc4ccc(Cl)cc4F)c3)CC2)CC1] |
| 42 | 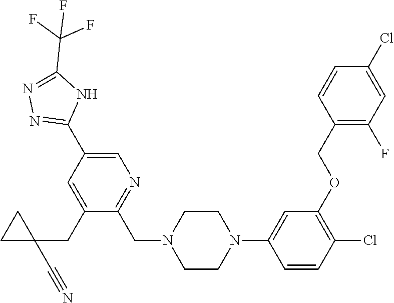 [SMILES: N#CC1(Cc2cc(-c3nnc(C(F)(F)F)[nH]3)cnc2CN2CCN(c3ccc(Cl)c(OCc4ccc(Cl)cc4F)c3)CC2)CC1] |
| 43 | 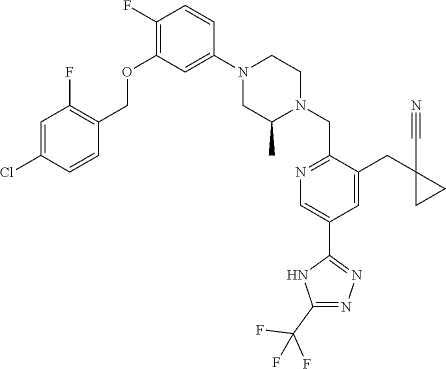 [SMILES: C[C@H]1CN(c2ccc(F)c(OCc3ccc(Cl)cc3F)c2)CCN1Cc1ncc(-c2nnc(C(F)(F)F)[nH]2)cc1CC1(C#N)CC1] |
| 44 | 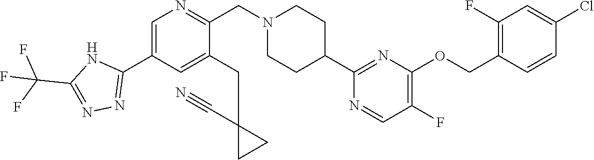 [SMILES: N#CC1(Cc2cc(-c3nnc(C(F)(F)F)[nH]3)cnc2CN2CCC(c3ncc(F)c(OCc4ccc(Cl)cc4F)n3)CC2)CC1] |
| 45 | 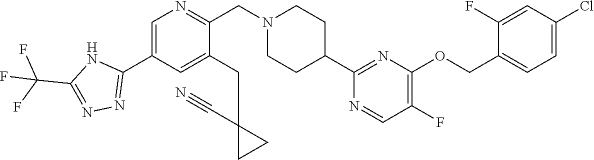 [SMILES: N#CC1(Cc2cc(-c3nnc(C(F)(F)F)[nH]3)cnc2CN2CCC(c3ncc(F)c(OCc4ccc(Cl)cc4F)n3)CC2)CC1] |
| 46 | 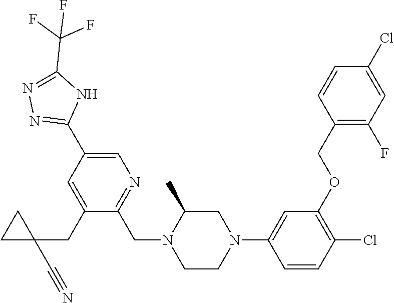 [SMILES: C[C@H]1CN(c2ccc(Cl)c(OCc3ccc(Cl)cc3F)c2)CCN1Cc1ncc(-c2nnc(C(F)(F)F)[nH]2)cc1CC1(C#N)CC1] |
| 47 | 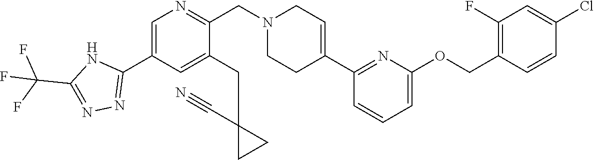 [SMILES: N#CC1(Cc2cc(-c3nnc(C(F)(F)F)[nH]3)cnc2CN2CC=C(c3cccc(OCc4ccc(Cl)cc4F)n3)CC2)CC1] |
| 48 | 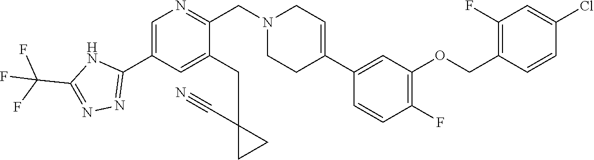 [SMILES: N#CC1(Cc2cc(-c3nnc(C(F)(F)F)[nH]3)cnc2CN2CC=C(c3ccc(F)c(OCc4ccc(Cl)cc4F)c3)CC2)CC1] |
| 49 | 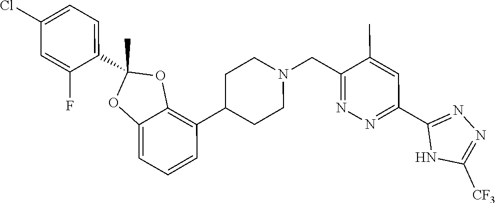 [SMILES: Cc1cc(-c2nnc(C(F)(F)F)[nH]2)nnc1CN1CCC(c2cccc3c2O[C@@](C)(c2ccc(Cl)cc2F)O3)CC1] |
| 50 | 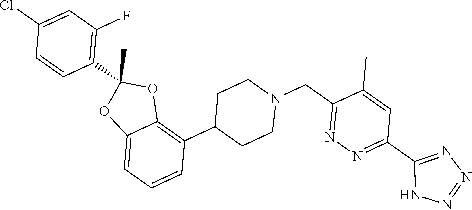 [SMILES: Cc1cc(-c2nnn[nH]2)nnc1CN1CCC(c2cccc3c2O[C@@](C)(c2ccc(Cl)cc2F)O3)CC1] |
| 51 | 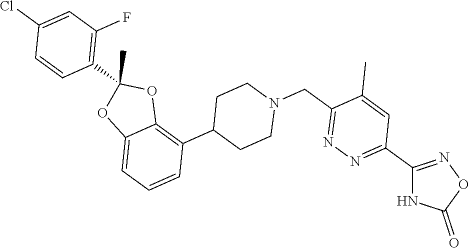 [SMILES: Cc1cc(-c2noc(=O)[nH]2)nnc1CN1CCC(c2cccc3c2O[C@@](C)(c2ccc(Cl)cc2F)O3)CC1] |
| 52 | 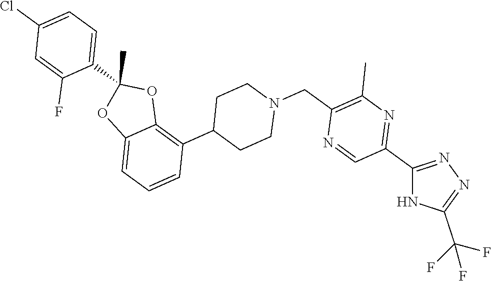 [SMILES: Cc1nc(-c2nnc(C(F)(F)F)[nH]2)cnc1CN1CCC(c2cccc3c2O[C@@](C)(c2ccc(Cl)cc2F)O3)CC1] |
| 53 | 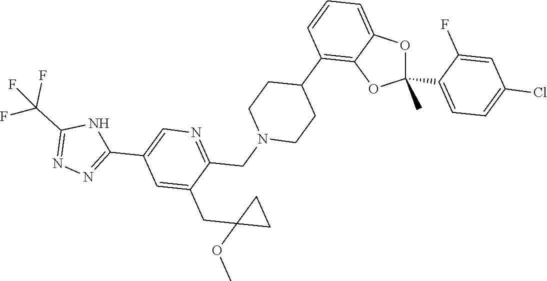 [SMILES: COC1(Cc2cc(-c3nnc(C(F)(F)F)[nH]3)cnc2CN2CCC(c3cccc4c3O[C@@](C)(c3ccc(Cl)cc3F)O4)CC2)CC1] |
| 54 | 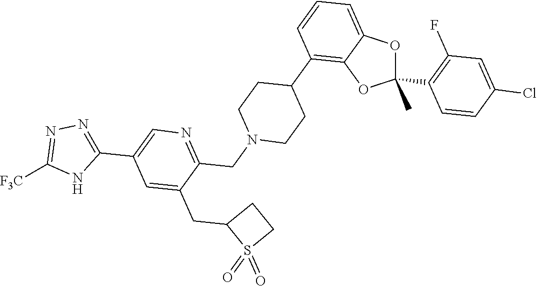 [SMILES: C[C@]1(c2ccc(Cl)cc2F)Oc2cccc(C3CCN(Cc4ncc(-c5nnc(C(F)(F)F)[nH]5)cc4CC4CCS4(=O)=O)CC3)c2O1] |
| 55 | 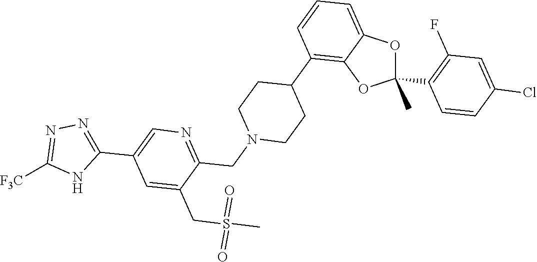 [SMILES: C[C@]1(c2ccc(Cl)cc2F)Oc2cccc(C3CCN(Cc4ncc(-c5nnc(C(F)(F)F)[nH]5)cc4CS(C)(=O)=O)CC3)c2O1] |
| 56 | 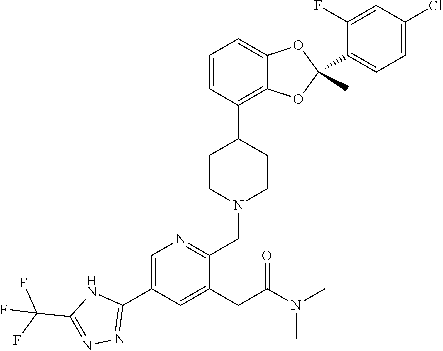 [SMILES: CN(C)C(=O)Cc1cc(-c2nnc(C(F)(F)F)[nH]2)cnc1CN1CCC(c2cccc3c2O[C@@](C)(c2ccc(Cl)cc2F)O3)CC1] |
| 57 | 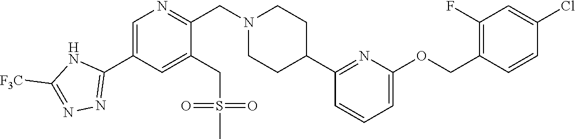 [SMILES: CS(=O)(=O)Cc1cc(-c2nnc(C(F)(F)F)[nH]2)cnc1CN1CCC(c2cccc(OCc3ccc(Cl)cc3F)n2)CC1] |
| 58 | 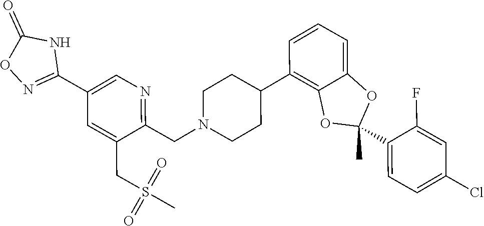 [SMILES: C[C@]1(c2ccc(Cl)cc2F)Oc2cccc(C3CCN(Cc4ncc(-c5noc(=O)[nH]5)cc4CS(C)(=O)=O)CC3)c2O1] |
| 59 | 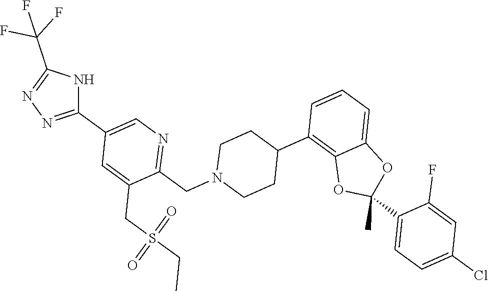 [SMILES: CCS(=O)(=O)Cc1cc(-c2nnc(C(F)(F)F)[nH]2)cnc1CN1CCC(c2cccc3c2O[C@@](C)(c2ccc(Cl)cc2F)O3)CC1] |
| 60 | 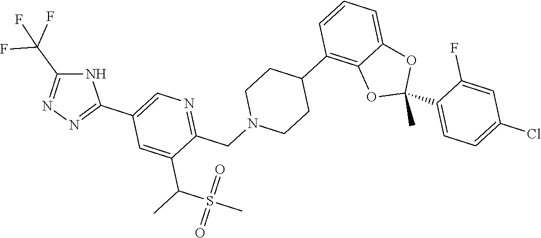 [SMILES: CC(c1cc(-c2nnc(C(F)(F)F)[nH]2)cnc1CN1CCC(c2cccc3c2O[C@@](C)(c2ccc(Cl)cc2F)O3)CC1)S(C)(=O)=O] |
| 61 | 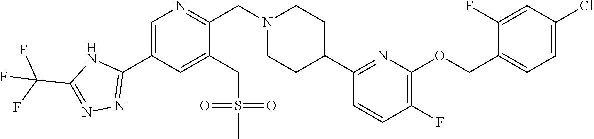 [SMILES: CS(=O)(=O)Cc1cc(-c2nnc(C(F)(F)F)[nH]2)cnc1CN1CCC(c2ccc(F)c(OCc3ccc(Cl)cc3F)n2)CC1] |
| 62 | 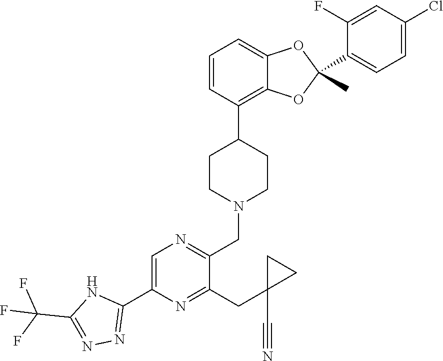 [SMILES: C[C@]1(c2ccc(Cl)cc2F)Oc2cccc(C3CCN(Cc4ncc(-c5nnc(C(F)(F)F)[nH]5)nc4CC4(C#N)CC4)CC3)c2O1] |
| 63 | 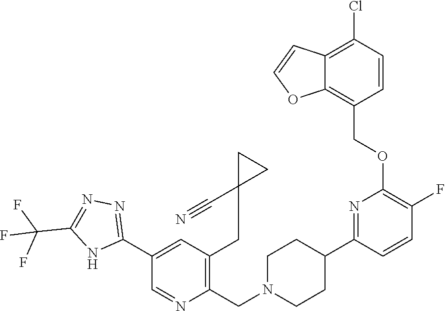 [SMILES: N#CC1(Cc2cc(-c3nnc(C(F)(F)F)[nH]3)cnc2CN2CCC(c3ccc(F)c(OCc4ccc(Cl)c5ccoc45)n3)CC2)CC1] |
| 64 | 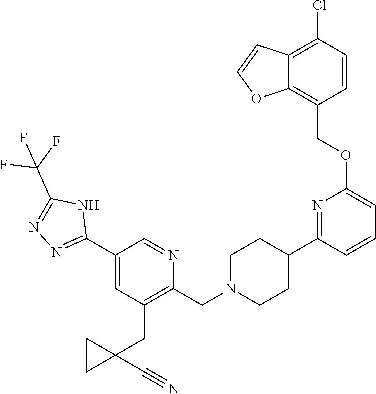 [SMILES: N#CC1(Cc2cc(-c3nnc(C(F)(F)F)[nH]3)cnc2CN2CCC(c3cccc(OCc4ccc(Cl)c5ccoc45)n3)CC2)CC1] |
| 65 | 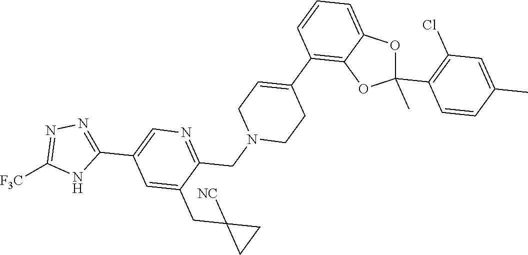 [SMILES: Cc1ccc(C2(C)Oc3cccc(C4=CCN(Cc5ncc(-c6nnc(C(F)(F)F)[nH]6)cc5CC5(C#N)CC5)CC4)c3O2)c(Cl)c1] |
| 66 | 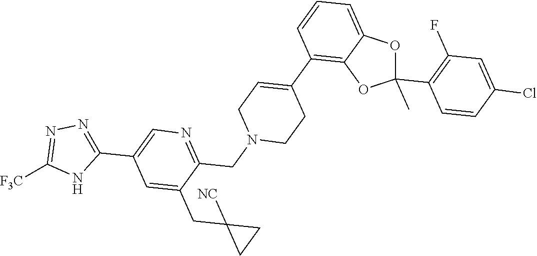 [SMILES: CC1(c2ccc(Cl)cc2F)Oc2cccc(C3=CCN(Cc4ncc(-c5nnc(C(F)(F)F)[nH]5)cc4CC4(C#N)CC4)CC3)c2O1] |
| 67 | 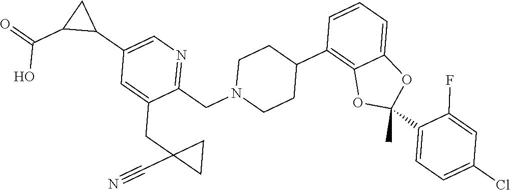 [SMILES: C[C@]1(c2ccc(Cl)cc2F)Oc2cccc(C3CCN(Cc4ncc(C5CC5C(=O)O)cc4CC4(C#N)CC4)CC3)c2O1] |
| 71 | 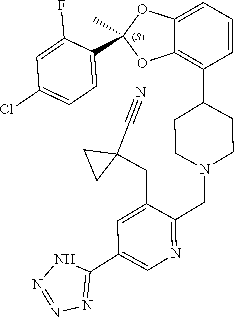 |
| 73 | 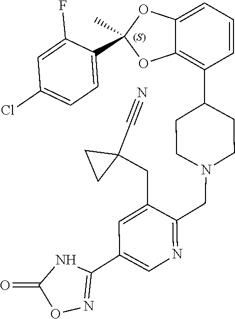 |
| 75 | 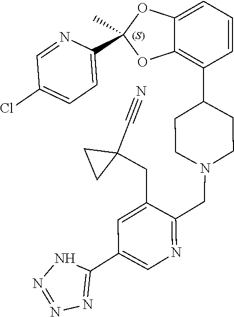 |
| 77 | 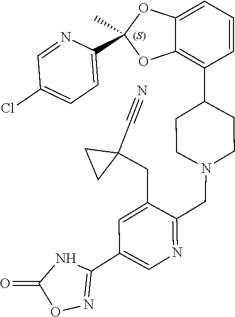 |
| 78 | 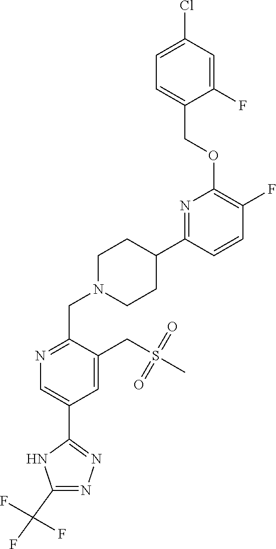 [SMILES: CS(=O)(=O)Cc1cc(-c2nnc(C(F)(F)F)[nH]2)cnc1CN1CCC(c2ccc(F)c(OCc3ccc(Cl)cc3F)n2)CC1] |
| 79 | 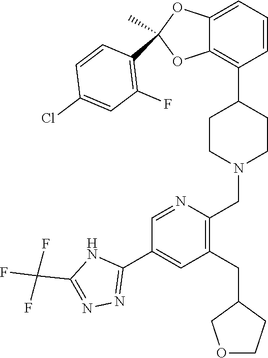 [SMILES: C[C@]1(c2ccc(Cl)cc2F)Oc2cccc(C3CCN(Cc4ncc(-c5nnc(C(F)(F)F)[nH]5)cc4CC4CCOC4)CC3)c2O1] |
| 80 | 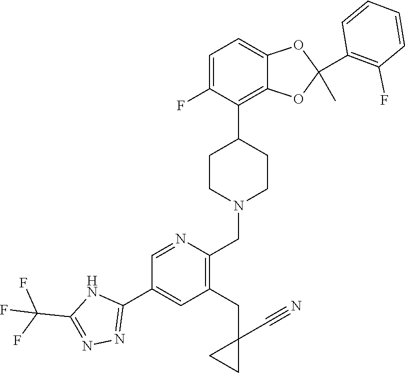 [SMILES: CC1(c2ccccc2F)Oc2ccc(F)c(C3CCN(Cc4ncc(-c5nnc(C(F)(F)F)[nH]5)cc4CC4(C#N)CC4)CC3)c2O1] |
| 81 | 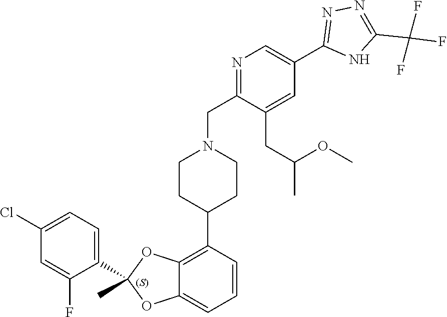 |
| 82 | 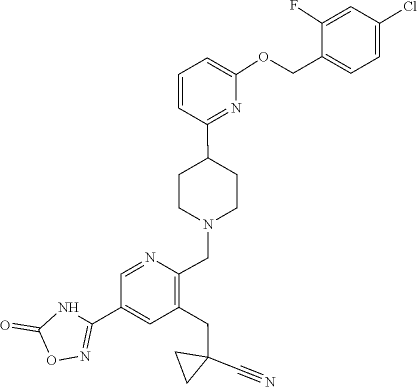 [SMILES: N#CC1(Cc2cc(-c3noc(=O)[nH]3)cnc2CN2CCC(c3cccc(OCc4ccc(Cl)cc4F)n3)CC2)CC1] |
| 83 | 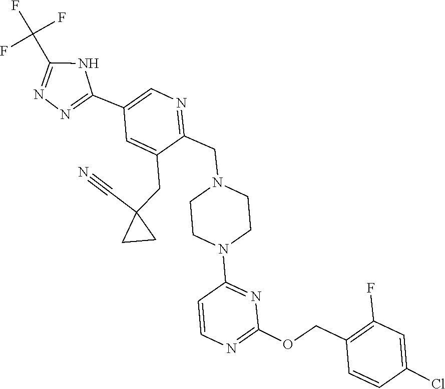 [SMILES: N#CC1(Cc2cc(-c3nnc(C(F)(F)F)[nH]3)cnc2CN2CCN(c3ccnc(OCc4ccc(Cl)cc4F)n3)CC2)CC1] |
| 85 | 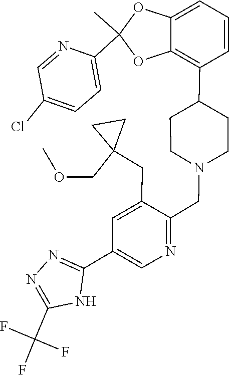 [SMILES: COCC1(Cc2cc(-c3nnc(C(F)(F)F)[nH]3)cnc2CN2CCC(c3cccc4c3OC(C)(c3ccc(Cl)cn3)O4)CC2)CC1] |
| 86 | 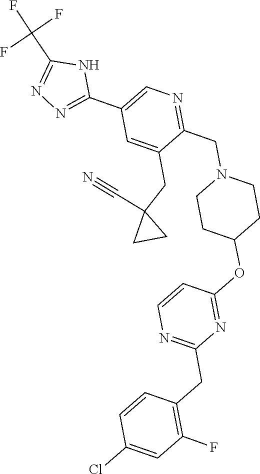 [SMILES: N#CC1(Cc2cc(-c3nnc(C(F)(F)F)[nH]3)cnc2CN2CCC(Oc3ccnc(Cc4ccc(Cl)cc4F)n3)CC2)CC1] |
| 87 | 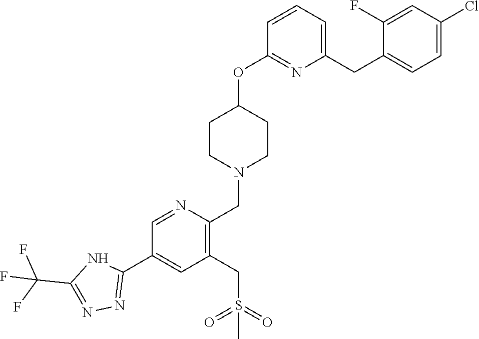 [SMILES: CS(=O)(=O)Cc1cc(-c2nnc(C(F)(F)F)[nH]2)cnc1CN1CCC(Oc2cccc(Cc3ccc(Cl)cc3F)n2)CC1] |
| 89 | 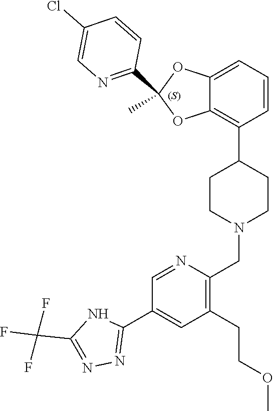 |
| 90 | 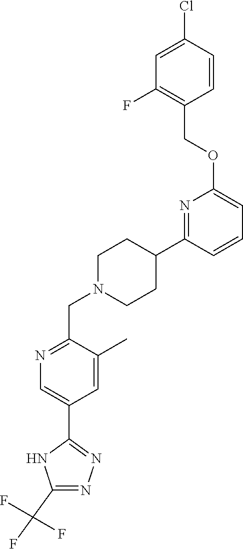 [SMILES: Cc1cc(-c2nnc(C(F)(F)F)[nH]2)cnc1CN1CCC(c2cccc(OCc3ccc(Cl)cc3F)n2)CC1] |
| 91 | 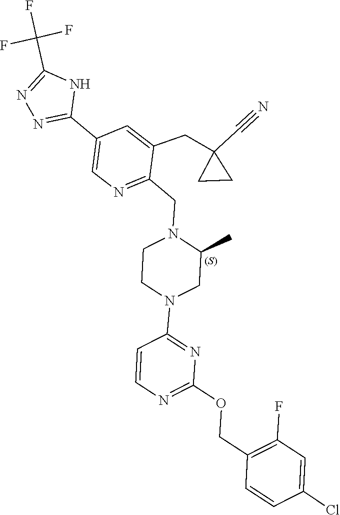 |
| 92 | 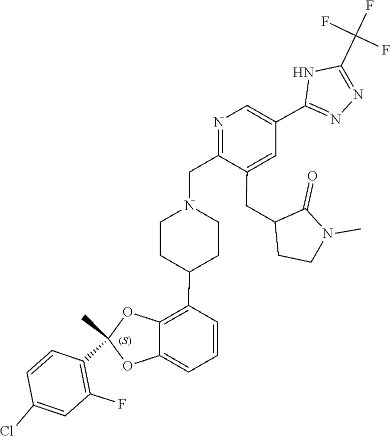 |
| 93 | 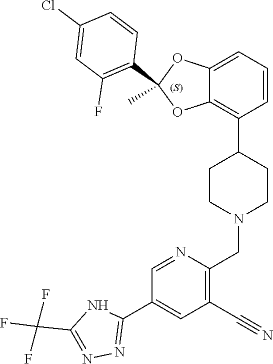 |
| 94 | 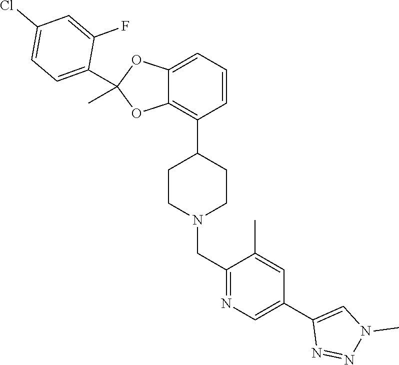 [SMILES: Cc1cc(-c2cn(C)nn2)cnc1CN1CCC(c2cccc3c2OC(C)(c2ccc(Cl)cc2F)O3)CC1] |
| 95 | 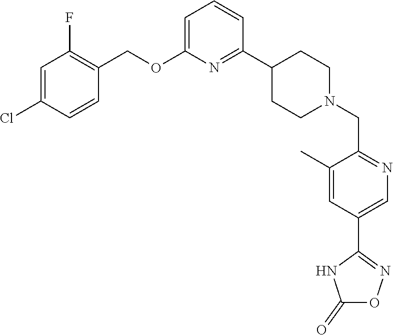 [SMILES: Cc1cc(-c2noc(=O)[nH]2)cnc1CN1CCC(c2cccc(OCc3ccc(Cl)cc3F)n2)CC1] |
| 96 | 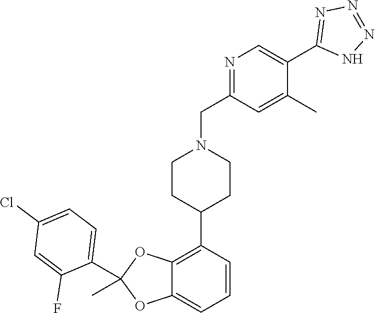 [SMILES: Cc1cc(CN2CCC(c3cccc4c3OC(C)(c3ccc(Cl)cc3F)O4)CC2)ncc1-c1nnn[nH]1] |
| 97 | 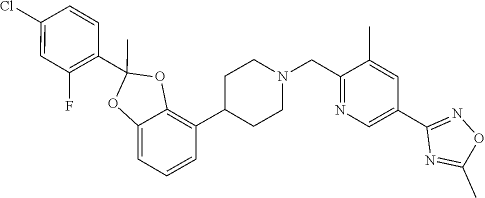 [SMILES: Cc1nc(-c2cnc(CN3CCC(c4cccc5c4OC(C)(c4ccc(Cl)cc4F)O5)CC3)c(C)c2)no1] |
| 98 | 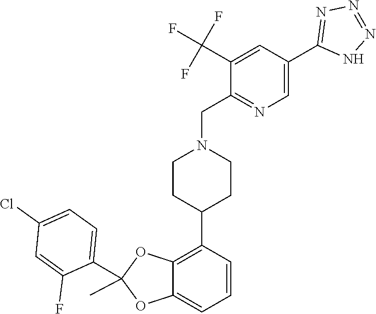 [SMILES: CC1(c2ccc(Cl)cc2F)Oc2cccc(C3CCN(Cc4ncc(-c5nnn[nH]5)cc4C(F)(F)F)CC3)c2O1] |
| 99 | 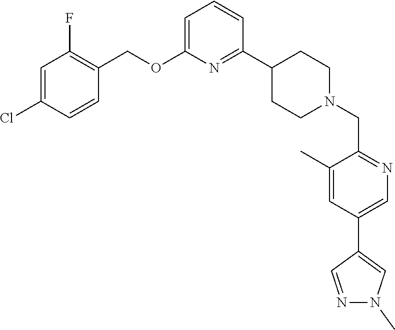 [SMILES: Cc1cc(-c2cnn(C)c2)cnc1CN1CCC(c2cccc(OCc3ccc(Cl)cc3F)n2)CC1] |
| 100 | 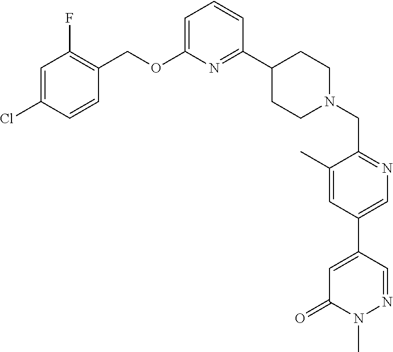 [SMILES: Cc1cc(-c2cnn(C)c(=O)c2)cnc1CN1CCC(c2cccc(OCc3ccc(Cl)cc3F)n2)CC1] |

In some embodiments, provided is a compound selected from Table 2, or a pharmaceutically acceptable salt, isotopically enriched analog, stereoisomer, mixture of stereoisomers, or prodrug thereof.

TABLE 2
Structure

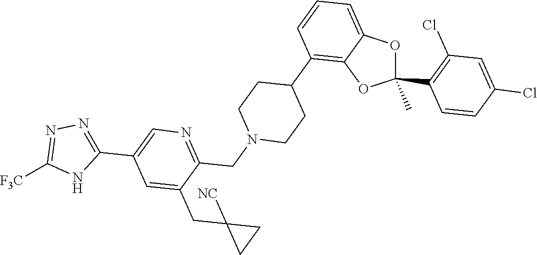 [SMILES: C[C@@]1(c2ccc(Cl)cc2Cl)Oc2cccc(C3CCN(Cc4ncc(-c5nnc(C(F)(F)F)[nH]5)cc4CC4(C#N)CC4)CC3)c2O1]

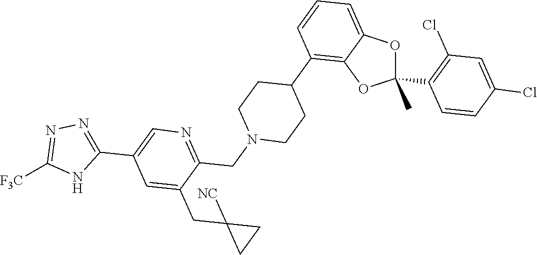 [SMILES: C[C@]1(c2ccc(Cl)cc2Cl)Oc2cccc(C3CCN(Cc4ncc(-c5nnc(C(F)(F)F)[nH]5)cc4CC4(C#N)CC4)CC3)c2O1]

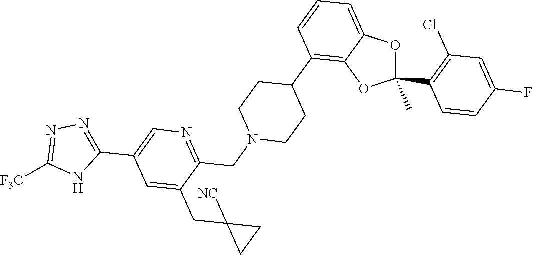 [SMILES: C[C@@]1(c2ccc(F)cc2Cl)Oc2cccc(C3CCN(Cc4ncc(-c5nnc(C(F)(F)F)[nH]5)cc4CC4(C#N)CC4)CC3)c2O1]

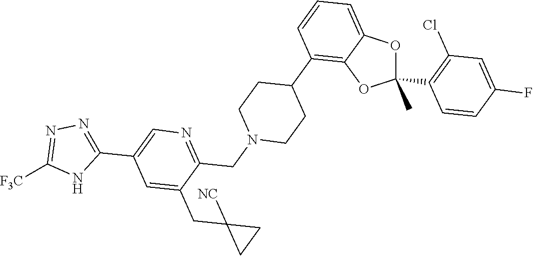 [SMILES: C[C@]1(c2ccc(F)cc2Cl)Oc2cccc(C3CCN(Cc4ncc(-c5nnc(C(F)(F)F)[nH]5)cc4CC4(C#N)CC4)CC3)c2O1]

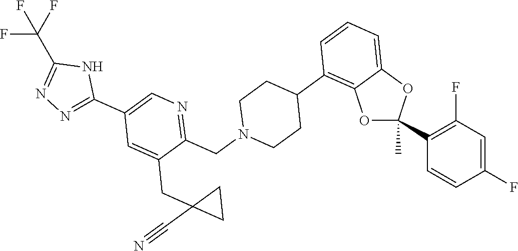 [SMILES: C[C@@]1(c2ccc(F)cc2F)Oc2cccc(C3CCN(Cc4ncc(-c5nnc(C(F)(F)F)[nH]5)cc4CC4(C#N)CC4)CC3)c2O1]

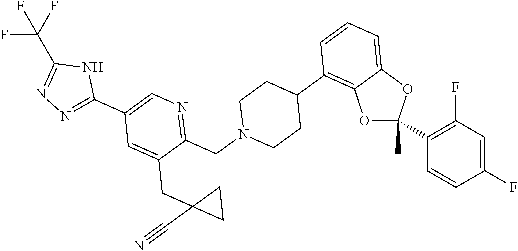 [SMILES: C[C@]1(c2ccc(F)cc2F)Oc2cccc(C3CCN(Cc4ncc(-c5nnc(C(F)(F)F)[nH]5)cc4CC4(C#N)CC4)CC3)c2O1]

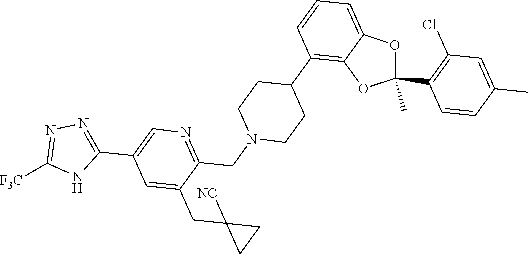 [SMILES: Cc1ccc([C@]2(C)Oc3cccc(C4CCN(Cc5ncc(-c6nnc(C(F)(F)F)[nH]6)cc5CC5(C#N)CC5)CC4)c3O2)c(Cl)c1]

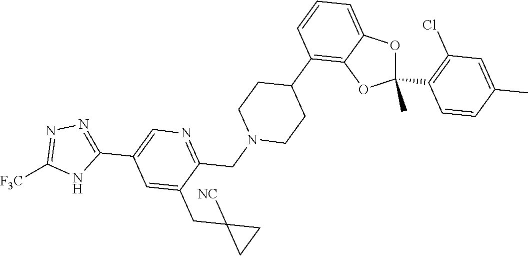 [SMILES: Cc1ccc([C@@]2(C)Oc3cccc(C4CCN(Cc5ncc(-c6nnc(C(F)(F)F)[nH]6)cc5CC5(C#N)CC5)CC4)c3O2)c(Cl)c1]

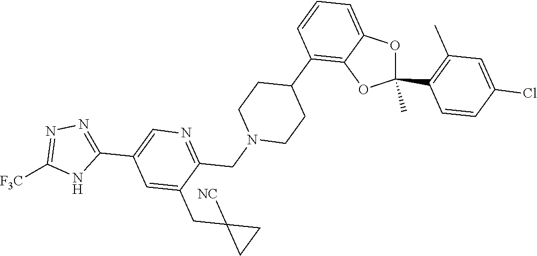 [SMILES: Cc1cc(Cl)ccc1[C@]1(C)Oc2cccc(C3CCN(Cc4ncc(-c5nnc(C(F)(F)F)[nH]5)cc4CC4(C#N)CC4)CC3)c2O1]

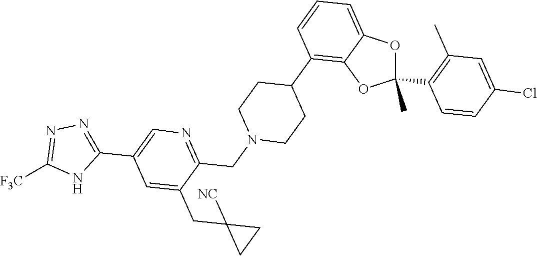 [SMILES: Cc1cc(Cl)ccc1[C@@]1(C)Oc2cccc(C3CCN(Cc4ncc(-c5nnc(C(F)(F)F)[nH]5)cc4CC4(C#N)CC4)CC3)c2O1]

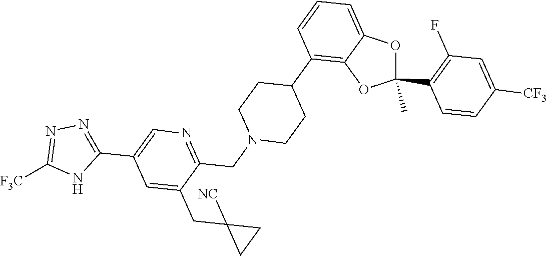 [SMILES: C[C@@]1(c2ccc(C(F)(F)F)cc2F)Oc2cccc(C3CCN(Cc4ncc(-c5nnc(C(F)(F)F)[nH]5)cc4CC4(C#N)CC4)CC3)c2O1]

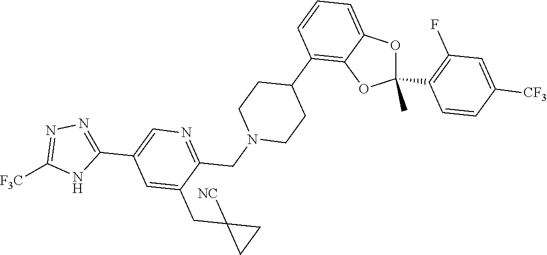 [SMILES: C[C@]1(c2ccc(C(F)(F)F)cc2F)Oc2cccc(C3CCN(Cc4ncc(-c5nnc(C(F)(F)F)[nH]5)cc4CC4(C#N)CC4)CC3)c2O1]

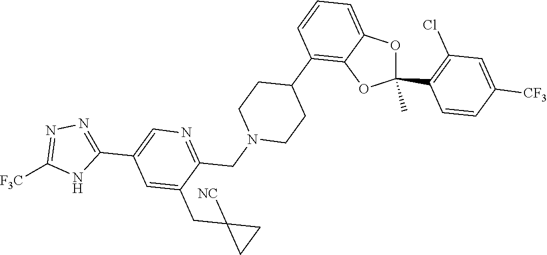 [SMILES: C[C@@]1(c2ccc(C(F)(F)F)cc2Cl)Oc2cccc(C3CCN(Cc4ncc(-c5nnc(C(F)(F)F)[nH]5)cc4CC4(C#N)CC4)CC3)c2O1]

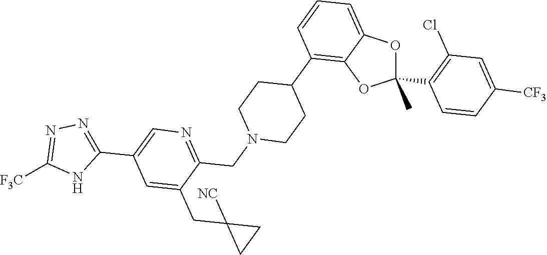 [SMILES: C[C@]1(c2ccc(C(F)(F)F)cc2Cl)Oc2cccc(C3CCN(Cc4ncc(-c5nnc(C(F)(F)F)[nH]5)cc4CC4(C#N)CC4)CC3)c2O1]

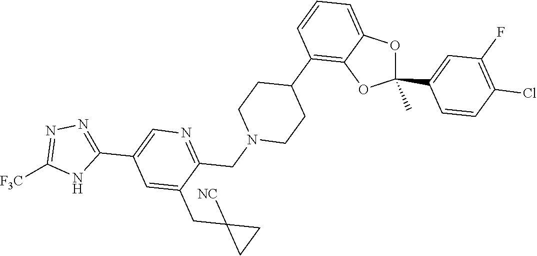 [SMILES: C[C@@]1(c2ccc(Cl)c(F)c2)Oc2cccc(C3CCN(Cc4ncc(-c5nnc(C(F)(F)F)[nH]5)cc4CC4(C#N)CC4)CC3)c2O1]

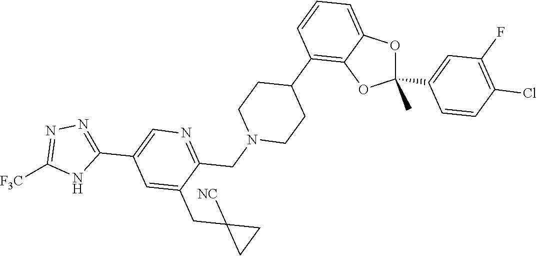 [SMILES: C[C@]1(c2ccc(Cl)c(F)c2)Oc2cccc(C3CCN(Cc4ncc(-c5nnc(C(F)(F)F)[nH]5)cc4CC4(C#N)CC4)CC3)c2O1]

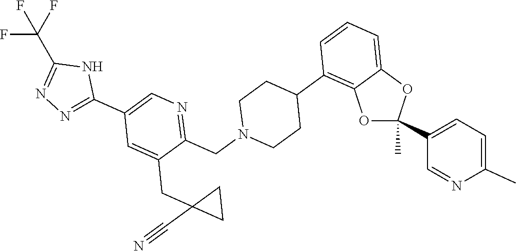 [SMILES: Cc1ccc([C@]2(C)Oc3cccc(C4CCN(Cc5ncc(-c6nnc(C(F)(F)F)[nH]6)cc5CC5(C#N)CC5)CC4)c3O2)cn1]

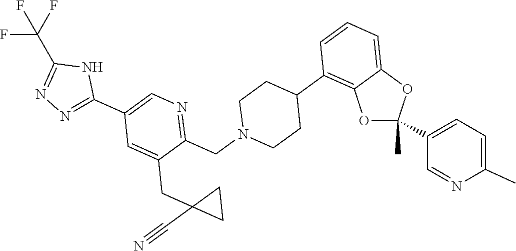 [SMILES: Cc1ccc([C@@]2(C)Oc3cccc(C4CCN(Cc5ncc(-c6nnc(C(F)(F)F)[nH]6)cc5CC5(C#N)CC5)CC4)c3O2)cn1]

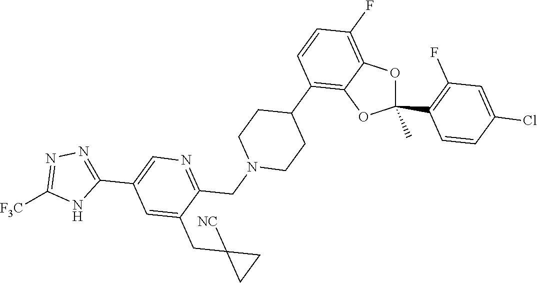 [SMILES: C[C@@]1(c2ccc(Cl)cc2F)Oc2c(F)ccc(C3CCN(Cc4ncc(-c5nnc(C(F)(F)F)[nH]5)cc4CC4(C#N)CC4)CC3)c2O1]

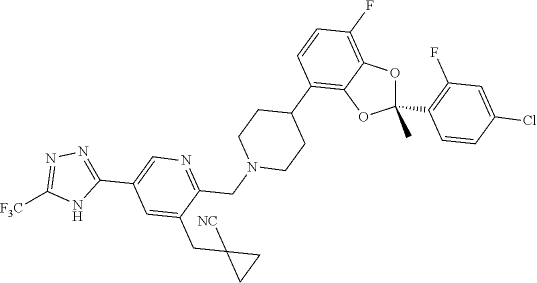 [SMILES: C[C@]1(c2ccc(Cl)cc2F)Oc2c(F)ccc(C3CCN(Cc4ncc(-c5nnc(C(F)(F)F)[nH]5)cc4CC4(C#N)CC4)CC3)c2O1]

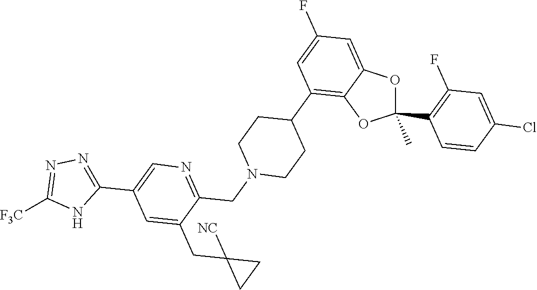 [SMILES: C[C@@]1(c2ccc(Cl)cc2F)Oc2cc(F)cc(C3CCN(Cc4ncc(-c5nnc(C(F)(F)F)[nH]5)cc4CC4(C#N)CC4)CC3)c2O1]

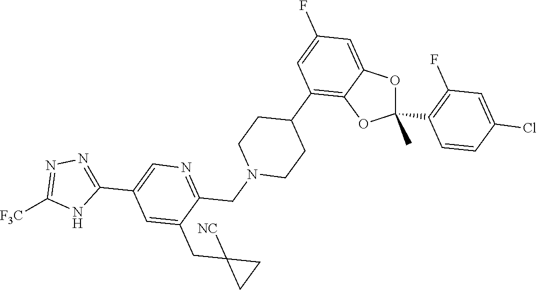 [SMILES: C[C@]1(c2ccc(Cl)cc2F)Oc2cc(F)cc(C3CCN(Cc4ncc(-c5nnc(C(F)(F)F)[nH]5)cc4CC4(C#N)CC4)CC3)c2O1]

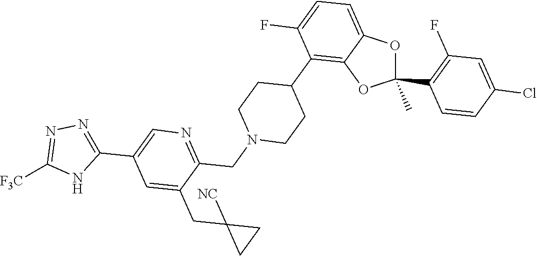 [SMILES: C[C@@]1(c2ccc(Cl)cc2F)Oc2ccc(F)c(C3CCN(Cc4ncc(-c5nnc(C(F)(F)F)[nH]5)cc4CC4(C#N)CC4)CC3)c2O1]

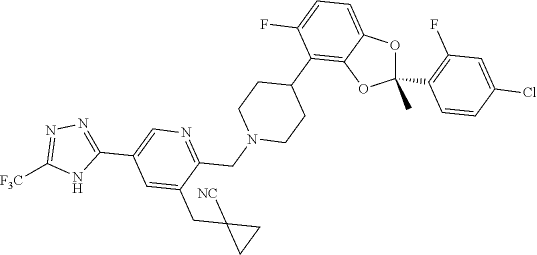 [SMILES: C[C@]1(c2ccc(Cl)cc2F)Oc2ccc(F)c(C3CCN(Cc4ncc(-c5nnc(C(F)(F)F)[nH]5)cc4CC4(C#N)CC4)CC3)c2O1]

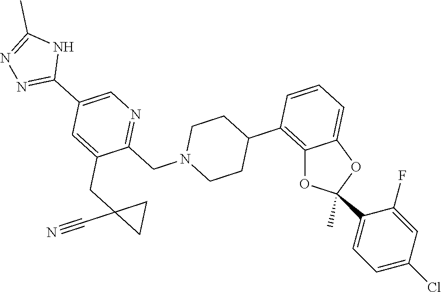 [SMILES: Cc1nnc(-c2cnc(CN3CCC(c4cccc5c4O[C@](C)(c4ccc(Cl)cc4F)O5)CC3)c(CC3(C#N)CC3)c2)[nH]1]

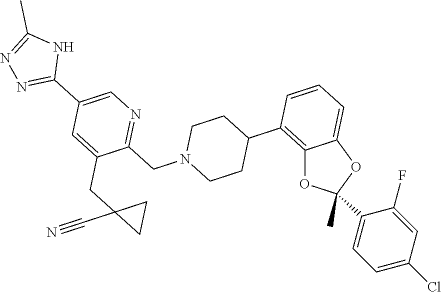 [SMILES: Cc1nnc(-c2cnc(CN3CCC(c4cccc5c4O[C@@](C)(c4ccc(Cl)cc4F)O5)CC3)c(CC3(C#N)CC3)c2)[nH]1]

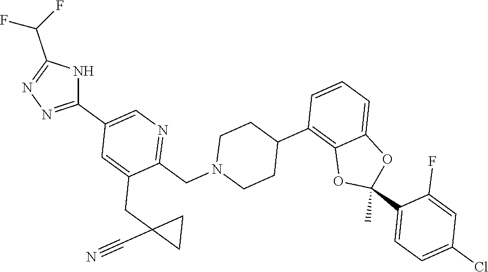 [SMILES: C[C@@]1(c2ccc(Cl)cc2F)Oc2cccc(C3CCN(Cc4ncc(-c5nnc(C(F)F)[nH]5)cc4CC4(C#N)CC4)CC3)c2O1]

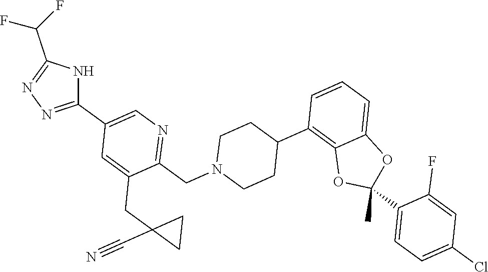 [SMILES: C[C@]1(c2ccc(Cl)cc2F)Oc2cccc(C3CCN(Cc4ncc(-c5nnc(C(F)F)[nH]5)cc4CC4(C#N)CC4)CC3)c2O1]

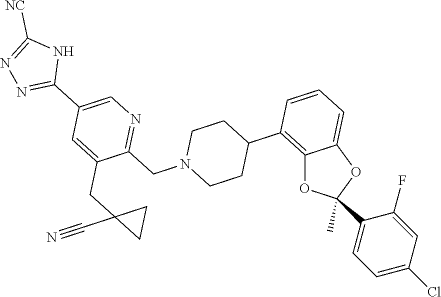 [SMILES: C[C@@]1(c2ccc(Cl)cc2F)Oc2cccc(C3CCN(Cc4ncc(-c5nnc(C#N)[nH]5)cc4CC4(C#N)CC4)CC3)c2O1]

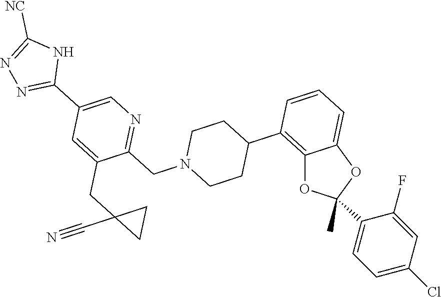 [SMILES: C[C@]1(c2ccc(Cl)cc2F)Oc2cccc(C3CCN(Cc4ncc(-c5nnc(C#N)[nH]5)cc4CC4(C#N)CC4)CC3)c2O1]

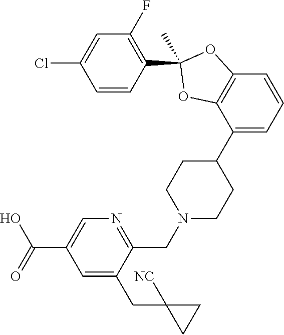 [SMILES: C[C@]1(c2ccc(Cl)cc2F)Oc2cccc(C3CCN(Cc4ncc(C(=O)O)cc4CC4(C#N)CC4)CC3)c2O1]

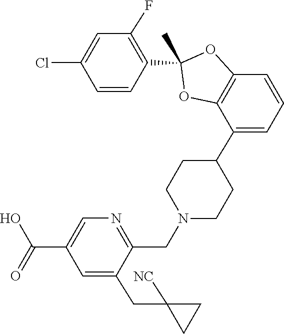 [SMILES: C[C@@]1(c2ccc(Cl)cc2F)Oc2cccc(C3CCN(Cc4ncc(C(=O)O)cc4CC4(C#N)CC4)CC3)c2O1]

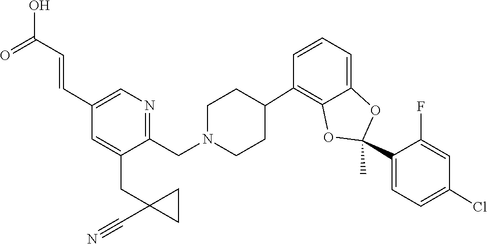 [SMILES: C[C@@]1(c2ccc(Cl)cc2F)Oc2cccc(C3CCN(Cc4ncc(/C=C/C(=O)O)cc4CC4(C#N)CC4)CC3)c2O1]

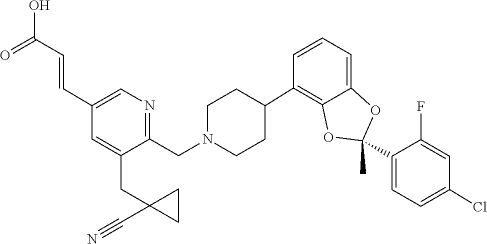 [SMILES: C[C@]1(c2ccc(Cl)cc2F)Oc2cccc(C3CCN(Cc4ncc(/C=C/C(=O)O)cc4CC4(C#N)CC4)CC3)c2O1]

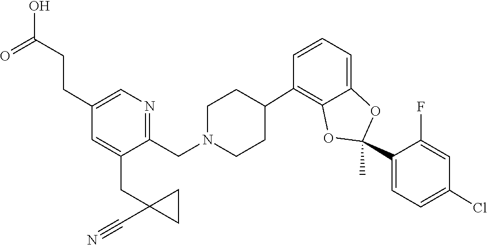 [SMILES: C[C@@]1(c2ccc(Cl)cc2F)Oc2cccc(C3CCN(Cc4ncc(CCC(=O)O)cc4CC4(C#N)CC4)CC3)c2O1]

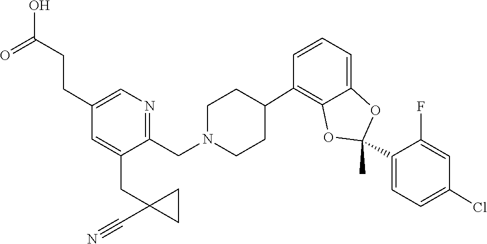 [SMILES: C[C@]1(c2ccc(Cl)cc2F)Oc2cccc(C3CCN(Cc4ncc(CCC(=O)O)cc4CC4(C#N)CC4)CC3)c2O1]

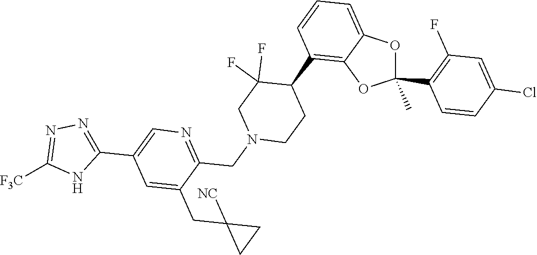 [SMILES: C[C@@]1(c2ccc(Cl)cc2F)Oc2cccc([C@H]3CCN(Cc4ncc(-c5nnc(C(F)(F)F)[nH]5)cc4CC4(C#N)CC4)CC3(F)F)c2O1]

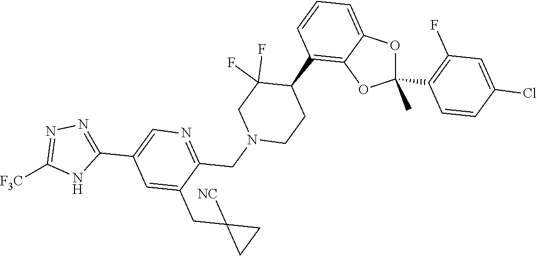 [SMILES: C[C@]1(c2ccc(Cl)cc2F)Oc2cccc([C@H]3CCN(Cc4ncc(-c5nnc(C(F)(F)F)[nH]5)cc4CC4(C#N)CC4)CC3(F)F)c2O1]

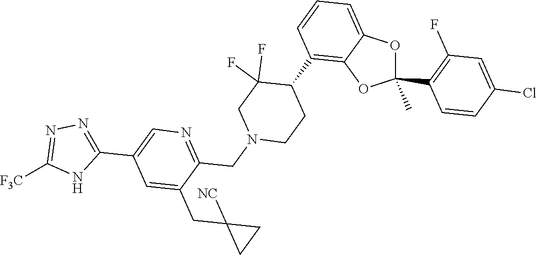 [SMILES: C[C@@]1(c2ccc(Cl)cc2F)Oc2cccc([C@@H]3CCN(Cc4ncc(-c5nnc(C(F)(F)F)[nH]5)cc4CC4(C#N)CC4)CC3(F)F)c2O1]

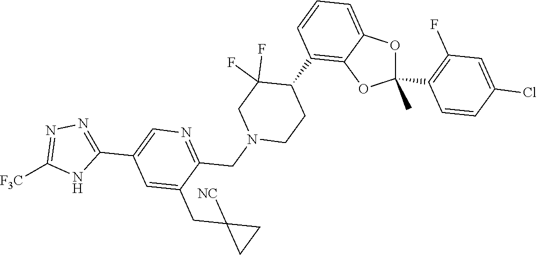 [SMILES: C[C@]1(c2ccc(Cl)cc2F)Oc2cccc([C@@H]3CCN(Cc4ncc(-c5nnc(C(F)(F)F)[nH]5)cc4CC4(C#N)CC4)CC3(F)F)c2O1]

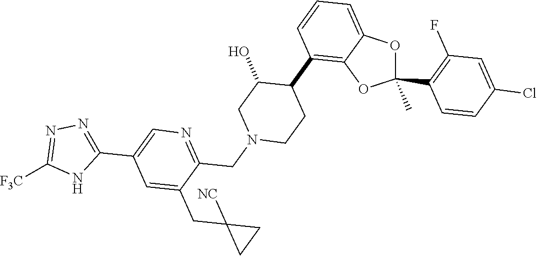

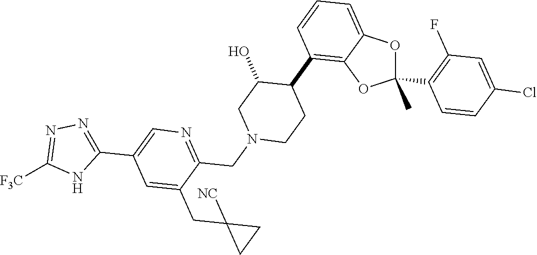

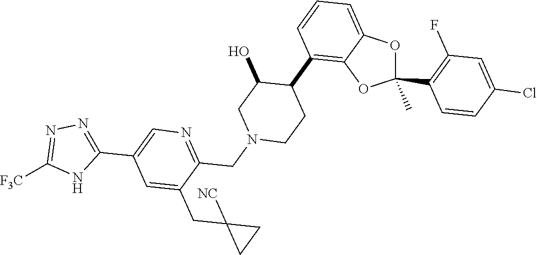

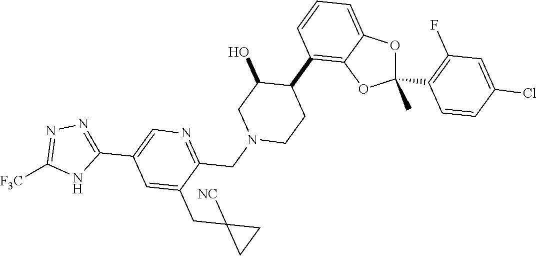

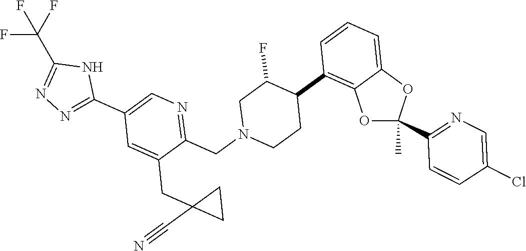

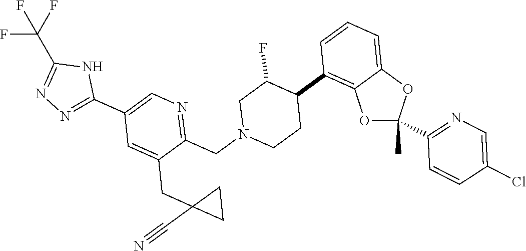

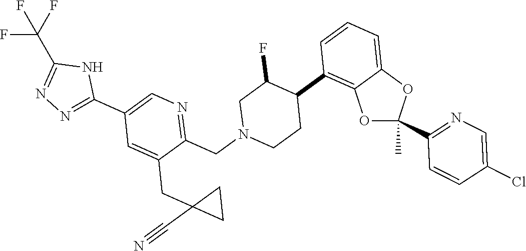

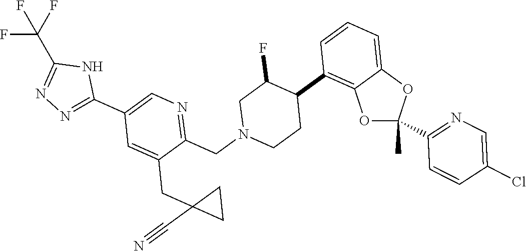

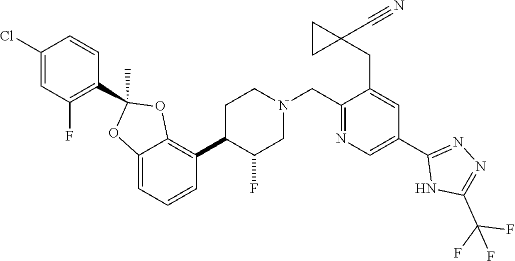 [SMILES: C[C@@]1(c2ccc(Cl)cc2F)Oc2cccc([C@H]3CCN(Cc4ncc(-c5nnc(C(F)(F)F)[nH]5)cc4CC4(C#N)CC4)C[C@@H]3F)c2O1]

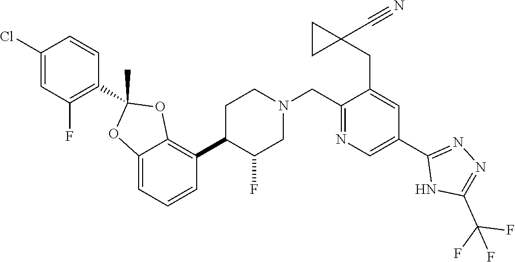 [SMILES: C[C@]1(c2ccc(Cl)cc2F)Oc2cccc([C@H]3CCN(Cc4ncc(-c5nnc(C(F)(F)F)[nH]5)cc4CC4(C#N)CC4)C[C@@H]3F)c2O1]

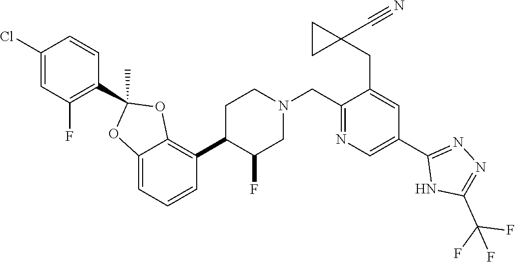 [SMILES: C[C@@]1(c2ccc(Cl)cc2F)Oc2cccc([C@H]3CCN(Cc4ncc(-c5nnc(C(F)(F)F)[nH]5)cc4CC4(C#N)CC4)C[C@H]3F)c2O1]

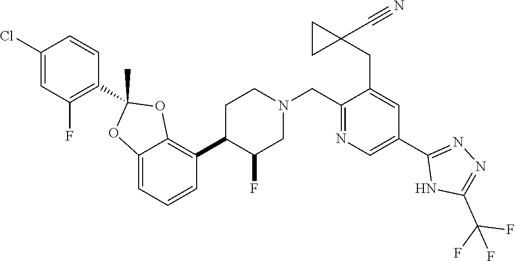 [SMILES: C[C@]1(c2ccc(Cl)cc2F)Oc2cccc([C@H]3CCN(Cc4ncc(-c5nnc(C(F)(F)F)[nH]5)cc4CC4(C#N)CC4)C[C@H]3F)c2O1]

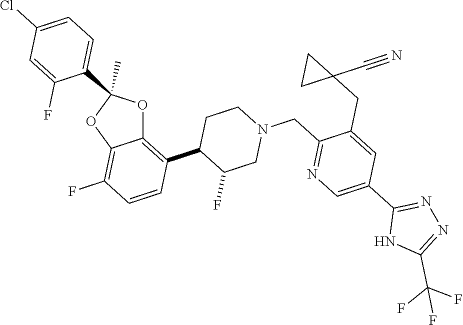 [SMILES: C[C@@]1(c2ccc(Cl)cc2F)Oc2c(F)ccc([C@H]3CCN(Cc4ncc(-c5nnc(C(F)(F)F)[nH]5)cc4CC4(C#N)CC4)C[C@@H]3F)c2O1]

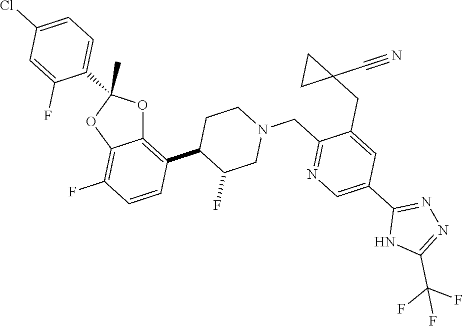 [SMILES: C[C@]1(c2ccc(Cl)cc2F)Oc2c(F)ccc([C@H]3CCN(Cc4ncc(-c5nnc(C(F)(F)F)[nH]5)cc4CC4(C#N)CC4)C[C@@H]3F)c2O1]

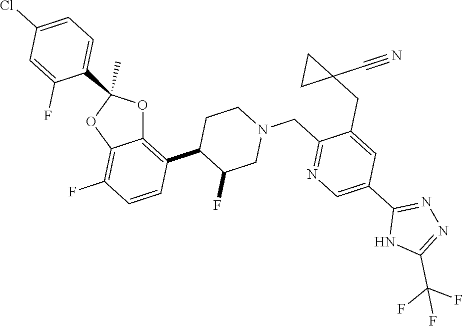 [SMILES: C[C@@]1(c2ccc(Cl)cc2F)Oc2c(F)ccc([C@H]3CCN(Cc4ncc(-c5nnc(C(F)(F)F)[nH]5)cc4CC4(C#N)CC4)C[C@H]3F)c2O1]

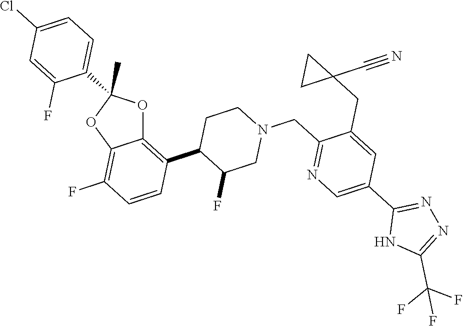 [SMILES: C[C@]1(c2ccc(Cl)cc2F)Oc2c(F)ccc([C@H]3CCN(Cc4ncc(-c5nnc(C(F)(F)F)[nH]5)cc4CC4(C#N)CC4)C[C@H]3F)c2O1]

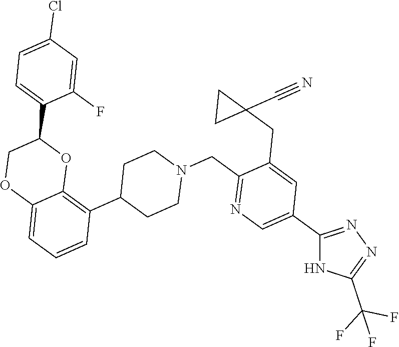 [SMILES: N#CC1(Cc2cc(-c3nnc(C(F)(F)F)[nH]3)cnc2CN2CCC(c3cccc4c3O[C@H](c3ccc(Cl)cc3F)CO4)CC2)CC1]

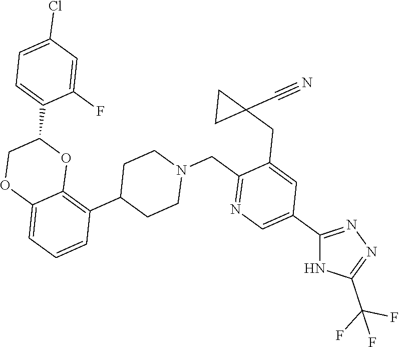 [SMILES: N#CC1(Cc2cc(-c3nnc(C(F)(F)F)[nH]3)cnc2CN2CCC(c3cccc4c3O[C@@H](c3ccc(Cl)cc3F)CO4)CC2)CC1]

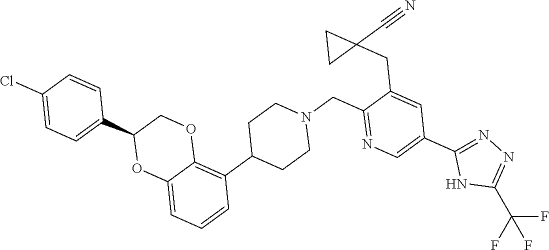 [SMILES: N#CC1(Cc2cc(-c3nnc(C(F)(F)F)[nH]3)cnc2CN2CCC(c3cccc4c3OC[C@H](c3ccc(Cl)cc3)O4)CC2)CC1]

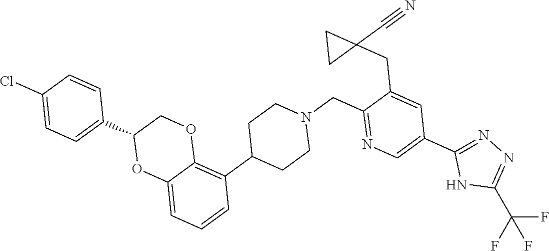 [SMILES: N#CC1(Cc2cc(-c3nnc(C(F)(F)F)[nH]3)cnc2CN2CCC(c3cccc4c3OC[C@@H](c3ccc(Cl)cc3)O4)CC2)CC1]

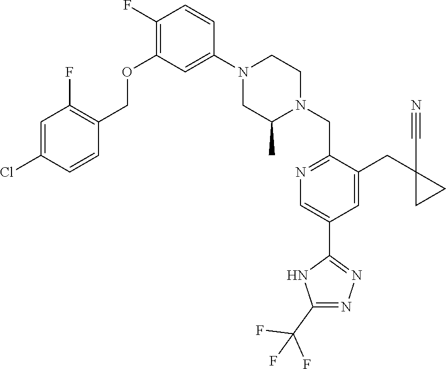 [SMILES: C[C@H]1CN(c2ccc(F)c(OCc3ccc(Cl)cc3F)c2)CCN1Cc1ncc(-c2nnc(C(F)(F)F)[nH]2)cc1CC1(C#N)CC1]

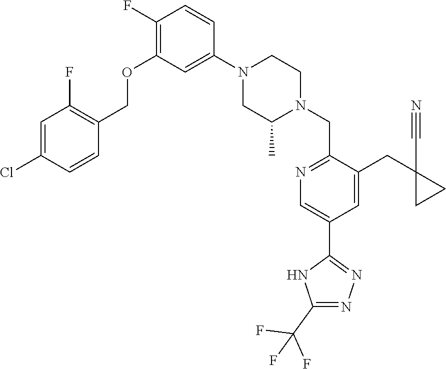 [SMILES: C[C@@H]1CN(c2ccc(F)c(OCc3ccc(Cl)cc3F)c2)CCN1Cc1ncc(-c2nnc(C(F)(F)F)[nH]2)cc1CC1(C#N)CC1]

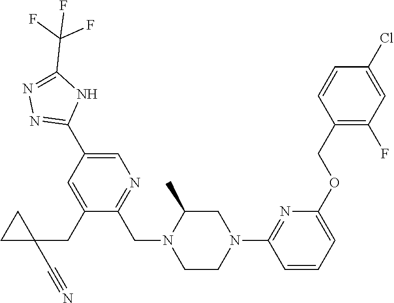 [SMILES: C[C@H]1CN(c2cccc(OCc3ccc(Cl)cc3F)n2)CCN1Cc1ncc(-c2nnc(C(F)(F)F)[nH]2)cc1CC1(C#N)CC1]

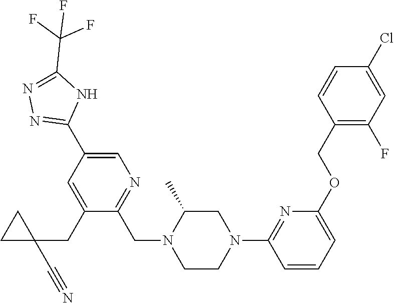 [SMILES: C[C@@H]1CN(c2cccc(OCc3ccc(Cl)cc3F)n2)CCN1Cc1ncc(-c2nnc(C(F)(F)F)[nH]2)cc1CC1(C#N)CC1]

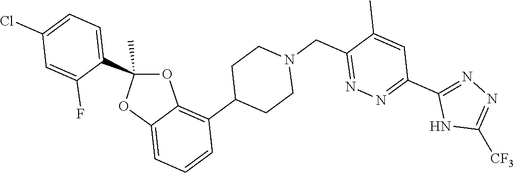 [SMILES: Cc1cc(-c2nnc(C(F)(F)F)[nH]2)nnc1CN1CCC(c2cccc3c2O[C@](C)(c2ccc(Cl)cc2F)O3)CC1]

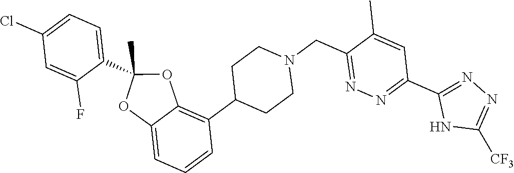 [SMILES: Cc1cc(-c2nnc(C(F)(F)F)[nH]2)nnc1CN1CCC(c2cccc3c2O[C@@](C)(c2ccc(Cl)cc2F)O3)CC1]

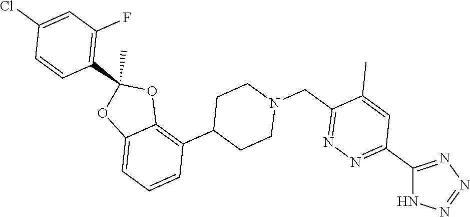 [SMILES: Cc1cc(-c2nnn[nH]2)nnc1CN1CCC(c2cccc3c2O[C@](C)(c2ccc(Cl)cc2F)O3)CC1]

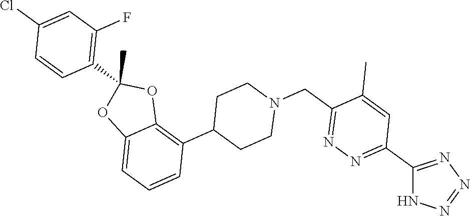 [SMILES: Cc1cc(-c2nnn[nH]2)nnc1CN1CCC(c2cccc3c2O[C@@](C)(c2ccc(Cl)cc2F)O3)CC1]

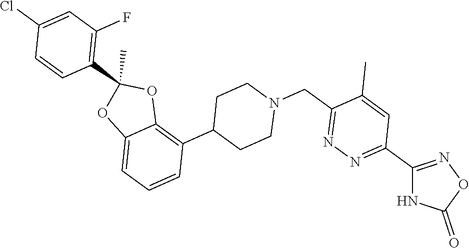 [SMILES: Cc1cc(-c2noc(=O)[nH]2)nnc1CN1CCC(c2cccc3c2O[C@](C)(c2ccc(Cl)cc2F)O3)CC1]

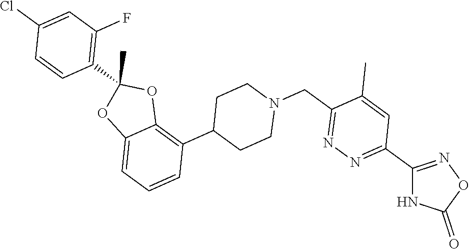 [SMILES: Cc1cc(-c2noc(=O)[nH]2)nnc1CN1CCC(c2cccc3c2O[C@@](C)(c2ccc(Cl)cc2F)O3)CC1]

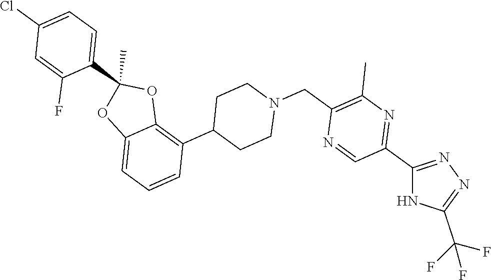 [SMILES: Cc1nc(-c2nnc(C(F)(F)F)[nH]2)cnc1CN1CCC(c2cccc3c2O[C@](C)(c2ccc(Cl)cc2F)O3)CC1]

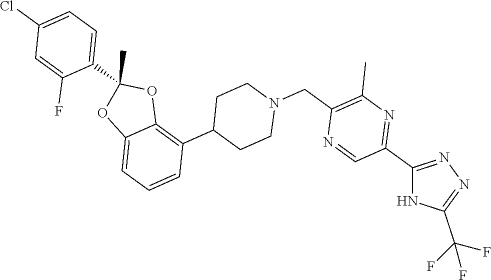 [SMILES: Cc1nc(-c2nnc(C(F)(F)F)[nH]2)cnc1CN1CCC(c2cccc3c2O[C@@](C)(c2ccc(Cl)cc2F)O3)CC1]

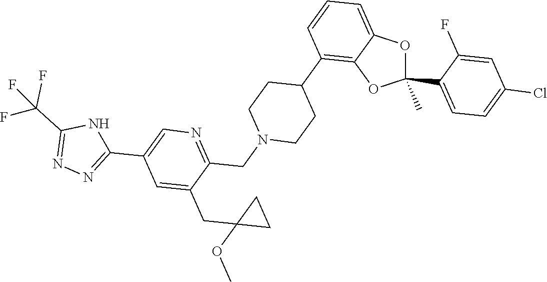 [SMILES: COC1(Cc2cc(-c3nnc(C(F)(F)F)[nH]3)cnc2CN2CCC(c3cccc4c3O[C@](C)(c3ccc(Cl)cc3F)O4)CC2)CC1]

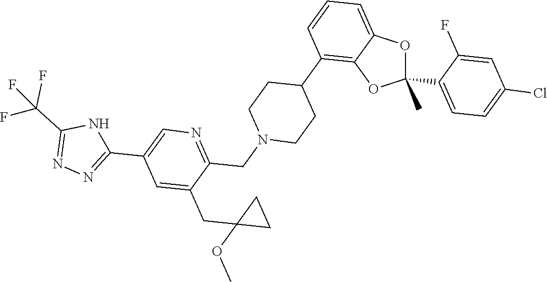 [SMILES: COC1(Cc2cc(-c3nnc(C(F)(F)F)[nH]3)cnc2CN2CCC(c3cccc4c3O[C@@](C)(c3ccc(Cl)cc3F)O4)CC2)CC1]

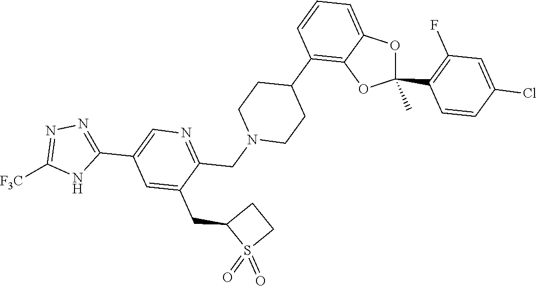 [SMILES: C[C@@]1(c2ccc(Cl)cc2F)Oc2cccc(C3CCN(Cc4ncc(-c5nnc(C(F)(F)F)[nH]5)cc4C[C@H]4CCS4(=O)=O)CC3)c2O1]

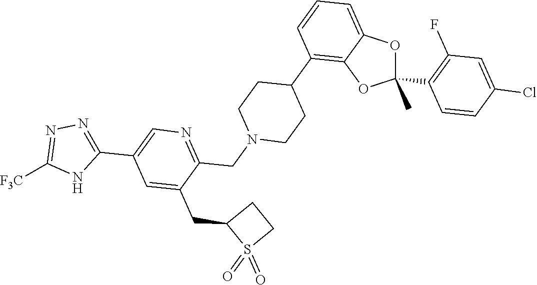 [SMILES: C[C@]1(c2ccc(Cl)cc2F)Oc2cccc(C3CCN(Cc4ncc(-c5nnc(C(F)(F)F)[nH]5)cc4C[C@H]4CCS4(=O)=O)CC3)c2O1]

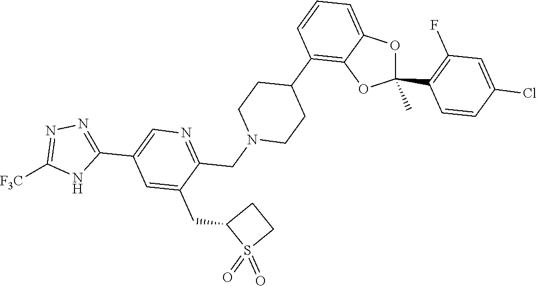 [SMILES: C[C@@]1(c2ccc(Cl)cc2F)Oc2cccc(C3CCN(Cc4ncc(-c5nnc(C(F)(F)F)[nH]5)cc4C[C@@H]4CCS4(=O)=O)CC3)c2O1]

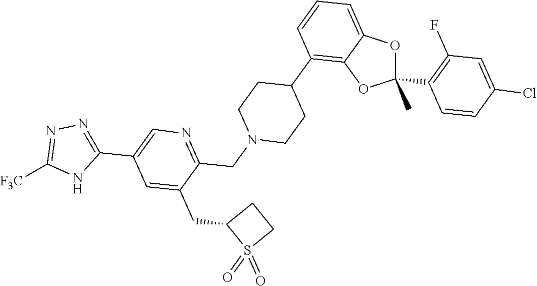 [SMILES: C[C@]1(c2ccc(Cl)cc2F)Oc2cccc(C3CCN(Cc4ncc(-c5nnc(C(F)(F)F)[nH]5)cc4C[C@@H]4CCS4(=O)=O)CC3)c2O1]

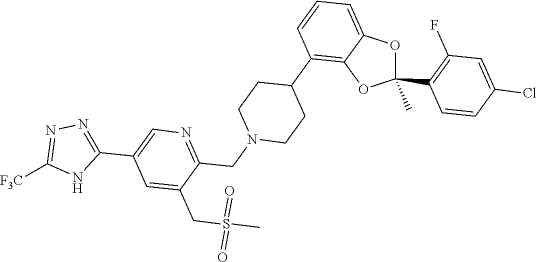 [SMILES: C[C@@]1(c2ccc(Cl)cc2F)Oc2cccc(C3CCN(Cc4ncc(-c5nnc(C(F)(F)F)[nH]5)cc4CS(C)(=O)=O)CC3)c2O1]

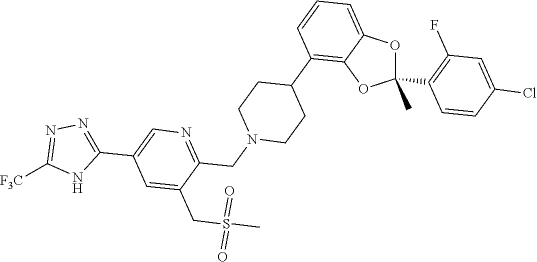 [SMILES: C[C@]1(c2ccc(Cl)cc2F)Oc2cccc(C3CCN(Cc4ncc(-c5nnc(C(F)(F)F)[nH]5)cc4CS(C)(=O)=O)CC3)c2O1]

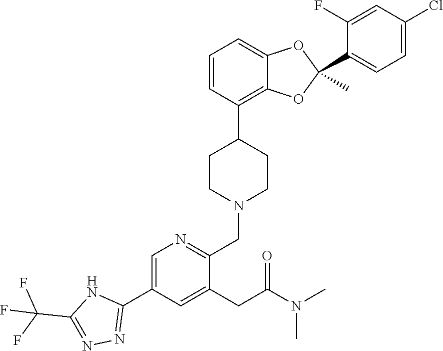 [SMILES: CN(C)C(=O)Cc1cc(-c2nnc(C(F)(F)F)[nH]2)cnc1CN1CCC(c2cccc3c2O[C@](C)(c2ccc(Cl)cc2F)O3)CC1]

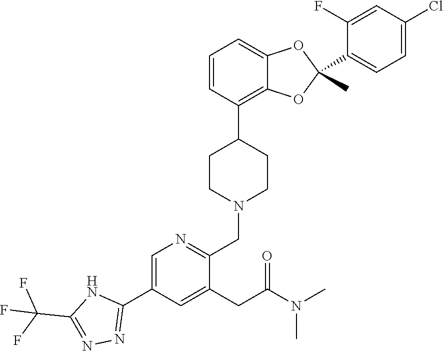 [SMILES: CN(C)C(=O)Cc1cc(-c2nnc(C(F)(F)F)[nH]2)cnc1CN1CCC(c2cccc3c2O[C@@](C)(c2ccc(Cl)cc2F)O3)CC1]

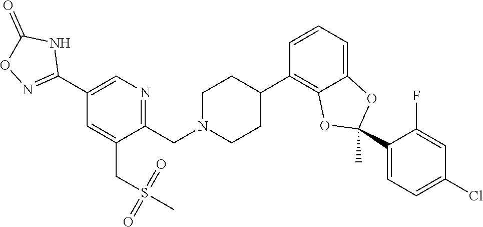 [SMILES: C[C@@]1(c2ccc(Cl)cc2F)Oc2cccc(C3CCN(Cc4ncc(-c5noc(=O)[nH]5)cc4CS(C)(=O)=O)CC3)c2O1]

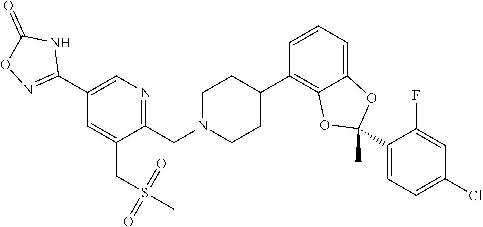 [SMILES: C[C@]1(c2ccc(Cl)cc2F)Oc2cccc(C3CCN(Cc4ncc(-c5noc(=O)[nH]5)cc4CS(C)(=O)=O)CC3)c2O1]

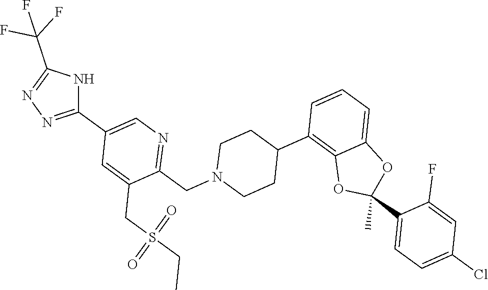 [SMILES: CCS(=O)(=O)Cc1cc(-c2nnc(C(F)(F)F)[nH]2)cnc1CN1CCC(c2cccc3c2O[C@](C)(c2ccc(Cl)cc2F)O3)CC1]

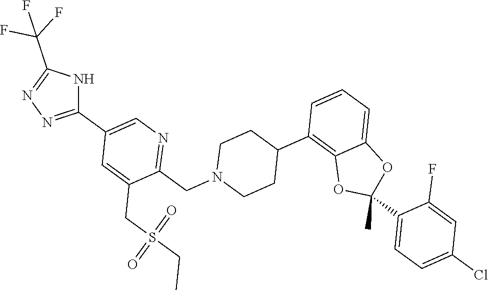 [SMILES: CCS(=O)(=O)Cc1cc(-c2nnc(C(F)(F)F)[nH]2)cnc1CN1CCC(c2cccc3c2O[C@@](C)(c2ccc(Cl)cc2F)O3)CC1]

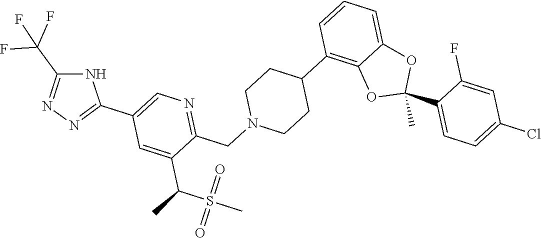 [SMILES: C[C@@H](c1cc(-c2nnc(C(F)(F)F)[nH]2)cnc1CN1CCC(c2cccc3c2O[C@](C)(c2ccc(Cl)cc2F)O3)CC1)S(C)(=O)=O]

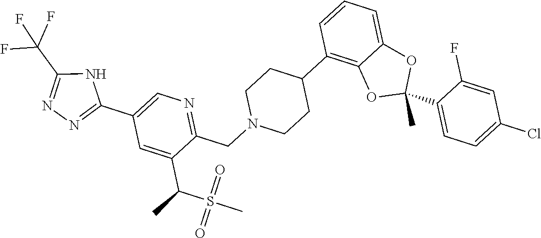 [SMILES: C[C@@H](c1cc(-c2nnc(C(F)(F)F)[nH]2)cnc1CN1CCC(c2cccc3c2O[C@@](C)(c2ccc(Cl)cc2F)O3)CC1)S(C)(=O)=O]

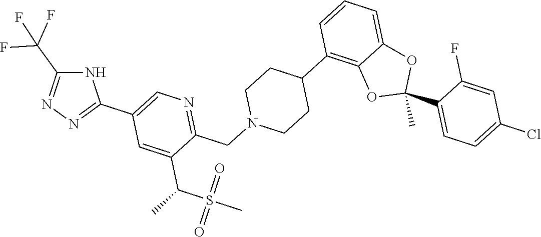 [SMILES: C[C@H](c1cc(-c2nnc(C(F)(F)F)[nH]2)cnc1CN1CCC(c2cccc3c2O[C@](C)(c2ccc(Cl)cc2F)O3)CC1)S(C)(=O)=O]

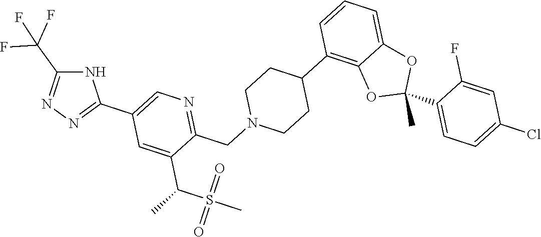 [SMILES: C[C@H](c1cc(-c2nnc(C(F)(F)F)[nH]2)cnc1CN1CCC(c2cccc3c2O[C@@](C)(c2ccc(Cl)cc2F)O3)CC1)S(C)(=O)=O]

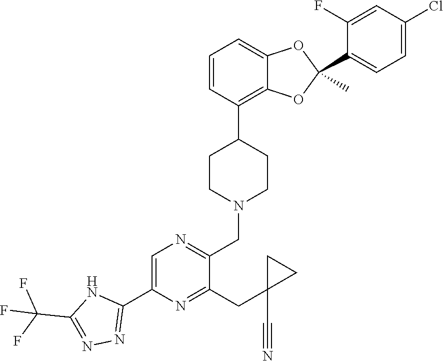 [SMILES: C[C@@]1(c2ccc(Cl)cc2F)Oc2cccc(C3CCN(Cc4ncc(-c5nnc(C(F)(F)F)[nH]5)nc4CC4(C#N)CC4)CC3)c2O1]

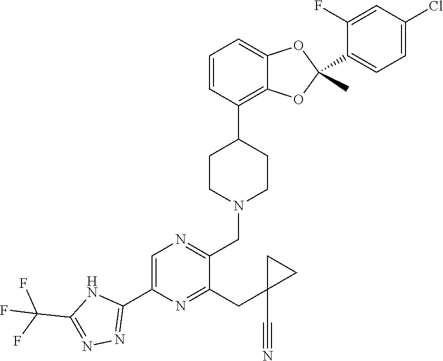 [SMILES: C[C@]1(c2ccc(Cl)cc2F)Oc2cccc(C3CCN(Cc4ncc(-c5nnc(C(F)(F)F)[nH]5)nc4CC4(C#N)CC4)CC3)c2O1]

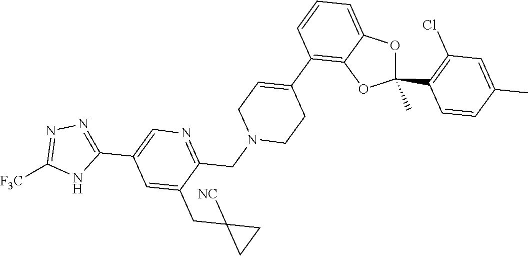 [SMILES: Cc1ccc([C@]2(C)Oc3cccc(C4=CCN(Cc5ncc(-c6nnc(C(F)(F)F)[nH]6)cc5CC5(C#N)CC5)CC4)c3O2)c(Cl)c1]

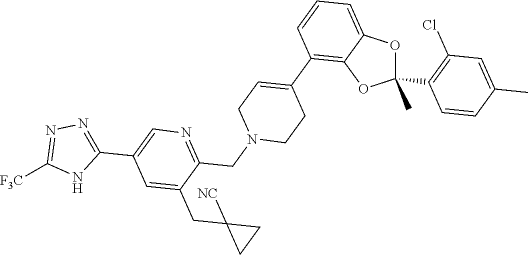 [SMILES: Cc1ccc([C@@]2(C)Oc3cccc(C4=CCN(Cc5ncc(-c6nnc(C(F)(F)F)[nH]6)cc5CC5(C#N)CC5)CC4)c3O2)c(Cl)c1]

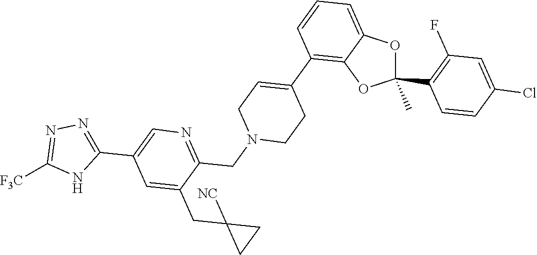 [SMILES: C[C@@]1(c2ccc(Cl)cc2F)Oc2cccc(C3=CCN(Cc4ncc(-c5nnc(C(F)(F)F)[nH]5)cc4CC4(C#N)CC4)CC3)c2O1]

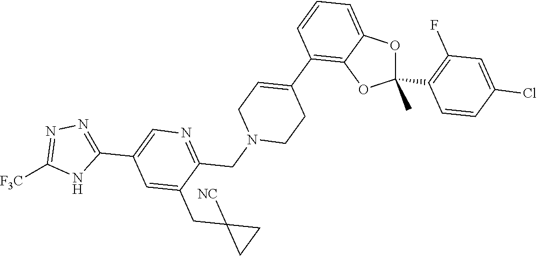 [SMILES: C[C@]1(c2ccc(Cl)cc2F)Oc2cccc(C3=CCN(Cc4ncc(-c5nnc(C(F)(F)F)[nH]5)cc4CC4(C#N)CC4)CC3)c2O1]

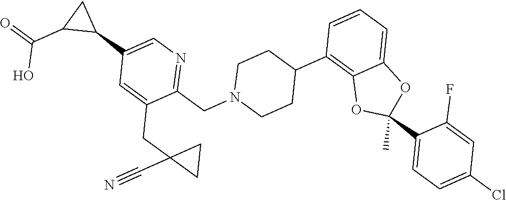 [SMILES: C[C@@]1(c2ccc(Cl)cc2F)Oc2cccc(C3CCN(Cc4ncc([C@@H]5CC5C(=O)O)cc4CC4(C#N)CC4)CC3)c2O1]

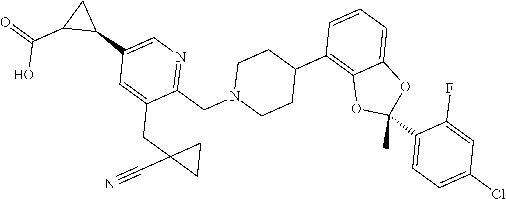 [SMILES: C[C@]1(c2ccc(Cl)cc2F)Oc2cccc(C3CCN(Cc4ncc([C@@H]5CC5C(=O)O)cc4CC4(C#N)CC4)CC3)c2O1]

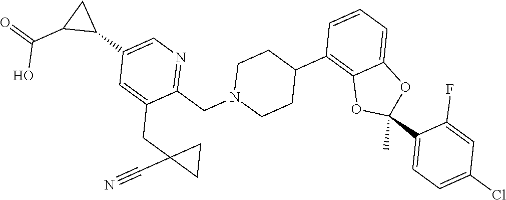 [SMILES: C[C@@]1(c2ccc(Cl)cc2F)Oc2cccc(C3CCN(Cc4ncc([C@H]5CC5C(=O)O)cc4CC4(C#N)CC4)CC3)c2O1]

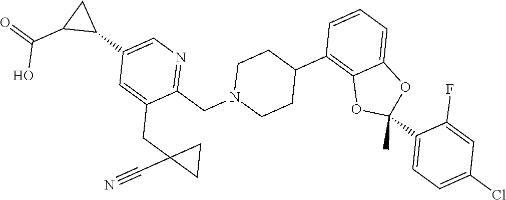 [SMILES: C[C@]1(c2ccc(Cl)cc2F)Oc2cccc(C3CCN(Cc4ncc([C@H]5CC5C(=O)O)cc4CC4(C#N)CC4)CC3)c2O1]

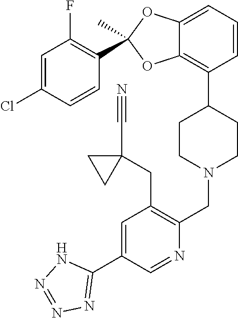 [SMILES: C[C@]1(c2ccc(Cl)cc2F)Oc2cccc(C3CCN(Cc4ncc(-c5nnn[nH]5)cc4CC4(C#N)CC4)CC3)c2O1]

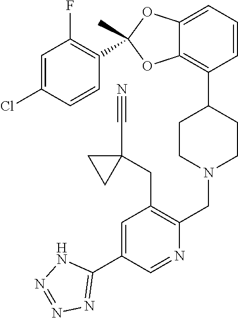 [SMILES: C[C@@]1(c2ccc(Cl)cc2F)Oc2cccc(C3CCN(Cc4ncc(-c5nnn[nH]5)cc4CC4(C#N)CC4)CC3)c2O1]

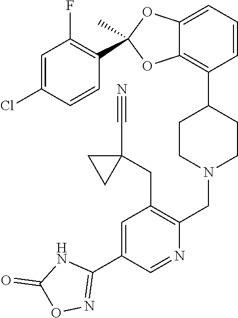 [SMILES: C[C@]1(c2ccc(Cl)cc2F)Oc2cccc(C3CCN(Cc4ncc(-c5noc(=O)[nH]5)cc4CC4(C#N)CC4)CC3)c2O1]

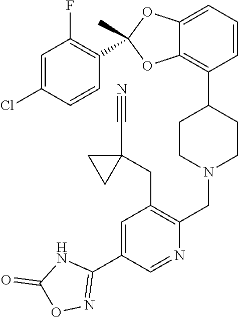 [SMILES: C[C@@]1(c2ccc(Cl)cc2F)Oc2cccc(C3CCN(Cc4ncc(-c5noc(=O)[nH]5)cc4CC4(C#N)CC4)CC3)c2O1]

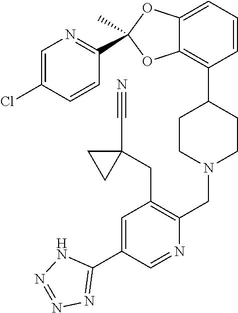 [SMILES: C[C@]1(c2ccc(Cl)cn2)Oc2cccc(C3CCN(Cc4ncc(-c5nnn[nH]5)cc4CC4(C#N)CC4)CC3)c2O1]

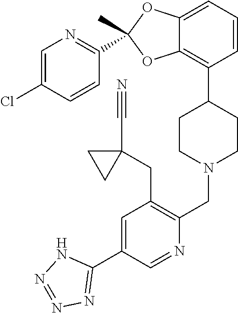 [SMILES: C[C@@]1(c2ccc(Cl)cn2)Oc2cccc(C3CCN(Cc4ncc(-c5nnn[nH]5)cc4CC4(C#N)CC4)CC3)c2O1]

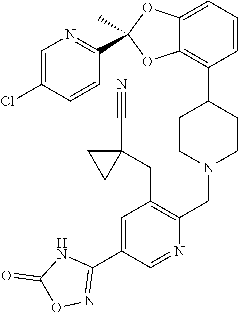 [SMILES: C[C@]1(c2ccc(Cl)cn2)Oc2cccc(C3CCN(Cc4ncc(-c5noc(=O)[nH]5)cc4CC4(C#N)CC4)CC3)c2O1]

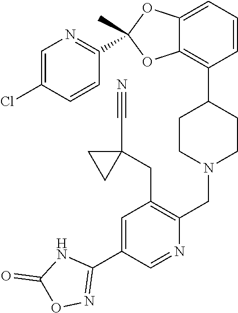 [SMILES: C[C@@]1(c2ccc(Cl)cn2)Oc2cccc(C3CCN(Cc4ncc(-c5noc(=O)[nH]5)cc4CC4(C#N)CC4)CC3)c2O1]

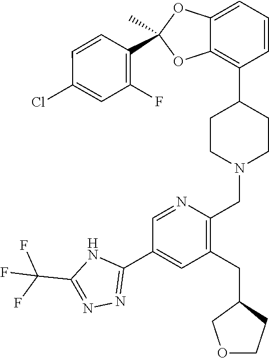 [SMILES: C[C@]1(c2ccc(Cl)cc2F)Oc2cccc(C3CCN(Cc4ncc(-c5nnc(C(F)(F)F)[nH]5)cc4C[C@H]4CCOC4)CC3)c2O1]

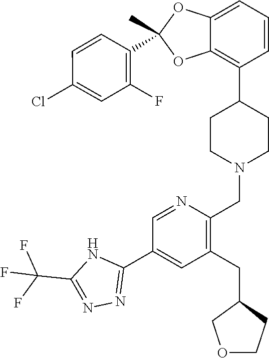 [SMILES: C[C@@]1(c2ccc(Cl)cc2F)Oc2cccc(C3CCN(Cc4ncc(-c5nnc(C(F)(F)F)[nH]5)cc4C[C@H]4CCOC4)CC3)c2O1]

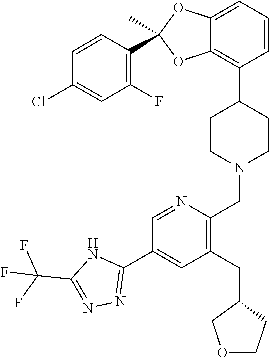 [SMILES: C[C@]1(c2ccc(Cl)cc2F)Oc2cccc(C3CCN(Cc4ncc(-c5nnc(C(F)(F)F)[nH]5)cc4C[C@@H]4CCOC4)CC3)c2O1]

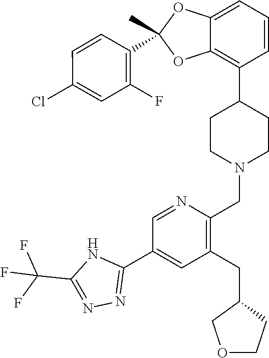 [SMILES: C[C@@]1(c2ccc(Cl)cc2F)Oc2cccc(C3CCN(Cc4ncc(-c5nnc(C(F)(F)F)[nH]5)cc4C[C@@H]4CCOC4)CC3)c2O1]

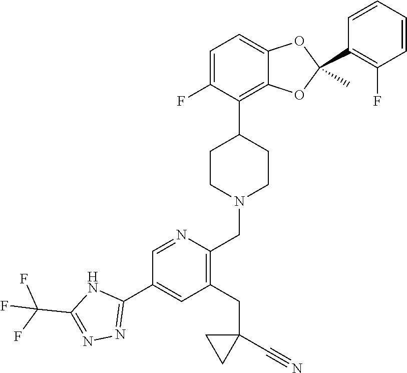 [SMILES: C[C@@]1(c2ccccc2F)Oc2ccc(F)c(C3CCN(Cc4ncc(-c5nnc(C(F)(F)F)[nH]5)cc4CC4(C#N)CC4)CC3)c2O1]

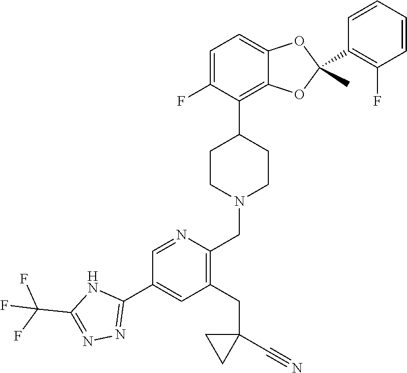 [SMILES: C[C@]1(c2ccccc2F)Oc2ccc(F)c(C3CCN(Cc4ncc(-c5nnc(C(F)(F)F)[nH]5)cc4CC4(C#N)CC4)CC3)c2O1]

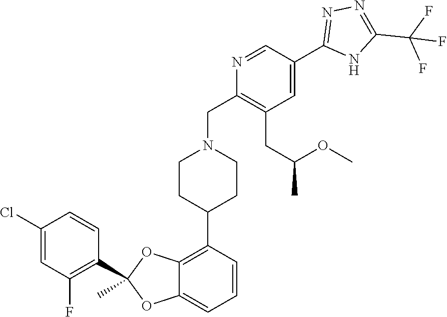 [SMILES: CO[C@@H](C)Cc1cc(-c2nnc(C(F)(F)F)[nH]2)cnc1CN1CCC(c2cccc3c2O[C@](C)(c2ccc(Cl)cc2F)O3)CC1]

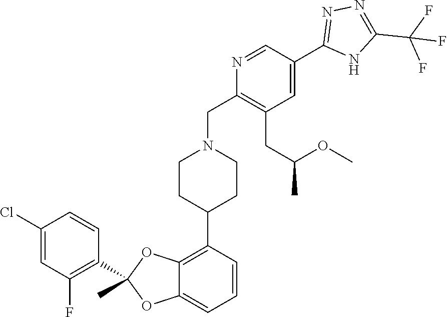 [SMILES: CO[C@@H](C)Cc1cc(-c2nnc(C(F)(F)F)[nH]2)cnc1CN1CCC(c2cccc3c2O[C@@](C)(c2ccc(Cl)cc2F)O3)CC1]

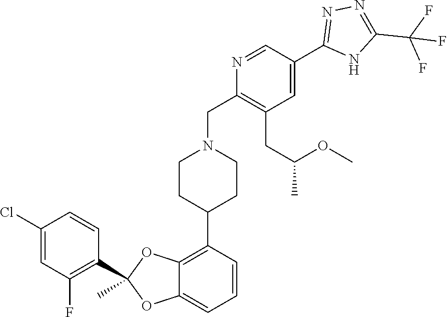 [SMILES: CO[C@H](C)Cc1cc(-c2nnc(C(F)(F)F)[nH]2)cnc1CN1CCC(c2cccc3c2O[C@](C)(c2ccc(Cl)cc2F)O3)CC1]

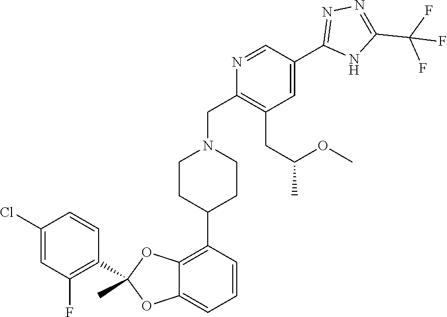 [SMILES: CO[C@H](C)Cc1cc(-c2nnc(C(F)(F)F)[nH]2)cnc1CN1CCC(c2cccc3c2O[C@@](C)(c2ccc(Cl)cc2F)O3)CC1]

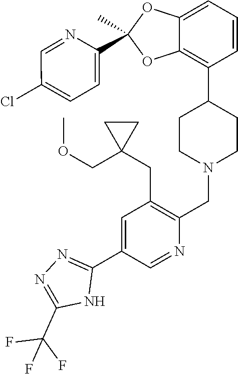 [SMILES: COCC1(Cc2cc(-c3nnc(C(F)(F)F)[nH]3)cnc2CN2CCC(c3cccc4c3O[C@@](C)(c3ccc(Cl)cn3)O4)CC2)CC1]

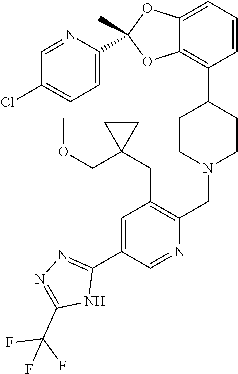 [SMILES: COCC1(Cc2cc(-c3nnc(C(F)(F)F)[nH]3)cnc2CN2CCC(c3cccc4c3O[C@](C)(c3ccc(Cl)cn3)O4)CC2)CC1]

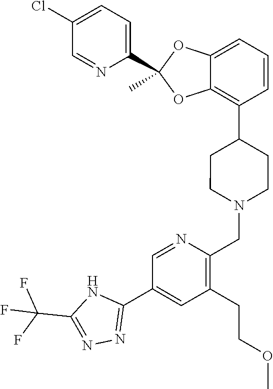 [SMILES: COCCc1cc(-c2nnc(C(F)(F)F)[nH]2)cnc1CN1CCC(c2cccc3c2O[C@@](C)(c2ccc(Cl)cn2)O3)CC1]

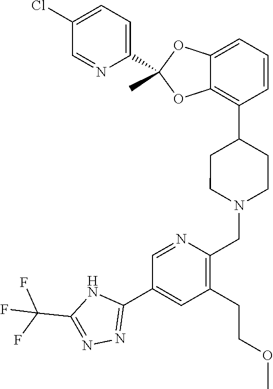 [SMILES: COCCc1cc(-c2nnc(C(F)(F)F)[nH]2)cnc1CN1CCC(c2cccc3c2O[C@](C)(c2ccc(Cl)cn2)O3)CC1]

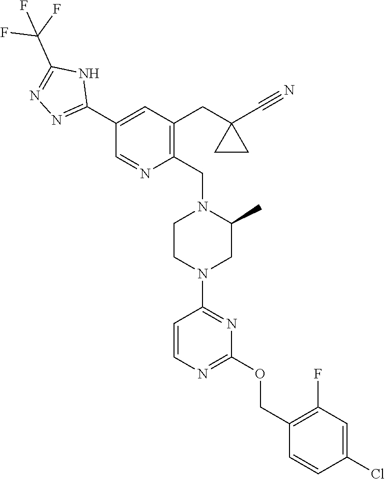 [SMILES: C[C@H]1CN(c2ccnc(OCc3ccc(Cl)cc3F)n2)CCN1Cc1ncc(-c2nnc(C(F)(F)F)[nH]2)cc1CC1(C#N)CC1]

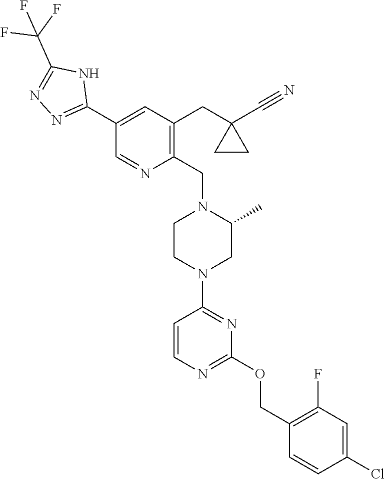 [SMILES: C[C@@H]1CN(c2ccnc(OCc3ccc(Cl)cc3F)n2)CCN1Cc1ncc(-c2nnc(C(F)(F)F)[nH]2)cc1CC1(C#N)CC1]

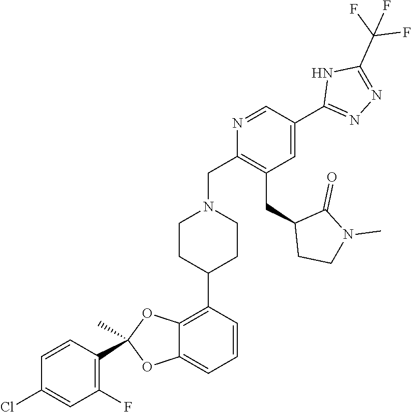 [SMILES: CN1CC[C@@H](Cc2cc(-c3nnc(C(F)(F)F)[nH]3)cnc2CN2CCC(c3cccc4c3O[C@](C)(c3ccc(Cl)cc3F)O4)CC2)C1=O]

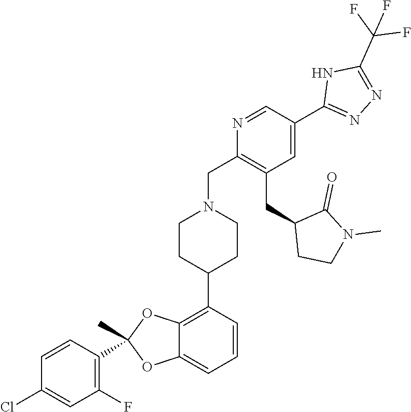 [SMILES: CN1CC[C@@H](Cc2cc(-c3nnc(C(F)(F)F)[nH]3)cnc2CN2CCC(c3cccc4c3O[C@@](C)(c3ccc(Cl)cc3F)O4)CC2)C1=O]

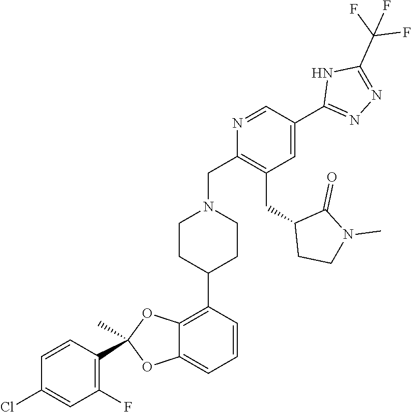 [SMILES: CN1CC[C@H](Cc2cc(-c3nnc(C(F)(F)F)[nH]3)cnc2CN2CCC(c3cccc4c3O[C@](C)(c3ccc(Cl)cc3F)O4)CC2)C1=O]

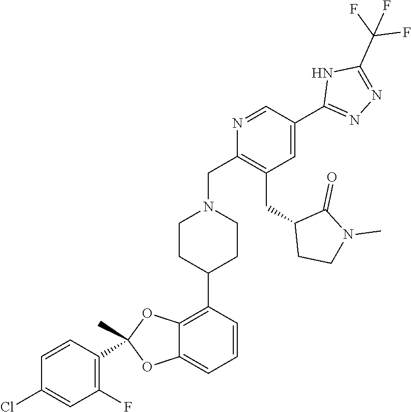 [SMILES: CN1CC[C@H](Cc2cc(-c3nnc(C(F)(F)F)[nH]3)cnc2CN2CCC(c3cccc4c3O[C@@](C)(c3ccc(Cl)cc3F)O4)CC2)C1=O]

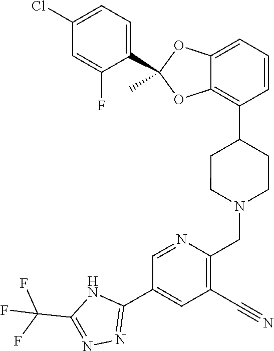 [SMILES: C[C@]1(c2ccc(Cl)cc2F)Oc2cccc(C3CCN(Cc4ncc(-c5nnc(C(F)(F)F)[nH]5)cc4C#N)CC3)c2O1]

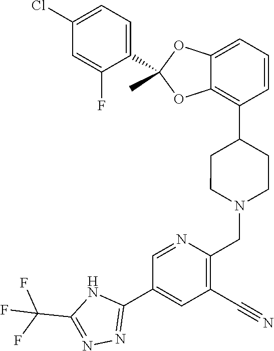 [SMILES: C[C@@]1(c2ccc(Cl)cc2F)Oc2cccc(C3CCN(Cc4ncc(-c5nnc(C(F)(F)F)[nH]5)cc4C#N)CC3)c2O1]

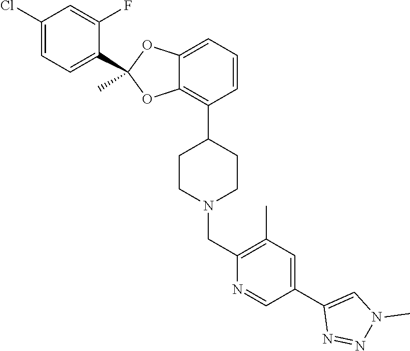 [SMILES: Cc1cc(-c2cn(C)nn2)cnc1CN1CCC(c2cccc3c2O[C@@](C)(c2ccc(Cl)cc2F)O3)CC1]

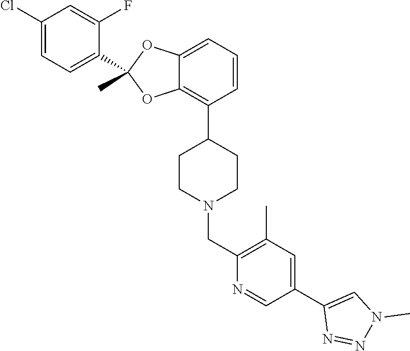 [SMILES: Cc1cc(-c2cn(C)nn2)cnc1CN1CCC(c2cccc3c2O[C@](C)(c2ccc(Cl)cc2F)O3)CC1]

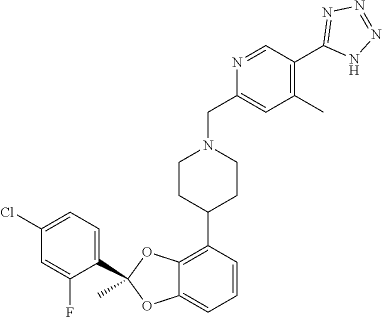 [SMILES: Cc1cc(CN2CCC(c3cccc4c3O[C@](C)(c3ccc(Cl)cc3F)O4)CC2)ncc1-c1nnn[nH]1]

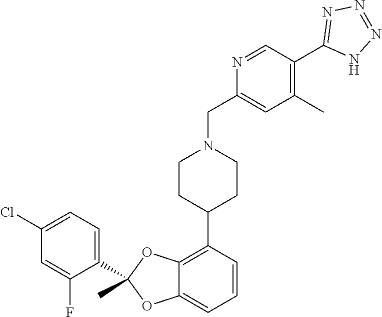 [SMILES: Cc1cc(CN2CCC(c3cccc4c3O[C@@](C)(c3ccc(Cl)cc3F)O4)CC2)ncc1-c1nnn[nH]1]

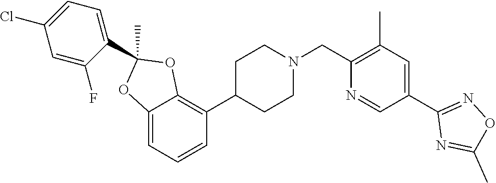 [SMILES: Cc1nc(-c2cnc(CN3CCC(c4cccc5c4O[C@](C)(c4ccc(Cl)cc4F)O5)CC3)c(C)c2)no1]

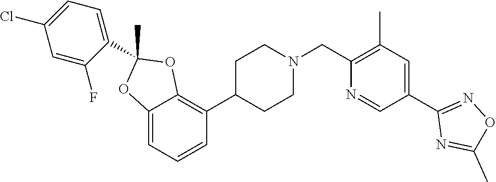 [SMILES: Cc1nc(-c2cnc(CN3CCC(c4cccc5c4O[C@@](C)(c4ccc(Cl)cc4F)O5)CC3)c(C)c2)no1]

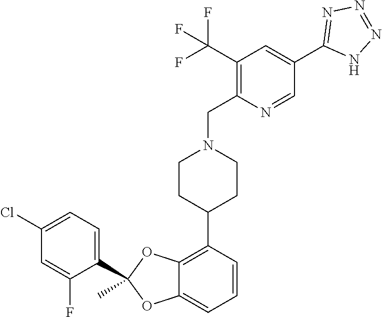 [SMILES: C[C@@]1(c2ccc(Cl)cc2F)Oc2cccc(C3CCN(Cc4ncc(-c5nnn[nH]5)cc4C(F)(F)F)CC3)c2O1]

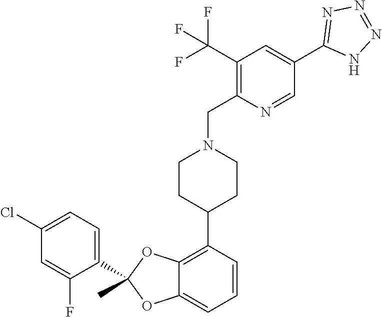 [SMILES: C[C@]1(c2ccc(Cl)cc2F)Oc2cccc(C3CCN(Cc4ncc(-c5nnn[nH]5)cc4C(F)(F)F)CC3)c2O1]

The compounds of Table 1 include pharmaceutically acceptable salts thereof. In addition, the compounds of Table 1 or 2 also include other salts of such compounds which are not necessarily pharmaceutically acceptable salts, and which may be useful as intermediates for preparing and/or purifying compounds of Table 1 or 2 and/or for separating enantiomers of compounds of Table 1 or 2. Non-limiting examples of pharmaceutically acceptable salts of compounds of Table 1 or 2 include trifluoroacetic acid salts.

It will further be appreciated that the compounds of Table 1 or 2 or their salts may be isolated in the form of solvates, and accordingly that any such solvate is included within the scope of the present disclosure. A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine. For example, compounds of Table 1 or 2 and salts thereof can exist in unsolvated as well as solvated forms with pharmaceutically acceptable solvents such as water, ethanol, and the like.

### Pharmaceutical Compositions and Administration

When employed as pharmaceuticals, compounds as described herein (e.g., a compound of Table 1, or a pharmaceutically acceptable salt or solvate thereof) can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Table 1, or a pharmaceutically acceptable salt or solvate thereof, in combination with one or more pharmaceutically acceptable excipients (carriers). For example, a pharmaceutical composition prepared using a compound of Table 1, or a pharmaceutically acceptable salt or solvate thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Table 1, or a pharmaceutically acceptable salt or solvate thereof with a pharmaceutically acceptable excipient. Pharmaceutical compositions containing a compound of Table 1, or a pharmaceutically acceptable salt or solvate thereof as the active ingredient can be prepared by intimately mixing the compound of Table 1, or a pharmaceutically acceptable salt or solvate thereof with a pharmaceutical carrier according to conventional pharmaceutical compounding techniques. The carrier can take a wide variety of forms depending upon the desired route of administration (e.g., oral, parenteral, etc.). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a route of administration is parenteral (e.g., intratumoral).

In some embodiments, a compound of Table 1, or a pharmaceutically acceptable salt or solvate thereof as described herein, or pharmaceutical compositions thereof, can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound of Table 1, or a pharmaceutically acceptable salt or solvate thereof in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing a compound of Table 1, or a pharmaceutically acceptable salt or solvate thereof, or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound of Table 1, or a pharmaceutically acceptable salt or solvate thereof, as described herein or a pharmaceutical composition thereof is formulated for local delivery to the digestive or GI tract by way of oral administration (e.g., solid or liquid dosage forms.).

In some embodiments, solid dosage forms for oral administration include capsules, tablets, pills, powders, and granules. In some embodiments, a compound of Table 1, or a pharmaceutically acceptable salt or solvate thereof, is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound of Table 1, or a pharmaceutically acceptable salt or solvate thereof as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound of Table 1, or a pharmaceutically acceptable salt or solvate thereof as described herein or a pharmaceutical composition thereof is formulated for ocular administration. In some embodiments, ocular compositions can include, without limitation, one or more of any of the following: viscogens (e.g., carboxymethylcellulose, glycerin, polyvinylpyrrolidone, polyethylene glycol); stabilizers (e.g., pluronic (triblock copolymers), cyclodextrins); preservatives (e.g., benzalkonium chloride, EDTA, SofZia (boric acid, propylene glycol, sorbitol, and zinc chloride; Alcon Laboratories, Inc.), Purite (stabilized oxychloro complex; Allergan, Inc.)).

In some embodiments, a compound of Table 1, or a pharmaceutically acceptable salt or solvate thereof as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### Formulation Example 1—Tablet Formulation

The following ingredients are mixed intimately and pressed into single scored tablets.

| Ingredient | Quantity per tablet, mg |
|---|---|
| compound of this disclosure | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule

| Ingredient | Quantity per capsule, mg |
|---|---|
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| coloring | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol®H-15 (triglycerides of saturated vegetable fatty acid; Riches-Nelson, Inc., New York), and has the following composition:

| Ingredient | Amount |
|---|---|
| compound of this disclosure | 500 mg |
| Witepsol ® H-15 | balance |

In some embodiments, the dosage for a compound of Table 1, or a pharmaceutically acceptable salt or solvate thereof, is determined based on a multiple factors including, but not limited to, type, age, weight, sex, medical condition of the patient, severity of the medical condition of the patient, route of administration, and activity of the compound or pharmaceutically acceptable salt or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound of Table 1, or a pharmaceutically acceptable salt or solvate thereof, is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound of Table 1, or a pharmaceutically acceptable salt or solvate thereof as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound of Table 1, or a pharmaceutically acceptable salt or solvate thereof as described herein is administered as a dosage of about 100 mg/Kg.

In some embodiments, the foregoing dosages of a compound of Table 1, or a pharmaceutically acceptable salt or solvate thereof, can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound of Table 1, or a pharmaceutically acceptable salt or solvate thereof as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound of Table 1, or a pharmaceutically acceptable salt or solvate thereof is administered to a patient for a period of time followed by a separate period of time where administration of the compound of Table 1, or a pharmaceutically acceptable salt or solvate thereof is stopped. In some embodiments, a compound of Table 1, or a pharmaceutically acceptable salt or solvate thereof is administered for a first period and a second period following the first period, with administration stopped during the second period, followed by a third period where administration of the compound of Table 1, or a pharmaceutically acceptable salt or solvate thereof is started and then a fourth period following the third period where administration is stopped. For example, the period of administration of a compound of Table 1, or a pharmaceutically acceptable salt or solvate thereof followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound of Table 1, or a pharmaceutically acceptable salt or solvate thereof, is orally administered to the patient one or more times per day (e.g., one time per day, two times per day, three times per day, four times per day per day or a single daily dose).

In some embodiments, a compound of Table 1, or a pharmaceutically acceptable salt or solvate thereof, is administered by parenteral administration to the patient one or more times per day (e.g., 1 to 4 times, one time per day, two times per day, three times per day, four times per day or a single daily dose).

In some embodiments, a compound of Table 1, or a pharmaceutically acceptable salt or solvate thereof, is administered by parenteral administration to the patient weekly.

### Methods of Treatment

In some embodiments, this disclosure features methods for treating a patient (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In some embodiments, the methods described herein can include or further include treating one or more conditions associated, co-morbid or sequela with any one or more of the conditions described herein.

Provided herein is a method for treating a GLP-1 associated disease, disorder, or condition, the method comprising administering to a patient in need thereof an effective amount of a compound of Table 1, or a pharmaceutically acceptable salt or solvate thereof, or a pharmaceutical composition as disclosed herein.

In some embodiments, the disease, disorder, or condition includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity (including hypothalamic obesity and monogenic obesity), weight gain from use of other agents, idiopathic intracranial hypertension, Wolfram syndrome, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease, disorder, or condition includes, but is not limited to type 2 diabetes mellitus, early onset type 2 diabetes mellitus, obesity, idiopathic intracranial hypertension, Wolfram syndrome, weight gain from use of other agents, gout, excessive sugar craving, hypertriglyceridemia, dyslipidemia, gestational diabetes, kidney disease (e.g., acute kidney disorder, tubular dysfunction, proinflammatory changes to the proximal tubules), adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease, disorder, or condition includes, but is not limited to type 2 diabetes mellitus, early onset type 2 diabetes mellitus, obesity, idiopathic intracranial hypertension, Wolfram syndrome, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of a reduction of blood glucose levels (e.g., reduce blood glucose levels), a reduction of blood hemoglobin A1c (HbA1c) levels, a promotion of insulin synthesis, a stimulation of insulin secretion, an increase in the mass of β-cells, a modulation of gastric acid secretion, a modulation of gastric emptying, a decrease in the body mass index (BMI), and/or a decrease in glucagon production (e.g., level). In some embodiments, the compounds and pharmaceutical compositions and methods for treating a patient described herein can reduce blood glucose levels, reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), decrease glucagon production (e.g., level), or any combination thereof. In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of a compound of Table 1, or a pharmaceutically acceptable salt or solvate thereof, or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of a compound of Table 1, or a pharmaceutically acceptable salt or solvate thereof, or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

### Indications

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, urine incontinence, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of a compound of Table 1, or a pharmaceutically acceptable salt or solvate thereof, or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of a compound of Table 1, or a pharmaceutically acceptable salt or solvate thereof, or a pharmaceutical composition as disclosed herein.

Also provided herein is a method of treating type 2 diabetes mellitus in a patient in need thereof, the method comprising administering to the patient a therapeutically effective amount of a compound of Table 1, or a pharmaceutically acceptable salt or solvate thereof, or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, hyperlipoproteinemia, glycogen storage diseases, Weber-Christian disease, Wolman's disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90).

Other non-limiting examples of disorders in metabolically important tissues include joint disorders (e.g., osteoarthritis, secondary osteoarthritis), steatosis (e.g., in the liver); fibrosis (e.g., in the liver); cirrhosis (e.g., in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or hemodialysis, bone fracture, bone surgery, aging, pregnancy, protection against bone fractures, and malnutrition polycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the condition, disease or disorder is a neurological disorder (e.g., neurodegenerative disorder) or a psychiatric disorder. Non-limiting examples of neurological disorders include idiopathic intracranial hypertension (IIH), brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., U.S. Publication No. 20060275288A1.

In some embodiments, the condition, disease or disorder is idiopathic intracranial hypertension. Idiopathic intracranial hypertension is characterized by increased intracranial pressure and papilloedema. See, e.g., Virdee et al. Ophthalmol Ther. 2020; 9(4):767-781. In some embodiments, the compounds and pharmaceutical compositions and methods described herein reduce cerebrospinal fluid secretion in a patient with idiopathic intracranial hypertension. In some embodiments, the compounds and pharmaceutical compositions and methods described herein reduce intracranial pressure in a patient with idiopathic intracranial hypertension. In some embodiments, the compounds and pharmaceutical compositions and methods described herein reduce one or more symptoms in a patient with idiopathic intracranial hypertension. Symptoms of idiopathic intracranial hypertension can include severe headaches and visual impairment. In some embodiments, the patient with idiopathic intracranial hypertension is female. In some embodiments, the patient with idiopathic intracranial hypertension is about 20 to about 30 years old. In some embodiments, the patient with idiopathic intracranial hypertension is obese.

In some embodiments, the condition, disease or disorder is Wolfram syndrome. Wolfram syndrome is caused by biallelic mutations of the Wolframin ER transmembrane glycoprotein (Wfs1) gene. See, e.g., Seppa et al. Sci Rep 9, 15742 (2019). Wolfram syndrome can first appear as diabetes mellitus, followed by optic nerve atrophy, deafness, and symptoms of neurodegeneration. Patients with Wolfram syndrome can have symptoms of ataxia, sleep apnea, dysphagia, hearing loss, and loss of taste due to brainstem atrophy. In some embodiments, the compounds and pharmaceutical compositions and methods described herein reduce neuroinflammation in a patient with Wolfram syndrome. In some embodiments, the neuroinflammation is reduced in the inferior olive in the patient. In some embodiments, the compounds and pharmaceutical compositions and methods described herein reduce retinal ganglion cell death in a patient with Wolfram syndrome. In some embodiments, the compounds and pharmaceutical compositions and methods described herein reduce axonal degeneration in a patient with Wolfram syndrome. In some embodiments, the compounds and pharmaceutical compositions and methods described herein reduce one or more symptoms (e.g., any of the symptoms described herein) in a patient with Wolfram syndrome.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., U.S. Publication No. 20120021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson. Insulin-Related Conditions and Disorders

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperprolactinemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves' disease. See, e.g., U.S. Publication No. 20120148586A1.

### Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking. In some embodiments, the weight gain is induced by the use of steroids or antipsychotics.

In some embodiments, the condition, disease or disorder is an eating disorder, such as hyperphagia, binge eating, bulimia, compulsive eating, or syndromic obesity such as Prader-Willi and Bardet-Biedl syndromes.

### Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and

Cushing's disease.

In some embodiments, the condition, disease or disorder associated with diabetes is related to the hypothalamic-pituitary-gonadal axis.

### Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

### Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds of Table 1, or a pharmaceutically acceptable salt or solvate thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), peptide YY or an analogue thereof, cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498, naltrexone), orexin receptor antagonists, melanocortin 4 receptor agonists, 11β-hydroxysteroid dehydrogenase inhibitors (e.g., AZD-4017, BVT-3498, INCB-13739), pancreatic lipase inhibitors (e.g., orlistat, cetilistat), P3 agonists (e.g., N-5984), diacylglycerol acyltransferase 1 (DGAT1) inhibitors, acetylCoA carboxylase (ACC) inhibitors (e.g., compounds described in International Publication Nos. WO 2020/234726, WO 2020/044266, and U.S. Pat. No. 8,859,577), stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin, empagliflozin, canagliflozin, ipragliflozin, tofogliflozin, sergliflozin etabonate, remogliflozin etabonate, or ertugliflozin), SGLT-1 inhibitors, MCR-4 agonists, monoamine reuptake inhibitors, melanocytestimulating hormone analogs, 5HT2c agonists, galanin antagonists, anorectic agents (such as a bombesin agonist), thyromimetic agents, dehydroepiandrosterone or analogs thereof, human agouti-related protein (AGRP) inhibitors, neuromedin U agonists, NFK inhibitors (e.g., HE-3286), PPAR agonists (e.g., GFT-505, DRF-11605, gemfibrozil, fenofibrate, balaglitazone, ciglitazone, darglitazone, englitazone, isaglitazone, pioglitazone, rosiglitazone, CLX-0940, GW-1536, GW-1 929, GW-2433, KRP-297, L-796449, LR-90, MK-0767, and SB-21 9994), phosphotyrosine phosphatase inhibitors (e.g., sodium vanadate, trodusquemin), GPR119 agonists (e.g., PSN-821, MBX-2982, APD597, compounds described in International Publication Nos. WO 2010/140092, WO 2010/128425, WO 2010/128414, WO 2010/106457), glucokinase activators (e.g., piragliatin, AZD-1656, AZD6370, TTP-355, TTP-399, TTP547, ARRY403, MK-0599, TAK-329, AZD5658 or GKM-001 compounds described in International Publication Nos. WO 2010/103437, WO 2010/103438, WO 2010/013161, WO 2007/122482, WO 2006/112549, WO 2007/028135, WO 2008/047821, WO 2008/050821, WO 2008/136428 and WO 2008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), melanocortin receptor 4 agonist (e.g., setmelanotide), melanin concentrating hormone receptor 1 antagonist, serotonergic agents (e.g. sibutramine, lorcaserin), farnesoid X receptor (FXR) agonist (e.g., obeticholic acid, tropifexor, cilofexor, LY2562175, Met409, TERN-101, EDP305, compounds described in International Publication Nos. WO 2020/234726 and WO 2020/044266), phentermine, zonisamide, norepinephrine/dopamine reuptake inhibitor (e.g., buproprion), GDF-15 analog, methionine aminopeptidase 2 (MetAP2) inhibitor (e.g., beloranib or ZGN-1061), diethylpropion, phendimetrazine, benzphetamine, fibroblast growth factor receptor (FGFR) modulator, biotin, a MAS receptor modulator, glucagon receptor agonist, CCKa agonists (e.g., compounds described in International Publication No. WO 2005/116034 and U.S. Publication No. 2005/0287100), and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, glimepiride, tolbutamide, glibenclamide, gliclazide, acetohexamide, glyclopyramide, glybuzole, glyburide, glipizide), thiazolidinedione agents (e.g. rosiglitazone, lobeglitazone, troglitazone, balaglitazone, rivoglitazone, lobeglitazone or pioglitazone), glitazars (e.g., aleglitazar, chiglitazar, saroglitazar, muraglitazar, tesaglitazar), SGLT2 inhibitors (e.g., JNJ-28431754, dapagliflozin, AVE2268, TS-033, YM543, TA-7284, ASP1941, THR1474, TS-071, ISIS388626, LX4211, remogliflozin, empagliflozin, canagliflozin, ipragliflozin, tofogliflozin, sergliflozin etabonate, remogliflozin etabonate, ertugliflozin, compounds described in WO 2010/023594), GPR40 agonists (e.g., a FFAR1/FFA1 agonist, e.g. fasiglifam), α-glucosidase inhibitors (e.g., adiposin, camiglibose, pradimicin-Q, salbostatin, voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, anagliptin (SK-0403), teneligliptin, omarigliptin, B11356, GRC8200, MP-513, PF-00734200, PHX1149, ALS2-0426, TA-6666, TS-021, KRP-104, trelagliptin).

In some embodiments, the one or more additional therapeutic agents include those useful, for example, for treating NAFL and NASH. Non-limiting examples include FXR agonists (e.g., obeticholic acid), PF-05221304, PPAR a/S agonists (e.g., elafibranor), a synthetic fatty acid-bile conjugate (e.g., aramchol), an anti-lysyl oxidase homologue 2 (LOXL2) monoclonal antibody (e.g., simtuzumab), a caspase inhibitor (e.g., emricasan), a MAPK5 inhibitor, a galectin 3 inhibitor (e.g., GR-MD-02), a fibroblast growth factor 21 (FGF21) (e.g., BMS-986036), a niacin analogue (e.g., ARJ 3037MO), a leukotriene D4 (LTD4) receptor antagonist (e.g., tipelukast), an acetyl-CoA carboxylase (ACC) inhibitor (e.g., NDI 010976 and compounds described in International Publication Nos. WO 2009/144554, WO 2003/072197, WO 2009/144555, and WO 2008/065508), a ketohexokinase (KHK) inhibitor (e.g., compounds described in WO 2020/234726), an apoptosis signal-regulating kinase 1 (ASKI) inhibitor (selonsertib), an ileal bile acid transporter (IBAT) inhibitor, a dual antagonist of chemokine receptor 2 (CCR2) and CCR5 (e.g., cenicriviroc), diacylglyceryl acyltransferase 2 (DGAT2) inhibitor (e.g., compounds described in WO 2020/234726 and U.S. Publication No. 20180051012), a CB1 receptor antagonist, an anti-CB1R antibody, glycyrrhizin, schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone, balaglitazone, rivoglitazone, lobeglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (γ-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, zofenopril, fbsinopril, enalapril, ceranopril, cilazopril, delapril, pentopril, quinapril, ramipril, lisinopril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and β-blockers (e.g., metoprolol, atenolol, propranolol, carvedilol, pindolol). Further non-limiting examples of antihypertensive agents include: diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone), alpha adrenergic blockers, beta adrenergic blockers, calcium channel blockers (e.g., diltiazem, verapamil, nifedipine and amlodipine), vasodilators (e.g., hydralazine), renin inhibitors, AT-1 receptor antagonists (e.g., losartan, irbesartan, valsartan), ET receptor antagonists (e.g., sitaxsentan, atrsentan, compounds disclosed in U.S. Pat. Nos. 5,612,359 and 6,043,265), dual ET/AII antagonist (e.g., compounds disclosed in WO 2000/01389), neutral endopeptidase (NEP) inhibitors, If channel blocker ivabradinand, vasopepsidase inhibitors (dual NEP-ACE inhibitors) (e.g., gemopatrilat and nitrates).

In some embodiments, the one or more additional therapeutic agents include those useful, for example, as diuretics. Non-limiting examples include xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran, boroarginine derivatives, boropeptides, heparins, hirudin, and melagatran), FXa inhibitors (e.g., rivaroxaban, apixaban, edoxaban, betrixaban, YM150, compounds described in WO02/06234, WO2004/048363, WO2005/030740, WO2005/058823, and WO2005/113504) thrombolytic agents (e.g., anistreplase, streptokinase, tenecteplase (TNK), lanoteplase (nPA), urokinase, tisokinase, alteplase, nateplase, monteplase, pamiteplase, factor VIla inhibitors, PAT-1 inhibitors, alpha2-antiplasmin inhibitors, and anisoylated plasminogen streptokinase activator complex), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin, pravastatin, pitavastatin, lovastatin, atorvastatin, simvastatin, fluvastatin, itavastatin, ZD-4522), HMG-CoA synthase inhibitors, cholesterol-lowering agents, bile acid sequestrants (e.g., cholestyramine, questran, colestipol, and colesevelam), cholesterol absorption inhibitors (e.g. plant sterols such as phytosterols), cholesteryl ester transfer protein (CETP) inhibitors, inhibitors of the ileal bile acid transport system (IBAT inhibitors), diacylglyceryl acyltransferase 1 (DGAT1) inhibitors (e.g., AZD7687, LCQ908, compounds described in WO 2009/016462, WO 2010/086820), monoacylglycerol O-acyltransferase inhibitors, α-amylase inhibitors (e.g., tendamistat, trestatin, AL-3688), α-glucoside hydrolase inhibitors, SIRT-1 activators, c-Jun N-terminal kinase (JNK) inhibitors, a VPAC2 receptor agonist, TGR5 receptor modulators (e.g., compounds described in), GPBARI receptor modulators, GPR120 modulators, high affinity nicotinic acid receptor (HM74A) activators, carnitine palmitoyl transferase enzyme inhibitors, mineralocorticoid receptor inhibitors, inhibitors of TORC2, fatty acid synthetase inhibitors, serine palmitoyl transferase inhibitors, GPR81 modulators, GPR39 modulators, GPR43 modulators, GPR41 modulators, GPR105 modulators, KvL.3 modulators, retinol binding protein 4 modulators, somatostain receptor modulators, PDHK2 modulators, PDHK4 modulators, MAP4K4 inhibitors, IL1 family modulators (e.g., ILI beta modulators), ACAT inhibitors, MTP inhibitors (e.g., diriotapide, mitratapide, and implitapide), lipooxygenase inhibitors, PCSK9 modulators (e.g., alirocumab and evolocumab), RXRalpha modulators, cysteamine, cystamine, an RNA antisense construct to inhibit protein tyrosine phosphatase PTPRU, vitamin B complex, pentraxin proteins, a protein tyrosine phosphatase-1 B (PTP-1 B) inhibitor (e.g., trodusquemine, hyrtiosal extract, and compounds described by Zhang et al. Drug Discovery Today. 2007, 12(9-10): 373-381), ezitimbe, betaine, pentoxifylline, alpha delta-9 desaturase, BCKDK inhibitors, branched-chain alpha keto acid dehydrogenase kinase (BCBK) inhibitors, PNPLA3 inhibitors, FGF1 9 analogs, SCD1 inhibitors, bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone, spironolactone), centrally acting adrenergic drugs, such as central alpha agonists (e.g. clonidine), diuretic agents (e.g. furosemide, torsemide, bemetanide, ethacrynic acid, thiazide-type diuretics (e.g., chlorothiazide, hydrochlorothiazide, benzthiazide, hydroflumethiazide, bendroflumethiazide, methychlorthiazide, polythiazide, trichlormethiazide, indapamide), phthalimidine-type diuretics (e.g., chlorthalidone, metolazone), quinazoline-type diuretics (e.g., quinethazone), potassium-sparing diuretics (e.g., triamterene and amiloride), thyroid receptor agonists (e.g., compounds described in WO 2020/117987), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin), non-steroidal anti-inflammatory drugs (NSAIDS), thromboxane-A2-receptor antagonists (e.g., ifetroban), thromboxane-A2-synthetase inhibitors, PDE inhibitors (e.g., Pletal, dipyridamole)), antagonists of purinergic receptors (e.g., P2Y1 and P2Y12), adenosine diphosphate (ADP) receptor inhibitors (e.g. clopidogrel), phosphodiesterase inhibitors (e.g. cilostazol), glycoprotein IIB/IIA inhibitors (e.g. tirofiban, eptifibatide, and abcixima), adenosine reuptake inhibitors (e.g. dipyridamole), noradrenergic agents (e.g. phentermine), serotonergic agents (e.g. sibutramine, lorcaserin), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in WO 2007/013694, WO 2007/018314, WO 2008/093639 and WO 2008/099794, GPR40 agonists (e.g., fasiglifam or a hydrate thereof, compounds described in WO 2004/041266, WO 2004/106276, WO 2005/063729, WO 2005/063725, WO 2005/087710, WO 2005/095338, WO 2007/013689 and WO 2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor a (PPARa), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., K⁺ channel openers, mexiletine, propafenone, metoprolol, atenolol, carvadiol, propranolol, sotalol, dofetilide, amiodarone, azimilide, ibutilide, ditiazem, and verapamil), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), metaglidasen, AMG-131, balaglitazone, MBX-2044, rivoglitazone, aleglitazar, chiglitazar, saroglitazar, muraglitazar, tesaglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide-, piroxicam, NO donating agents (e.g., organonitrates), NO promoting agents (e.g., phosphodiesterase inhibitors).

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). While not wishing to be bound by theory, it is believed that administering one or more anti-emetic agents in combination with the Table 1 compounds described herein may allow higher dosages of the Table 1 compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, and cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: Granisetron (Kytril), Dolasetron, Ondansetron (Zofran), Tropisetron, Ramosetron, Palonosetron, Alosetron, azasetron, Bemesetron, Zatisetron, Batanopirde, MDL-73147EF; Metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, Tropanyl-3,5-dimethylbenzoate, 3-(4-Allylpiperazin-1-yl)-2-quinoxalinecarbonitrile maleate, Zacopride hydrochloride, and Mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+netupitant), quetiapine, qamosetron, ramosteron, ricasetron, risperidone, ziprasidone, and zatosetron.

In certain embodiments, the 5HT-3-receptor antagonist is Granisetron, Dolasetron, Ondansetron hydrochloride, Tropisetron, Ramosetron, Palonosetron, Alosetron, Bemesetron, Zatisetron, Batanopirde, MDL-73147EF, Metoclopramide, N-3389, Y—25130 hydrochloride, MDL 72222, Tropanyl-3,5-dimethylbenzoate 3-(4-Allyl-piperazin-1-yl)-2-quinoxalinecarbonitrile maleate, Zacopride hydrochloride and Mirtazepine.

In certain embodiments, the 5HT-3-receptor antagonist is Granisetron, Dolasetron, Ondansetron hydrochloride, Tropisetron, Ramosetron, Palonosetron, Alosetron, Bemesetron, and Zatisetron.

In certain embodiments, the 5HT-3-receptor antagonist is Granisetron, Dolasetron and Ondansetron.

In certain embodiments, the 5HT-3-receptor antagonist is Granisetron.

In certain embodiments, the 5HT-3-receptor antagonist is Ondansetron.

In some embodiments, the anti-emetic agent is an antihistamine. Non-limiting examples of antihistamines include: piperazine derivatives (e.g., cyclizine, meclizine, and cinnarizine); Promethazine; Dimenhydrinate (Dramamine, Gravol); Diphenhydramine; Hydroxyzine; Buclizine; and Meclizine hydrochloride (Bonine, Antivert), doxylamine, and mirtazapine.

In some embodiments, the anti-emetic agent is an anticholinergic agent (Inhibitors of the acetylcholine receptors). Non-limiting examples of anticholinergic agents include: atropine, Scopolamine, Glycopyrron, Hyoscine, Artane (Trihexy-5 trihexyphenidyl hydrochloride), Cogentin (benztropine mesylate), Akineton (biperiden hydrochloride), Disipal (Norflex orphenadrine citrate), diphenhydramine, hydroxyzine, hyoscyamine, and Kemadrin (procyclidine hydrochloride).

In some embodiments, the anti-emetic agent is a steroid (e.g., a corticosteroid). Non-limiting examples of steroids include: betamethasone, Dexamethasone, Methylprednisolone, Prednisone®, and Trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopa k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione], PD 154075, ([(2-benzofuran)-CH₂OCO]—(R)-alpha-MeTrp-(S)—NHCH(CH₃) Ph), FK888, and (D-Pro4, D-Trp7,9,10, Phell)SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine,); benzamides (e.g., Metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., Cannabidiol (CBD), Cannabidiol dimethylheptyl (CBD-DMH), Tetra-hydro-cannabinol (THC), Cannabinoid agonists such as WIN 55-212 (a CB1 and CB2 receptor agonist), Dronabinol (Marinol®), and Nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and Prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B6; peppermint, lavender, and lemon essential oils; and ginger.

Still other exemplary anti-emetic agents include those disclosed in US 20120101089A1; U.S. Pat. No. 10,071,088 B2; U.S. Pat. No. 6,673,792 B1; U.S. Pat. No. 6,197,329 B1; U.S. Pat. No. 10,828,297 B2; U.S. Pat. No. 10,322,106 B2; U.S. Pat. No. 10,525,033 B2; WO 2009080351 A1; WO 2019203753 A2; WO 2002020001 A2; U.S. Pat. No. 8,119,697 B2; U.S. Pat. No. 5,039,528; US20090305964A1; and WO 2006/111169, each of which is incorporated by reference in its entirety.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

### Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin A1c (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m2 to greater than or about 100 kg/m2. In some embodiments, the BMI of the patient is greater than or about 30 kg/m2 to greater than or about 90 kg/m2. In some embodiments, the BMI of the patient is greater than or about 40 kg/m2 to greater than or about 80 kg/m2. In some embodiments, the BMI of the patient is greater than or about 50 kg/m2 to greater than or about 70 kg/m2.

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

### Examples

General information: All evaporations were carried out in vacuo with a rotary evaporator. Analytical samples were dried in vacuo (1-5 mm Hg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. 1H chemical shifts are reported in 6 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

This disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

### Example 1: 1-{[2-({4-[2-(2,4-dichlorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (1)

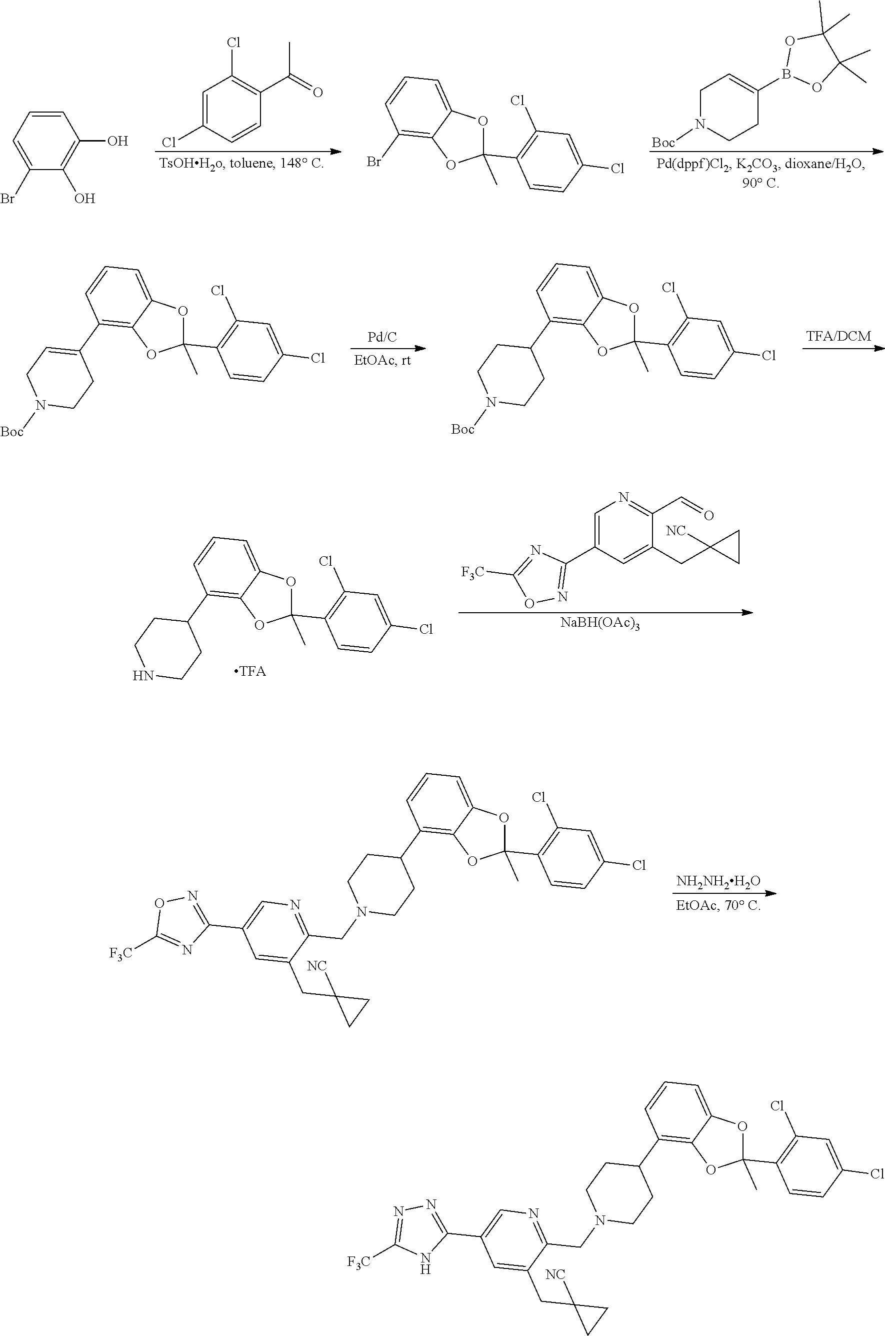

Step A: 4-bromo-2-(2,4-dichlorophenyl)-2-methylbenzo[d][1,3]dioxole. A mixture of 3-bromobenzene-1,2-diol (15.0 g, 79.8 mmol), 1-(2,4-dichlorophenyl)ethan-1-one (18.6 g, 95.9 mmol) and TsOH·H₂O (3.0 g, 15.8 mmol) in toluene (120 mL) was stirred at 148° C. under Argon for 48 h. The mixture was filtered, and the residue was purified by column chromatography on silica gel to give 4-bromo-2-(2,4-dichlorophenyl)-2-methylbenzo[d][1,3]dioxole (11.2 g, yield: 39.3%).

Step B: tert-butyl 4-(2-(2,4-dichlorophenyl)-2-methylbenzo[d][1,3]dioxol-4-yl)-3,6-dihydropyridine-1(2H)-carboxylate. A mixture of 4-bromo-2-(2,4-dichlorophenyl)-2-methylbenzo[d][1,3]dioxole (5.0 g, 14.0 mmol), tert-butyl 4-(4,4,5,5-tetramethyl-1,3,2-dioxaborolan-2-yl)-3,6-dihydropyridine-1(2H)-carboxylate (4.3 g, 14.0 mmol), Pd(dppf)Cl₂ (2.0 g, 2.9 mmol) and K₂CO₃ (7.7 g, 55.9 mmol) in 1,4-dioxane/H₂O (100 mL/10 mL) was stirred at 90° C. under Argon for 16 h. The mixture was filtered, and the residue was purified by column chromatography on silica gel to give tert-butyl 4-(2-(2,4-dichlorophenyl)-2-methylbenzo[d][1,3]dioxol-4-yl)-3,6-dihydropyridine-1(2H)-carboxylate (4.6 g, yield: 71.3%).

Step C: tert-butyl 4-(2-(2,4-dichlorophenyl)-2-methylbenzo[d][1,3]dioxol-4-yl)piperidine-1-carboxylate. A mixture of tert-butyl 4-(2-(2,4-dichlorophenyl)-2-methylbenzo[d][1,3]dioxol-4-yl)-3,6-dihydropyridine-1(2H)-carboxylate (1.0 g, 2.2 mmol) and Pd/C (10% w/w, 100 mg) in EtOAc (100 mL) was stirred at room temperature under H₂ for 16 h. The mixture was filtered, and the filtrate was concentrated to give tert-butyl 4-(2-(2,4-dichlorophenyl)-2-methylbenzo[d][1,3]dioxol-4-yl)piperidine-1-carboxylate (1.0 g). MS Calcd: 463.1; MS Found: 408.2 [M+H−56]⁺.

Step D: 4-(2-(2,4-dichlorophenyl)-2-methylbenzo[d][1,3]dioxol-4-yl)piperidine. A mixture of tert-butyl 4-(2-(2,4-dichlorophenyl)-2-methylbenzo[d][1,3]dioxol-4-yl)piperidine-1-carboxylate (75 mg, 0.16 mmol) and TFA (0.5 mL) in DCM (3 mL) was stirred at room temperature for 1 h. The mixture was concentrated to give 4-(2-(2,4-dichlorophenyl)-2-methylbenzo[d][1,3]dioxol-4-yl)piperidine (56 mg, yield: 96.8%). MS Calcd: 363.1; MS Found: 364.1 [M+H]⁺.

Step E: 1-((2-((4-(2-(2,4-dichlorophenyl)-2-methylbenzo[d][1,3]dioxol-4-yl)piperidin-1-yl)methyl)-5-(5-(trifluoromethyl)-1,2,4-oxadiazol-3-yl)pyridin-3-yl)methyl)cyclopropane-1-carbonitrile. A mixture of 4-(2-(2,4-dichlorophenyl)-2-methylbenzo[d][1,3]dioxol-4-yl)piperidine (56 mg, 0.155 mmol) and TEA (78 mg, 0.78 mmol) in DCM (5 mL) was stirred at rt for 0.5 h. 3-((methylsulfonyl)methyl)-5-(5-(trifluoromethyl)-1,2,4-oxadiazol-3-yl)picolinaldehyde (50 mg, 0.15 mmol) was added and the mixture was stirred for 2 h. NaBH(OAc)₃ (132 mg, 0.62 mmol) was added and the mixture was stirred at rt for 16 h. The mixture was poured into water (50 mL) and extracted with DCM (2×50 mL), the combined organic layer was washed with brine, dried over sodium sulfate, filtered and the filtrate was concentrated. The residue was purified by TLC gel to give 1-((2-((4-(2-(2,4-dichlorophenyl)-2-methylbenzo[d][1,3]dioxol-4-yl)piperidin-1-yl)methyl)-5-(5-(trifluoromethyl)-1,2,4-oxadiazol-3-yl)pyridin-3-yl)methyl)cyclopropane-1-carbonitrile (70 mg, yield: 67.9%). MS Calcd: 669.2; MS Found: 670.3 [M+H]⁺.

Step F: 1-{[2-({4-[2-(2,4-dichlorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (1). A mixture of 1-((2-((4-(2-(2,4-dichlorophenyl)-2-methylbenzo[d][1,3]dioxol-4-yl)piperidin-1-yl)methyl)-5-(5-(trifluoromethyl)-1,2,4-oxadiazol-3-yl)pyridin-3-yl)methyl)cyclopropane-1-carbonitrile (70 mg, 0.10 mmol) and NH₂NH₂·H₂O (0.5 mL) in EtOH (2 mL) was stirred at 70° C. for 1 h. The mixture was purified by prep-HPLC to give 1-{[2-({4-[2-(2,4-dichlorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (1) (25.3 mg, yield: 35.7%). MS Calcd: 668.2; MS Found: 669.3 [M+H]⁺. ¹H NMR (400 MHz, CD₃OD): δ 9.21 (s, 1H), 8.54 (s, 1H), 7.77 (d, J=8 Hz, 1H), 7.55 (s, 1H), 7.36 (dd, J=14.4 Hz, 6 Hz, 1H), 6.85-6.75 (m, 3H), 4.60-4.40 (m, 2H), 3.65-3.47 (m, 2H), 3.13-2.95 (m, 5H), 2.40-2.17 (m, 2H), 2.11 (s, 3H), 2.10-1.98 (m, 2H), 1.44-1.40 (m, 2H), 1.32-1.25 (m, 2H). ¹⁹F NMR (377 MHz, CD₃OD): δ −65.31.

### Examples 2 and 3: 1-{[2-({4-[2-(2,4-dichlorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (2) (enantiomer 1) and 1-{[2-({4-[2-(2,4-dichlorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (3) (enantiomer 2)

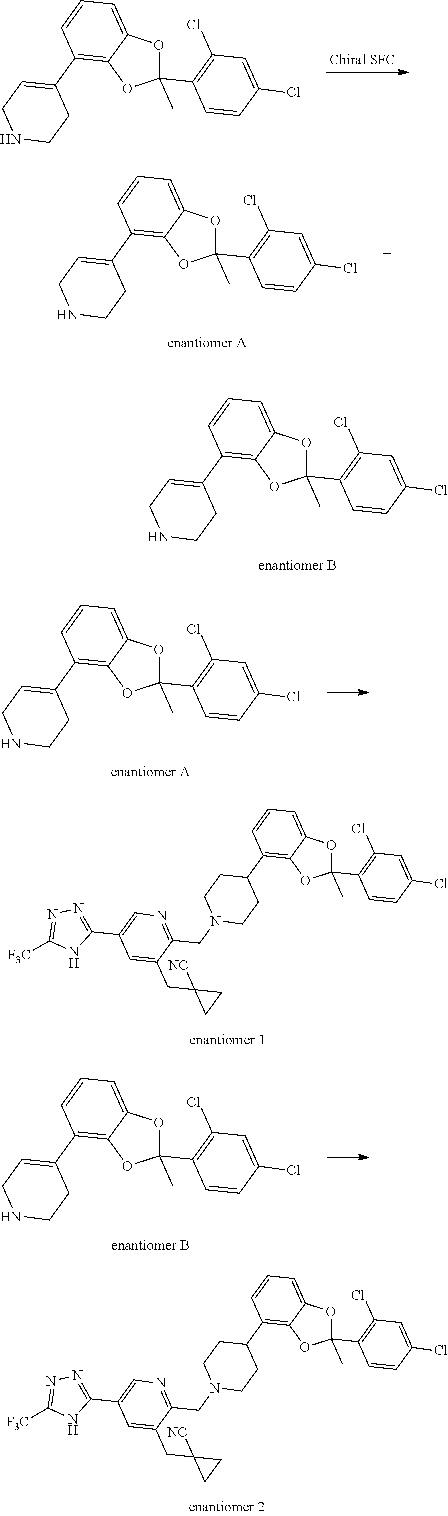

Step A: (4-(2-(2,4-dichlorophenyl)-2-methylbenzo[d][1,3]dioxol-4-yl)-1,2,3,6-tetrahydropyridine (enantiomer A and enantiomer B). (4-(2-(2,4-dichlorophenyl)-2-methylbenzo[d][1,3]dioxol-4-yl)-1,2,3,6-tetrahydropyridine was submitted for chiral separation to obtain two enantiomers (DAICEL CHIRALCEL®AD, Supercritical CO₂/IPA(+0.1% 7.0 mol/L ammonia in IPA)=85/15). Enantiomer A: MS Calcd.: 361.1; MS Found: 362.2 [M+H]⁺. SFC Retention Time: 3.40 min. Enantiomer B: MS Calcd.: 361.1; MS Found: 362.1 [M+H]⁺. SFC Retention Time: 3.89 min.

Step B: 1-{[2-({4-[2-(2,4-dichlorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (enantiomer 1 and enantiomer 2). 1-{[2-({4-[2-(2,4-dichlorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (2, enantiomer 1) (2.2 mg) was obtained with the similar method of Example 1. MS Calcd: 668.2; MS Found: 669.2 [M+H]⁺. ¹H NMR (400 MHz, CD₃OD): δ 9.24 (s, 1H), 8.54 (s, 1H), 7.76 (d, J=8.4 Hz, 1H), 7.56 (s, 1H), 7.36 (dd, J₁=8.4 Hz/J₂=6.4 Hz, 1H), 6.86-6.75 (m, 3H), 4.63 (s, 2H), 3.70-3.60 (m, 2H), 3.30-3.00 (m, 5H), 2.39-2.18 (m, 2H), 2.18-2.03 (m, 5H), 1.47-1.41 (m, 2H), 1.30-1.25 (m, 2H). ¹⁹F NMR (377 MHz, CD₃OD): δ −65.79, −79.63.

1-{[2-({4-[2-(2,4-dichlorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (3, enantiomer 2) was prepared using the similar procedure. MS Calcd: 668.2; MS Found: 669.2 [M+H]⁺. ¹H NMR (400 MHz, CD₃OD): δ 9.23 (s, 1H), 8.53 (s, 1H), 7.76 (d, J=8.8 Hz, 1H), 7.56 (s, 1H), 7.36 (dd, J=8.8 Hz/J₂=6.4 Hz, 1H), 6.86-6.74 (m, 3H), 4.57 (s, 2H), 3.66-3.58 (m, 2H), 3.21-3.10 (m, 2H), 3.08 (s, 2H), 3.08-2.98 (m, 1H), 2.35-2.18 (m, 2H), 2.18-2.00 (m, 5H), 1.45-1.40 (m, 2H), 1.29-1.22 (m, 2H). ¹⁹F NMR (377 MHz, CD₃OD): δ −65.45.

### Example 4: 1-{[2-({4-[2-(2-chloro-4-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (4)

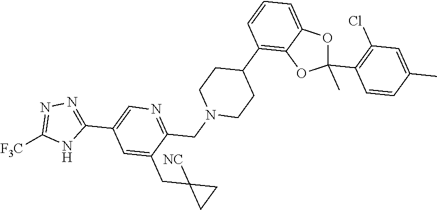 [SMILES: Cc1ccc(C2(C)Oc3cccc(C4CCN(Cc5ncc(-c6nnc(C(F)(F)F)[nH]6)cc5CC5(C#N)CC5)CC4)c3O2)c(Cl)c1]

1-{[2-({4-[2-(2-chloro-4-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (4) (15.5 mg) was obtained with the similar method of Example 1. MS Calcd: 652.2; MS Found: 653.4 [M+H]⁺. ¹H NMR (400 MHz, CD₃OD): δ 9.20 (s, 1H), 8.52 (s, 1H), 7.81 (dd, J₁=6.0 Hz/J 8.8 Hz, 1H), 7.31 (dd, J=2.4 Hz/J=8.8 Hz, 1H), 7.13-7.07 (m, 1H), 6.85-6.73 (m, 3H), 4.46 (s, 2H), 3.59-3.46 (m, 2H), 3.15-2.90 (m, 5H), 2.31-2.15 (m, 2H), 2.11 (s, 3H), 2.09-1.94 (m, 2H), 1.47-1.40 (m, 2H), 1.32-1.26 (m, 2H). ¹⁹F NMR (377 MHz, CD₃OD): δ −65.17, −113.07.

### Example 5: 1-{[2-({4-[2-(2,4-difluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (5)

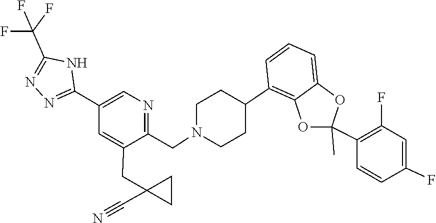 [SMILES: CC1(c2ccc(F)cc2F)Oc2cccc(C3CCN(Cc4ncc(-c5nnc(C(F)(F)F)[nH]5)cc4CC4(C#N)CC4)CC3)c2O1]

1-{[2-({4-[2-(2,4-Difluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (5) (31.2 mg) was obtained with the similar method of Example 1. MS Calcd: 636.2; MS Found: 637.4 [M+H]⁺. ¹H NMR (400 MHz, CD₃OD): δ 9.22 (s, 1H), 8.52 (s, 1H), 7.70-7.62 (m, 1H), 7.05 (t, J=11.2 Hz, 1H), 6.97 (t, J₁=8.4 Hz, 1H), 6.88-6.72 (m, 3H), 4.54 (s, 2H), 3.59 (d, J=12.0 Hz, 2H), 3.20-2.96 (m, 5H), 2.30-2.05 (m, 7H), 1.46-1.40 (m, 2H), 1.30-1.25 (m, 2H). ¹⁹F NMR (377 MHz, CD₃OD): δ −65.37, −110.63.

### Example 6: 1-((2-((4-(2-(2-Chloro-4-methylphenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile (6)

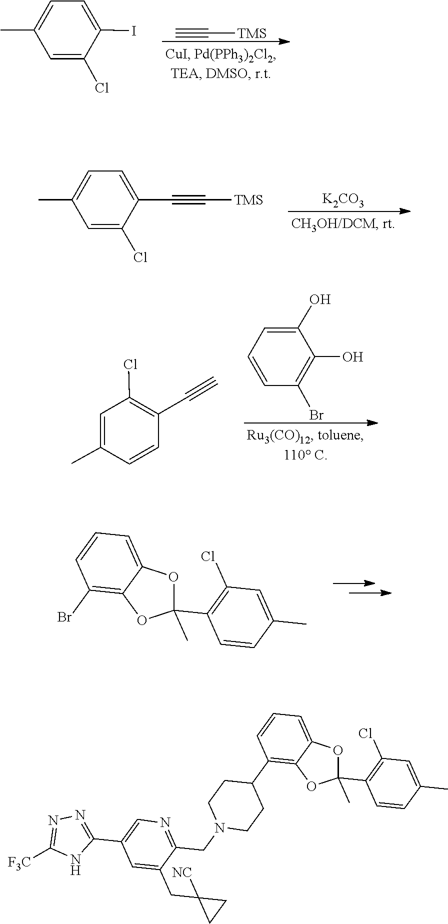

Step A: ((2-Chloro-4-methylphenyl)ethynyl)trimethylsilane. A mixture of 2-chloro-1-iodo-4-methylbenzene (5.0 g, 19.8 mmol), ethynyltrimethylsilane (1.9 g, 19.4 mmol), CuI (189 mg, 0.99 mmol), Pd(PPh₃)₂Cl₂ (1393 mg, 1.98 mmol) and TEA (3.5 mL) in DMSO (30 mL) was stirred at room temperature under Argon for 2 h. The mixture was poured into water (300 mL) and extracted with EtOAC (2×300 mL), the combined organic layer was washed with brine, dried over sodium sulfate, filtered and the residue was concentrated. The residue was purified by column chromatography (PE) on silica gel to obtain ((2-chloro-4-methylphenyl)ethynyl)trimethylsilane (4.1 g, yield: 92.7%). ¹H NMR (400 MHz, CDCl₃): δ 7.18 (d, J=8.0 Hz, 1H), 7.01 (s, 1H), 6.80 (d, J=7.6 Hz, 1H), 2.13 (s, 3H), 0.08 (s, 9H).

Step B: 2-Chloro-1-ethynyl-4-methylbenzene. A mixture of ((2-chloro-4-methylphenyl)ethynyl)trimethylsilane (4.1 g, 18.47 mmol) and K₂CO₃ (12.7 g, 92.03 mmol) in MeOH (10 mL) and DCM (10 mL) was stirred at room temperature for 2 h. The mixture was filtered, and the residue was concentrated to obtain 2-chloro-1-ethynyl-4-methylbenzene (2.6 g, yield: 93.9%). ¹H NMR (400 MHz, CDCl3): δ 7.41 (d, J=8.0 Hz, 1H), 7.22 (s, 1H), 7.02 (dd, J1=0.8 Hz/J₂=0.8 Hz, 1H), 3.32 (s, 1H), 2.34 (s, 3H).

Step C: 4—Bromo-2-(2-chloro-4-methylphenyl)-2-methylbenzo[d][1,3]dioxole. A mixture of 3-bromobenzene-1,2-diol (1.5 g, 8.0 mmol), 2-chloro-1-ethynyl-4-methylbenzene (1.2 g, 8.0 mmol) and Ru₃(CO)₁₂ (1.0 g, 1.6 mmol) in toluene (15 mL) was stirred at 110° C. in a sealed tube under Ar for 16 h. The mixture was poured into water (200 mL) and extracted with EtOAC (2×200 mL), the combined organic layer was washed with brine, dried over sodium sulfate, filtered and the residue was concentrated. The residue was purified by column chromatography (PE) on silica gel to obtain 4-bromo-2-(2-chloro-4-methylphenyl)-2-methylbenzo[d][1,3]dioxole (360 mg, yield: 13.3%).

Step D: 1-((2-((4-(2-(2-chloro-4-methylphenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile. 1-((2-((4-(2-(2-chloro-4-methylphenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile (10.2 mg) was obtained with the similar method of Example 1. MS Calcd: 648.2; MS Found: 649.5 [M+H]⁺. ¹H NMR (400 MHz, CD₃OD): δ 9.09 (d, J=2.0 Hz, 1H), 8.51 (d, J=2.0 Hz, 1H), 7.60 (d, J=8.0 Hz, 1H), 7.28 (s, 1H), 7.10 (d, J=8.0 Hz, 1H), 6.76-6.64 (m, 3H), 3.87 (s, 2H), 3.26 (s, 2H), 3.08-2.98 (m, 2H), 2.76-2.66 (m, 1H), 2.39-2.27 (m, 5H), 2.07 (s, 3H), 2.02-1.83 (m, 2H), 1.79 (d, J=11.6 Hz, 2H), 1.41 (dd, J1 7.2 Hz/J₂=4.8 Hz, 2H), 1.25 (dd, J₁=10.8 Hz/J₂=4.8 Hz, 2H). ¹⁹F NMR (377 MHz, CD₃OD): δ −64.58.

### Example 7: 1-((2-((4-(2-(4-chloro-2-methylphenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile (7)

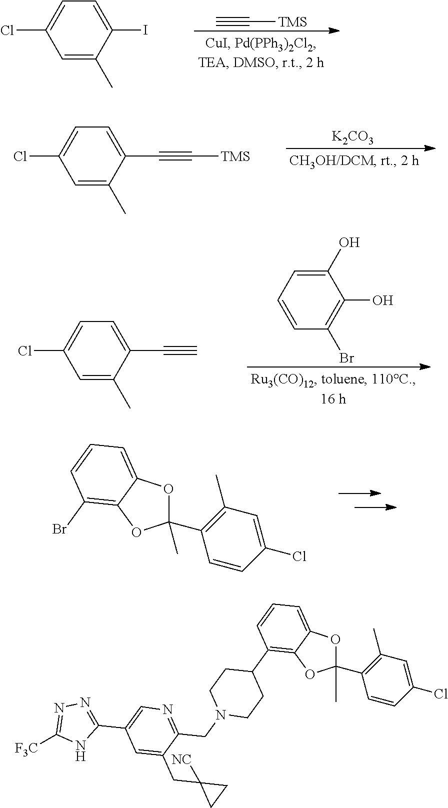

Step A: ((4-Chloro-2-methylphenyl)ethynyl)trimethylsilane. A mixture of 4-chloro-1-iodo-2-methylbenzene (5.0 g, 19.8 mmol), ethynyltrimethylsilane (3.4 mL, 23.6 mmol), CuI (75 mg, 0.40 mmol), Pd(PPh₃)₂Cl₂ (556 mg, 0.79 mmol) and TEA (2.5 mL) in toluene (20 mL) was stirred at room temperature under Ar for 2 h. The mixture was poured into water (300 mL) and extracted with EtOAC (2×300 mL), the combined organic layer was washed with brine, dried over sodium sulfate, filtered and the residue was concentrated. The residue was purified by column chromatography (PE) on silica gel to obtain ((4-chloro-2-methylphenyl)ethynyl)trimethylsilane (4.2 g, yield: 95.4%). ¹H NMR (400 MHz, CDCl₃): 7.15 (d, J=8.4 Hz, 1H), 6.99 (d, J=2.0 Hz, 1H), 6.90 (m, 1H), 2.21 (s, 3H), 0.06 (s, 9H).

Step B: The synthesis of 4-chloro-1-ethynyl-2-methylbenzene. A mixture of ((4-chloro-2-methylphenyl)ethynyl)trimethylsilane (4.2 g, 18.8 mmol) and K₂CO₃ (13.0 g, 94.17 mmol) in MeOH (10 mL) and DCM (10 mL) was stirred at room temperature for 2 h. The mixture was filtered, and the residue was concentrated to obtain 4-chloro-1-ethynyl-2-methylbenzene (2.8 g). Used directly for the next reaction. ¹H NMR (400 MHz, CDCl₃): 7.37 (d, J=8.4 Hz, 1H), 7.22-7.20 (m, 1H), 7.12 (dd, J₁=2.0 Hz/J₂=8.0 Hz, 1H), 3.30 (s, 1H), 2.42 (s, 3H).

Step C: The synthesis of 4-bromo-2-(4-chloro-2-methylphenyl)-2-methylbenzo[d][1,3]dioxole. A mixture of 3-bromobenzene-1,2-diol (1.5 g, 7.94 mmol), 4-chloro-1-ethynyl-2-methylbenzene (1.2 g, 7.94 mmol) and Ru₃(CO)₁₂ (254 mg, 0.4 mmol) in toluene (8 mL) was stirred at 110° C. in a sealed tube under Ar for 16 h. The mixture was poured into water (200 mL) and extracted with EtOAC (2×200 mL), the combined organic layer was washed with brine, dried over sodium sulfate, filtered and the residue was concentrated. The residue was purified by column chromatography (PE) on silica gel to obtain 4-bromo-2-(4-chloro-2-methylphenyl)-2-methylbenzo[d][1,3]dioxole (820 mg, yield: 30.6%). ¹H NMR (400 MHz, CDCl₃): 7.59 (d, J=8.4 Hz, 1H), 7.19-7.14 (m, 2H), 6.92 (dd, J₁=1.2 Hz/J₂=8.4 Hz, 1H), 6.74-6.66 (m, 2H), 2.56 (s, 3H), 2.02 (s, 3H).

Step D: 1-((2-((4-(2-(4-chloro-2-methylphenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile. 1-((2-((4-(2-(4-chloro-2-methylphenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile (13.3 mg) was obtained with the similar method of Example 1. MS Calcd: 648.2; MS Found: 649.3 [M+H]⁺. ¹H NMR (400 MHz, CD₃OD-d₄): δ 9.19 (d, J=2.0 Hz, 1H), 8.51 (d, J=2.0 Hz, 1H), 7.64 (d, J=8.4 Hz, 1H), 7.26 (d, J=2.0 Hz, 1H), 7.20 (dd, J, =2.0 Hz/J₂=8.8 Hz, 1H), 6.83-6.73 (m, 3H), 4.42 (brs, 2H), 3.56-3.47 (m, 2H), 3.11 (s, 2H), 3.06-2.93 (m, 3H), 2.57 (s, 3H), 2.25-2.14 (m, 2H), 2.08-2.01 (m, 2H), 1.98 (s, 3H), 1.44-1.41 (m, 2H), 1.28-1.25 (m, 2H). ¹⁹F NMR (377 MHz, CD₃OD-d₄): δ (−65.16).

### Example 8: 1-((2-((4-(2-(2-fluoro-4-(trifluoromethyl)phenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile (8)

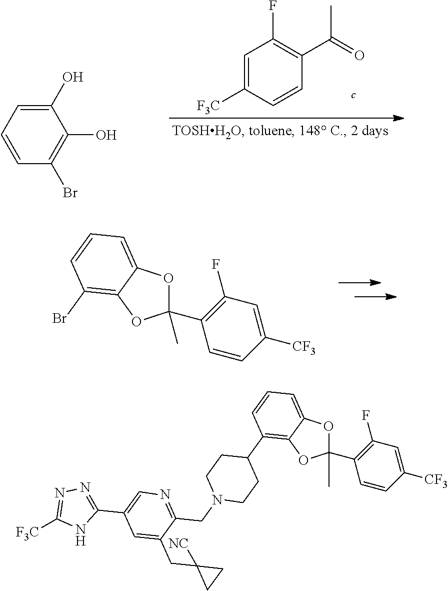 [SMILES: Oc1cccc(Br)c1O ; CC(=O)c1ccc(C(F)(F)F)cc1F ; CC1(c2ccc(C(F)(F)F)cc2F)Oc2cccc(Br)c2O1 ; CC1(c2ccc(C(F)(F)F)cc2F)Oc2cccc(C3CCN(Cc4ncc(-c5nnc(C(F)(F)F)[nH]5)cc4CC4(C#N)CC4)CC3)c2O1]

Step A: 4—Bromo-2-(2-fluoro-4-(trifluoromethyl)phenyl)-2-methylbenzo[d][1,3]dioxole. A mixture of 3-bromobenzene-1,2-diol (2.0 g, 10.6 mmol) and 1-(2-fluoro-4-(trifluoromethyl)phenyl)ethan-1-one (2.6 g, 12.6 mmol), TsOH·H₂O (1.0 g, 5.62 mmol) in toluene (50 mL) was stirred at 148° C. under Argon for 48 h in water knockout drum. The mixture was poured into NaHCO₃ aq (100 mL) and extracted with EtOAC (2×100 mL), the combined organic layer was washed with brine, dried over sodium sulfate, filtered and the residue was concentrated. The residue was purified by column chromatography (PE) on silica gel to obtain 4-bromo-2-(2-fluoro-4-(trifluoromethyl)phenyl)-2-methylbenzo[d][1,3]dioxole (1.4 g, yield: 35.0%). ¹H NMR (400 MHz, CD₃OD): δ 7.75 (t, J=7.8 Hz, 1H), 7.41 (t, J=9.6 Hz, 2H), 6.97 (dd, J₁=1.2 Hz/J₂=8.0 Hz, 1H), 6.84 (d, J=2.8 Hz, 1H), 6.78-6.69 (m, 1H), 2.14 (s, 3H).

Step B: 1-((2-((4-(2-(2—Fluoro-4-(trifluoromethyl)phenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile. 1-((2-((4-(2-(2—Fluoro-4-(trifluoromethyl)phenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile (14.1 mg) was obtained with the similar method of Example 1. MS Calcd: 686.2; MS Found: 687.2 [M+H]. ¹H NMR (400 MHz, CD₃OD): δ 9.22 (d, J=0.8 Hz, 1H), 8.53 (s, 1H), 8.19 (t, J=18 Hz, 1H), 7.87-7.82 (m, 1H), 7.60-7.49 (s, 1H), 6.87-6.77 (m, 3H), 4.59-4.53 (m, 2H), 3.66-3.59 (m, 2H), 3.16-3.12 (m, 2H), 3.09 (s, 2H), 2.95-2.87 (m, 1H), 2.26-2.19 (m, 2H), 2.10-2.03 (m, 5H), 1.47-1.41 (m, 2H), 1.30-1.25 (m, 2H). ¹⁹F NMR (377 MHz, CD₃OD): δ (−64.46), (−65.51), (−112.30).

### Example 9: 1-((2-((4-(2-(2-Chloro-4-(trifluoromethyl)phenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile (9)

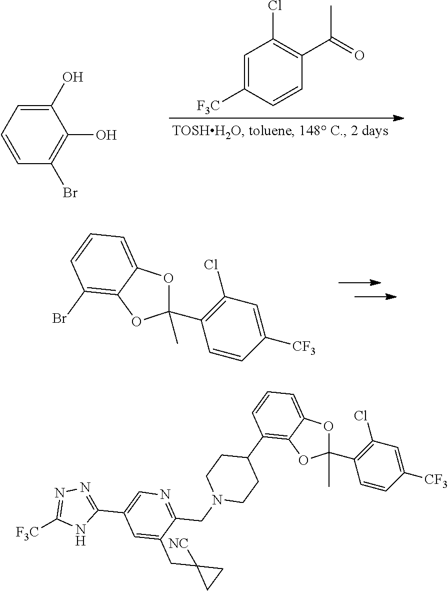 [SMILES: Oc1cccc(Br)c1O ; CC(=O)c1ccc(C(F)(F)F)cc1Cl ; CC1(c2ccc(C(F)(F)F)cc2Cl)Oc2cccc(Br)c2O1 ; CC1(c2ccc(C(F)(F)F)cc2Cl)Oc2cccc(C3CCN(Cc4ncc(-c5nnc(C(F)(F)F)[nH]5)cc4CC4(C#N)CC4)CC3)c2O1]

Step A: 4—Bromo-2-(2-chloro-4-(trifluoromethyl)phenyl)-2-methylbenzo[d][1,3]dioxole. A mixture of 3-bromobenzene-1,2-diol (2.0 g, 10.6 mmol) and 1-(2-chloro-4-(trifluoromethyl)phenyl)ethan-1-one (2.8 g, 12.6 mmol), TsOH·H₂O (1.0 g, 5.3 mmol) in toluene (50 mL) was stirred at 148° C. under Argon for 48 h in water knockout drum. The mixture was poured into NaHCO₃.aq (100 mL) and extracted with EtOAC (2×100 mL), the combined organic layer was washed with brine, dried over sodium sulfate, filtered and the residue was concentrated. The residue was purified by column chromatography (PE) on silica gel to obtain 4-bromo-2-(2-chloro-4-(trifluoromethyl)phenyl)-2-methylbenzo[d][1,3]dioxole (1.5 g, yield: 36.0%). ¹H NMR (400 MHz, CD₃OD): δ 7.89 (d, J=8.4 Hz, 1H), 7.69 (s, 1H), 7.53 (d, J=8.0H, 1H), 6.96 (d, J=8.0 Hz, 1H), 6.77 (d, J=8.0 Hz, 1H), 6.71 (d, J=8.0 Hz, 1H), 2.20 (s, 3H).

Step B: 1-((2-((4-(2-(2-Chloro-4-(trifluoromethyl)phenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile. 1-((2-((4-(2-(2-chloro-4-(trifluoromethyl)phenyl)-2-methylbenzo[d][1,3]dioxol-4-yl)piperidin-1-yl)methyl)-5-(5-(trifluoromethyl)-4H-1,2,4-triazol-3-yl)pyridin-3-yl)methyl)cyclopropane-1-carbonitrile (28.0 mg) was obtained with the similar method of Example 1. MS Calcd: 702.2; MS Found: 703.2 [M+H]⁺. ¹H NMR (400 MHz, CD₃OD): δ 9.28 (d, J=1. Hz, 1H), 8.58 (d, J=2.0 Hz, 1 H), 8.00 (d, J=8.0 Hz, 1H), 7.81 (s, 1H), 7.67 (d, J=8.0 Hz, 1H), 6.90-6.78 (m, 3H), 3.83 (s, 2H), 3.48-3.35 (m, 2H), 3.20-3.10 (m, 3H), 3.08 (s, 2H), 2.45-2.28 (m, 2H), 2.17 (s, 5H), 1.51-1.44 (m, 2H), 1.32-1.26 (m, 2H). ¹⁹F NMR (377 MHz, CD₃OD): δ (−64.57), (−66.64), (−76.87).

### Example 10: 1-{[2-({4-[2-(4-Chloro-3-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (10)

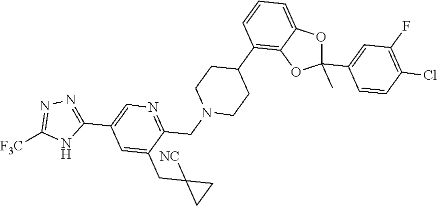 [SMILES: CC1(c2ccc(Cl)c(F)c2)Oc2cccc(C3CCN(Cc4ncc(-c5nnc(C(F)(F)F)[nH]5)cc4CC4(C#N)CC4)CC3)c2O1]

1-{[2-({4-[2-(4-Chloro-3-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (10) (6.9 mg) was obtained with the similar method of Example 1. MS Calcd: 652.2; MS Found: 653.3 [M+H]⁺. ¹H NMR (400 MHz, CD₃OD): δ 9.21 (d, J=0.4 Hz, 1H), 8.51 (d, J=1.6 Hz, 1H), 7.54-7.42 (m, 3H), 6.85-6.72 (m, 3H), 4.58-4.44 (m, 2H), 3.62-3.52 (m, 2H), 3.20-2.90 (m, 5H), 2.32-2.17 (m, 2H), 2.08-1.98 (m, 5H), 1.46-1.40 (m, 2H), 1.30-1.22 (m, 2H). ¹⁹F NMR (377 MHz, CD₃OD): δ −65.30, −65.34, −116.79.

### Example 11: 1-{[2-({4-[2-Methyl-2-(6-methylpyridin-3-yl)-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (11)

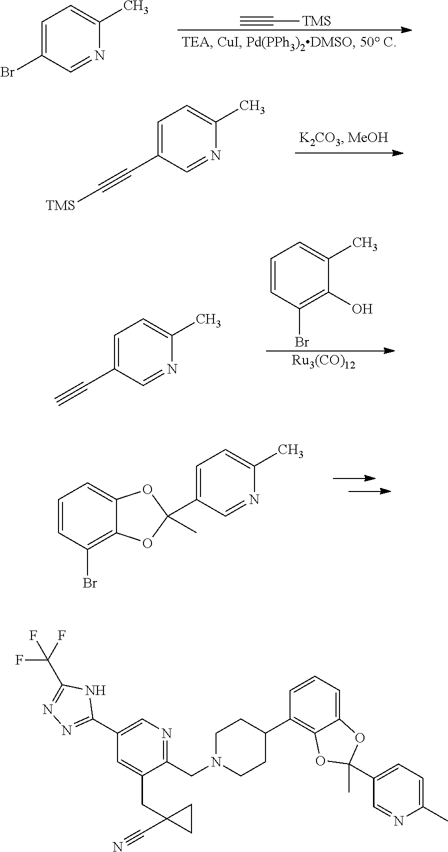

Step A: 2-Methyl-5-((trimethylsilyl)ethynyl)pyridine. A mixture of 5-bromo-2-methylpyridine (30.0 g, 174 mmol), ethynyltrimethylsilane (18.8 g, 192 mmol), CuI (663 mg, 3.5 mmol), Pd(PPh₃)₂Cl₂ (4.9 g, 7.0 mmol) in DMSO (300 mL) and TEA (24 ml) was stirred at 50° C. for 16 h. After the reaction was completed, the mixture was washed with H₂O (3 L×2), extracted with EA (1.5 L). The organic layer was dried over anhydrous sodium sulfate, filtered and evaporated to dryness. The residue was purified by column chromatography on silica gel (PE/EA=10/1) to give 2-methyl-5-((trimethylsilyl)ethynyl)pyridine (29.5 g, 90% yield). MS Calcd.: 189.1; MS Found: 190.1 [M+H]⁺.

Step B: 5-Ethynyl-2-methylpyridine. A mixture of 2-methyl-5-((trimethylsilyl)ethynyl)pyridine (29.5 g, 156 mmol), K₂CO₃ (107.7 g, 780 mmol) in MeOH (300 mL) was stirred at rt overnight. After the reaction was completed, the mixture was filtered and the filtrate evaporated to dryness and the residue was purified by column chromatography on silica gel (PE/EA=1 5/1) to give 5-ethynyl-2-methylpyridine (4.1 g, 22.6% yield). MS Calcd.: 117.1; MS Found: 118.2 [M+H]⁺.

Step C: 5-(4—Bromo-2-methylbenzo[d][1,3]dioxol-2-yl)-2-methylpyridine. A mixture of 3-bromobenzene-1,2-diol (5.4 g, 28.5 mmol), 5-ethynyl-2-methylpyridine (4.0 g, 34.2 mmol) and Ru₃(CO)₁₂ (910 mg, 1.425 mmol) in toluene (40 mL) was stirred at 110° C. for 16 hours under N₂. After the reaction was completed, the reaction mixture was filtered, and the filtrate evaporated to dryness. The residue was purified by column chromatography on silica gel (PE/EA=20/1) to give 5-(4-bromo-2-methylbenzo[d][1,3]dioxol-2-yl)-2-methylpyridine (2.3 g, yield: 26.3%). MS Calcd.: 305.0; MS Found: 305.9 [M+H]⁺.

Step D: 1-{[2-({4-[2-Methyl-2-(6-methylpyridin-3-yl)-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (11). 1-{[2-({4-[2-methyl-2-(6-methylpyridin-3-yl)-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (11) (12.8 mg) was obtained with the similar method of Example 1. MS Calcd: 615.3; MS Found: 616.4 [M+H]⁺.

¹H NMR (400 MHz, CD₃OD): δ 9.22 (d, J=1.6 Hz, 1H), 8.64 (d, J=2.0 Hz, 1H), 8.52 (d, J=2.0 Hz, 1H), 7.96 (dd, J1=8.0 Hz/J₂=2.4 Hz, 1H), 7.35 (d, J=4.0 Hz, 1H), 6.86-6.73 (m, 3H), 4.53 (s, 2H), 3.60 (d, J=10.0 Hz, 2H), 3.24-2.95 (m, 5H), 2.54 (s, 3H), 2.32-2.16 (m, 2H), 2.10-1.99 (m, 5H), 1.46-1.38 (m, 2H), 1.33-1.25 (m, 2H). ¹⁹F NMR (377 MHz, CD₃OD): δ −65.35.

### Example 12: 1-{[2-({4-[2-(4-chloro-2-fluorophenyl)-7-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (12)

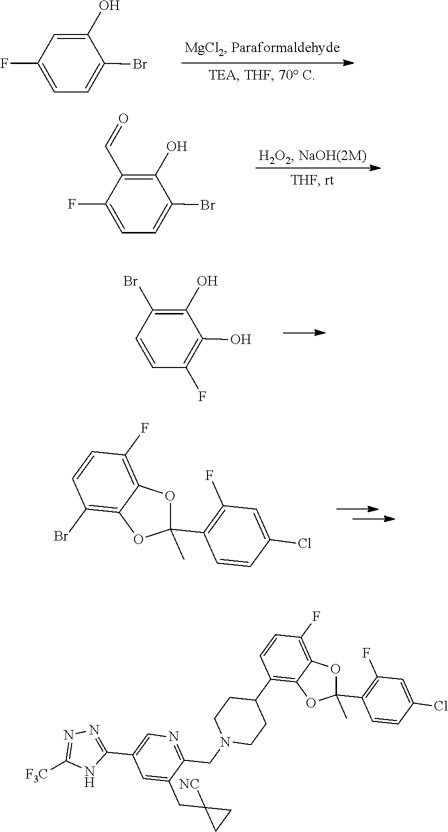

Step A: 3—Bromo-6-fluoro-2-hydroxybenzaldehyde. To a solution of MgCl₂ (7.9 g, 83.8 mmol) and paraformaldehyde (3.8 g, 125.6 mmol) in THF (100 mL) was added TEA (12.7 g, 125.6 mmol) at room temperature. The reaction was stirred at room temperature for 1 hour. 2-bromo-5-fluorophenol (8.0 g, 41.9 mmol) was added and the reaction was stirred at 70° C. for 4 hours. After the reaction was completed, the reaction was quenched with HCl (1M, 30 mL) and extracted with ethyl acetate (100 mL×3). The organic layer was combined and washed with brine (50 mL×2), dried over sodium sulfate, filtered, and concentrated in vacuum. The residue was purified by column chromatography (PE:EA=10:1) to give 3-bromo-6-fluoro-2-hydroxybenzaldehyde (4.8 g, 49% yield). ¹H NMR (400 MHz, DMSO-d₆): δ 10.74 (s, 1H), 7.50 (dd, J=8.8, 6.4 Hz, 1H), 6.75 (dd, J=10.5, 2.9 Hz, 1H), 6.62 (td, J=8.6, 2.9 Hz, 1H).

Step B: 3—Bromo-6-fluorobenzene-1,2-diol. To a solution of 3-bromo-6-fluoro-2-hydroxybenzaldehyde (4.8 g, 22.0 mmol) in THF (50 mL) was added H₂O₂(30% aqueous, 2 mL) and NaOH (2 M, 2 mL) at room temperature. The reaction was stirred at room temperature for 4 hours. After the reaction was completed, the reaction was quenched with saturated NaHSO₃ (30 mL) and extracted with ethyl acetate (50 mL×3). The organic layer was combined and washed with brine (30 mL×2), dried over sodium sulfate, filtered, and concentrated in vacuum. The residue was purified by column chromatography on silica gel (PE) to give 3-bromo-6-fluorobenzene-1,2-diol (2.0 g, 44% yield). ¹H NMR (400 MHz, DMSO-d₆): δ 9.81 (s, 1H), 9.65 (s, 1H), 6.94 (dd, J=9.0, 5.6 Hz, 1H), 6.64 (dd, J=10.2, 9.0 Hz, 1H).

Step C: 4-bromo-2-(4-chloro-2-fluorophenyl)-7-fluoro-2-methylbenzo[d][1,3]dioxole. A mixture of 3-bromo-6-fluorobenzene-1,2-diol (1.0 g, 4.85 mmol) and 4-chloro-1-ethynyl-2-fluorobenzene (822 mg, 5.34 mmol) and Ru₃(CO)₁₂ (155 mg, 0.243 mmol) in toluene (20 mL) was degassed and charged with Ar. The reaction was stirred at 100° C. for 16 hours in sealed tube. After the reaction was completed, the reaction was concentrated in vacuum. The residue was purified by column chromatography (PE) to give 4-bromo-2-(4-chloro-2-fluorophenyl)-7-fluoro-2-methylbenzo[d][1,3]dioxole (350 mg, 20.0% yield).

Step D: 1-{[2-({4-[2-(4-Chloro-2-fluorophenyl)-7-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile. 1-{[2-({4-[2-(4-chloro-2-fluorophenyl)-7-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (12) (6.9 mg) was obtained with the similar method of Example 1. MS Calcd: 670.2; MS Found: 671.3 [M+H]⁺. ¹H NMR (400 MHz, CD₃OD): δ 9.20 (d, J=1.6 Hz, 1H), 8.52 (d, J=1.6 Hz, 1H), 7.62 (t, J=8.4 Hz, 1H), 7.33 (dd, J=10.8 Hz, 1.6 Hz, 1H), 7.26 (d, J=8.4 Hz, 1H), 6.80-6.66 (m, 2H), 4.49 (s, 2H), 3.55 (d, J=10.8 Hz, 2H), 3.09 (s, 2H), 3.15-2.90 (m, 3H), 2.25-2.10 (m, 2H), 2.11 (s, 3H), 2.09-1.96 (m, 2H), 1.48-1.40 (m, 2H), 1.32-1.22 (m, 2H). ¹⁹F NMR (377 MHz, CD3OD): δ −65.42, −112.09, −142.88.

### Example 13 and 14: 1-{[2-({4-[2-(4-chloro-2-fluorophenyl)-6-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (enantiomer 1 (13) and enantiomer 2 (14))

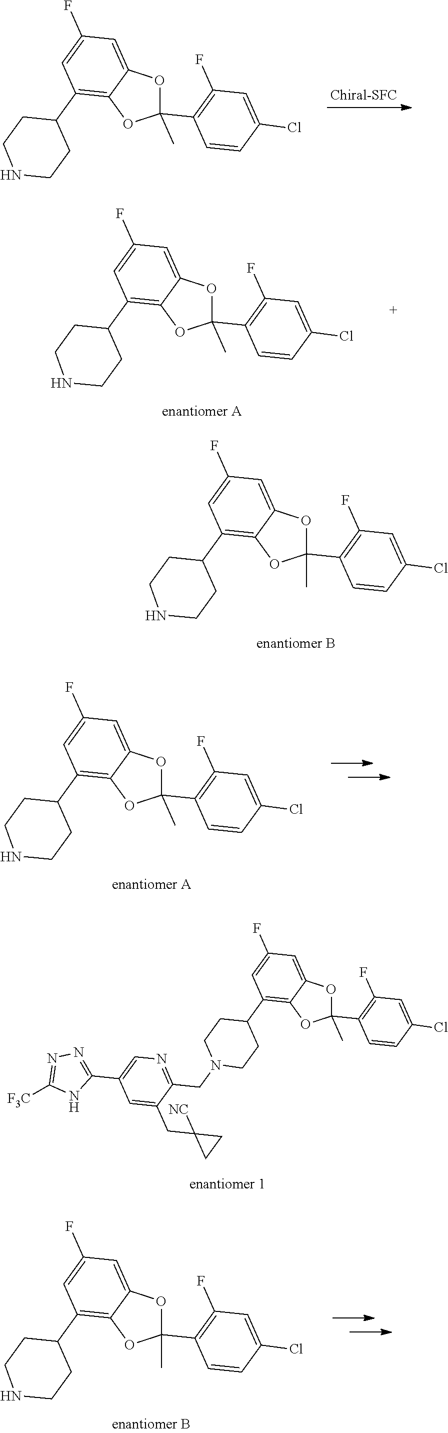 [SMILES: CC1(c2ccc(Cl)cc2F)Oc2cc(F)cc(C3CCNCC3)c2O1 ; CC1(c2ccc(Cl)cc2F)Oc2cc(F)cc(C3CCNCC3)c2O1 ; CC1(c2ccc(Cl)cc2F)Oc2cc(F)cc(C3CCNCC3)c2O1 ; CC1(c2ccc(Cl)cc2F)Oc2cc(F)cc(C3CCNCC3)c2O1 ; CC1(c2ccc(Cl)cc2F)Oc2cc(F)cc(C3CCN(Cc4ncc(-c5nnc(C(F)(F)F)[nH]5)cc4CC4(C#N)CC4)CC3)c2O1 ; CC1(c2ccc(Cl)cc2F)Oc2cc(F)cc(C3CCNCC3)c2O1]

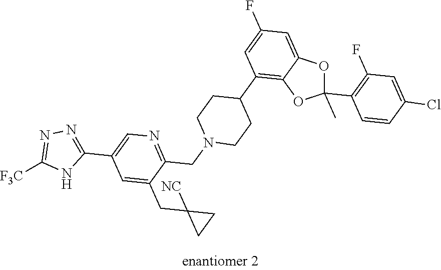 [SMILES: CC1(c2ccc(Cl)cc2F)Oc2cc(F)cc(C3CCN(Cc4ncc(-c5nnc(C(F)(F)F)[nH]5)cc4CC4(C#N)CC4)CC3)c2O1]

Step A: 4-(2-(4-Chloro-2-fluorophenyl)-6-fluoro-2-methylbenzo[d][1,3]dioxol-4-yl)piperidine (enantiomer A and enantiomer B). 4-(2-(4-Chloro-2-fluorophenyl)-6-fluoro-2-methylbenzo[d][1,3]dioxol-4-yl)piperidine was submitted to chiral separation (DAICELCHIRALCELRAD, Supercritical CO₂/MeOH(+0.1% 7.0 mol/1 Ammonia in MeOH)=60/40) to give 4-(2-(4-chloro-2-fluorophenyl)-6-fluoro-2-methylbenzo[d][1,3]dioxol-4-yl)piperidine enantiomer A and enantiomer B. Enantiomer A: MS Calcd.: 365.1; MS Found: 366.2 [M+H]⁺. SEC Retention Time: 2.68 min. Enantiomer B: MS Calcd.: 365.1; MS Found: 366.2 [M+H]⁺. SEC Retention Time: 3.01 min.

Step B: 1-{[2-({4-[2-(4-Chloro-2-fluorophenyl)-6-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (enantiomer 1, 13). 1-{[2-({4-[2-(4-Chloro-2-fluorophenyl)-6-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (13) (enantiomer 1) (25.1 mg). MS Calcd: 670.2; MS Found: 671.2 [M+H]⁺. ¹H NMR (400 MHz, CD₃OD-d₄): 9.17 (d, J=1.6 Hz, 1H), 8.51 (d, J=2.0 Hz, 1H), 7.60 (t, J=8.4 Hz, 1H), 7.30 (dd, J₁=2.0 Hz/J₂=11.2 Hz, 1H), 7.23 (dd, J₁=2.0 Hz/J₂=8.8 Hz, 1H), 6.58 (dd, J1 2.4 Hz/J₂=8.0 Hz, 1H), 6.51 (dd, J₁=2.4 Hz/J₂=10.8 Hz, 1H), 4.30 (s, 2H), 3.45-3.35 (m, 2H), 3.13 (s, 2H), 2.97-2.79 (m, 3H), 2.15-2.02 (m, 5H), 2.02-1.90 (m, 2H), 1.44-1.40 (m, 2H), 1.30-1.25 (m, 2H). ¹⁹F NMR (377 MHz, CD₃OD): δ −65.15, −112.22, −121.81.

1-{[2-({4-[2-(4-Chloro-2-fluorophenyl)-6-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (enantiomer 2, 14) was prepared using the similar procedure, using 4-(2-(4-chloro-2-fluorophenyl)-6-fluoro-2-methylbenzo[d][1,3]dioxol-4-yl)piperidine (enantiomer B) as starting material. MS Calcd: 670.2; MS Found: 671.2 [M+H]⁺. ¹H NMR (400 MHz, CD₃OD-d₄): δ 9.16 (d, J₁=2.0 Hz, 1H), 8.51 (d, J=2.0 Hz, 1H), 7.60 (t, J=8.4 Hz, 1H), 7.30 (dd, J₁=2.0 Hz/J₂=10.8 Hz, 1H), 7.23 (dd, J₁=2.0 Hz/J₂=8.4 Hz, 1H), 6.58 (dd, J₁=2.8 Hz/J₂=8.4 Hz, 1H), 6.51 (dd, J1=2.8 Hz/J₂=10.8 Hz, 1H), 4.27 (s, 2H), 3.43-3.32 (m, 2H), 3.14 (s, 2H), 2.94-2.75 (m, 3H), 2.15-2.00 (m, 5H), 2.00-1.90 (m, 2H), 1.44-1.40 (m, 2H), 1.30-1.25 (m, 2H). ¹⁹F NMR (377 MHz, CD₃OD): δ −65.11, −112.23, −121.84.

### Example 15: 1-{[2-({4-[2-(4-Chloro-2-fluorophenyl)-5-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (15)

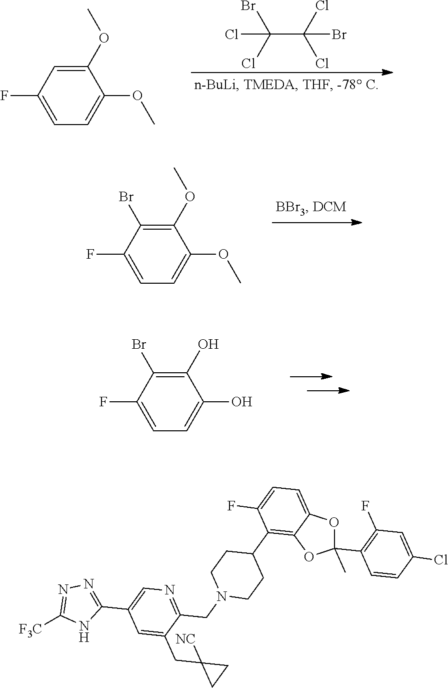 [SMILES: COc1ccc(F)cc1OC ; ClC(Cl)(Br)C(Cl)(Cl)Br ; COc1ccc(F)c(Br)c1OC ; Oc1ccc(F)c(Br)c1O ; CC1(c2ccc(Cl)cc2F)Oc2ccc(F)c(C3CCN(Cc4ncc(-c5nnc(C(F)(F)F)[nH]5)cc4CC4(C#N)CC4)CC3)c2O1]

Step A: 2—Bromo-1-fluoro-3,4-dimethoxybenzene. To a solution of 4-fluoro-1,2-dimethoxybenzene (2.0 g, 12.8 mmol) and TMEDA (1.5 g, 12.8 mmol) in THF (20 mL) was added n-BuLi (12 mL, 1.6 mol/L, 19.2 mmol) at −78° C. The reaction was stirred at −78° C. for 1.5 hours. Then 1,2-dibromo-1,1,2,2-tetrachloroethane (5.43 g, 16.7 mmol) was added and the reaction was stirred at −78° C. for 2 h. After the reaction was completed, the reaction was quenched with NH₄Cl solution (50 mL) and extracted with ethyl acetate (100 mL×3). The organic layer was combined and washed with brine (100 mL×2), dried over sodium sulfate, filtered, and concentrated in vacuum. The crude product was purified by column chromatography on silica gel (PE:EA=10:1) to give 2-bromo-1-fluoro-3,4-dimethoxybenzene (2.5 g, crude).

Step B: 3—Bromo-4-fluorobenzene-1,2-diol. To a solution of 2-bromo-1-fluoro-3,4-dimethoxybenzene (2.5 g, 10.7 mmol) in DCM (25 mL) was added BBr₃ (5.3 g, 21.4 mmol) at room temperature. The reaction was stirred at room temperature for 2 hours. After the reaction was completed, the reaction was quenched with MeOH (100 mL) and concentrated in vacuum. The residue was purified by column chromatography (PE:EA=3:1) to give 3-bromo-4-fluorobenzene-1,2-diol (1.4 g, 52.8% yield in 2 steps).

Step C: 1-{[2-({4-[2-(4-Chloro-2-fluorophenyl)-5-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (15). 1-{[2-({4-[2-(4-chloro-2-fluorophenyl)-5-fluoro-2-methyl-2H-1,3-benzodioxol-4-yl]piperidin-1-yl}methyl)-5-[5-(trifluoromethyl)-4H-1,2,4-triazol-3-yl]pyridin-3-yl]methyl}cyclopropane-1-carbonitrile (15) (4.2 mg) was obtained with the similar method of Example 1. MS Calcd: 670.2; MS Found: 671.3 [M+H]⁺. ¹H NMR (400 MHz, CDCl₃): δ 9.20 (s, 1H), 8.52 (s, 1H), 7.62 (t, J=8.6 Hz, 1H), 7.32 (dd, J₁=2.0 Hz/J₂=10.8 Hz, 1H), 7.25 (dd, J₁=1.6 Hz/J₂=8.8 Hz, 1H), 6.75-6.68 (m, 1H), 6.61-6.52 (m, 1H), 4.44 (s, 2H), 3.57-3.45 (m, 2H), 3.25-3.15 (m, 1H), 3.12 (s, 2H), 3.08-2.92 (m, 2H), 2.53-2.38 (m, 2H), 2.10-2.03 (m, 3H), 2.00-1.87 (m, 2H), 1.47-1.40 (m, 2H), 1.31-1.24 (m, 2H). ¹⁹F NMR (377 MHz, CDCl₃): δ −65.38, −112.17, −128.37.

### Example 16: 1-{[2-({4-[(2S)-2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]pyridin-3-yl]methyl}cyclopropane-1-carbonitrile (16)

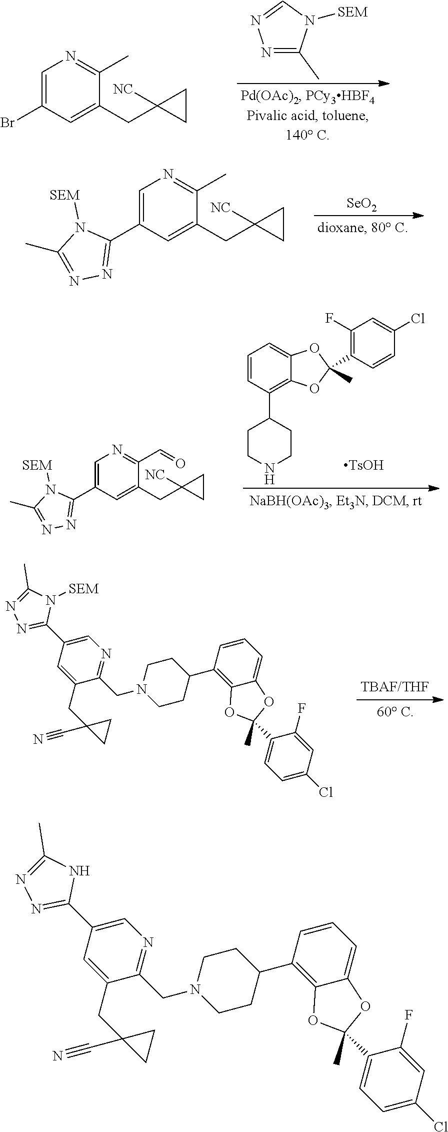

Step A: 1-((2-Methyl-5-(5-methyl-4-((2-(trimethylsilyl)ethoxy)methyl)-4H-1,2,4-triazol-3-yl)pyridin-3-yl)methyl)cyclopropane-1-carbonitrile. A mixture of 1-((5-bromo-2-methylpyridin-3-yl)methyl)cyclopropane-1-carbonitrile (140 mg, 0.56 mmol), 3-methyl-4-((2-(trimethylsilyl)ethoxy)methyl)-4H-1,2,4-triazole (358 mg, 1.68 mmol), Pd(OAc)₂ (13 mg, 0.06 mmol), Pivalic acid (38 mg, 0.37 mmol), K₂CO₃ (464 mg, 3.36 mmol) and PCy₃·HBF₄ (41 mg, 0.11 mmol) in toluene (10 mL) was stirred at 140° C. under Argon for 16 h. The mixture was filtered, and the filtrate was concentrated. The residue was purified by prep-TLC (DCM:MeOH=20:1) to give 1-((2-methyl-5-(5-methyl-4-((2-(trimethylsilyl)ethoxy)methyl)-4H-1,2,4-triazol-3-yl)pyridin-3-yl)methyl)cyclopropane-1-carbonitrile (20 mg, yield: 9.3%). MS Calcd: 383.2; MS Found: 384.5 [M+H]⁺.

Step B: 1-((2—Formyl-5-(5-methyl-4-((2-(trimethylsilyl)ethoxy)methyl)-4H-1,2,4-triazol-3-yl)pyridin-3-yl)methyl)cyclopropane-1-carbonitrile. A mixture of 1-((2-methyl-5-(5-methyl-4-((2-(trimethylsilyl)ethoxy)methyl)-4H-1,2,4-triazol-3-yl)pyridin-3-yl)methyl)cyclopropane-1-carbonitrile (20 mg, 0.05 mmol) and SeO₂ (58 mg, 0.52 mmol) in 1,4-dioxane (3 mL) was stirred at 80° C. for 16 h. The mixture was filtered, and the filtrate was concentrated to give 1-((2-formyl-5-(5-methyl-4-((2-(trimethylsilyl)ethoxy)methyl)-4H-1,2,4-triazol-3-yl)pyridin-3-yl)methyl)cyclopropane-1-carbonitrile (18 mg, yield: 90.68%). MS Calcd: 397.2; MS Found: 398.3 [M+H]⁺.

Step C: (S)-1-((2-((4-(2-(4-Chloro-2-fluorophenyl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)pyridin-3-yl)methyl)cyclopropane-1-carbonitrile. A mixture of (S)-4-(2-(4-chloro-2-fluorophenyl)-2-methylbenzo[d][1,3]dioxol-4-yl)piperidine (26 mg, 0.05 mmol) and TEA (23 mg, 0.23 mmol) in DCM (5 mL) was stirred at rt for 0.5 h. The mixture was added 1-((2-formyl-5-(5-methyl-4-((2-(trimethylsilyl)ethoxy)methyl)-4H-1,2,4-triazol-3-yl)pyridin-3-yl)methyl)cyclopropane-1-carbonitrile (18 mg, 0.05 mmol) and the mixture was stirred for 2 h. The mixture was added NaBH(OAc)₃ (38 mg, 0.18 mmol) and the mixture was stirred at rt for 16 h. The mixture was poured into water (30 mL) and extracted with DCM (2×30 mL), the combined organic layer was washed with brine, dried over sodium sulfate, filtered and the filtrate was concentrated to give (S)-1-((2-((4-(2-(4-chloro-2-fluorophenyl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)pyridin-3-yl)methyl)cyclopropane-1-carbonitrile (50 mg). MS Calcd: 728.3; MS Found: 729.4 [M+H]⁺.

Step D: 1-{[2-({4-[(2S)-2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]pyridin-3-yl]methyl}cyclopropane-1-carbonitrile (16). A mixture of (S)-1-((2-((4-(2-(4-chloro-2-fluorophenyl)-2-methylbenzo[d][1,3]dioxol-4-yl)piperidin-1-yl)methyl)-5-(5-methyl-4-((2-(trimethylsilyl)ethoxy)methyl)-4H-1,2,4-triazol-3-yl)pyridin-3-yl)methyl)cyclopropane-1-carbonitrile (50 mg, 0.07 mmol) and TABF (1 M in THF, 2 mL) in THF (1 mL) was stirred at 60° C. for 8 h. The mixture was purified by prep-HPLC to give 1-{[2-({4-[(2S)-2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]pyridin-3-yl]methyl}cyclopropane-1-carbonitrile (16) (6.0 mg, yield: 14.5%). MS Calcd: 598.2; MS Found: 599.4 [M+H]⁺. ¹HNMR (400 MHz, CD₃OD): δ 9.08 (s, 1H), 8.53 (s, 1H), 7.59 (t, J₁=8.2 Hz, 1H), 7.27 (dd, J=1.6 Hz/J=10.8 Hz, 1H), 7.20 (d, J=8.8 Hz, 1H), 6.80-6.65 (m, 3H), 4.10-3.90 (br.s, 2H), 3.25-3.08 (m, 6H), 2.85-2.70 (m, 1H), 2.53 (s, 3H), 2.03 (s, 3H), 2.05-1.79 (m, 4H), 1.47-1.40 (m, 2H), 1.27-1.21 (m, 2H). ¹⁹F NMR (377 MHz, CD₃OD): δ −112.28.

### Example 17: 1-{[2-({4-[(2S)-2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]pyridin-3-yl]methyl}cyclopropane-1-carbonitrile (17)

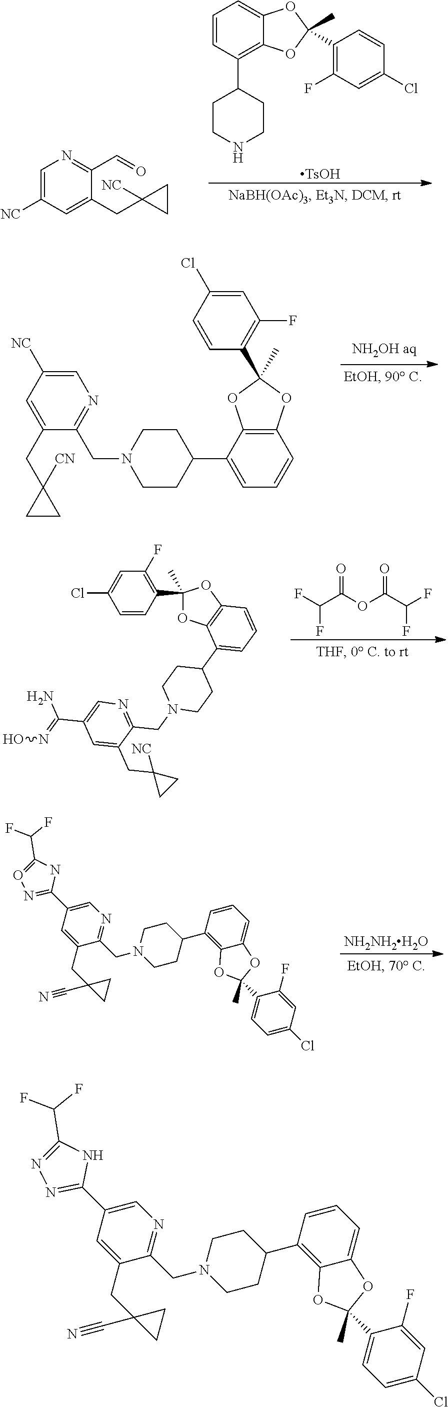

Step A: (S)-6-((4-(2-(4-chloro-2-fluorophenyl)-2-methylbenzo[d][1,3]dioxol-4-yl)piperidin-1-yl)methyl)-5-((1-cyanocyclopropyl)methyl)nicotinonitrile. A mixture of(S)-4-(2-(4-chloro-2-fluorophenyl)-2-methylbenzo[d][1,3]dioxol-4-yl)piperidine 4-methylbenzenesulfonic acid salt (99 mg, 0.19 mmol) and TEA (96 mg, 0.95 mmol) in DCM (5 mL) was stirred at rt for 0.5 h. 5-((1-cyanocyclopropyl)methyl)-6-formylnicotinonitrile (40 mg, 0.76 mmol) was added and the mixture was stirred for 2 h. NaBH(OAc)₃ (161 mg, 0.57 mmol) was added and the mixture was stirred at rt for 16 h. The mixture was poured into water (50 mL) and extracted with DCM (2×50 mL), the combined organic layer was washed with brine, dried over sodium sulfate, filtered and the filtrate was concentrated. The residue was purified by column chromatography on silica gel to give (S)-6-((4-(2-(4-chloro-2-fluorophenyl)-2-methylbenzo[d][1,3]dioxol-4-yl)piperidin-1-yl)methyl)-5-((1-cyanocyclopropyl)methyl)nicotinonitrile (44 mg, yield: 42.8%).

Step B: (S)-6-((4-(2-(4-chloro-2-fluorophenyl)-2-methylbenzo[d][1,3]dioxol-4-yl)piperidin-1-yl)methyl)-5-((1-cyanocyclopropyl)methyl)-N′-hydroxynicotinimidamide. A mixture of (S)-6-((4-(2-(4-chloro-2-fluorophenyl)-2-methylbenzo[d][1,3]dioxol-4-yl)piperidin-1-yl)methyl)-5-((1-cyanocyclopropyl)methyl)nicotinonitrile (44 mg, 0.081 mmol) and NH₂OH aqueous solution (50%) (11 mg, 0.16 mmol) in EtOH (5 mL) was stirred at 90° C. for 1 h. The mixture was concentrated to give (S)-6-((4-(2-(4-chloro-2-fluorophenyl)-2-methylbenzo[d][1,3]dioxol-4-yl)piperidin-1-yl)methyl)-5-((1-cyanocyclopropyl)methyl)-N′-hydroxynicotinimidamide (47 mg, crude). MS Calcd: 575.2; MS Found: 576.2 [M+H]⁺.

Step C: (S)-1-((2-((4-(2-(4-chloro-2-fluorophenyl)-2-methylbenzo[d][1,3]dioxol-4-yl)piperidin-1-yl)methyl)-5-(5-(difluoromethyl)-1,2,4-oxadiazol-3-yl)pyridin-3-yl)methyl)cyclopropane-1-carbonitrile. To a solution of (S)-6-((4-(2-(4-chloro-2-fluorophenyl)-2-methylbenzo[d][1,3]dioxol-4-yl)piperidin-1-yl)methyl)-5-((1-cyanocyclopropyl)methyl)-N′-hydroxynicotinimidamide (47 mg, 0.082 mmol) in THF (3 mL) were added 2,2-difluoroacetic anhydride (71 mg, 0.41 mmol) in THF (0.5 mL) in a dropwise manner at 0° C. The mixture was stirred at rt for 16 h. The mixture was poured into sodium bicarbonate aqueous solution (30 mL) and extracted with EtOAc (2×30 mL), the combined organic layer was washed with brine, dried over sodium sulfate, filtered and the filtrate was concentrated. The residue was purified by prep-TLC to give (S)-1-((2-((4-(2-(4-chloro-2-fluorophenyl)-2-methylbenzo[d][1,3]dioxol-4-yl)piperidin-1-yl)methyl)-5-(5-(difluoromethyl)-1,2,4-oxadiazol-3-yl)pyridin-3-yl)methyl)cyclopropane-1-carbonitrile (52 mg, crude). MS Calcd: 635.2; MS Found: 636.4 [M+H]⁺.

Step D: 1-{[2-({4-[(2S)-2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]pyridin-3-yl]methyl}cyclopropane-1-carbonitrile (17). A mixture of (S)-1-((2-((4-(2-(4-chloro-2-fluorophenyl)-2-methylbenzo[d][1,3]dioxol-4-yl)piperidin-1-yl)methyl)-5-(5-(difluoromethyl)-1,2,4-oxadiazol-3-yl)pyridin-3-yl)methyl)cyclopropane-1-carbonitrile (52 mg, 0.082 mmol) and NH₂NH₂·H₂O (0.3 mL) in EtOH (2 mL) was stirred at 70° C. for 4 h. The mixture was purified by prep-HPLC to give 1-{[2-({4-[(2S)-2-(4-chloro-2-fluorophenyl)-2-methyl-2H-1,3-benzodioxol-4-yl]piperidin-1-yl}methyl)-5-[5-(difluoromethyl)-4H-1,2,4-triazol-3-yl]pyridin-3-yl]methyl}cyclopropane-1-carbonitrile (17) (17.6 mg, yield: 34.1%). MS Calcd: 634.2; MS Found: 635.4 [M+H]⁺. ¹H NMR (400 MHz, CD₃OD): δ 9.14 (d, J=1.2 Hz, 1H), 8.54 (s, 1H), 7.60 (t, J=8.4 Hz, 1H), 7.28 (d, J=10.8 Hz, 1H), 7.21 (dd, J₁=1.2 Hz/J₂=8.4 Hz, 1H), 7.07-6.82 (m, 1H), 6.82-6.70 (m, 3H), 4.21 (s, 2H), 3.39-3.31 (m, 2H), 3.18 (s, 2H), 2.91-2.80 (m, 1H), 2.80-2.68 (m, 2H), 2.16-2.02 (m, 2H), 2.04 (s, 3H), 1.98-1.87 (m, 2H), 1.47-1.40 (m, 2H), 1.30-1.22 (m, 2H). ¹⁹F NMR (377 MHz, CD₃OD): δ −112.27, −117.61.

### Example 18: (S)-5-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)-4H-1,2,4-triazole-3-carbonitrile (18)

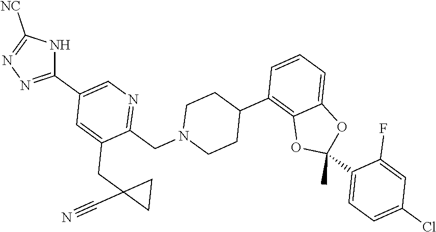 [SMILES: C[C@]1(c2ccc(Cl)cc2F)Oc2cccc(C3CCN(Cc4ncc(-c5nnc(C#N)[nH]5)cc4CC4(C#N)CC4)CC3)c2O1]

MS Calcd.: 609.2; MS Found: 610.2 [M+H]⁺. ¹H NMR (400 MHz, MeOD-d4): δ 9.24 (s, 1H), 8.51 (s, 1H), 7.63 (t, J=4.8 Hz, 1H), 7.30 (d, J=11.2 Hz, 1H), 7.23 (d, J=8.4 Hz, 1H), 6.88-6.78 (m, 3H), 4.72 (s, 2H), 3.81-3.71 (m, 2H), 3.40-3.37 (m, 2H), 3.15-3.03 (m, 3H), 2.38-2.07 (m, 7H), 1.47-1.42 (m, 2H), 1.32-1.26 (m, 2H). ¹⁹F NMR (377 MHz, MeOD-d4): δ (−112.23).

### Example 19: (S)-6-((4-(2-(4-chloro-2-fluorophenyl)-2-methylbenzo[d][1,3]dioxol-4-yl)piperidin-1-yl)methyl)-5-((1-cyanocyclopropyl)methyl)nicotinic acid (19)

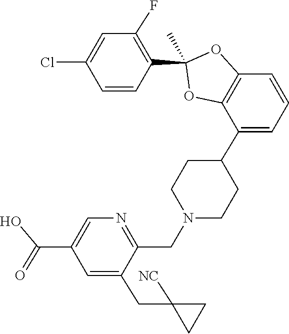 [SMILES: C[C@]1(c2ccc(Cl)cc2F)Oc2cccc(C3CCN(Cc4ncc(C(=O)O)cc4CC4(C#N)CC4)CC3)c2O1]

A mixture of (S)-6-((4-(2-(4-chloro-2-fluorophenyl)-2-methylbenzo[d][1,3]dioxol-4-yl)piperidin-1-yl)methyl)-5-((1-cyanocyclopropyl)methyl)nicotinonitrile (20 mg, 0.037 mmol) and NaOH (6 mg, 0.15 mmol) in EtOH (2 mL) was stirred at 70° C. for 6 h. The mixture was filtered, and the residue was purified by prep-HPLC to obtain compound 19 (5.0 mg, yield: 21.2%). MS Calcd: 561.2; MS Found: 562.2 [M+H]⁺. ¹H NMR (400 MHz, DMSO-d₆): δ 8.73 (s, 1H), 8.19 (s, 1H), 7.58-7.54 (m, 2H), 7.34 (dd, J=2.0 Hz/J=8.8 Hz, 1H), 6.78 (d, J=4.4 Hz, 2H), 6.74-6.71 (m, 1H), 3.64 (s, 2H), 3.11 (s, 2H), 2.88-2.83 (m, 2H), 2.66-2.61 (m, 1H), 2.15-2.08 (m, 2H), 2.02 (s, 3H), 1.74-1.69 (m, 4H), 1.33-1.30 (m, 2H), 1.16-1.13 (m, 2H). ¹⁹F NMR (377 MHz, CD₃OD): δ (−110.78).

### Example 20: (S,E)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)acrylic acid (20)

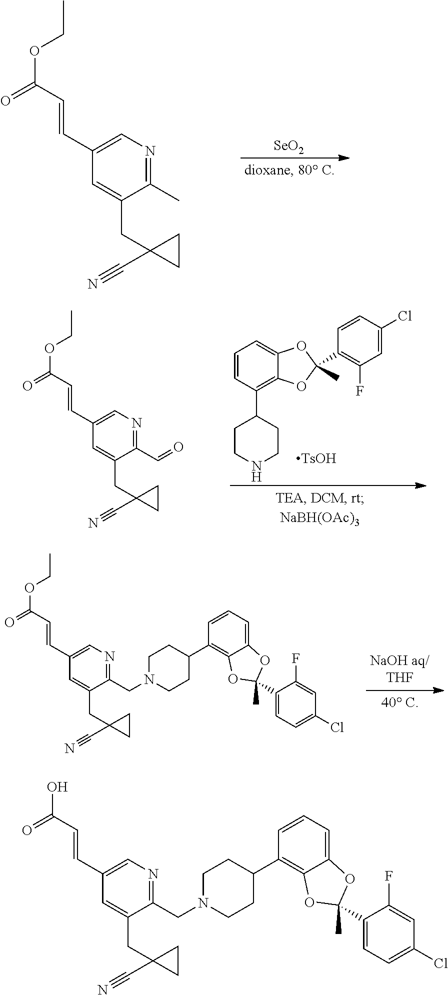

Step A: ethyl (E)-3-(5-((1-cyanocyclopropyl)methyl)-6-formylpyridin-3-yl)acrylate. A mixture of ethyl (E)-3-(5-((1-cyanocyclopropyl)methyl)-6-methylpyridin-3-yl)acrylate (50 mg, 0.185 mmol) and SeO₂ (205 mg, 1.85 mmol) in 1,4-dioxane (5 mL) was stirred at 80° C. for 16 h. The mixture was filtered, and the residue was concentrated to obtain ethyl (E)-3-(5-((1-cyanocyclopropyl)methyl)-6-formylpyridin-3-yl)acrylate (30 mg, yield: 39%). MS Calcd: 284.2; MS Found: 285.0 [M+H]T.

Step B: ethyl (S,E)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)acrylate. A mixture of (S)-4-(2-(4-chloro-2-fluorophenyl)-2-methylbenzo[d][1,3]dioxol-4-yl)piperidine 4-methylbenzenesulfonate (107 mg, 0.21 mmol) and TEA (98 mg, 0.97 mmol) in DCM (5 mL) was stirred at rt for 0.5 h. The mixture was added ethyl (E)-3-(5-((1-cyanocyclopropyl)methyl)-6-formylpyridin-3-yl)acrylate (55 mg, 0.19 mmol) and the mixture was stirred for 2 h. The mixture was added NaBH(OAC)₃ (165 mg, 0.78 mmol) and the mixture was stirred at rt for 16 h. The mixture was poured into water (30 mL) and extracted with DCM (2×30 mL), the combined organic layer was washed with brine, dried over sodium sulfate, filtered and the residue was concentrated to obtain ethyl (S,E)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)acrylate (30 mg, yield: 40.0%). MS Calcd: 615.3; MS Found: 616.2 [M+H]⁺.

Step C: (S,E)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)acrylic acid. A mixture of ethyl (S,E)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)acrylate (15 mg, 0.024 mmol) and NaOH aq (3 M, 0.1 mL) in THF (2 mL) was stirred at rt for 2 h. The mixture was purified by prep-HPLC to obtain (S,E)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)acrylic acid (2.6 mg, yield: 18.5%). MS Calcd: 587.2; MS Found: 588.4 [M+H]⁺. ¹H NMR (400 MHz, CD₃OD-d₄): δ 8.60 (s, 1H), 8.48 (s, 1H), 8.11 (s, 1H), 7.59 (t, J=8.4 Hz, 1H), 7.47-7.43 (m, 1H), 7.29 (dd, J₁=2.0 Hz/J₂ 10.8 Hz, 1H), 7.21 (dd, J=7.2 Hz/J₂ 8.8 Hz, 1H), 6.83-6.63 (m, 4H), 4.10-4.03 (m, 2H), 3.24-3.11 (m, 4H), 2.86-2.74 (m, 1H), 2.67-2.48 (m, 2H), 2.09-1.94 (m, 5H), 1.90-1.80 (m, 2H), 1.42-1.19 (m, 4H). ¹⁹F NMR (377 MHz, CD₃OD-d₄): δ (−112.28).

### Example 21: (S)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)propanoic acid (21)

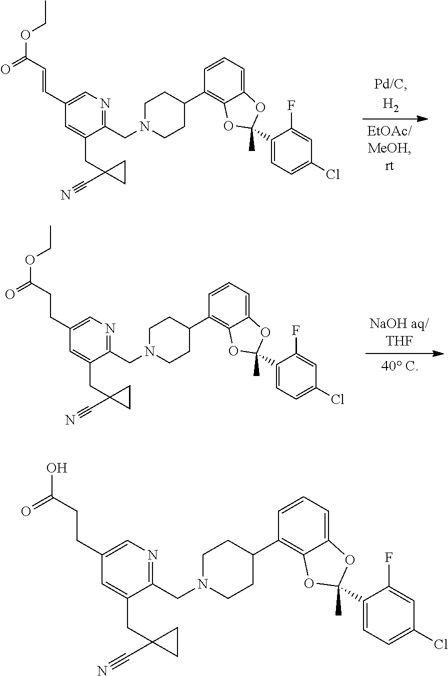 [SMILES: CCOC(=O)/C=C/c1cnc(CN2CCC(c3cccc4c3O[C@@](C)(c3ccc(Cl)cc3F)O4)CC2)c(CC2(C#N)CC2)c1 ; CCOC(=O)CCc1cnc(CN2CCC(c3cccc4c3O[C@@](C)(c3ccc(Cl)cc3F)O4)CC2)c(CC2(C#N)CC2)c1 ; C[C@]1(c2ccc(Cl)cc2F)Oc2cccc(C3CCN(Cc4ncc(CCC(=O)O)cc4CC4(C#N)CC4)CC3)c2O1]

Step A: ethyl (S)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)propanoate. A mixture of ethyl (S,E)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)acrylate (15 mg, 0.024 mmol) and Pd/C (0.3 mg, 0.096 mmol) in EtOAc (2 ml)/MeOH (2 ml) and H₂ was stirred at rt for 2 h. The mixture was filtered, and the residue was concentrated to obtain ethyl (S)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)propanoate (15 mg, yield: >99%). MS Calcd: 617.3; MS Found: 618.4 [M+H]⁺.

Step B: (S)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)propanoic acid. A mixture of ethyl (S)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)propanoate (15 mg, 0.024 mmol) and NaOH aq (3 M, 0.1 mL) in THF (2 mL) was stirred at rt for 2 h. The mixture was purified by prep-HPLC to obtain (S)-3-(6-((4-(2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)propanoic acid (3.8 mg, yield: 26.9%). MS Calcd: 589.2; MS Found: 590.4 [M+H]⁺. ¹H NMR (400 MHz, CD₃OD): δ 8.39 (s, 1H), 7.84 (s, 1H), 7.59 (t, J=8 Hz, 1H), 7.28 (dd, J=8.8 Hz/J=10.8 Hz, 1H), 7.20 (dd, J=4.0 Hz/J=8.0 Hz, 1H), 6.79 (t, J=8.4 Hz, 1H), 6.72 (d, J=7.2 Hz, 2H), 4.05 (s, 1H), 3.24-3.18 (m, 2H), 3.13 (s, 2H), 2.98 (t, J₁=6.8 Hz, 2H), 2.65-2.49 (m, 4H), 2.03 (s, 5H), 1.86 (t, J=11.2 Hz, 2H), 1.41-1.32 (m, 2H), 1.24-1.14 (m, 2H). ¹⁹F NMR (377 MHz, CD₃OD): δ (−112.26).

### Example 22: 1-{[2-({4-[2-(4-chloro-2-fluorophenyl)-2-methyl-2H-1,3-benzodioxol-4-yl]-3,3-difluoropiperidin-1-yl}methyl)-5-[5-(trifluoromethyl)-4H-1,2,4-triazol-3-yl]pyridin-3-yl]methyl}cyclopropane-1-carbonitrile (22)

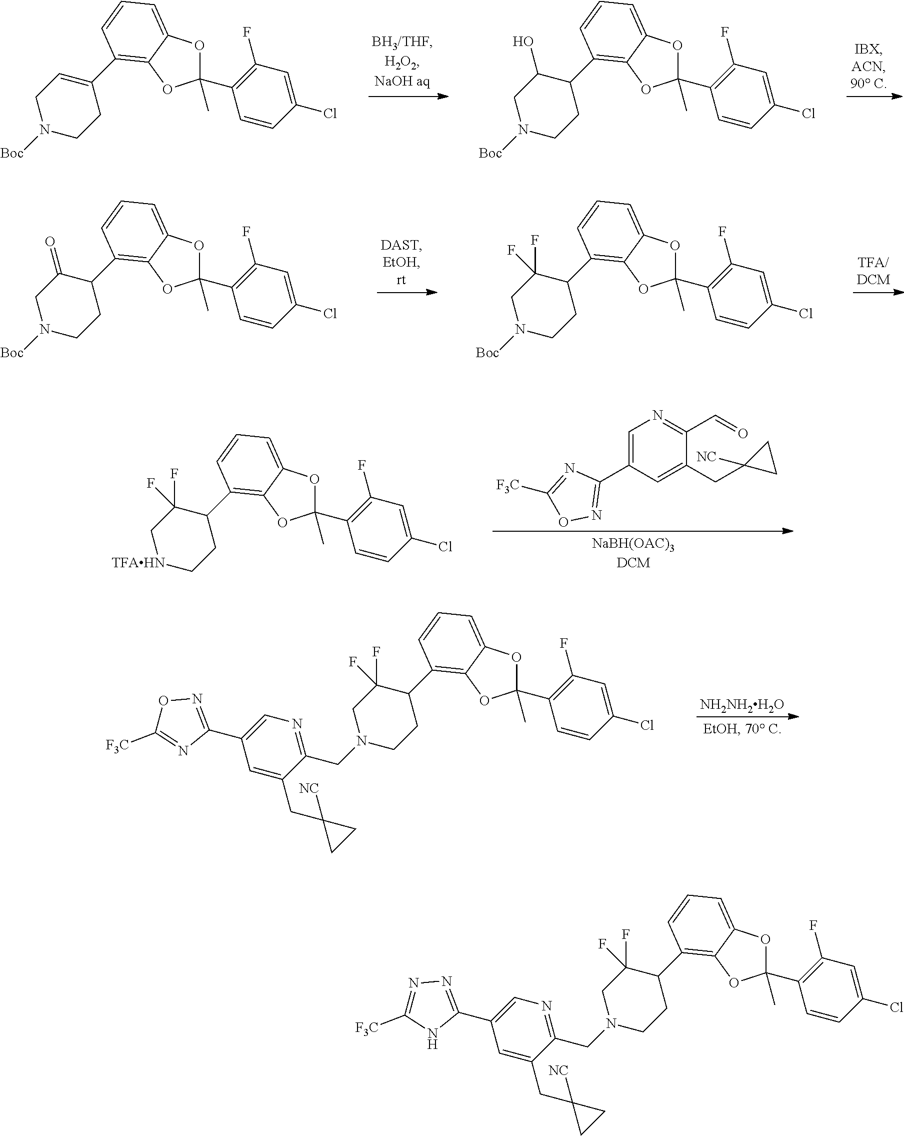

Step A: tert-butyl 4-(2-(4-chloro-2-fluorophenyl)-2-methylbenzo[d][1,3]dioxol-4-yl)-3-hydroxypiperidine-1-carboxylate. A mixture of tert-butyl 4-(2-(4-chloro-2-fluorophenyl)-2-methylbenzo[d][1,3]dioxol-4-yl)-3,6-dihydropyridine-1(2H)-carboxylate (1.0 g, 2.25 mmol) in THF (20 mL) was added BH₃·THF (1 M in THF, 4.5 ml, 4.5 mmol) was stirred at rt for 2 h. NaOH.aq (2 M, 3.4 mL, 6.75 mmol) was added and the mixture was stirred for 10 min. H₂O₂(30%, 3.4 mL) was added and the mixture was stirred at 50° C. for 1.5 h. MeOH (10 mL) was added and the mixture was stirred rt for 16 h. The mixture was poured into water (100 mL) and extracted with EA (2×100 mL), the combined organic layer was washed with brine, dried over sodium sulfate, filtered and the filtrate was concentrated to give tert-butyl 4-(2-(4-chloro-2-fluorophenyl)-2-methylbenzo[d][1,3]dioxol-4-yl)-3-hydroxypiperidine-1-carboxylate (0.70 g, yield: 67.3%). MS Calcd: 463.2; MS Found: 486.3 [M+23]⁺.

Step B: tert-Butyl tert-butyl 4-(2-(4-chloro-2-fluorophenyl)-2-methylbenzo[d][1,3]dioxol-4-yl)-3-oxopiperidine-1-carboxylate. A mixture of tert-butyl 4-(2-(4-chloro-2-fluorophenyl)-2-methylbenzo[d][1,3]dioxol-4-yl)-3-hydroxypiperidine-1-carboxylate (200 mg, 0.43 mmol) and IBX (242 mg, 0.86 mmol) in ACN (5 mL) was stirred at 90° C. for 1 h. The mixture was filtered, and the filtrate was concentrated to give tert-butyl 4-(2-(4-chloro-2-fluorophenyl)-2-methylbenzo[d][1,3]dioxol-4-yl)-3-oxopiperidine-1-carboxylate (160 mg, yield: 80.3%). MS Calcd: 461.1; MS Found: 484.2 [M+23]⁺.

Step C: tert-butyl 4-(2-(4-chloro-2-fluorophenyl)-2-methylbenzo[d][1,3]dioxol-4-yl)-3,3-difluoropiperidine-1-carboxylate. To a mixture of tert-butyl 4-(2-(4-chloro-2-fluorophenyl)-2-methylbenzo[d][1,3]dioxol-4-yl)-3-oxopiperidine-1-carboxylate (140 mg, 0.30 mmol) in DAST (5 mL) was added EtOH (7 drops) and the mixture was stirred at rt for 16 h under Ar. The mixture was poured into NaHCO₃.aq (70 mL) at 0° C. and extracted with EtOAC (2×50 mL), the combined organic layer was washed with brine, dried over sodium sulfate, filtered and the filtrate was concentrated. The residue was purified by column chromatography (PE:EA 6:1) on silica gel to give tert-butyl 4-(2-(4-chloro-2-fluorophenyl)-2-methylbenzo[d][1,3]dioxol-4-yl)-3,3-difluoropiperidine-1-carboxylate (95 mg, yield: 64.8%). MS Calcd: 483.1; MS Found: 428.3 [M+H−56]⁺.

Step D: 4-[2-(4-chloro-2-fluorophenyl)-2-methyl-2H-1,3-benzodioxol-4-yl]-3,3-difluoropiperidine. A mixture of tert-butyl 4-(2-(4-chloro-2-fluorophenyl)-2-methylbenzo[d][1,3]dioxol-4-yl)-3,3-difluoropiperidine-1-carboxylate (45 mg, 0.09 mmol) and 2,2,2-trifluoroacetic acid (2 mL) in DCM (2 mL) was stirred at rt for 1 h. The mixture was concentrated to give 4-[2-(4-chloro-2-fluorophenyl)-2-methyl-2H-1,3-benzodioxol-4-yl]-3,3-difluoropiperidine, TFA salt (60 mg). MS Calcd: 383.1; MS Found: 384.1 [M+H]⁺.

Step E: 1-((2-((4-(2-(4-chloro-2-fluorophenyl)-2-methylbenzo[d][1,3]dioxol-4-yl)-3,3-difluoropiperidin-1-yl)methyl)-5-(5-(trifluoromethyl)-1,2,4-oxadiazol-3-yl)pyridin-3-yl)methyl)cyclopropane-1-carbonitrile. A mixture of 4-[2-(4-chloro-2-fluorophenyl)-2-methyl-2H-1,3-benzodioxol-4-yl]-3,3-difluoropiperidine, TFA salt (45 mg) and TEA (46 mg, 0.46 mmol) in DCM (5 mL) was stirred at rt for 0.5 h. 1-((2-formyl-5-(5-(trifluoromethyl)-1,2,4-oxadiazol-3-yl)pyridin-3-yl)methyl)cyclopropane-1-carbonitrile (29 mg, 0.09 mmol) was added and the mixture was stirred for 2 h. NaBH(OAc)₃ (77 mg, 0.36 mmol) was added and the mixture was stirred at rt for 16 h. The mixture was poured into water (50 mL) and extracted with DCM (2×50 mL), the combined organic layer was washed with brine, dried over sodium sulfate, filtered and the filtrate was concentrated to give 1-((2-((4-(2-(4-chloro-2-fluorophenyl)-2-methylbenzo[d][1,3]dioxol-4-yl)-3,3-difluoropiperidin-1-yl)methyl)-5-(5-(trifluoromethyl)-1,2,4-oxadiazol-3-yl)pyridin-3-yl)methyl)cyclopropane-1-carbonitrile (60 mg). MS Calcd: 689.2; MS Found: 690.3 [M+H]⁺.

Step F: 1-{[2-({4-[2-(4-chloro-2-fluorophenyl)-2-methyl-2H-1,3-benzodioxol-4-yl]-3,3-difluoropiperidin-1-yl}methyl)-5-[5-(trifluoromethyl)-4H-1,2,4-triazol-3-yl]pyridin-3-yl]methyl}cyclopropane-1-carbonitrile (22). A mixture of 1-((2-((4-((S)-2-(4-chloro-2-fluorophenyl)-2-methylbenzo[d][1,3]dioxol-4-yl)-3,3-difluoropiperidin-1-yl)methyl)-5-(5-(trifluoromethyl)-1,2,4-oxadiazol-3-yl)pyridin-3-yl)methyl)cyclopropane-1-carbonitrile (60 mg, 0.09 mmol) and NH₂NH₂·H₂O (0.2 mL) in EtOH (2 mL) was stirred at 70° C. for 1 h. The mixture was purified by prep-HPLC to give 1-{[2-({4-[2-(4-chloro-2-fluorophenyl)-2-methyl-2H-1,3-benzodioxol-4-yl]-3,3-difluoropiperidin-1-yl}methyl)-5-[5-(trifluoromethyl)-4H-1,2,4-triazol-3-yl]pyridin-3-yl]methyl}cyclopropane-1-carbonitrile (22) (4.2 mg, yield: 6.9%). MS Calcd: 688.2; MS Found: 689.3 [M+H]⁺. ¹H NMR (400 MHz, CD₃OD): δ 9.06 (s, 1H), 8.58 (s, 1H), 7.60 (dd, J₁=18.8 Hz/J₂=8.4 Hz, 1H), 7.29-7.13 (m, 2H), 6.83-6.70 (m, 3H), 3.95 (s, 2H), 3.50-3.10 (m, 4H), 3.02-2.92 (m, 1H), 2.69-2.53 (m, 1H), 2.48-2.21 (m, 2H), 2.05-2.20 (m, 3H), 1.85-1.74 (m, 1H), 1.50-1.40 (m, 2H), 1.27-1.21 (m, 2H). ¹⁹F-NMR (377 MHz, CD₃OD): δ −66.42, −102.36, −102.66, −103.00, −103.30, −112.20, −112.32, −112.66, −112.92, −113.30, −113.56.

### Example 23 and 24: 1-[(2-{[(3,4-trans)-4-[2-(4-chloro-2-fluorophenyl)-2-methyl-2H-1,3-benzodioxol-4-yl]-3-hydroxypiperidin-1-yl]methyl}-5-[5-(trifluoromethyl)-4H-1,2,4-triazol-3-yl]pyridin-3-yl)methyl]cyclopropane-1-carbonitrile (diastereomer 1) (23) and 1-[(2-{[(3,4-trans)-4-[2-(4-chloro-2-fluorophenyl)-2-methyl-2H-1,3-benzodioxol-4-yl]-3-hydroxypiperidin-1-yl]methyl}-5-[5-(trifluoromethyl)-4H-1,2,4-triazol-3-yl]pyridin-3-yl)methyl]cyclopropane-1-carbonitrile (diastereomer 2) (24)

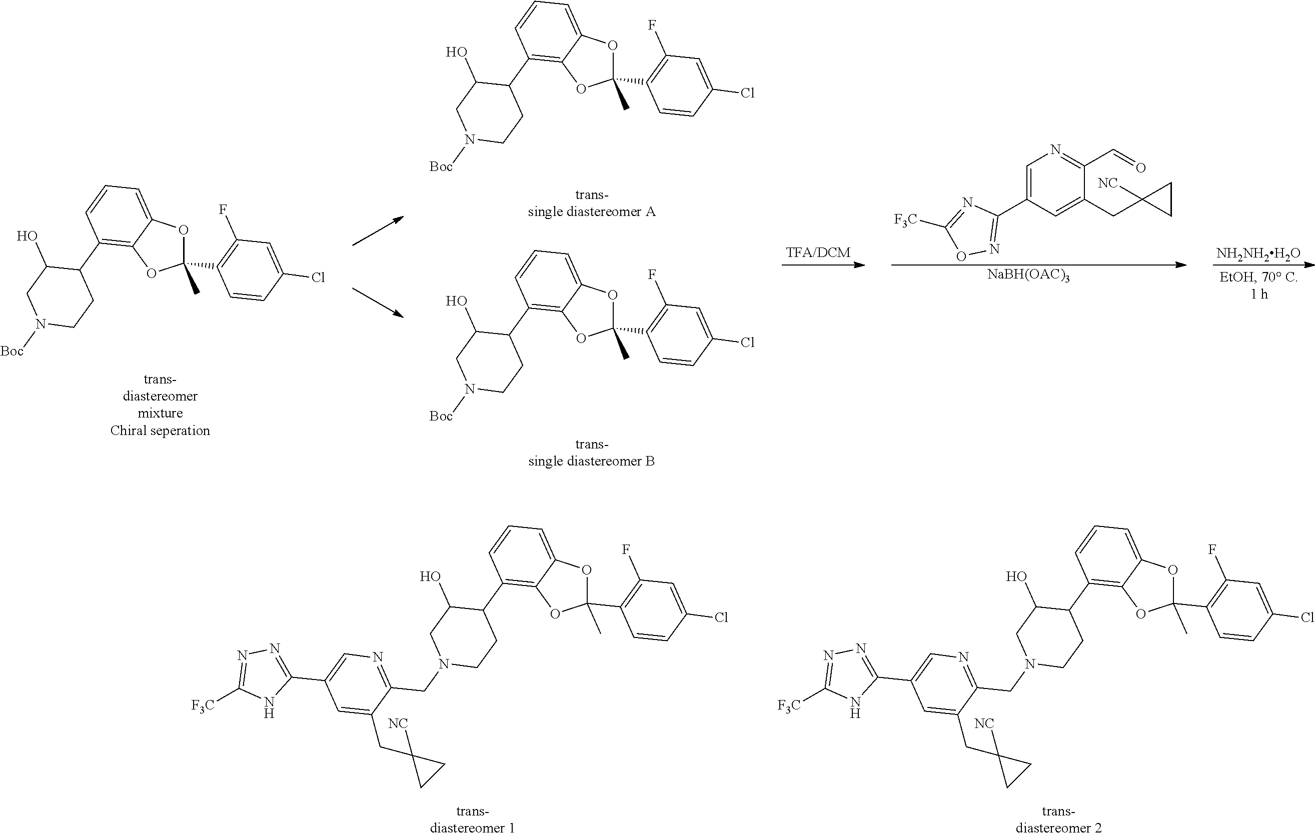

Step A: tert-butyl (3,4-trans)-4-((S)-2-(4-chloro-2-fluorophenyl)-2-methylbenzo[d][1,3]dioxol-4-yl)-3-hydroxypiperidine-1-carboxylate (diastereomer A) and tert-butyl (3,4-trans)-4-((S)-2-(4-chloro-2-fluorophenyl)-2-methylbenzo[d][1,3]dioxol-4-yl)-3-hydroxypiperidine-1-carboxylate (diastereomer B)

The product of tert-butyl (3,4-trans)-4-((S)-2-(4-chloro-2-fluorophenyl)-2-methylbenzo[d][1,3]dioxol-4-yl)-3-hydroxypiperidine-1-carboxylate was submitted for chiral separation (DAICELCHIRALPAK®IG, Supercritical CO₂/IPA (+0.10% 7.0 mol/L ammonia in IPA)=50/50) to obtain two single diastereomers. Diastereomer A: MS Calcd.: 463.2; MS Found: 486.3 [M+23]⁺. SFC Retention Time: 4.17 min. Diastereomer B: MS Calcd.: 463.2; MS Found: 486.3 [M+23]⁺. SFC Retention Time: 6.15 min.

Step B: 1-[(2-{[(3,4-trans)-4-[2-(4-chloro-2-fluorophenyl)-2-methyl-2H-1,3-benzodioxol-4-yl]-3-hydroxypiperidin-1-yl]methyl}-5-[5-(trifluoromethyl)-4H-1,2,4-triazol-3-yl]pyridin-3-yl)methyl]cyclopropane-1-carbonitrile (diastereomer 1) (23)

Starting from the diastereomer A, 1-[(2-{[(3,4-trans)-4-[2-(4-chloro-2-fluorophenyl)-2-methyl-2H-1,3-benzodioxol-4-yl]-3-hydroxypiperidin-1-yl]methyl}-5-[5-(trifluoromethyl)-4H-1,2,4-triazol-3-yl]pyridin-3-yl)methyl]cyclopropane-1-carbonitrile (diastereomer 1, 23) (14.3 mg, yield: 28.6%) was obtained with the similar method of Example 1. MS Calcd: 668.2; MS Found: 669.2 [M+H]⁺. ¹H NMR (400 MHz, CD₃OD): δ 9.28 (s, 1H), 8.58 (d, J₁=1.2 Hz, 1H), 7.75-7.63 (m, 1H), 7.33-7.17 (m, 2H), 6.90-6.75 (m, 3H), 4.48-4.30 (m, 1H), 3.90-3.68 (m, 2H), 3.30-3.28 (m, 3H), 3.19-3.11 (m, 1H), 3.09 (s, 2H), 3.07-2.92 (m, 1H), 2.44-2.05 (m, 5H), 1.50-1.44 (m, 2H), 1.31-1.24 (m, 2H). ¹⁹F NMR (377 MHz, CD₃OD): δ −66.68, −76.96, −112.13.

Step C: 1-[(2-{[(3,4-trans)-4-[2-(4-chloro-2-fluorophenyl)-2-methyl-2H-1,3-benzodioxol-4-yl]-3-hydroxypiperidin-1-yl]methyl}-5-[5-(trifluoromethyl)-4H-1,2,4-triazol-3-yl]pyridin-3-yl)methyl]cyclopropane-1-carbonitrile (diastereomer 2) (24). MS Calcd: 668.2; MS Found: 669.2 [M+H]⁺. ¹H NMR (400 MHz, CD₃OD): δ 9.28 (s, 1H), 8.58 (s, 1H), 7.75-7.62 (m, 1H), 7.33-7.17 (m, 2H), 6.90-6.74 (m, 3H), 4.45-4.29 (m, 1H), 3.88-3.68 (m, 2H), 3.30-3.26 (m, 3H), 3.19-3.08 (m, 1H), 3.07 (s, 2H), 3.06-2.91 (m, 1H), 2.43-2.05 (m, 5H), 1.51-1.44 (m, 2H), 1.32-1.24 (m, 2H). ¹⁹F NMR (377 MHz, CD₃OD): δ −66.68, −77.03, −112.12.

### Example 25 and 26: 1-[(2-{[(3,4-trans)-4-[(2S)-2-(5-chloropyridin-2-yl)-2-methyl-2H-1,3-benzodioxol-4-yl]-3-fluoropiperidin-1-yl]methyl}-5-[5-(trifluoromethyl)-4H-1,2,4-triazol-3-yl]pyridin-3-yl)methyl]cyclopropane-1-carbonitrile (diastereomer 1) (25) and 1-[(2-{[(3,4-trans)-4-[(2S)-2-(5-chloropyridin-2-yl)-2-methyl-2H-1,3-benzodioxol-4-yl]-3-fluoropiperidin-1-yl]methyl}-5-[5-(trifluoromethyl)-4H-1,2,4-triazol-3-yl]pyridin-3-yl)methyl]cyclopropane-1-carbonitrile (diastereomer 2) (26)

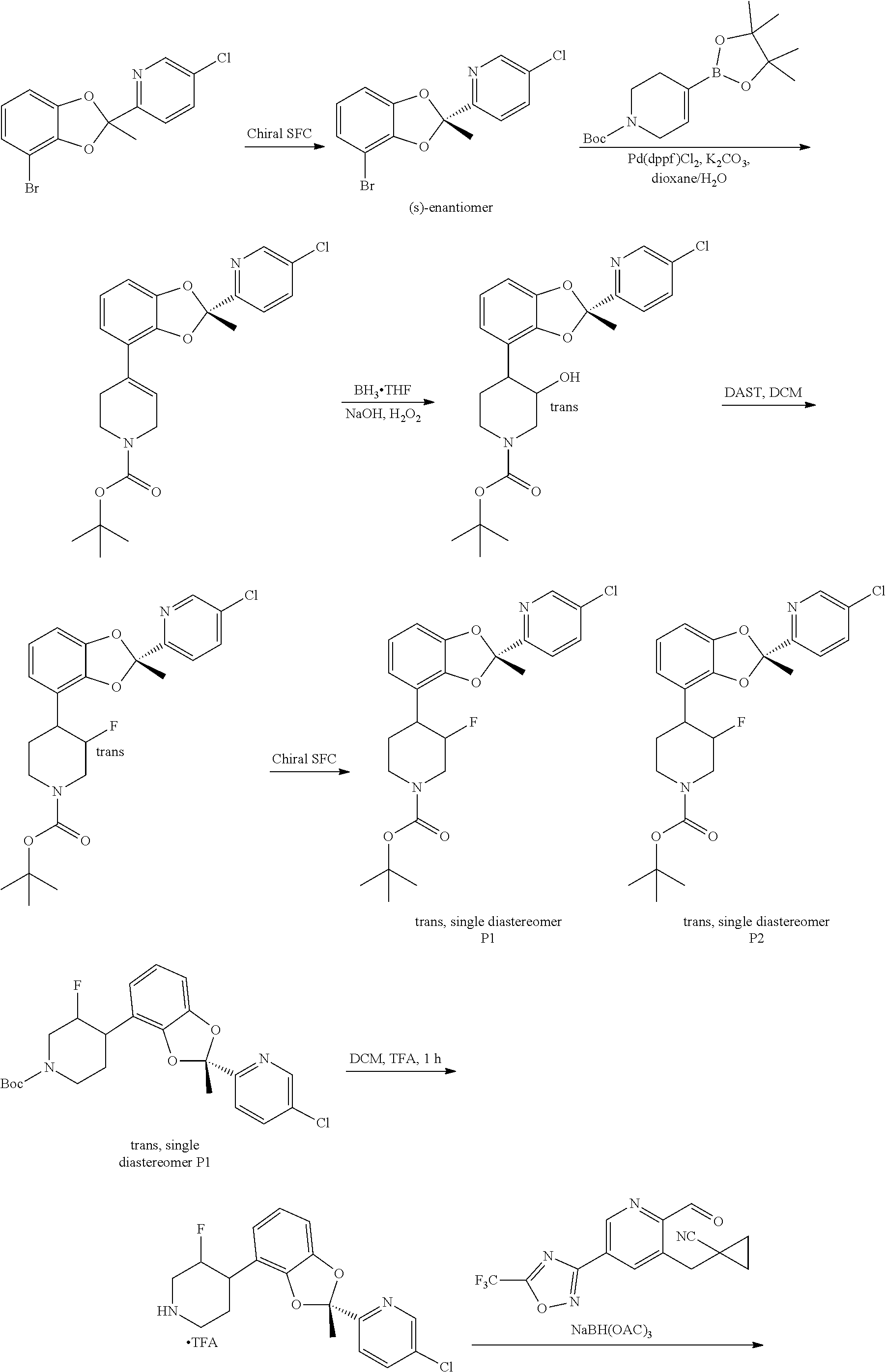

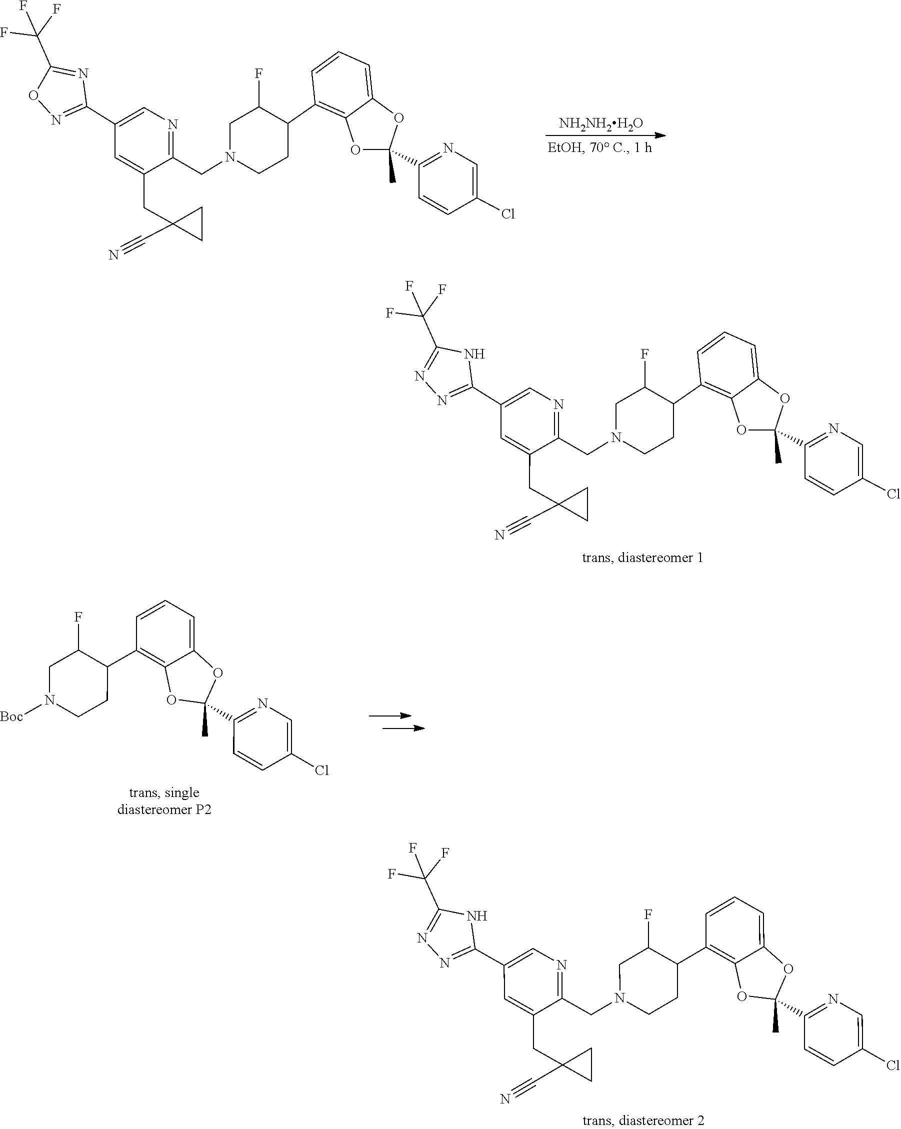 [SMILES: C[C@]1(c2ccc(Cl)cn2)Oc2cccc(C3CCN(Cc4ncc(-c5noc(C(F)(F)F)n5)cc4CC4(C#N)CC4)CC3F)c2O1 ; "NH2NH2·H2O" ; C[C@]1(c2ccc(Cl)cn2)Oc2cccc(C3CCN(Cc4ncc(-c5nnc(C(F)(F)F)[nH]5)cc4CC4(C#N)CC4)CC3F)c2O1 ; CC(C)(C)OC(=O)N1CCC(c2cccc3c2O[C@@](C)(c2ccc(Cl)cn2)O3)C(F)C1 ; C[C@]1(c2ccc(Cl)cn2)Oc2cccc(C3CCN(Cc4ncc(-c5nnc(C(F)(F)F)[nH]5)cc4CC4(C#N)CC4)CC3F)c2O1]

Step A: (S)-2-(4-bromo-2-methylbenzo[d][1,3]dioxol-2-yl)-5-chloropyridine. 2-(4-bromo-2-methylbenzo[d][1,3]dioxol-2-yl)-5-chloropyridine (4.4 g, 13.5 mmol) was separated by SFC (DAICELCHIRALCEL®AD, Supercritical CO₂/MEOH(+0.1% 7.0 mol/1 Ammonia in MEOH) 65/35) to give (S)-2-(4-bromo-2-methylbenzo[d][1,3]dioxol-2-yl)-5-chloropyridine (2.2 g, yield: 50%). MS Calcd.: 325.0; MS Found: 325.9 [M+H]⁺. SFC Retention Time: 0.82 min.

And (R)-2-(4-bromo-2-methylbenzo[d][1,3]dioxol-2-yl)-5-chloropyridine (2.2 g, yield: 50%). MS Calcd.: 325.0; MS Found: 326.1 [M+H]⁺. SFC Retention Time: 0.91 min.

Step B: tert-butyl (S)-4-(2-(5-chloropyridin-2-yl)-2-methylbenzo[d][1,3]dioxol-4-yl)-3,6-dihydropyridine-1(2H)-carboxylate. A mixture of (S)-2-(4-bromo-2-methylbenzo[d][1,3]dioxol-2-yl)-5-chloropyridine (2.2 g, 6.8 mmol), tert-butyl 4-(4,4,5,5-tetramethyl-1,3,2-dioxaborolan-2-yl)-3,6-dihydropyridine-1(2H)-carboxylate (2.1 g, 6.8 mmol), Pd(dppf)Cl₂ (495 mg, 0.68 mmol), K₂CO₃ (2.8 g, 20.4 mmol) in dioxane/H₂O (20.0 mL/2.0 mL) was stirred at 90° C. for 3 hours. After the reaction was completed, the mixture was diluted with H₂O (100 mL), extracted with EA (50 mL×2). The combined organic layer was dried over anhydrous sodium sulfate, filtered, and evaporated to dryness. The residue was purified by column chromatography on silica gel (PE/EA=20/1) to give tert-butyl (S)-4-(2-(5-chloropyridin-2-yl)-2-methylbenzo[d][1,3]dioxol-4-yl)-3,6-dihydropyridine-1(2H)-carboxylate (2.1 g, yield: 73%). MS Calcd.: 428.2; MS Found: 373.0 [M-56+H]⁺.

Step C: tert-butyl (3,4-trans)-4-[(2S)-2-(5-chloropyridin-2-yl)-2-methyl-2H-1,3-benzodioxol-4-yl]-3-hydroxypiperidine-1-carboxylate. To a solution of tert-butyl (S)-4-(2-(5-chloropyridin-2-yl)-2-methylbenzo[d][1,3]dioxol-4-yl)-3,6-dihydropyridine-1(2H)-carboxylate (2.0 g, 4.7 mmol) in THE (40 mL) was added BH₃THF (9.35 mL, 9.4 mmol) slowly at 0° C. The mixture was stirred at 0° C. for 2 hours. Then NaOH solution (7 mL, 2 M in water) was added, followed by the addition of H₂O₂(7 mL, 30% in water). The reaction was stirred at 50° C. for 1 hour. After the reaction was completed, the mixture was diluted with EA (100 mL) and wished with water (30 mL×2). The organic layer was dried over Na₂SO₄, filtered, and evaporated to dryness. The residue was purified by column chromatography on silica gel (PE:EA=2:1) to give tert-butyl (3,4-trans)-4-[(2S)-2-(5-chloropyridin-2-yl)-2-methyl-2H-1,3-benzodioxol-4-yl]-3-hydroxypiperidine-1-carboxylate (1.5 g, yield: 75%).

MS Calcd.: 446.2; MS Found: 469.1 [M+Na]⁺.

Step D: tert-butyl (3,4-trans)-4-[(2S)-2-(5-chloropyridin-2-yl)-2-methyl-2H-1,3-benzodioxol-4-yl]-3-fluoropiperidine-1-carboxylate. To a mixture of tert-butyl (3,4-trans)-4-[(2S)-2-(5-chloropyridin-2-yl)-2-methyl-2H-1,3-benzodioxol-4-yl]-3-hydroxypiperidine-1-carboxylate (1.35 g, 3.03 mmol) in DCM (20 mL) was added DAST (975 mg, 6.05 mmol) slowly at 0° C. The mixture was stirred at 0° C. for 2 minutes. After the reaction was completed, the mixture was diluted with EA (100 mL) and wished with water (30 mL×2). The organic layer was dried over Na₂SO₄, filtered, and evaporated to dryness. The residue was purified by column chromatography on silica gel (PE:EA=10:1) to give to give tert-butyl (3,4-trans)-4-[(2S)-2-(5-chloropyridin-2-yl)-2-methyl-2H-1,3-benzodioxol-4-yl]-3-fluoropiperidine-1-carboxylate (1.2 g, yield: 88.9%). MS Calcd.: 448.2; MS Found: 449.2 [M+H]⁺.

Step E: tert-butyl 4-(3,4-trans)-((S)-2-(5-chloropyridin-2-yl)-2-methylbenzo[d][1,3]dioxol-4-yl)-3-fluoropiperidine-1-carboxylate, single diastereomer P1 and P2. tert-butyl (3,4-trans)-4-[(2S)-2-(5-chloropyridin-2-yl)-2-methyl-2H-1,3-benzodioxol-4-yl]-3-fluoropiperidine-1-carboxylate (1.2 g, 2.68 mmol) was separated by SFC (DAICELCHIRALCEL®AD, Supercritical CO₂/MEOH(+0.1% 7.0 mol/l Ammonia in MEOH)=60/40) to give diastereomer P1 (670 mg, yield: 55.8%) and diastereomer P2 (430 mg, yield: 35.8%).

diastereomer P1 (670 mg, yield: 55.8%). MS Calcd.: 448.2; MS Found: 449.1 [M+H]⁺. SFC Retention Time: 1.02 min. ¹⁹F NMR: (377 MHz, DMSO-d6): δ −182.46.

diastereomer P2 (430 mg, yield: 35.8%). MS Calcd.: 448.16; MS Found: 449.1 [M+H]⁺. SFC Retention Time: 1.37 min.

¹H NMR: δ 8.72 (d, J=0.2 Hz, 1H), 8.02 (dd, J=2.4 Hz, 8.4 Hz, 1H), 7.59 (d, J=8.4 Hz, 1H), 6.80-6.90 (m, 3H), 4.56-4.81 (m, 1H), 4.21-4.34 (m, 1H), 3.87-4.03 (m, 1H), 2.96-3.07 (m, 1H), 2.80-2.95 (m, 2H), 2.02 (s, 3H), 1.65-1.85 (m, 2H), 1.42 (s, 9H). ¹⁹F NMR: (377 MHz, DMSO−d6): δ: −182.27.

Step F: 5-chloro-2-((S)-4-((3,4-trans)-3-fluoropiperidin-4-yl)-2-methylbenzo[d][1,3]dioxol-2-yl)pyridine TFA Salt (from diastereomer P1). To a solution of tert-butyl (3,4-trans)-4-((S)-2-(5-chloropyridin-2-yl)-2-methylbenzo[d][1,3]dioxol-4-yl)-3-fluoropiperidine-1-carboxylate (isomer P1)(100 mg, 0.22 mmol) in DCM (4 mL) was added TFA (1.0 mL). The solution was stirred at room temperature for 1 hour. The reaction was removed in vacuo to give 5-chloro-2-((S)-4-((3,4-trans)-3-fluoropiperidin-4-yl)-2-methylbenzo[d][1,3]dioxol-2-yl)pyridine TFA salt (from diastereomer P1)(120 mg, crude). MS Calcd.: 348.1; MS Found: 349.1 [M+H]⁺.

Step G: 1-((2-(((3,4-trans)-4-((S)-2-(5-chloropyridin-2-yl)-2-methylbenzo[d][1,3]dioxol-4-yl)-3-fluoropiperidin-1-yl)methyl)-5-(5-(trifluoromethyl)-1,2,4-oxadiazol-3-yl)pyridin-3-yl)methyl)cyclopropane-1-carbonitrile (from diastereomer P1). A mixture of 5-chloro-2-((S)-4-((3,4-trans)-3-fluoropiperidin-4-yl)-2-methylbenzo[d][1,3]dioxol-2-yl)pyridine (from isomer P1) (64 mg, 0.184 mmol) and TEA (93 mg, 0.919 mmol) in DCM (5 mL) was stirred at rt for 0.5 h. 3-((methylsulfonyl)methyl)-5-(5-(trifluoromethyl)-1,2,4-oxadiazol-3-yl)picolinaldehyde (59 mg, 0.184 mmol) was added and the mixture was stirred for 2 h. NaBH(OAc)₃ (156 mg, 0.736 mmol) was added and the mixture was stirred at rt for 16 h. The mixture was poured into water (50 mL) and extracted with DCM (2×50 mL), the combined organic layer was washed with brine, dried over sodium sulfate, filtered and the filtrate was concentrated. The residue was purified by TLC gel to give 1-((2-(((3,4-trans)-4-((S)-2-(5-chloropyridin-2-yl)-2-methylbenzo[d][1,3]dioxol-4-yl)-3-fluoropiperidin-1-yl)methyl)-5-(5-(trifluoromethyl)-1,2,4-oxadiazol-3-yl)pyridin-3-yl)methyl)cyclopropane-1-carbonitrile (from diastereomer P1) (100 mg, yield: 83.33%). MS Calcd: 654.2; MS Found: 655.3 [M+H]⁺.

Step H: 1-[(2-{[(3,4-trans)-4-[(2S)-2-(5-chloropyridin-2-yl)-2-methyl-2H-1,3-benzodioxol-4-yl]-3-fluoropiperidin-1-yl]methyl}-5-[5-(trifluoromethyl)-4H-1,2,4-triazol-3-yl]pyridin-3-yl)methyl]cyclopropane-1-carbonitrile (diastereomer 1) (25)

A mixture of 1-((2-(((3,4-trans)-4-((S)-2-(5-chloropyridin-2-yl)-2-methylbenzo[d][1,3]dioxol-4-yl)-3-fluoropiperidin-1-yl)methyl)-5-(5-(trifluoromethyl)-1,2,4-oxadiazol-3-yl)pyridin-3-yl)methyl)cyclopropane-1-carbonitrile (from diastereomer P1) (100 mg, 0.15 mmol) and NH₂NH₂·H₂O (2.0 mL) in EtOH (2 mL) was stirred at 70° C. for 1 h. The mixture was purified by prep-HPLC to give 1-[(2-{[(3,4-trans)-4-[(2S)-2-(5-chloropyridin-2-yl)-2-methyl-2H-1,3-benzodioxol-4-yl]-3-fluoropiperidin-1-yl]methyl}-5-[5-(trifluoromethyl)-4H-1,2,4-triazol-3-yl]pyridin-3-yl)methyl]cyclopropane-1-carbonitrile (diastereomer 1) (6.0 mg, yield: 6.0%).

MS Calcd: 653.2; MS Found: 654.3 [M+H]⁺.

¹H NMR (400 MHz, CD₃OD): δ 9.07 (s, 1H), 8.59 (s, 1H), 8.54 (s, 1H), 7.86 (dd, J1=8.4 Hz/J₂=6.0 Hz, 1H), 7.65 (d, J=8.4 Hz, 1H), 6.84-6.69 (m, 3H), 4.99-4.90 (m, 1H), 3.99-3.82 (m, 2H), 3.32-3.21 (m, 3H), 2.95-2.80 (m, 2H), 2.36-2.24 (m, 2H), 2.05 (s, 3H), 2.06-1.90 (m, 1H), 1.88-1.77 (m, 1H), 1.47-1.40 (m, 2H), 1.38-1.20 (m, 2H). ¹⁹F NMR (377 MHz, CD₃OD): δ −66.40, −184.98.

1-[(2-{[(3,4-trans)-4-[(2S)-2-(5-chloropyridin-2-yl)-2-methyl-2H-1,3-benzodioxol-4-yl]-3-fluoropiperidin-1-yl]methyl}-5-[5-(trifluoromethyl)-4H-1,2,4-triazol-3-yl]pyridin-3-yl)methyl]cyclopropane-1-carbonitrile (diastereomer 2) (26) was obtained using the similar procedure described for Example 25 by using tert-butyl (3,4-trans)-4-((S)-2-(5-chloropyridin-2-yl)-2-methylbenzo[d][1,3]dioxol-4-yl)-3-fluoropiperidine-1-carboxylate (diastereomer P2) as starting material.

MS Calcd: 653.2; MS Found: 654.3 [M+H]⁺.

¹H NMR (400 MHz, CD₃OD): δ 9.07 (s, 1H), 8.60 (d, J=2.0 Hz, 1H), 8.54 (s, 1H), 7.84 (dd, J₁=2.4 Hz/J 8.8 Hz, 1H), 7.66 (d, J₁=8.4 Hz, 1H), 6.81-6.73 (m, 2H), 6.71 (d, J₁=7.2 Hz, 1H), 4.82-4.73 (m, 1H), 3.98-3.85 (m, 2H), 3.24 (s, 2H), 3.15-3.10 (m, 1H), 2.95-2.80 (m, 2H), 2.35-2.25 (t, J=10.6 Hz, 2H), 2.03 (s, 3H), 2.05-1.90 (m, 1H), 1.88-1.78 (m, 1H), 1.47-1.42 (m, 2H), 1.28-1.21 (m, 2H). ¹⁹F NMR (377 MHz, CD₃OD): δ −66.18, −184.67.

### Example 27 and 28: 1-((2-(((3,4-trans)-4-((S)-2-(4-chloro-2-fluorophenyl)-2-methylbenzo[d][1,3]dioxol-4-yl)-3-fluoropiperidin-1-yl)methyl)-5-(5-(trifluoromethyl)-4H-1,2,4-triazol-3-yl)pyridin-3-yl)methyl)cyclopropane-1-carbonitrile (27) (diastereomer 1) and 1-((2-(((3,4-trans)-4-((S)-2-(4-chloro-2-fluorophenyl)-2-methylbenzo[d][1,3]dioxol-4-yl)-3-fluoropiperidin-1-yl)methyl)-5-(5-(trifluoromethyl)-4H-1,2,4-triazol-3-yl)pyridin-3-yl)methyl)cyclopropane-1-carbonitrile (28) (diastereomer 2)

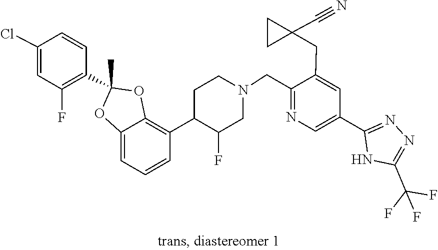 [SMILES: C[C@]1(c2ccc(Cl)cc2F)Oc2cccc(C3CCN(Cc4ncc(-c5nnc(C(F)(F)F)[nH]5)cc4CC4(C#N)CC4)CC3F)c2O1]

MS Calcd: 670.2; MS Found: 671.2 [M+H]⁺. ¹H NMR (400 MHz, CD₃OD-d₄): δ 9.07 (s, 1H), 8.56 (s, 1H), 7.63-7.54 (m, 1H), 7.30-7.23 (m, 1H), 7.23-7.15 (m, 1H), 6.82-6.69 (m, 3H), 4.95-4.93 (m, 1H), 4.00-3.86 (m, 2H), 3.27-3.20 (m, 2H), 2.96-2.78 (m, 3H), 2.35-2.25 (m, 2H), 2.10-1.75 (m, 5H), 1.48-1.40 (m, 2H), 1.30-1.24 (m, 2H). ¹⁹F NMR (377 MHz, CD₃OD): δ −66.47, −112.31, −112.38, −184.66, −185.00.

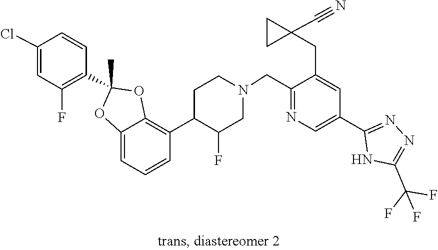 [SMILES: C[C@]1(c2ccc(Cl)cc2F)Oc2cccc(C3CCN(Cc4ncc(-c5nnc(C(F)(F)F)[nH]5)cc4CC4(C#N)CC4)CC3F)c2O1]

MS Calcd: 670.2; MS Found: 671.2 [M+H]⁺. ¹H NMR (400 MHz, CD₃OD): δ 9.07 (d, J=1.6 Hz, 1H), 8.56 (s, 1H), 7.64-7.55 (m, 1H), 7.31-7.23 (m, 1H), 7.23-7.15 (m, 1H), 6.83-6.69 (m, 3H), 4.94-4.91 (m, 1H), 4.01-3.86 (m, 2H), 3.28-3.20 (m, 2H), 3.00-2.75 (m, 3H), 2.31 (t, J=11.6 Hz, 2H), 2.10-1.78 (m, 5H), 1.48-1.42 (m, 2H), 1.27-1.22 (m, 2H). ¹⁹F NMR (377 MHz, CD₃OD): δ −66.58, −112.31, −112.38, −184.57, −184.68, −184.70, −184.95, −185.02.

### Example 29 and 30: 1-((2-((trans)-4-((R)-2-(4-chloro-2-fluorophenyl)-7-fluoro-2-methylbenzo[d][1,3]dioxol-4-yl)-3-fluoropiperidin-1-yl)methyl)-5-(5-(trifluoromethyl)-4H-1,2,4-triazol-3-yl)pyridin-3-yl)methyl)cyclopropane-1-carbonitrile diastereomer 1 (29) and 1-((2-((trans)-4-((R)-2-(4-chloro-2-fluorophenyl)-7-fluoro-2-methylbenzo[d][1,3]dioxol-4-yl)-3-fluoropiperidin-1-yl)methyl)-5-(5-(trifluoromethyl)-4H-1,2,4-triazol-3-yl)pyridin-3-yl)methyl)cyclopropane-1-carbonitrile diastereomer 2 (30)

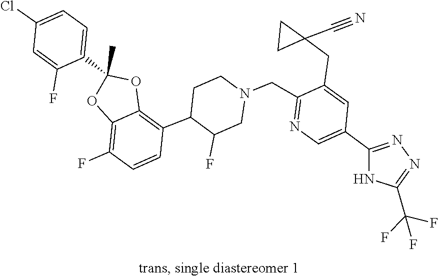 [SMILES: C[C@]1(c2ccc(Cl)cc2F)Oc2c(F)ccc(C3CCN(Cc4ncc(-c5nnc(C(F)(F)F)[nH]5)cc4CC4(C#N)CC4)CC3F)c2O1]

MS Calcd: 688.2; MS Found: 689.4 [M+H]⁺. ¹H NMR (400 MHz, CD₃OD): δ 9.09 (d, J=2.0 Hz, 1H), 8.58 (d, J=2.0 Hz, 1H), 7.62 (t, J=8.4 Hz, 1H), 7.33 (dd, J₁=2.0 Hz/J₂ 11.2 Hz, 1H), 7.26 (dd, J₁=2.0 Hz/1J₂=8.4 Hz, 1H), 6.81-6.65 (m, 2H), 4.94 (dd, J=4.8 Hz/J₂=9.2 Hz, 0.5H), 4.81 (dd, J₁=4.8 Hz/J₂=9.6 Hz, 0.5H), 4.02-3.87 (m, 2H), 3.28 (s, 3H), 2.97-2.78 (m, 2H), 2.36-2.26 (m, 2H), 2.10 (s, 3H), 2.02-1.93 (m, 1H), 1.87-1.79 (m, 1H), 1.49-1.44 (m, 2H), 1.31-1.24 (m, 2H). ¹⁹F NMR (377 MHz, CD₃OD): S (−66.50), (−112.22), (−143.14), (−185.19).

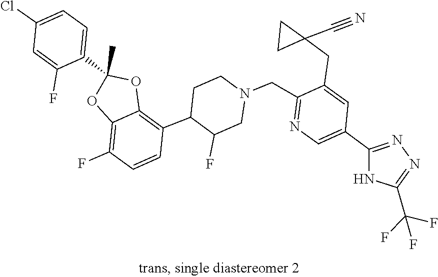 [SMILES: C[C@]1(c2ccc(Cl)cc2F)Oc2c(F)ccc(C3CCN(Cc4ncc(-c5nnc(C(F)(F)F)[nH]5)cc4CC4(C#N)CC4)CC3F)c2O1]

MS Calcd: 688.2; MS Found: 689.3 [M+H]⁺. ¹H NMR (400 MHz, CD₃OD): δ 9.09 (d, J=1.6 Hz, 1H), 8.58 (d, J=1.6 Hz, 1H), 7.63 (t, J=8.4 Hz, 1H), 7.32 (dd, J₁=2.0 Hz/J₂=11.2 Hz, 1H), 7.23 (dd, J, =1.6 Hz/112=8.8 Hz, 1H), 6.81-6.64 (m, 2H), 4.92 (dd, J₁=4.0 Hz/J₂=8.8 Hz, 0.5H), 4.76 (dd, J, =5.2 Hz/J₂=10.0 Hz, 0.5H), 4.00-3.89 (m, 2H), 3.29 (d, J=7.2 Hz, 3H), 2.98-2.80 (m, 2H), 2.36-2.25 (m, 2H), 2.11 (s, 3H), 2.07-1.94 (m, 1H), 1.87-1.78 (m, 1H), 1.50-1.44 (m, 2H), 1.32-1.25 (m, 2H). ¹⁹F NMR (377 MHz, CD₃OD): δ (−66.37), (−112.12), (−143.09), (−184.81).

### Example 31: 1-((2-((4-(2-(4-chloro-2-fluorophenyl)-2-methylbenzo[d][1,3]dioxol-4-yl)-3,6-dihydropyridin-1(2H)-yl)methyl)-5-(5-(trifluoromethyl)-4H-1,2,4-triazol-3-yl)pyridin-3-yl)methyl)cyclopropane-1-carbonitrile (31)

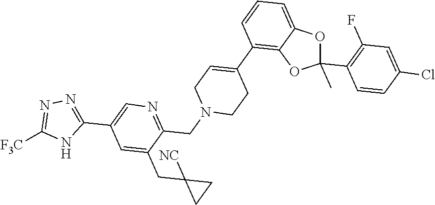 [SMILES: CC1(c2ccc(Cl)cc2F)Oc2cccc(C3=CCN(Cc4ncc(-c5nnc(C(F)(F)F)[nH]5)cc4CC4(C#N)CC4)CC3)c2O1]

Starting from 4-(2-(4-chloro-2-fluorophenyl)-2-methylbenzo[d][1,3]dioxol-4-yl)-1,2,3,6-tetrahydropyridine TFA salt, 1-((2-((4-(2-(4-chloro-2-fluorophenyl)-2-methylbenzo[d][1,3]dioxol-4-yl)-3,6-dihydropyridin-1(2H)-yl)methyl)-5-(5-(trifluoromethyl)-4H-1,2,4-triazol-3-yl)pyridin-3-yl)methyl)cyclopropane-1-carbonitrile was obtained with the similar method of Example 1 (3.7 mg). MS Calcd: 650.2; MS Found: 651.2 [M+H]⁺. ¹H NMR (400 MHz, CD₃OD): δ 9.17 (s, 1H), 8.52 (s, 1H), 7.58 (t, J=8.4 Hz, 1H), 7.31-7.18 (m, 2H), 6.92-6.78 (m, 3H), 6.43 (s, 1H), 4.41 (s, 2H), 3.74 (s, 2H), 3.25-3.21 (m, 2H), 3.13 (s, 2H), 2.82 (s, 2H), 2.05 (s, 3H), 1.44-1.39 (m, 2H), 1.28-1.24 (m, 2H). ¹⁹F NMR (377 MHz, CD₃OD): δ (−66.56), (−112.46).

### Example 32: 1-({2-[(4-{6-[(4-chloro-2-fluorophenyl)methoxy]pyridin-2-yl}piperidin-1-yl)methyl]-5-[5-(trifluoromethyl)-4H-1,2,4-triazol-3-yl]pyridin-3-yl}methyl)cyclopropane-1-carbonitrile (32)

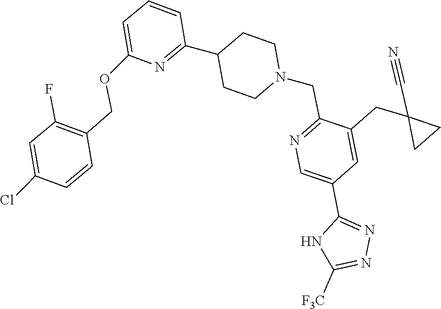 [SMILES: N#CC1(Cc2cc(-c3nnc(C(F)(F)F)[nH]3)cnc2CN2CCC(c3cccc(OCc4ccc(Cl)cc4F)n3)CC2)CC1]

Starting from 2-((4-chloro-2-fluorobenzyl)oxy)-6-(piperidin-4-yl)pyridine hydrochloride, 1-({2-[(4-{6-[(4-chloro-2-fluorophenyl)methoxy]pyridin-2-yl}piperidin-1-yl)methyl]-5-[5-(trifluoromethyl)-4H-1,2,4-triazol-3-yl]pyridin-3-yl}methyl)cyclopropane-1-carbonitrile (32) (7.2 mg, yield: 38.3%) was obtained with the similar method of Example 1. MS Calcd: 625.2; MS Found: 626.2 [M+H]⁺. ¹H NMR (400 MHz, CD₃OD): δ 9.21 (d, J=2.0 Hz, 1H), 8.50 (d, J=1.6 Hz, 1H), 7.62 (t, J=3.8 Hz, 1H), 7.52 (t, J=4.0 Hz, 1H), 7.27-7.19 (m, 2H), 6.90 (d, J=7.2 Hz, 1H), 6.70 (d, J=8.4 Hz, 1H), 5.45 (s, 2H), 4.55-4.45 (m, 2H), 3.61-3.56 (m, 2H), 3.17-2.90 (m, 5H), 2.28-2.05 (m, 4H), 1.44-1.39 (m, 2H), 1.33-1.25 (m, 2H), ¹⁹F-NMR (377 MHz, CD₃OD): δ −65.07, −117.58.

### Example 33: 1-((2-((4-(6-((4-chloro-2-fluorobenzyl)oxy)-5-fluoropyridin-2-yl)piperidin-1-yl)methyl)-5-(5-(trifluoromethyl)-4H-1,2,4-triazol-3-yl)pyridin-3-yl)methyl)cyclopropane-1-carbonitrile (33)

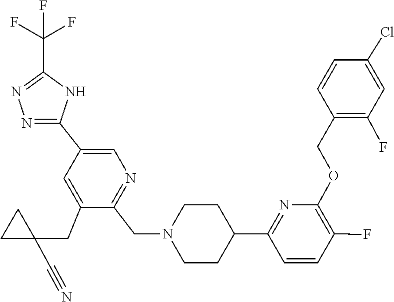 [SMILES: N#CC1(Cc2cc(-c3nnc(C(F)(F)F)[nH]3)cnc2CN2CCC(c3ccc(F)c(OCc4ccc(Cl)cc4F)n3)CC2)CC1]

Starting from 1-(4-(6-((4-chloro-2-fluorobenzyl)oxy)-5-fluoropyridin-2-yl)-1|4-piperidin-1-yl) TFA salt, 1-((2-((4-(6-((4-chloro-2-fluorobenzyl)oxy)-5-fluoropyridin-2-yl)piperidin-1-yl)methyl)-5-(5-(trifluoromethyl)-4H-1,2,4-triazol-3-yl)pyridin-3-yl)methyl)cyclopropane-1-carbonitrile (23.2 mg, yield: 19.4%) was obtained with the similar method of Example 1. MS Calcd: 643.1; MS Found: 644.1 [M+H]⁺. ¹H NMR (400 MHz, CD₃OD): δ 9.21 (s, 1H), 8.50 (d, J=1.6 Hz, 1H), 7.54 (t, J=8.4 Hz, 1H), 7.46-7.42 (m, 1H), 7.27-7.22 (m, 2H), 6.93-6.86 (m, 1H), 5.54 (s, 2H), 4.57 (s, 2H), 3.60-3.52 (m, 1H), 3.36-3.33 (m, 2H), 3.27-3.23 (m, 2H), 3.07 (s, 2H), 2.20-2.03 (m, 4H), 1.43-1.40 (m, 2H), 1.34-1.28 (m, 2H). ¹⁹F NMR (377 MHz, CD₃OD): δ −65.21, −86.99, −117.35.

### Example 34: 1-({2-[(4-{2-[(4-chloro-2-fluorophenyl)methoxy]pyrimidin-4-yl}piperidin-1-yl)methyl]-5-[5-(trifluoromethyl)-4H-1,2,4-triazol-3-yl]pyridin-3-yl}methyl)cyclopropane-1-carbonitrile (34)

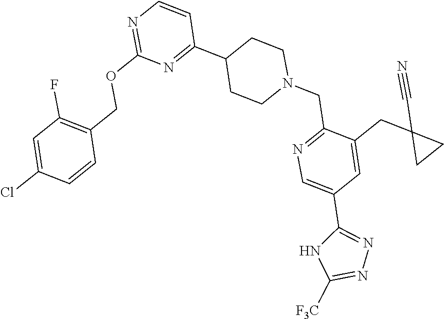 [SMILES: N#CC1(Cc2cc(-c3nnc(C(F)(F)F)[nH]3)cnc2CN2CCC(c3ccnc(OCc4ccc(Cl)cc4F)n3)CC2)CC1]

Starting from 2-[(4-chloro-2-fluorophenyl)methoxy]-4-(piperidin-4-yl)pyrimidine TFA salt, 1-({2-[(4-{2-[(4-chloro-2-fluorophenyl)methoxy]pyrimidin-4-yl}piperidin-1-yl)methyl]-5-[5-(trifluoromethyl)-4H-1,2,4-triazol-3-yl]pyridin-3-yl}methyl)cyclopropane-1-carbonitrile (34) (6.5 mg, yield: 26.1%) was obtained with the similar method. MS Calcd: 626.2; MS Found: 627.0 [M+H]⁺. ¹H NMR (400 MHz, CD₃OD): δ 9.27 (s, 1H), 8.57 (s, 1H), 8.54 (d, J=5.2 Hz, 1H), 7.57 (t, J=8.0 Hz, 1H), 7.27 (t, J=8.0 Hz, 2H), 7.13 (d, J=4.0 Hz, 1H), 5.53 (s, 2H), 4.80 (s, 2H), 3.93-3.77 (m, 2H), 3.46-3.30 (m, 2H), 3.19-3.05 (m, 1H), 3.07 (s, 2H), 2.40-2.21 (m, 4H), 1.50-1.42 (m, 2H), 1.33-1.25 (m, 2H). ¹⁹F-NMR (377 MHz, CD₃OD): δ −66.6, −76.95, −117.24.

### Example 35: 1-({2-[(4-{2-[(4-chloro-2-fluorophenyl)methoxy]-5-fluoropyrimidin-4-yl}piperidin-1-yl)methyl]-5-[5-(trifluoromethyl)-4H-1,2,4-triazol-3-yl]pyridin-3-yl}methyl)cyclopropane-1-carbonitrile (35)

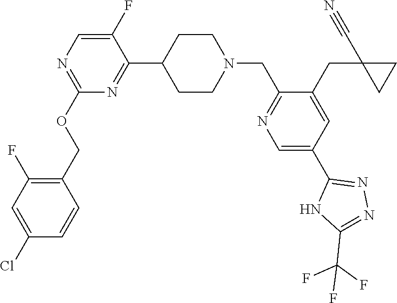 [SMILES: N#CC1(Cc2cc(-c3nnc(C(F)(F)F)[nH]3)cnc2CN2CCC(c3nc(OCc4ccc(Cl)cc4F)ncc3F)CC2)CC1]

Starting from 2-[(4-chloro-2-fluorophenyl)methoxy]-5-fluoro-4-(piperidin-4-yl)pyrimidine TFA salt, 1-({2-[(4-{2-[(4-chloro-2-fluorophenyl)methoxy]-5-fluoropyrimidin-4-yl}piperidin-1-yl)methyl]-5-[5-(trifluoromethyl)-4H-1,2,4-triazol-3-yl]pyridin-3-yl}methyl)cyclopropane-1-carbonitrile (35) (19.5 mg, yield: 22.2%) was obtained with the similar method. MS Calcd: 644.2; MS Found: 645.2 [M+H]⁺.

¹H NMR (400 MHz, CD₃OD): δ 9.27 (s, 1H), 8.57 (t, J=2.0 Hz, 1H), 8.48 (s, 1H), 7.56 (t, J=8.4 Hz, 1H), 7.26 (t, J=8.8 Hz, 2H), 5.49 (s, 2H), 4.80 (s, 2H), 3.94-3.76 (m, 2H), 3.54-3.36 (m, 3H), 3.06 (s, 2H), 2.44-2.31 (m, 2H), 2.27-2.14 (m, 2H), 1.51-1.42 (m, 2H), 1.32-1.24 (m, 2H). ¹⁹F-NMR (377 MHz, CD₃OD): δ −66.68, −77.08, −117.25, −152.46.

### Example 36: 1-((2-((4-(4-((4-chloro-2-fluorobenzyl)oxy)-5-fluoropyrimidin-2-yl)piperidin-1-yl)methyl)-5-(5-(trifluoromethyl)-4H-1,2,4-triazol-3-yl)pyridin-3-yl)methyl)cyclopropane-1-carbonitrile (36)

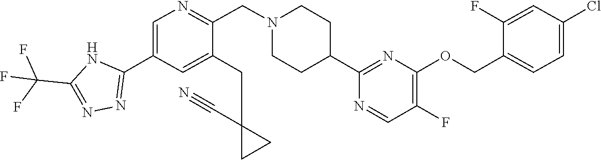 [SMILES: N#CC1(Cc2cc(-c3nnc(C(F)(F)F)[nH]3)cnc2CN2CCC(c3ncc(F)c(OCc4ccc(Cl)cc4F)n3)CC2)CC1]

Staring from 1-(4-(4-((4-chloro-2-fluorobenzyl)oxy)-5-fluoropyrimidin-2-yl)-1|4-piperidin-1-yl) TFA salt, 1-((2-((4-(4-((4-chloro-2-fluorobenzyl)oxy)-5-fluoropyrimidin-2-yl)piperidin-1-yl)methyl)-5-(5-(trifluoromethyl)-4H-1,2,4-triazol-3-yl)pyridin-3-yl)methyl)cyclopropane-1-carbonitrile (16.6 mg) was obtained with the similar method of Example 1. MS Calcd: 644.2; MS Found: 645.2 [M+H]⁺. ¹H NMR (400 MHz, CD₃OD): δ 9.19 (s, 1H), 8.51 (s, 1H), 8.39 (s, 1H), 7.54 (t, J=7.6 Hz, 1H), 7.26 (t, J=9.2 Hz, 2H), 5.60 (s, 2H), 4.44 (s, 2H), 3.53-3.44 (m, 2H), 3.17-3.13 (m, 1H), 3.09 (s, 2H), 3.05-2.99 (m, 2H), 2.28-2.15 (m, 4H), 1.46-1.38 (m, 1H), 1.30-1.21 (m, 2H). ¹⁹F NMR (377 MHz, CD₃OD-d₄): δ −65.29, −116.87, −158.41.

### Example 37: 1-({2-[(4-{2-[(4-chloro-2-fluorophenyl)methoxy]pyridin-3-yl}piperidin-1-yl)methyl]-5-[5-(trifluoromethyl)-4H-1,2,4-triazol-3-yl]pyridin-3-yl}methyl)cyclopropane-1-carbonitrile (37)

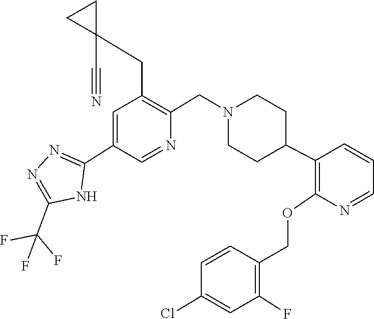 [SMILES: N#CC1(Cc2cc(-c3nnc(C(F)(F)F)[nH]3)cnc2CN2CCC(c3cccnc3OCc3ccc(Cl)cc3F)CC2)CC1]

Starting from 2-((4-chloro-2-fluorobenzyl)oxy)-3-(piperidin-4-yl)pyridine HCl salt, 1-({2-[(4-{2-[(4-chloro-2-fluorophenyl)methoxy]pyridin-3-yl}piperidin-1-yl)methyl]-5-[5-(trifluoromethyl)-4H-1,2,4-triazol-3-yl]pyridin-3-yl}methyl)cyclopropane-1-carbonitrile (37) (8.1 mg) was obtained with the similar method of Example 1. MS Calcd.: 625.2; MS Found: 626.2 [M+H]⁺. ¹H NMR (400 MHz, CDCl₃): δ 8.99 (s, 1H), 8.20 (s, 1H), 8.11 (d, J=4.8 Hz, 1H), 7.55-7.65 (m, 1H), 7.35-7.45 (m, 1H), 7.10-7.18 (m, 2H), 6.97-7.03 (m, 1H), 5.46 (s, 2H), 4.47 (s, 2H), 4.05 (s, 2H), 3.01-3.24 (m, 3H), 2.38-2.60 (m, 4H), 2.10-2.21 (m, 2H), 1.22-1.38 (m, 4H). ¹⁹F NMR (377 MHz, CDCl₃): δ −64.62, −115.27.

### Example 38: 1-{[2-({4-[3-(4-chloro-2-fluorophenyl)-2,3-dihydro-1,4-benzodioxin-5-yl]piperidin-1-yl}methyl)-5-[5-(trifluoromethyl)-4H-1,2,4-triazol-3-yl]pyridin-3-yl]methyl}cyclopropane-1-carbonitrile (38)

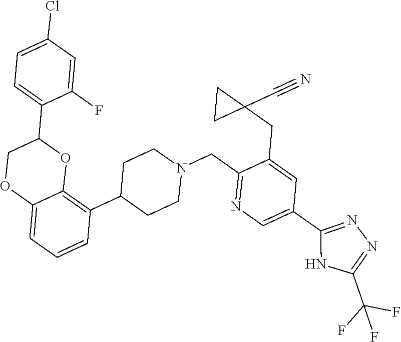 [SMILES: N#CC1(Cc2cc(-c3nnc(C(F)(F)F)[nH]3)cnc2CN2CCC(c3cccc4c3OC(c3ccc(Cl)cc3F)CO4)CC2)CC1]

Starting from 4-(3-(4-chloro-2-fluorophenyl)-2,3-dihydrobenzo[b][1,4]dioxin-5-yl)piperidine hydrochloride, 1-{[2-({4-[3-(4-chloro-2-fluorophenyl)-2,3-dihydro-1,4-benzodioxin-5-yl]piperidin-1-yl}methyl)-5-[5-(trifluoromethyl)-4H-1,2,4-triazol-3-yl]pyridin-3-yl]methyl}cyclopropane-1-carbonitrile (38) (19.7 mg) was obtained with the similar method. MS Calcd: 652.2; MS Found: 653.1 [M+H]⁺. ¹H NMR (400 MHz, DMSO-d₆) δ 9.07 (d, J=2.0 Hz, 1H), 8.44 (d, J=2.0 Hz, 1H), 7.60-7.51 (m, 2H), 7.43 (dd, J=1.6 Hz/J=8.4 Hz, 1H), 6.88-6.77 (m, 3H), 5.47 (dd, J=1.6 Hz/J=7.6 Hz, 1H), 4.45 (dd, J=2.0 Hz/J=11.2 Hz, 1H), 4.11 (dd, J=8.0 Hz/J=11.6 Hz, 1H), 3.89 (brs, 2H), 3.18 (s, 2H), 3.05-2.95 (m, 2H), 2.94-2.86 (m, 1H), 2.40-2.27 (m, 2H), 1.85-1.62 (m, 4H), 1.40-1.36 (m, 2H), 1.25-1.09 (m, 2H). ¹⁹F-NMR (377 MHz, DMSO-d₆): δ −63.01, −115.09.

### Example 39: 1-{[2-({4-[(2R)-2-(4-chlorophenyl)-2,3-dihydro-1,4-benzodioxin-5-yl]piperidin-1-yl}methyl)-5-[5-(trifluoromethyl)-4H-1,2,4-triazol-3-yl]pyridin-3-yl]methyl}cyclopropane-1-carbonitrile (39)

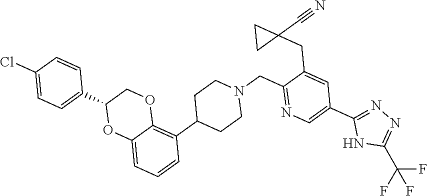 [SMILES: N#CC1(Cc2cc(-c3nnc(C(F)(F)F)[nH]3)cnc2CN2CCC(c3cccc4c3OC[C@@H](c3ccc(Cl)cc3)O4)CC2)CC1]

Starting from (R)-4-(2-(4-chlorophenyl)-2,3-dihydrobenzo[b][1,4]dioxin-5-yl)piperidine HCl salt, 1-{[2-({4-[(2R)-2-(4-chlorophenyl)-2,3-dihydro-1,4-benzodioxin-5-yl]piperidin-1-yl}methyl)-5-[5-(trifluoromethyl)-4H-1,2,4-triazol-3-yl]pyridin-3-yl]methyl}cyclopropane-1-carbonitrile (39) (2.1 mg) was obtained with the similar method. MS Calcd: 634.2; MS Found: 635.2 [M+H]⁺. ¹H NMR (400 MHz, CD₃OD): δ 9.20 (s, 1H), 8.50 (d, J=1.6 Hz, 1H), 7.49-7.40 (m, 4H), 6.89-6.82 (m, 3H), 5.20-5.14 (m, 1H), 4.57 (s, 2H), 4.49-4.42 (m, 1H), 4.06-4.00 (m, 1H), 3.65-3.50 (m, 1H), 3.25-3.12 (m, 4H), 3.10-3.05 (m, 2H), 2.20-1.90 (m, 4H), 1.44-1.40 (m, 2H), 1.30-1.26 (m, 2H). ¹⁹F-NMR (377 MHz, CD₃OD): δ −65.07.

### Example 40: 1-({2-[(4-{3-[(4-chloro-2-fluorophenyl)methoxy]-4-fluorophenyl}piperidin-1-yl)methyl]-5-[5-(trifluoromethyl)-4H-1,2,4-triazol-3-yl]pyridin-3-yl}methyl)cyclopropane-1-carbonitrile (40)

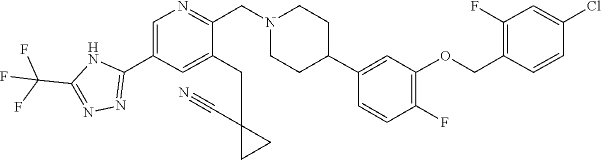 [SMILES: N#CC1(Cc2cc(-c3nnc(C(F)(F)F)[nH]3)cnc2CN2CCC(c3ccc(F)c(OCc4ccc(Cl)cc4F)c3)CC2)CC1]

Starting from 4-{3-[(4-chloro-2-fluorophenyl)methoxy]-4-fluorophenyl}piperidine TFA salt, 1-({2-[(4-{3-[(4-chloro-2-fluorophenyl)methoxy]-4-fluorophenyl}piperidin-1-yl)methyl]-5-[5-(trifluoromethyl)-4H-1,2,4-triazol-3-yl]pyridin-3-yl}methyl)cyclopropane-1-carbonitrile (40) (16.3 mg) was obtained with the similar method. MS Calcd: 642.2; MS Found: 643.3 [M+H]⁺. ¹H NMR (400 MHz, CD₃OD): δ 9.20 (s, 1H), 8.51 (s, 1H), 7.53 (t, J=8.2 Hz, 1H), 7.28-7.22 (m, 2H), 7.11-7.03 (m, 2H), 6.93-6.87 (m, 1H), 5.19 (s, 2H), 4.49 (s, 2H), 3.55 (d, J=11.6 Hz, 2H), 3.14-2.95 (m, 4H), 2.90-2.77 (m, 1H), 2.10-1.98 (m, 4H), 1.47-1.40 (m, 2H), 1.32-1.25 (m, 2H). ¹⁹F-NMR (377 MHz, CD₃OD): δ −65.29, −117.60, −138.49.

### Example 41: 1-({2-[(4-{3-[(4-chloro-2-fluorophenyl)methoxy]-4-fluorophenyl}piperazin-1-yl)methyl]-5-[5-(trifluoromethyl)-4H-1,2,4-triazol-3-yl]pyridin-3-yl}methyl)cyclopropane-1-carbonitrile (41)

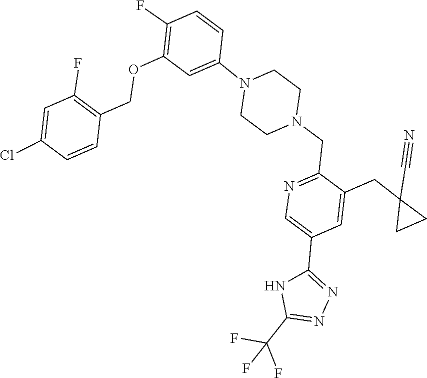 [SMILES: N#CC1(Cc2cc(-c3nnc(C(F)(F)F)[nH]3)cnc2CN2CCN(c3ccc(F)c(OCc4ccc(Cl)cc4F)c3)CC2)CC1]

Starting from 1-{3-[(4-chloro-2-fluorophenyl)methoxy]-4-fluorophenyl}piperazine TFA salt, 1-({2-[(4-{3-[(4-chloro-2-fluorophenyl)methoxy]-4-fluorophenyl}piperazin-1-yl)methyl]-5-[55-(trifluoromethyl)-4H-1,2,4-triazol-3-yl]pyridin-3-yl}methyl)cyclopropane-1-carbonitrile (41) (14.8 mg) was obtained with the similar method. MS Calcd: 643.2; MS Found: 644.3 [M+H]⁺. ¹H NMR (400 MHz, CD₃OD): δ 9.09 (d, J=2.0 Hz, 1H), 8.53 (d, J=2.0 Hz, 1H), 7.51 (t, J=8.2 Hz, 1H), 7.23 (d, J=9.2 Hz, 2H), 7.01-6.93 (m, 1H), 6.75 (dd, J₁=7.2 Hz/J₂ 2.8 Hz, 1H), 6.58-6.50 (m, 1H), 5.15 (s, 2H), 4.01 (s, 2H), 3.20 (s, 2H), 3.20-3.11 (m, 4H), 2.90-2.74 (m, 4H), 1.47-1.41 (m, 2H), 1.28-1.22 (m, 2H). ¹⁹F-NMR (377 MHz, CD₃OD): δ −66.18, −117.58, −146.19.

### Example 42: 1-({2-[(4-{4-chloro-3-[(4-chloro-2-fluorophenyl)methoxy]phenyl}piperazin-1-yl)methyl]-5-[5-(trifluoromethyl)-4H-1,2,4-triazol-3-yl]pyridin-3-yl}methyl)cyclopropane-1-carbonitrile (42)

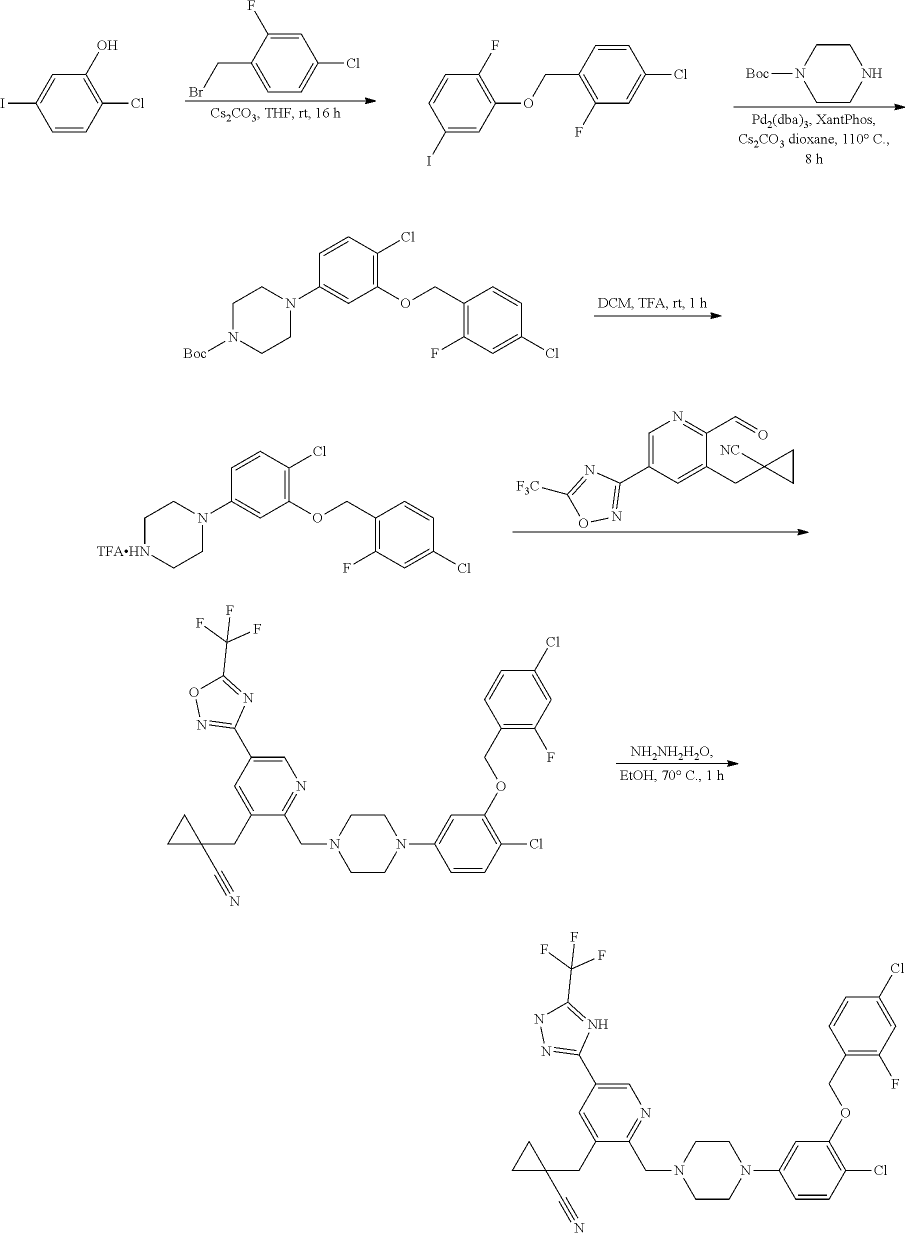

### Step A: 4-chloro-2-fluoro-1-((2-fluoro-5-iodophenoxy)methyl)benzene

To a solution of 2-chloro-5-iodophenol (500 mg, 2.0 mmol) in THF (35 mL) was added 1-(bromomethyl)-4-chloro-2-fluorobenzene (437 mg, 2.0 mmol) and Cs₂CO₃ (2.57 g, 7.88 mmol). The mixture was stirred at rt for 16 h. The mixture was poured into water 50 mL) and extracted with EA (2×50 mL), the combined organic layer was washed with brine, dried over sodium sulfate, filtered and the filtrate was concentrated. The mixture was purified by column chromatography on silica gel (PE:EA=1:0) to give 4-chloro-2-fluoro-1-((2-fluoro-5-iodophenoxy)methyl)benzene (524 mg, yield: 92.1%). ¹H NMR (400 MHz, CD₃OD): δ 7.64-7.59 (m, 2H), 7.52 (dd, J=4.0 Hz/J=10.0 Hz, 1H), 7.39-7.34 (m, 2H), 7.23 (d, J=2.0 Hz, 1H), 5.25 (s, 2H).

### Step B: tert-butyl 4-(4-chloro-3-((4-chloro-2-fluorobenzyl)oxy)phenyl)piperazine-1-carboxylate

A mixture of 4-chloro-2-fluoro-1-((2-fluoro-5-iodophenoxy)methyl)benzene (234 mg, 1.05 mmol), tert-butyl piperazine-1-carboxylate (196 mg, 1.05 mmol), Pd₂(dba)₃ (97 mg, 0.11 mmol), Xantphos (122 mg, 0.21 mmol) and Cs₂CO₃ (1.03 g, 3.16 mmol) in dioxane (15 mL) was stirred at 110° C. under Ar for 8 h. The mixture was poured into water (50 mL) and extracted with EA (2×50 mL), the combined organic layer was washed with brine, dried over sodium sulfate, filtered and the filtrate was concentrated. The residue was purified by column chromatography silica gel (PE:EA=15:1) to give tert-butyl 4-(4-chloro-3-((4-chloro-2-fluorobenzyl)oxy)phenyl)piperazine-1-carboxylate (190 mg, yield: 40.0%). MS Calcd: 454.1; MS Found: 455.0 [M+H]⁺.

### Step C: 1-{4-chloro-3-[(4-chloro-2-fluorophenyl)methoxy]phenyl}piperazine, TFA salt

To a mixture of tert-butyl 4-(4-chloro-3-((4-chloro-2-fluorobenzyl)oxy)phenyl)piperazine-1-carboxylate (190 mg, 0.42 mmol) in DCM (8 ml) was added TFA (2 mL) in a dropwise manner. The mixture was stirred at rt for 1 h. The mixture was concentrated with DCM to give 1-{4-chloro-3-[(4-chloro-2-fluorophenyl)methoxy]phenyl}piperazine, TFA salt (170 mg). MS Calcd: 354.1; MS Found: 355.2 [M+H]⁺.

### Step D: 1-((2-((4-(4-chloro-3-((4-chloro-2-fluorobenzyl)oxy)phenyl)piperazin-1-yl)methyl)-5-(5-(trifluoromethyl)-1,2,4-oxadiazol-3-yl)pyridin-3-yl)methyl)cyclopropane-1-carbonitrile

A mixture of 1-{4-chloro-3-[(4-chloro-2-fluorophenyl)methoxy]phenyl}piperazine, TFA salt (170 mg, 0.48 mmol) and TEA (298 mg, 2.95 mmol) in DCM (20 mL) was stirred at rt for 0.5 h. 1-((2-formyl-5-(5-(trifluoromethyl)-1,2,4-oxadiazol-3-yl)pyridin-3-yl)methyl)cyclopropane-1-carbonitrile (230 mg, 0.71 mmol) was added and the mixture was stirred for 2 h. NaBH(OAc)₃ (500 mg, 2.36 mmol) was added and the mixture was stirred at rt for 16 h. The mixture was poured into water (50 mL) and extracted with DCM (2×50 mL), the combined organic layer was washed with brine, dried over sodium sulfate, filtered and the filtrate was concentrated to give 1-((2-((4-(4-chloro-3-((4-chloro-2-fluorobenzyl)oxy)phenyl)piperazin-1-yl)methyl)-5-(5-(trifluoromethyl)-1,2,4-oxadiazol-3-yl)pyridin-3-yl)methyl)cyclopropane-1-carbonitrile (30 mg, yield: 7.7%). MS Calcd: 660.1; MS Found: 661.0 [M+H]⁺.

### Step E: 1-({2-[(4-{4-chloro-3-[(4-chloro-2-fluorophenyl)methoxy]phenyl}piperazin-1-yl)methyl]-5-[5-(trifluoromethyl)-4H-1,2,4-triazol-3-yl]pyridin-3-yl}methyl)cyclopropane-1-carbonitrile (42)

A mixture of 1-((2-((4-(4-chloro-3-((4-chloro-2-fluorobenzyl)oxy)phenyl)piperazin-1-yl)methyl)-5-(5-(trifluoromethyl)-1,2,4-oxadiazol-3-yl)pyridin-3-yl)methyl)cyclopropane-1-carbonitrile (30 mg, 0.05 mmol) and NH₂NH₂·H₂O (2 drops) in EtOH (2 mL) was stirred at 70° C. for 1 h. The mixture was purified by prep-HPLC to give 1-({2-[(4-{4-chloro-3-[(4-chloro-2-fluorophenyl)methoxy]phenyl}piperazin-1-yl)methyl]-5-[5-(trifluoromethyl)-4H-1,2,4-triazol-3-yl]pyridin-3-yl}methyl)cyclopropane-1-carbonitrile (42) (5.59 mg, yield: 17.0%). MS Calcd: 659.2; MS Found: 660.0 [M+H]⁺. HNMR (400 MHz, CD₃OD): δ 9.10 (d, J=2.0 Hz, 1H), 8.55 (s, 1H), 7.58 (t, J=8.2 Hz, 1H), 7.30-7.15 (m, 3H), 6.71 (d, J=2.4 Hz, 1H), 6.60-6.53 (dd, J=2.8 Hz/J=8.8 Hz, 1H), 5.16 (s, 2H), 4.01 (s, 2H), 3.28-3.20 (m, 6H), 2.85-2.75 (m, 4H), 1.46-1.39 (m, 2H), 1.38-1.21 (m, 2H). ¹⁹F-NMR (377 MHz, CD₃OD): δ −67.86, −117.60.

### Example 43: 1-[(2-{[(2S)-4-{3-[(4-chloro-2-fluorophenyl)methoxy]-4-fluorophenyl}-2-methylpiperazin-1-yl]methyl}-5-[5-(trifluoromethyl)-4H-1,2,4-triazol-3-yl]pyridin-3-yl)methyl]cyclopropane-1-carbonitrile (43)

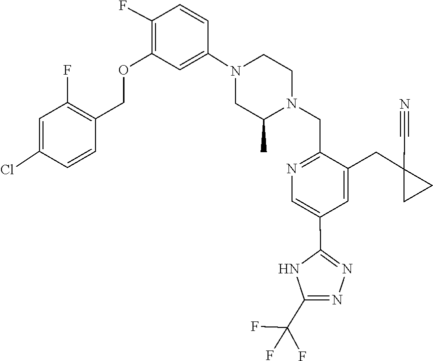 [SMILES: C[C@H]1CN(c2ccc(F)c(OCc3ccc(Cl)cc3F)c2)CCN1Cc1ncc(-c2nnc(C(F)(F)F)[nH]2)cc1CC1(C#N)CC1]

Starting from (3S)-1-{3-[(4-chloro-2-fluorophenyl)methoxy]-4-fluorophenyl}-3-methylpiperazine TFA salt, 1-[(2-{[(2S)-4-{3-[(4-chloro-2-fluorophenyl)methoxy]-4-fluorophenyl}-2-methylpiperazin-1-yl]methyl}-5-[5-(trifluoromethyl)-4H-1,2,4-triazol-3-yl]pyridin-3-yl)methyl]cyclopropane-1-carbonitrile (43) (2.9 mg) was obtained with the similar method. MS Calcd: 657.2; MS Found: 658.3 [M+H]⁺. ¹H NMR (400 MHz, CD₃OD): δ 9.11 (d, J=2.0 Hz, 1H), 8.52 (d, J=1.6 Hz, 1H), 7.52 (t, J=8.4 Hz, 1H), 7.23 (d, J=9.2 Hz, 2H), 7.01-6.93 (m, 1H), 6.74 (dd, J₁=4.8 Hz/J₂ 7.2 Hz, 1H), 6.57-6.50 (m, 1H), 5.15 (s, 2H), 4.58 (d, J=13.6 Hz, 1H), 3.75 (d, J=13.2 Hz, 1H), 3.49-3.40 (m, 1H), 3.36-3.31 (m, 2H), 3.23-3.15 (m, 1H), 2.99-2.85 (m, 3H), 2.81-2.59 (m, 2H), 1.47-1.39 (m, 2H), 1.36 (d, J=6.4 Hz, 3H), 1.27-1.22 (m, 2H). ¹⁹F-NMR (377 MHz, CD₃OD): δ −66.03, −117.55, −146.11.

### Example 44: 1-({2-[(4-{6-[(4-chloro-2-fluorophenyl)methoxy]-5-fluoropyridin-2-yl}piperazin-1-yl)methyl]-5-[5-(trifluoromethyl)-4H-1,2,4-triazol-3-yl]pyridin-3-yl}methyl)cyclopropane-1-carbonitrile (44)

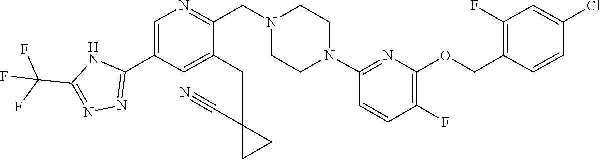 [SMILES: N#CC1(Cc2cc(-c3nnc(C(F)(F)F)[nH]3)cnc2CN2CCN(c3ccc(F)c(OCc4ccc(Cl)cc4F)n3)CC2)CC1]

Starting from 1-{6-[(4-chloro-2-fluorophenyl)methoxy]-5-fluoropyridin-2-yl}piperazine, TFA salt, 1-({2-[(4-{6-[(4-chloro-2-fluorophenyl)methoxy]-5-fluoropyridin-2-yl}piperazin-1-yl)methyl]-5-[5-(trifluoromethyl)-4H-1,2,4-triazol-3-yl]pyridin-3-yl}methyl)cyclopropane-1-carbonitrile (44) (4.7 mg) was obtained with the similar method. MS Calcd: 644.2; MS Found: 645.1 [M+H]⁺. ¹H NMR (400 MHz, CD₃OD): δ 8.98 (d, J=1.6 Hz, 1H), 8.38 (d, J=1.6 Hz, 1H), 7.54 (t, J=8.4 Hz, 1H), 7.50-7.42 (m, 2H), 7.31 (dd, J₁=8.0 Hz/J 6.4 Hz, 1H), 6.27 (d, J=8.4 Hz, 1H), 5.39 (s, 2H), 3.70 (s, 2H), 3.45-3.40 (m, 4H), 3.20-3.11 (m, 2H), 2.51-2.46 (m, 4H), 1.38-1.32 (m, 2H), 1.21-1.15 (m, 2H). ¹⁹F-NMR (377 MHz, CD₃OD): δ −60.78, −115.19, −156.90.

### Example 45: 1-((2-((4-(4-((4-chloro-2-fluorobenzyl)oxy)-5-fluoropyrimidin-2-yl)piperazin-1-yl)methyl)-5-(5-(trifluoromethyl)-4H-1,2,4-triazol-3-yl)pyridin-3-yl)methyl)cyclopropane-1-carbonitrile (45)

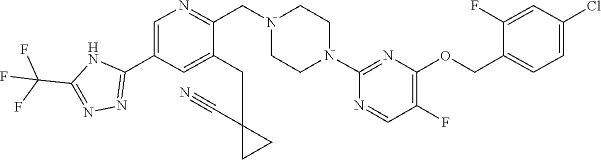 [SMILES: N#CC1(Cc2cc(-c3nnc(C(F)(F)F)[nH]3)cnc2CN2CCN(c3ncc(F)c(OCc4ccc(Cl)cc4F)n3)CC2)CC1]

Starting from 1-(4-(4-((4-chloro-2-fluorobenzyl)oxy)-5-fluoropyrimidin-2-yl)-1|4-piperazin-1-yl) TFA salt, 1-((2-((4-(4-((4-chloro-2-fluorobenzyl)oxy)-5-fluoropyrimidin-2-yl)piperazin-1-yl)methyl)-5-(5-(trifluoromethyl)-4H-1,2,4-triazol-3-yl)pyridin-3-yl)methyl)cyclopropane-1-carbonitrile (4.0 mg) was obtained with the similar method of Example 1. MS Calcd: 645.1; MS Found: 646.1 [M+H]. ¹H NMR (400 MHz, CD₃OD-d₄): δ 9.09 (s, 1H), 8.54 (s, 1H), 8.01 (s, 1H), 7.50 (t, J=7.6 Hz, 1H), 7.27-7.21 (m, 2H), 5.47 (s, 2H), 3.94 (s, 2H), 3.83-3.73 (m, 4H), 3.25 (s, 2H), 2.69-2.60 (m, 4H), 1.47-1.42 (m, 2H), 1.28-1.21 (m, 2H). ¹⁹F NMR (377 MHz, CD₃OD-d₄): δ (−66.25), (−117.15).

### Example 46: (S)-1-((2-((4-(6-((4-chloro-2-fluorobenzyl)oxy)pyridin-2-yl)-2-methylpiperazin-1-yl)methyl)-5-(5-(trifluoromethyl)-4H-1,2,4-triazol-3-yl)pyridin-3-yl)methyl)cyclopropane-1-carbonitrile (46)

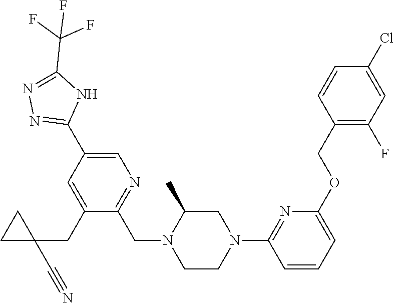 [SMILES: C[C@H]1CN(c2cccc(OCc3ccc(Cl)cc3F)n2)CCN1Cc1ncc(-c2nnc(C(F)(F)F)[nH]2)cc1CC1(C#N)CC1]

Starting from 1-((2S)-4-(6-((4-chloro-2-fluorobenzyl)oxy)pyridin-2-yl)-2-methyl-1|4-piperazin-1-yl)TFA salt, (S)-1-((2-((4-(6-((4-chloro-2-fluorobenzyl)oxy)pyridin-2-yl)-2-methylpiperazin-1-yl)methyl)-5-(5-(trifluoromethyl)-4H-1,2,4-triazol-3-yl)pyridin-3-yl)methyl)cyclopropane-1-carbonitrile (11.7 mg). MS Calcd: 640.2; MS Found: 641.2 [M+H]. ¹H NMR (400 MHz, CD₃OD): δ 9.03 (s, 1H), 8.34 (s, 1H), 7.64 (t, J=7.6 Hz, 1H), 7.51 (t, J=8 Hz, 1H), 7.26-7.21 (m, 2H), 6.91 (d, J=7.2 Hz, 1H), 6.71 (d, J=8.4 Hz, 1H), 5.46 (s, 2H), 4.64-4.52 (m, 2H), 3.71-3.57 (m, 2H), 3.23-3.13 (m, 1H), 3.09-2.95 (m, 4H), 2.33-2.22 (m, 2H), 2.20-2.09 (m, 2H), 1.44-1.29 (m, 2H), 1.27-1.21 (m, 2H). ¹⁹F NMR (377 MHz, CD₃OD): δ −117.59.

### Example 47: 1-{[2-({6-[(4-chloro-2-fluorophenyl)methoxy]-1′,2′,3′,6′-tetrahydro-[2,4′-bipyridin]-1′-yl}methyl)-5-[5-(trifluoromethyl)-4H-1,2,4-triazol-3-yl]pyridin-3-yl]methyl}cyclopropane-1-carbonitrile (47)

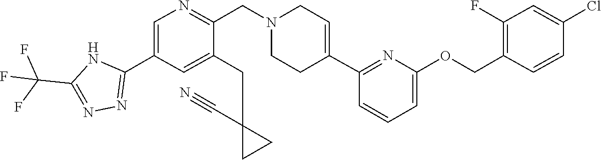 [SMILES: N#CC1(Cc2cc(-c3nnc(C(F)(F)F)[nH]3)cnc2CN2CC=C(c3cccc(OCc4ccc(Cl)cc4F)n3)CC2)CC1]

Starting from 2-[(4-chloro-2-fluorophenyl)methoxy]-6-(1,2,3,6-tetrahydropyridin-4-yl)pyridine TFA salt, 1-{[2-({6-[(4-chloro-2-fluorophenyl)methoxy]-1′,2′,3′,6′-tetrahydro-[2,4′-bipyridin]-1′-yl}methyl)-5-[5-(trifluoromethyl)-4H-1,2,4-triazol-3-yl]pyridin-3-yl]methyl}cyclopropane-1-carbonitrile (47) (2.8 mg) was obtained with the similar method. MS Calcd.: 623.2; MS Found: 624.2 [M+H]⁺. ¹H NMR (400 MHz, CD₃OD): δ 9.17 (s, 1H), 8.53 (s, 1H), 7.65 (t, J=7.8 Hz, 1H), 7.48 (t, J=8.0 Hz, 1H), 7.24-7.17 (m, 2H), 7.15-7.08 (m, 1H), 6.75-6.70 (m, 2H), 5.44 (s, 2H), 4.38 (s, 2H), 3.72 (s, 2H), 3.30-3.21 (m, 2H), 3.13 (s, 2H), 2.87-2.80 (s, 2H), 1.45-1.38 (m, 2H), 1.28-1.23 (m, 2H). ¹⁹F-NMR (377 MHz, CD₃OD): δ −65.48, −117.67.

### Example 48: 1-({2-[(4-{3-[(4-chloro-2-fluorophenyl)methoxy]-4-fluorophenyl}-1,2,3,6-tetrahydropyridin-1-yl)methyl]-5-[5-(trifluoromethyl)-4H-1,2,4-triazol-3-yl]pyridin-3-yl}methyl)cyclopropane-1-carbonitrile (48)

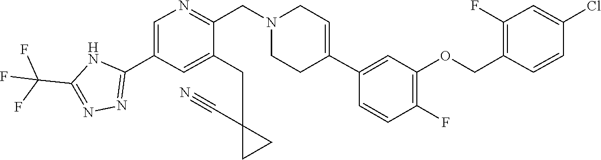 [SMILES: N#CC1(Cc2cc(-c3nnc(C(F)(F)F)[nH]3)cnc2CN2CC=C(c3ccc(F)c(OCc4ccc(Cl)cc4F)c3)CC2)CC1]

Starting from 4-{3-[(4-chloro-2-fluorophenyl)methoxy]-4-fluorophenyl}-1,2,3,6-tetrahydropyridine, TFA salt, 1-({2-[(4-{3-[(4-chloro-2-fluorophenyl)methoxy]-4-fluorophenyl}-1,2,3,6-tetrahydropyridin-1-yl)methyl]-5-[5-(trifluoromethyl)-4H-1,2,4-triazol-3-yl]pyridin-3-yl}methyl)cyclopropane-1-carbonitrile (48) (31.8 mg) was obtained with the similar method. MS Calcd: 640.2; MS Found: 641.0 [M+H]⁺. ¹H NMR (477 MHz, CD₃OD): δ 9.08 (s, 1H), 8.46 (s, 1H), 7.62-7.57 (m, 1H), 7.51 (d, J=10.0 Hz, 1H), 7.38-7.28 (m, 2H), 7.20-7.13 (m, 1H), 7.02-6.98 (m, 1H), 6.16 (s, 1H), 5.23 (s, 2H), 3.92 (s, 2H), 3.20 (s, 4H), 2.75 (s, 2H), 2.50-2.43 (m, 2H), 1.40-1.34 (m, 2H), 1.24-1.20 (m, 2H). ¹⁹F-NMR (377 MHz, CD₃OD): δ −63.44, −114.96, −136.51.

### Example 49: (S)-3-((4-(2-(4-chloro-2-fluorophenyl)-2-methylbenzo[d][1,3]dioxol-4-yl)piperidin-1-yl)methyl)-4-methyl-6-(5-(trifluoromethyl)-4H-1,2,4-triazol-3-yl)pyridazine (49)

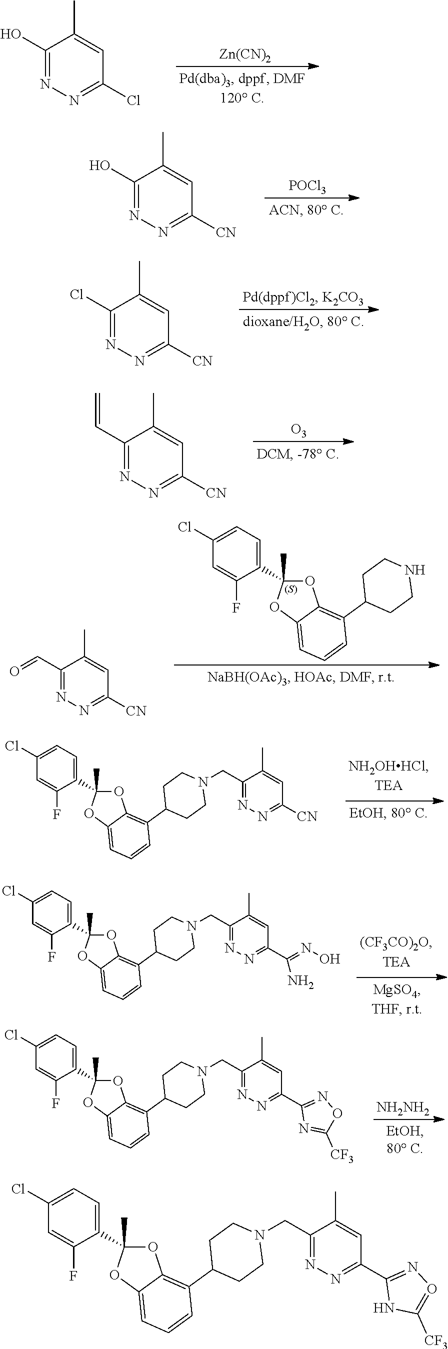

### Step A: 6-hydroxy-5-methylpyridazine-3-carbonitrile

A mixture of 6-chloro-4-methylpyridazin-3-ol (5.0 g, 34.5 mmol), Zn(CN)₂ (5.24 g, 44.8 mmol), Pd₂(dba)₃ (1.58 g, 1.72 mmol), and dppf (1.53 g, 2.76 mmol) in DMF (50 mL) was degassed and refilled with nitrogen for three times. The reaction mixture was heated at 120° C. for 1.5h. The resulted mixture was diluted with DCM (100 mL) and aq. Sat. NaHCO₃ (100 mL). The aqueous layer was separated and extracted with DCM (100 mL*3). The combined organic layer was dried, filtered, and concentrated. The residue was purified by silica gel column (PE/EtOAc=100/1 to 2/1) to give 6-hydroxy-5-methylpyridazine-3-carbonitrile (4.5 g, 97% yield). ¹H NMR (400 MHz, DMSO-d₆) δ 13.81 (s, 1H), 7.71 (d, J=1.6 Hz, 1H), 2.07 (d, J₁=1.2 Hz, 3H). LC-MS: m/z 136.0 (M+H)f.

### Step B: 6-chloro-5-methylpyridazine-3-carbonitrile

To a solution of 6-hydroxy-5-methylpyridazine-3-carbonitrile (4.5 g, 33.3 mmol) in ACN (50 mL) was added POCl₃ (15.3 g, 100 mmol). The mixture was stirred at 80° C. for 4 h. The mixture was concentrated. The residue was diluted with EtOAc (100 mL) and poured into ice-water (100 mL). The aqueous phase was extracted with EtOAc (100 mL*3). The combined organic phase was washed with brine, dried over Na₂SO₄, filtered and concentrated. The residue was purified by silica gel column (PE/EtOAc 100/1 to 8/1) to give 6-chloro-5-methylpyridazine-3-carbonitrile (3.0 g, 59% yield). ¹H NMR (400 MHz, DMSO-d₆) δ 8.46 (d, J=0.8 Hz, 1H), 2.44 (d, J=0.8 Hz, 3H). LC-MS: m/z 154.0 (M+H)+.

### Step C: 5-methyl-6-vinylpyridazine-3-carbonitrile

A solution of 6-chloro-5-methylpyridazine-3-carbonitrile (1.66 g, 10.9 mmol), 4,4,5,5-tetramethyl-2-vinyl-1,3,2-dioxaborolane (5.01 g, 32.6 mmol), Pd(dppf)Cl₂·DCM (893 mg, 1.09 mmol) and K₂CO₃ (4.49 g, 32.6 mmol) in dioxane/H₂O (30 mL/3 mL) was degassed and refilled with N₂ for three times. The mixture was stirred at 80° C. for 2.5h. The mixture was diluted with H₂O (50 mL) and extracted with EtOAc (50 mL*3). The combined organic phase was washed with brine, dried over Na₂SO₄, filtered and concentrated. The residue was purified by silica gel column (PE/EtOAc=50/1 to 4/1) to give 5-methyl-6-vinylpyridazine-3-carbonitrile (1.08 g, 69% yield). ¹H NMR (400 MHz, DMSO-d₆) δ 8.21 (d, J₁=0.4 Hz, 1H), 7.16 (dd, J₁=16.8, 10.8 Hz, 1H), 6.62 (dd, J₁=17.2, 2.0 Hz, 1H), 5.87 (dd, J=10.8, 2.0 Hz, 1H), 2.42 (d, J=0.8 Hz, 3H). LC-MS: m/z 146.1 (M+H)+.

### Step D: 6-formyl-5-methylpyridazine-3-carbonitrile

5-Methyl-6-vinylpyridazine-3-carbonitrile (1.08 g, 7.45 mmol) was dissolved in DCM (10 mL). The solution was cooled to −78° C. Ozone was bubbled through the solution until the solution turned light blue. The reaction was quenched with dimethyl sulfide. The mixture was concentrated and purified by silica gel column (PE/EtOAc 30/1 to 2/1) to give 6-formyl-5-methylpyridazine-3-carbonitrile (150 mg, 14% yield). ¹H NMR (400 MHz, DMSO-d₆) δ 10.35 (d, J=0.8 Hz, 1H), 8.47-8.50 (m, 1H), 2.63 (d, J=0.8 Hz, 3H). LC-MS: m/z 166.1 (M+18+H)+.

### Step E: (S)-6-((4-(2-(4-chloro-2-fluorophenyl)-2-methylbenzo[d][1,3]dioxol-4-yl)piperidin-1-yl)methyl)-5-methylpyridazine-3-carbonitrile

To a solution of 6-formyl-5-methylpyridazine-3-carbonitrile (197 mg, 1.34 mmol), and (S)-4-(2-(4-chloro-2-fluorophenyl)-2-methylbenzo[d][1,3]dioxol-4-yl)piperidine (310 mg, 0.890 mmol) in DMF (3 mL) was added three drops of AcOH. The mixture was stirred at room temperature for 1h. And then NaBH(OAc)₃ (377 mg, 1.78 mmol) was added. The reaction was stirred at room temperature for 1h. The reaction mixture was diluted with H₂O (30 mL) and extracted with EtOAc (30 mL*3). The organic layer was concentrated and purified by Prep-TLC (PE/EtOAc=1/1) to give (S)-6-((4-(2-(4-chloro-2-fluorophenyl)-2-methylbenzo[d][1,3]dioxol-4-yl)piperidin-1-yl)methyl)-5-methylpyridazine-3-carbonitrile (200 mg, 47% yield). LC-MS: m/z 479.3 (M+H)+.

### Step F: (S,Z)-6-((4-(2-(4-chloro-2-fluorophenyl)-2-methylbenzo[d][1,3]dioxol-4-yl)piperidin-1-yl)methyl)-N′-hydroxy-5-methylpyridazine-3-carboximidamide

A solution of(S)-6-((4-(2-(4-chloro-2-fluorophenyl)-2-methylbenzo[d][1,3]dioxol-4-yl)piperidin-1-yl)methyl)-5-methylpyridazine-3-carbonitrile (200 mg, 0.420 mmol), hydroxylamine hydrochloride (88.0 mg, 1.26 mmol) and TEA (85.0 mg, 0.840 mmol) in EtOH (5 mL) was stirred at 80° C. for 2 h. The mixture was concentrated. The residue was diluted with H₂O (20 mL) and extracted with EtOAc (30 mL*3). The organic phases were combined, dried over anhydrous Na₂SO₄ and concentrated to give (S,Z)-6-((4-(2-(4-chloro-2-fluorophenyl)-2-methylbenzo[d][1,3]dioxol-4-yl)piperidin-1-yl)methyl)-N′-hydroxy-5-methylpyridazine-3-carboximidamide (crude, 210 mg). LC-MS: m/z 512.3 (M+H)⁺.

### Step G: (S)-3-(6-((4-(2-(4-chloro-2-fluorophenyl)-2-methylbenzo[d][1,3]dioxol-4-yl)piperidin-1-yl)methyl)-5-methylpyridazin-3-yl)-5-(trifluoromethyl)-1,2,4-oxadiazole

To a solution of (S,Z)-6-((4-(2-(4-chloro-2-fluorophenyl)-2-methylbenzo[d][1,3]dioxol-4-yl)piperidin-1-yl)methyl)-N′-hydroxy-5-methylpyridazine-3-carboximidamide (crude 210 mg, 0.410 mmol) in THF (5 mL) were added TFAA (123 mg, 0.585 mmol), TEA (0.5 mL) and MgSO₄. The mixture was stirred at room temperature for 2 days. The mixture was filtered, and the filtrate was concentrated. The residue was purified by Prep-HPLC (0.10% NH₄HCO₃ in water and acetonitrile) to give (S)-3-(6-((4-(2-(4-chloro-2-fluorophenyl)-2-methylbenzo[d][1,3]dioxol-4-yl)piperidin-1-yl)methyl)-5-methylpyridazin-3-yl)-5-(trifluoromethyl)-1,2,4-oxadiazole (60.0 mg, 24% yield over two steps). LC-MS: m/z 590.2 (M+H)⁺.

### Step H: (S)-3-((4-(2-(4-chloro-2-fluorophenyl)-2-methylbenzo[d][1,3]dioxol-4-yl)piperidin-1-yl)methyl)-4-methyl-6-(5-(trifluoromethyl)-4H-1,2,4-triazol-3-yl)pyridazine

To a solution of (S)-3-(6-((4-(2-(4-chloro-2-fluorophenyl)-2-methylbenzo[d][1,3]dioxol-4-yl)piperidin-1-yl)methyl)-5-methylpyridazin-3-yl)-5-(trifluoromethyl)-1,2,4-oxadiazole (65.0 mg, 0.110 mmol) in EtOH (5 mL) was added hydrazine hydrate (98%, 55.0 mg, 1.10 mmol). The mixture was stirred at 80° C. for 2h. TLC showed the reaction was completed. The mixture was concentrated and purified by Prep-TLC (PE/EtOAc=1/1) and Prep-HPLC (0.1% formic acid in water and acetonitrile) to give (S)-3-((4-(2-(4-chloro-2-fluorophenyl)-2-methylbenzo[d][1,3]dioxol-4-yl)piperidin-1-yl)methyl)-4-methyl-6-(5-(trifluoromethyl)-4H-1,2,4-triazol-3-yl)pyridazine (18.5 mg, 29% yield). LC-MS: m/z 589.0 (M+H)⁺. ¹H NMR (400 MHz, DMSO-d₆) δ 8.22 (s, 1H), 7.52-7.60 (m, 2H), 7.34 (dd, J=8.4, 2.0 Hz, 1H), 6.77-6.81 (m, 2H), 6.71-6.76 (m, 1H), 3.94 (s, 2H), 2.92 (s, 2H), 2.61-2.72 (m, 1H), 2.58 (s, 3H), 2.24-2.34 (m, 2H), 2.02 (s, 3H), 1.66-1.78 (m, 4H).

### Example 50: 3-({4-[(2S)-2-(4-chloro-2-fluorophenyl)-2-methyl-1,3-benzodioxol-4-yl]piperidin-1-yl}methyl)-4-methyl-6-(1H-1,2,3,4-tetrazol-5-yl)pyridazine (50)

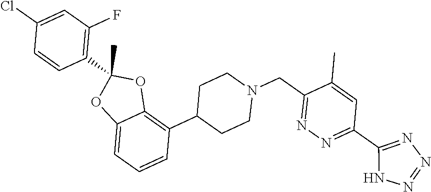 [SMILES: Cc1cc(-c2nnn[nH]2)nnc1CN1CCC(c2cccc3c2O[C@@](C)(c2ccc(Cl)cc2F)O3)CC1]

A solution of 6-({4-[(2S)-2-(4-chloro-2-fluorophenyl)-2-methyl-1,3-benzodioxol-4-yl]piperidin-1-yl}-methyl)-5-methylpyridazine-3-carbonitrile (70 mg, 0.15 mmol) in toluene (1 mL), were treated with di-n-butyl tin diacetate (103 mg, 0.29 mmol) and trimethylsilyl azide (67 mg, 0.58 mmol). The reaction mixture was stirred for 2.5 days at 30° C. The reaction mixture was concentrated under reduced pressure. The residue was purified by Prep-HPLC to afford 3-({4-[(2S)-2-(4-chloro-2-fluorophenyl)-2-methyl-1,3-benzodioxol-4-yl]piperidin-1-yl}methyl)-4-methyl-6-(1H-1,2,3,4-tetrazol-5-yl)pyridazine (7.7 mg, 10.0%). LC-MS (ES⁺): m/z 522.1 [MH⁺]. ¹H NMR (400 MHz, DMSO-d₆, ppm): δ 8.18 (s, 1H), 7.75-7.45 (m, 2H), 7.34 (d, J=8.5 Hz, 1H), 6.99-6.59 (m, 3H), 4.50-4.22 (m, 2H), 3.12-3.02 (m, 2H), 2.92-2.72 (m, 4H), 2.48-2.46 (m, 3H), 2.03 (s, 3H), 1.99-1.80 (m, 4H).

### Example 51: (S)-3-(6-((4-(2-(4-chloro-2-fluorophenyl)-2-methylbenzo[d][1,3]dioxol-4-yl)piperidin-1-yl)methyl)-5-methylpyridazin-3-yl)-1,2,4-oxadiazol-5(4H)-one (51)

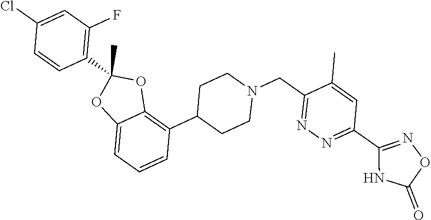 [SMILES: Cc1cc(-c2noc(=O)[nH]2)nnc1CN1CCC(c2cccc3c2O[C@@](C)(c2ccc(Cl)cc2F)O3)CC1]

A mixture of (Z)-6-({4-[(2S)-2-(4-chloro-2-fluorophenyl)-2-methyl-1,3-benzodioxol-4-yl]piperidin-1-yl}methyl)-N′-hydroxy-5-methylpyridazine-3-carboximidamide (80 mg, 0.16 mmol), DBU (71 mg, 0.47 mmol) and CDI (51 mg, 0.31 mmol) in dioxane (3 mL). The reaction mixture was stirred for 5 h at 80° C. under N₂ atmosphere. The reaction was monitored by LC/MS. The reaction mixture was concentrated under reduced pressure. The residue was dissolved in DMF (2 mL), and then purified by Prep-HPLC to afford 3-[6-({4-[(2S)-2-(4-chloro-2-fluorophenyl)-2-methyl-1,3-benzodioxol-4-yl]piperidin-1-yl}methyl)-5-methylpyridazin-3-yl]-4H-1,2,4-oxadiazol-5-one (40 mg, 46.7%). LC-MS (ES⁺): m/z 538.1 [MH⁺]. ¹H NMR (400 MHz, DMSO-d₆): δ 8.04 (s, 1H), 7.61-7.52 (m, 2H), 7.32-7.35 (m, 1H), 6.81-6.74 (m, 3H), 3.98 (s, 2H), 3.05-2.89 (m, 2H), 2.76-2.72 (m, 1H), 2.60-2.53 (m, 4H), 2.40-2.33 (m, 1H), 2.02 (s, 3H), 1.80-1.60 (m, 4H). ¹⁹F NMR (377 MHz, DMSO-d₆): δ −110.82.

### Example 52: 2-({4-[(2S)-2-(4-chloro-2-fluorophenyl)-2-methyl-1,3-benzodioxol-4-yl]piperidin-1-yl}methyl)-3-methyl-5-[5-(trifluoromethyl)-4H-1,2,4-triazol-3-yl]pyrazine (52)

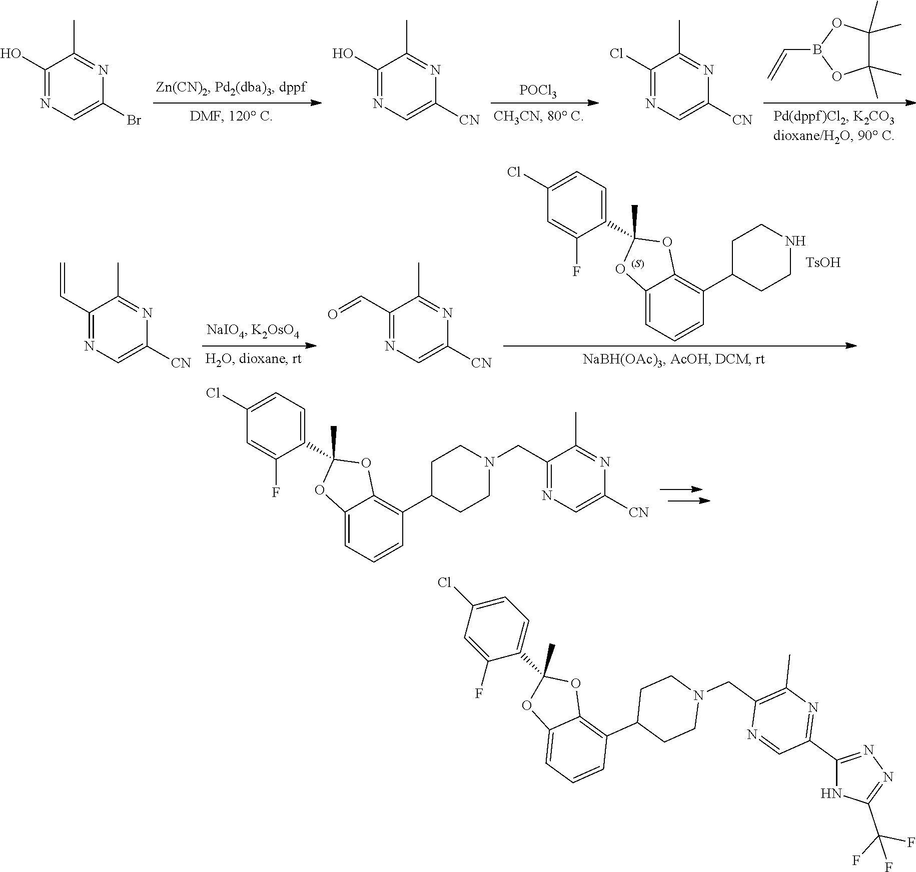

### Step A: Synthesis of 5-hydroxy-6-methylpyrazine-2-carbonitrile

Into a three-necked flask equipped reflux condenser, were added 5-bromo-3-methylpyrazin-2-ol (5.0 g, 26.4 mmol), zinc cyanide (4.1 g, 34.4 mmol), DMF (50 mL), Pd₂(dba)₃ (1.2 g, 1.32 mmol) and dppf (1.2 g, 2.12 mmol). The flask was purged and maintained with nitrogen for three times. The reaction mixture was stirred for 3 h at 120° C. under N₂ atmosphere. The reaction was monitored by LC/MS. The reaction mixture was cooled to room temperature, was then quenched with H₂O (100 mL). The resulting mixture was extracted with EA (3×100 mL). The combined organic layers were washed with saturated NaHCO₃ (aq, 2×100 mL) and then brine (2×100 mL). The organic layer was dried over Na₂SO₄. After filtration, the filtrate was concentrated under reduced pressure. The residue was purified by silica gel column chromatography, eluted with EA/PE (3:2) to afford 5-hydroxy-6-methylpyrazine-2-carbonitrile (1.7 g, 47.6%). LC-MS (ES⁻): m/z 134.00 [M−H⁺].

### Step B: Synthesis of 6-methyl-5-(morpholin-4-yl) pyridazine-3-carbonitrile

A mixture of 5-hydroxy-6-methylpyrazine-2-carbonitrile (1.7 g, 12.6 mmol), and phosphorus oxychloride (5.8 g, 37.7 mmol) in acetonitrile (20 mL) was stirred for 3 hr at 80° C. under N₂ atmosphere. The reaction was monitored by LC/MS. The reaction mixture was cooled to room temperature, and then concentrated under vacuum. The residue was poured into water (100 mL) at room temperature. The resulting mixture was basified to pH=8 by NaOH (aq., c=1M). The resulting mixture was extracted with EA (3×100 mL). The combined organic layers were washed with saturated aqueous solution of NaHCO₃ (2×50 mL) and then brine (2×50 mL). The organic layer was dried over Na₂SO₄. After filtration, the filtrate was concentrated under reduced pressure. The residue was purified by silica gel column chromatography, eluted with EA/PE (1:9) to afford 5-chloro-6-methylpyrazine-2-carbonitrile (1.1 g, 56.9%). LC-MS (ES⁺): m/z 153.8 [MH⁺].

### Step C: Synthesis of 5-ethenyl-6-methylpyrazine-2-carbonitrile

Into a mixture of 5-chloro-6-methylpyrazine-2-carbonitrile (1.1 g, 7.16 mmol) and 2-ethenyl-4,4,5,5-tetramethyl-1,3,2-dioxaborolane (1.7 g, 10.75 mmol) in dioxane/H₂O (5:1, 9.5 mL), were added Pd(dppf)Cl₂ (0.5 g, 0.72 mmol) and K₂CO₃ (3.0 g, 21.47 mmol). The reaction mixture was stirred for 2 h under N₂ atmosphere at 90° C. in an oil bath. The reaction mixture was monitored by LC/MS. The reaction mixture was cooled to room temperature, was then quenched with H₂O (20 mL). The resulting mixture was extracted with EA (3×50 mL). The combined organic layer was washed with saturated brine (2×50 mL) and dried over Na₂SO₄. After filtration, the filtrate was concentrated under reduced pressure. The residue was purified by silica gel column chromatography, eluted with EA/PE (1:9) to afford 5-ethenyl-6-methylpyrazine-2-carbonitrile (660 mg, 63.5%). LC-MS (ES⁺): m/z 145.9 [MH⁺].

### Step D: Synthesis of 5-formyl-6-methylpyrazine-2-carbonitrile

Into a solution of sodium periodate (1827 mg, 8.54 mmol) in water (2 mL), was added a solution of 5-ethenyl-6-methylpyrazine-2-carbonitrile (620 mg, 4.27 mmol) in dioxane (4 mL) dropwise, followed by the addition of K₂OsO₄ (14.2 mg, 0.04 mmol). The resulting mixture was stirred for 18 h at room temperature. The reaction mixture was diluted with H₂O (10 mL). The resulting mixture was extracted with EA (3×20 mL). The organic layers were combined, washed with brine (1×20 mL) and dried over Na₂SO₄. After filtration, the filtrate was concentrated under reduced pressure. The residue was purified by silica gel column chromatography, eluted with PE/EA (5:1) to afford 5-formyl-6-methylpyrazine-2-carbonitrile (220 mg, 32.1%).

### Step E: Synthesis of 5-({4-[(2S)-2-(4-chloro-2-fluorophenyl)-2-methyl-1,3-benzodioxol-4-yl]piperidin-1-yl}methyl)-6-methylpyrazine-2-carbonitrile

A stirred mixture of 5-formyl-6-methylpyrazine-2-carbonitrile (198 mg, 1.35 mmol), and 4-[(2S)-2-(4-chloro-2-fluorophenyl)-2-methyl-1,3-benzodioxol-4-yl]piperidine; para-toluene sulfonate (350 mg, 0.67 mmol) in DCM (5 mL) was basified to pH=8 with DIEA. The reaction mixture was stirred for 2 h at room temperature. Then, NaBH(OAc)₃ (428 mg, 2.02 mmol) was added to the above mixture. The reaction mixture was stirred for additional 1 h at room temperature under N₂ atmosphere. The reaction mixture was monitored by LC/MS. The reaction mixture was quenched with H₂O (10 mL). The resulting mixture was extracted with EA (3×10 mL). The organic layers were combined, washed with brine (1×10 mL), and dried over Na₂SO₄. After filtration, the filtrate was concentrated under reduced pressure. The residue was purified by silica gel column chromatography, eluted with EA:PE (2:3) to afford 5-({4-[(2S)-2-(4-chloro-2-fluorophenyl)-2-methyl-1,3-benzodioxol-4-yl]piperidin-1-yl}methyl)-6-methylpyrazine-2-carbonitrile (300 mg, 80.5%). LC-MS (ES⁺): m/z 479.0 [MH⁺].

### Step F: Synthesis of 2-({4-[(2S)-2-(4-chloro-2-fluorophenyl)-2-methyl-1,3-benzodioxol-4-yl]piperidin-1-yl}methyl)-3-methyl-5-[5-(trifluoromethyl)-4H-1,2,4-triazol-3-yl]pyrazine (52)

2-({4-[(2S)-2-(4-chloro-2-fluorophenyl)-2-methyl-1,3-benzodioxol-4-yl]piperidin-1-yl}methyl)-3-methyl-5-[5-(trifluoromethyl)-4H-1,2,4-triazol-3-yl]pyrazine (30.2 mg) was obtained with the similar method of Example 49. LC-MS (ES⁺): m/z 589.20 [MH⁺]. ¹H NMR (400 MHz, DMSO-d₆): (9.01 (s, 1H), 7.65-7.51 (m, 2H), 7.32-7.35 (m, 1H), 6.83-6.65 (m, 3H), 3.75 (s, 2H), 2.98-2.85 (m, 2H), 2.73 (s, 3H), 2.30-2.15 (m, 2H), 2.05-1.98 (m, 4H), 1.95-1.84 (m, 1H), 1.79-1.61 (m, 4H). ¹⁹F NMR (376 MHz, DMSO-d₆): (−62.84, −110.47.

### Example 53: 2-({4-[(2S)-2-(4-chloro-2-fluorophenyl)-2-methyl-2H-1,3-benzodioxol-4-yl]piperidin-1-yl}methyl)-3-[(1-methoxycyclopropyl)methyl]-5-[5-(trifluoromethyl)-4H-1,2,4-triazol-3-yl]pyridine (53)

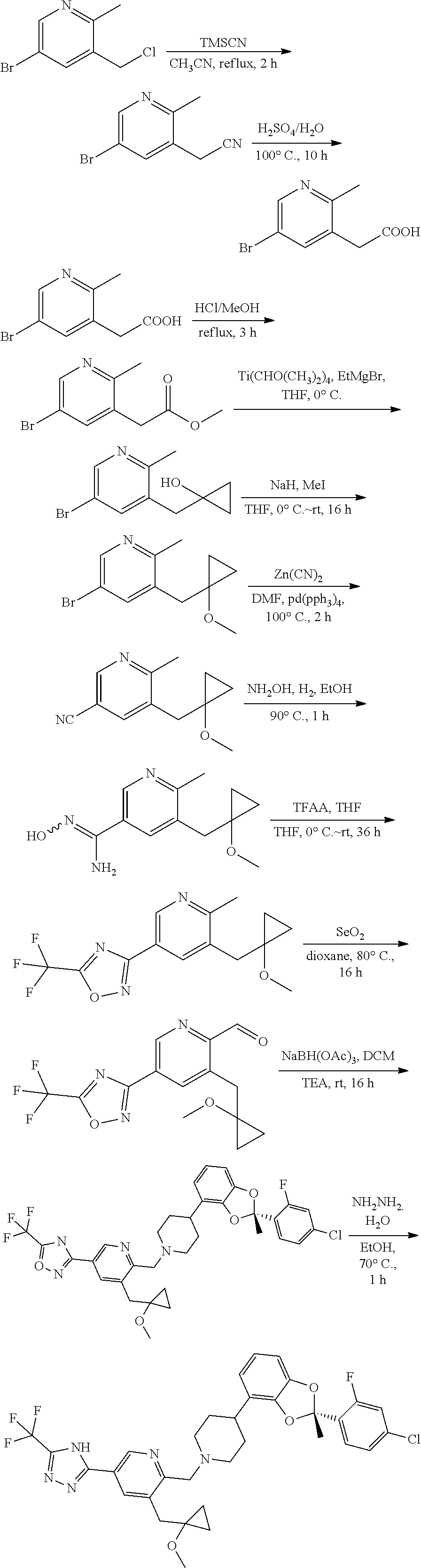

### Step A: 2-(5-bromo-2-methylpyridin-3-yl)acetonitrile

A mixture of 5-bromo-3-(chloromethyl)-2-methylpyridine (0.80 g, 3.65 mmol) in CH₃CN under an atmosphere of N₂ was added TMSCN (543 mg, 5.48 mmol) and TBAF aq (5.50 mL, 1.0 M in THF). The mixture was stirred at 80° C. for 2 h. Water and EtOAc were added to the mixture, the organic phase was separated, washed by brine, dried with Na₂SO₄, the solvent was concentrated to dryness to provide 2-(5-bromo-2-methylpyridin-3-yl)acetonitrile (700 mg, yield: 91.3%). MS Calcd: 210.0; MS Found: 210.9 [M+H]⁺.

### Step B: 2-(5-bromo-2-methylpyridin-3-yl)acetic acid

A mixture of 2-(5-bromo-2-methylpyridin-3-yl)acetonitrile (700 mg, 3.33 mmol) and H₂SO₄/H₂O (7 mL, v:v=1:1) at rt. The mixture was stirred at 100° C. for 10 h. The NaOH aq (15% NaOH in water) was added to adjust the PH of the mixture to 4.0˜5.0. Then, the EtOAc (200 mL) was added, and water (100 mL) was also added. The organic phase was separated, washed by brine, dried over Na₂SO₄. The filtrate was concentrated to dryness to give 2-(5-bromo-2-methylpyridin-3-yl)acetic acid (600 mg, yield: 78.70%). MS Calcd: 229.0; MS Found: 229.9 [M+H]⁺.

### Step C: methyl 2-(5-bromo-2-methylpyridin-3-yl)acetate

To a solution of 2-(5-bromo-2-methylpyridin-3-yl)acetic acid (600 mg, 2.63 mmol) in MeOH (15 mL) was added dropwise HCl/MeOH (25% HCl in MeOH) (1.2 mL, 31.59 mmol) at rt. The mixture was stirred to 80° C. for 3 h. The mixture was poured into NaHCO₃ aq (30 mL)/water (30 mL), extracted with EtOAc (3×50 mL), the combined organic layer was washed with brine, dried over sodium sulfate, filtered and the filtrate was concentrated to give methyl 2-(5-bromo-2-methylpyridin-3-yl)acetate (650 mg, crude). MS Calcd: 243.0; MS Found: 243.9 [M+H]⁺.

### Step D: 1-((5-bromo-2-methylpyridin-3-yl)methyl)cyclopropan-1-ol

To a solution of methyl 2-(5-bromo-2-methylpyridin-3-yl)acetate (650 mg, 2.67 mmol) in THF (100 mL) was added Ti(CHO(CH₃)₂)₄ (7.5 mL, 26.7 mmol) at 0° C. While stirring, EtMgBr (1 M in THF, 66.75 mL, 67.75 mmol) was added to the mixture in 20 min at 0° C. The black mixture was stirred at room temperature for about 30 min. The reaction was quenched with 5 mL H₂SO₄ aq (1M in water). The mixture was poured into water (30 mL) and extracted with EtOAc (2×30 mL), the combined organic layer was washed with brine, dried over sodium sulfate, filtered and the filtrate was concentrated. The residue was purified by pre-HPLC to give 1-((5-bromo-2-methylpyridin-3-yl)methyl)cyclopropan-1-ol (150 mg, yield: 23.3%). MS Calcd: 241.0; MS Found: 241.9 [M+H]⁺.

### Step E: 5-bromo-3-((1-methoxycyclopropyl)methyl)-2-methylpyridine

A mixture of 1-((5-bromo-2-methylpyridin-3-yl)methyl)cyclopropan-1-ol (150 mg, 0.622 mmol) in THF (30 mL) was added NaH (60% w/w) (75 mg, 1.87 mmol) at 0° C. and the mixture was stirred for 0.5 h. Mel (266 mg, 1.87 mmol) was added and the mixture was stirred at room temperature for 16 h. The mixture was poured into water (30 mL) and extracted with EtOAc (2×30 mL), the combined organic layer was washed with brine, dried over sodium sulfate, filtered and the filtrate was concentrated. The residue was purified by column chromatography on silica gel to give 5-bromo-3-((1-methoxycyclopropyl)methyl)-2-methylpyridine (150 mg, yield: 94.3%). MS Calcd: 255.0; MS Found: 256.0 [M+H]⁺.

### Step F: 5-((1-methoxycyclopropyl)methyl)-6-methylnicotinonitrile

A mixture of 5-bromo-3-((1-methoxycyclopropyl)methyl)-2-methylpyridine (150 mg, 0.59 mmol), Zn(CN)₂ (210 mg, 1.77 mmol) and Pd(PPh₃)₄(68 mg, 0.059 mmol) in DMF (5 mL) was stirred at 100° C. under Ar with microwave irradiation for 2 h. The mixture was poured into water (50 mL) and extracted with EtOAc (2×50 mL), the combined organic layer was washed with brine, dried over sodium sulfate, filtered and the filtrate was concentrated. The residue was purified by pre-HPLC to give 5-((1-methoxycyclopropyl)methyl)-6-methylnicotinonitrile (53 mg, yield: 44.4%). MS Calcd: 202.1; MS Found: 203.4 [M+H]⁺.

### Step G: N′-hydroxy-5-((1-methoxycyclopropyl)methyl)-6-methylnicotinimidamide

A mixture of 5-((1-methoxycyclopropyl)methyl)-6-methylnicotinonitrile (53 mg, 0.262 mmol) and NH₂OH aqueous solution (50% NH₂OH in water) (173 mg, 2.62 mmol) in EtOH (5 mL) was stirred at 90° C. for 1 h. The mixture was concentrated to give N′-hydroxy-5-((1-methoxycyclopropyl)methyl)-6-methylnicotinimidamide (66 mg, crude). MS Calcd: 235.1; MS Found: 236.2 [M+H]⁺.

### Step H: 3-(5-((1-methoxycyclopropyl)methyl)-6-methylpyridin-3-yl)-5-(trifluoromethyl)-1,2,4-oxadiazole

To a solution of N′-hydroxy-5-((1-methoxycyclopropyl)methyl)-6-methylnicotinimidamide (66 mg, crude) in THF (10 mL) were added dropwise TFAA (550 mg, 2.62 mmol) in THF (0.5 mL) at 0° C. The mixture was stirred at rt for 36 h. The mixture was poured into sodium bicarbonate aqueous solution (100 mL) and extracted with EtOAc (2×120 mL), the combined organic layer was washed with brine, dried over sodium sulfate, filtered and the filtrate was concentrated. The residue was purified by prep-TLC to give 3-(5-((1-methoxycyclopropyl)methyl)-6-methylpyridin-3-yl)-5-(trifluoromethyl)-1,2,4-oxadiazole (25 mg, yield: 30.5%). MS Calcd: 313.1; MS Found: 314.2 [M+H]⁺.

### Step I: 3-((1-methoxycyclopropyl)methyl)-5-(5-(trifluoromethyl)-1,2,4-oxadiazol-3-yl)picolinaldehyde

A mixture of 3-(5-((1-methoxycyclopropyl)methyl)-6-methylpyridin-3-yl)-5-(trifluoromethyl)-1,2,4-oxadiazole (10 mg, 0.080 mmol) and SeO₂ (37 mg, 0.80 mmol) in 1,4-dioxane (5 mL) was stirred at 80° C. for 16 h. The mixture was filtered, and the filtrate was concentrated to give 3-((1-methoxycyclopropyl)methyl)-5-(5-(trifluoromethyl)-1,2,4-oxadiazol-3-yl)picolinaldehyde (27 mg). MS Calcd: 327.1; MS Found: 328.2 [M+H]⁺.

### Step J: (S)-3-(6-((4-(2-(4-chloro-2-fluorophenyl)-2-methylbenzo[d][1,3]dioxol-4-yl)piperidin-1-yl)methyl)-5-((1-methoxycyclopropyl)methyl)pyridin-3-yl)-5-(trifluoromethyl)-1,2,4-oxadiazole

A mixture of 3-(5-(2-methoxypropyl)-6-methylpyridin-3-yl)-5-(trifluoromethyl)-1,2,4-oxadiazole (42 mg, 0.082 mmol) and TEA (0.06 mL, 0.41 mmol) in DCM (5 mL) was stirred at rt for 0.5 h. 3-(2-methoxypropyl)-5-(5-(trifluoromethyl)-1,2,4-oxadiazol-3-yl)picolinaldehyde (27 mg) was added and the mixture was stirred for 2 h. NaBH(OAc)₃ (70 mg, 0.328 mmol) was added and the mixture was stirred at rt for 16 h. The mixture was poured into water (30 mL) and extracted with DCM (2×30 mL), the combined organic layer was washed with brine, dried over sodium sulfate, filtered and the filtrate was concentrated. The residue was purified by pre-HPLC to give (S)-3-(6-((4-(2-(4-chloro-2-fluorophenyl)-2-methylbenzo[d][1,3]dioxol-4-yl)piperidin-1-yl)methyl)-5-((1-methoxycyclopropyl)methyl)pyridin-3-yl)-5-(trifluoromethyl)-1,2,4-oxadiazole (16 mg, yield: 29.6%%). MS Calcd: 658.2; MS Found: 659.0 [M+H]⁺.

### Step K: 2-({4-[(2S)-2-(4-chloro-2-fluorophenyl)-2-methyl-2H-1,3-benzodioxol-4-yl]piperidin-1-yl}methyl)-3-[(1-methoxycyclopropyl)methyl]-5-[5-(trifluoromethyl)-4H-1,2,4-triazol-3-yl]pyridine (53)

A mixture of (S)-3-(6-((4-(2-(4-chloro-2-fluorophenyl)-2-methylbenzo[d][1,3]dioxol-4-yl)piperidin-1-yl)methyl)-5-((1-methoxycyclopropyl)methyl)pyridin-3-yl)-5-(trifluoromethyl)-1,2,4-oxadiazole (16 mg, 0.023 mmol) and NH₂NH₂·H₂O (2 drops) in EtOH (1 mL) was stirred at 70° C. for 1 h. The mixture was purified by prep-HPLC to give 2-({4-[(2S)-2-(4-chloro-2-fluorophenyl)-2-methyl-2H-1,3-benzodioxol-4-yl]piperidin-1-yl}methyl)-3-[(1-methoxycyclopropyl)methyl]-5-[5-(trifluoromethyl)-4H-1,2,4-triazol-3-yl]pyridine (53) (2.03 mg, yield: 13.4%). MS Calcd: 657.2; MS Found: 658.3 [M+H]⁺.

¹H NMR (400 MHz, CD₃OD) δ 9.11 (s, 1H), 8.48 (s, 1H), 7.61 (t, J=8.4 Hz, 1H), 7.30 (dd, J₁=8.8 Hz/J₂ 12.8 Hz, 1H), 7.22 (dd, J₁=6.4 Hz/J₂ 7.6 Hz, 1H), 6.85-6.73 (m, 3H), 4.45-4.30 (m, 2H), 3.58-3.50 (m, 2H), 3.32 (s, 3H), 3.16 (s, 2H), 3.10-2.90 (m, 3H), 2.30-2.10 (m, 2H), 2.08-1.96 (m, 5H), 0.95-0.90 (m, 2H), 0.71-0.66 (m, 2H). ¹⁹F-NMR (377 MHz, CD₃OD): δ −65.12, −65.16, −112.23.

### Example 54: 2-{[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}-1λ⁶-thietane-1,1-dione (54)

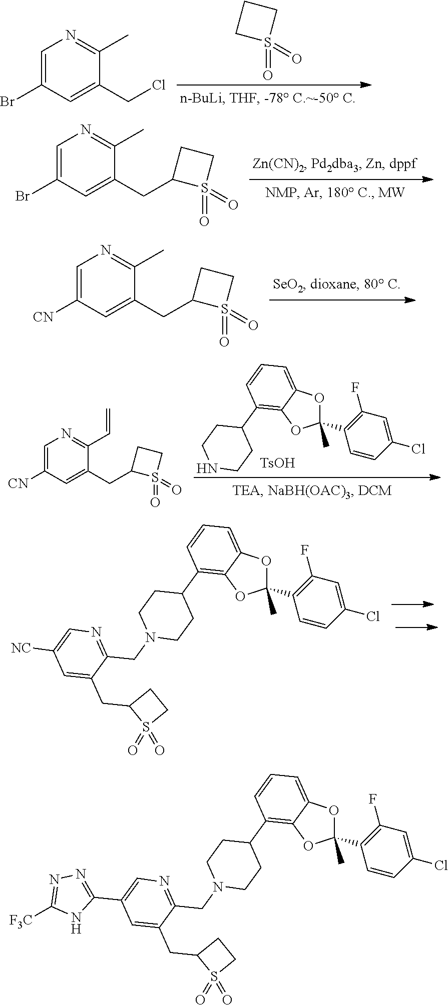

### Step A: 2-((5-bromo-2-methylpyridin-3-yl)methyl)thietane 1,1-dioxide

To a solution of thietane 1,1-dioxide (150 mg, 1.415 mmol) in THF (5 mL) was added dropwise n-BuLi (2.5 M in THF) (0.57 mL, 1.415 mmol) at −78° C. under Ar. The mixture was stirred at −78° C. for 15 min. 5-bromo-3-(chloromethyl)-2-methylpyridine (156 mg, 0.707 mmol) in THF (5 mL) was added in a dropwise manner and the mixture stirred at −50° C. for 1 h. The mixture was poured into water (12 mL) and filtered. The filtrate was purified by flash-reversed phase column chromatography to give 2-((5-bromo-2-methylpyridin-3-yl)methyl)thietane 1,1-dioxide (60 mg, yield: 29.3%). MS Calcd: 289.0; MS Found: 290.0 [M+H]⁺.

### Step B: 5-((1,1-dioxidothietan-2-yl)methyl)-6-methylnicotinonitrile

A mixture of 2-((5-bromo-2-methylpyridin-3-yl)methyl)thietane 1,1-dioxide (60 mg, 0.208 mmol), Zn(CN)₂ (121 mg, 1.038 mmol), Zn (3 mg, 0.052 mmol), Pd₂dba₃ (8 mg, 0.0083 mmol) and dppf (9 mg, 0.0166 mmol) in NMP (1 mL) was stirred at 180° C. under Ar with microwave irradiation for 2 h. The mixture was filtered and the residue was purified by column chromatography on silica gel to give 5-((1,1-dioxidothietan-2-yl)methyl)-6-methylnicotinonitrile (15 mg, yield: 30.6%). MS Calcd: 236.1; MS Found: 237.2 [M+H]⁺.

### Step C: 5-((1,1-dioxidothietan-2-yl)methyl)-6-formylnicotinonitrile

A mixture of 5-((1,1-dioxidothietan-2-yl)methyl)-6-methylnicotinonitrile (15 mg, 0.063 mmol) and SeO₂ (70 mg, 0.63 mmol) in 1,4-dioxane (5 mL) was stirred at 80° C. for 16 h. The mixture was filtered, and the filtrate was concentrated to give 5-((1,1-dioxidothietan-2-yl)methyl)-6-formylnicotinonitrile (10 mg, yield: 62.9%). MS Calcd: 250.0; MS Found: 251.1 [M+H]⁺.

### Step D: 6-((4-((S)-2-(4-chloro-2-fluorophenyl)-2-methylbenzo[d][1,3]dioxol-4-yl)piperidin-1-yl)methyl)-5-((1,1-dioxidothietan-2-yl)methyl)nicotinonitrile

A mixture of (S)-4-(2-(4-chloro-2-fluorophenyl)-2-methylbenzo[d][1,3]dioxol-4-yl)piperidine 4-methylbenzenesulfonic acid salt (21 mg, 0.04 mmol) and TEA (20 mg, 0.2 mmol) in DCM (3 mL) was stirred at rt for 0.5 h. 5-((1,1-dioxidothietan-2-yl)methyl)-6-formylnicotinonitrile (10 mg, 0.04 mmol) was added and the mixture was stirred for 2 h. NaBH(OAc)₃ (34 mg, 0.16 mmol) was added and the mixture was stirred at rt for 16 h. The mixture was poured into water (30 mL) and extracted with DCM (2×30 mL), the combined organic layer was washed with brine, dried over sodium sulfate, filtered and the filtrate was concentrated. The residue was purified by column chromatography on silica gel to give 6-((4-((S)-2-(4-chloro-2-fluorophenyl)-2-methylbenzo[d][1,3]dioxol-4-yl)piperidin-1-yl)methyl)-5-((1,1-dioxidothietan-2-yl)methyl)nicotinonitrile (10 mg, yield: 43%). MS Calcd: 581.2; MS Found: 582.3 [M+H]⁺.

### Step E: 2-{[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}-1λ⁶-thietane-1,1-dione (54)

2-{[2-({4-[(2S)-2-(4-chloro-2-fluorophenyl)-2-methyl-2H-1,3-benzodioxol-4-yl]piperidin-1-yl}methyl)-5-[5-(trifluoromethyl)-4H-1,2,4-triazol-3-yl]pyridin-3-yl]methyl}-1λ⁶-thietane-1,1-dione (54) (0.93 mg) was obtained with the similar method of Example 1. MS Calcd: 691.2; MS Found: 692.3 [M+H]⁺. ¹H NMR (400 MHz, CD₃OD): δ 9.11 (s, 1H), 8.39 (s, 1H), 7.61 (t, J=8.4 Hz, 1H), 7.29 (d, J=9.6 Hz, 1H), 7.22 (d, J=8.0 Hz, 1H), 6.85-6.70 (m, 3H), 4.56 (s, 2H), 4.42-4.26 (m, 2H), 4.14-3.94 (m, 3H), 3.50-3.46 (m, 2H), 3.00-2.80 (m, 2H), 2.55-2.41 (m, 1H), 2.24-2.10 (m, 3H), 2.05 (s, 3H), 2.05-1.93 (m, 3H). ¹⁹F-NMR (377 MHz, CD₃OD): δ −65.21, −112.26.

### Example 55: 2-({4-[(2S)-2-(4-chloro-2-fluorophenyl)-2-methyl-2H-1,3-benzodioxol-4-yl]piperidin-1-yl}methyl)-3-(methanesulfonylmethyl)-5-[5-(trifluoromethyl)-4H-1,2,4-triazol-3-yl]pyridine (55)

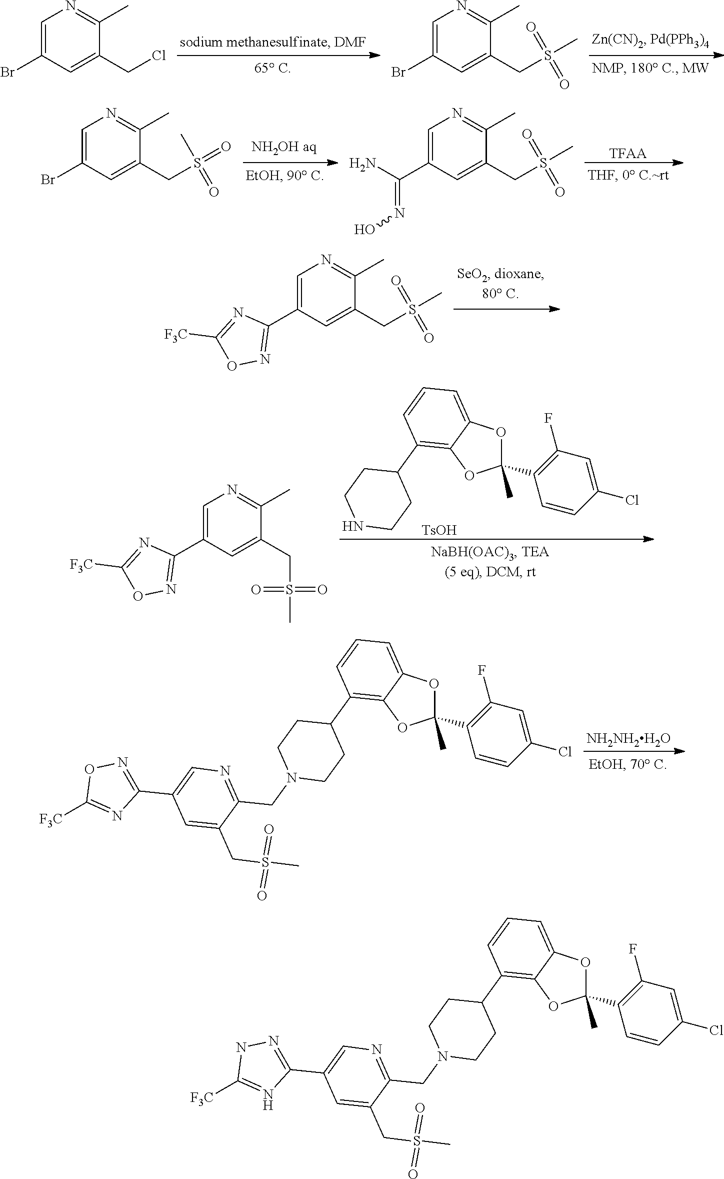

### Step A: 5-bromo-2-methyl-3-((methylsulfonyl)methyl)pyridine

A mixture of 5-bromo-3-(chloromethyl)-2-methylpyridine (1.1 g, 5.0 mmol) and sodium methanesulfinate (765 mg, 7.5 mmol) in DMF (10 mL) was stirred at 65° C. for 1 h. The mixture was poured into water (200 mL) and extracted with EtOAc (2×200 mL), the combined organic layer was washed with brine, dried over sodium sulfate, filtered and the filtrate was concentrated. The residue was purified by column chromatography on silica gel to give 5-bromo-2-methyl-3-((methylsulfonyl)methyl)pyridine (810 mg, yield: 61.6%). MS Calcd: 263.0; MS Found: 263.9 [M+H]⁺.

### Step B: 6-methyl-5-((methylsulfonyl)methyl)nicotinonitrile

A mixture of 5-bromo-2-methyl-3-((methylsulfonyl)methyl)pyridine (410 mg, 1.56 mmol), Zn(CN)₂ (547 mg, 4.68 mmol) and Pd(PPh₃)₄(180 mg, 0.156 mmol) in NMP (2 mL) was stirred at 180° C. under Ar with microwave irradiation for 2 h. The mixture was poured into water (100 mL) and extracted with EtOAc (2×100 mL), the combined organic layer was washed with brine, dried over sodium sulfate, filtered and the filtrate was concentrated. The residue was purified by column chromatography on silica gel to give 6-methyl-5-((methylsulfonyl)methyl)nicotinonitrile (850 mg, crude). MS Calcd: 210.0; MS Found: 210.9 [M+H]⁺.

### Step C: N′-hydroxy-6-methyl-5-((methylsulfonyl)methyl)nicotinimidamide

A mixture of 6-methyl-5-((methylsulfonyl)methyl)nicotinonitrile (750 mg, 3.57 mmol) and NH₂OH aqueous solution (50% NH₂OH in water) (2.4 mL, 35.7 mmol) in EtOH (10 mL) was stirred at 90° C. for 1 h. The mixture was concentrated to give N′-hydroxy-6-methyl-5-((methylsulfonyl)methyl)nicotinimidamide (750 mg, crude). MS Calcd: 243.1; MS Found: 244.0 [M+H]⁺.

### Step D: 3-(6-methyl-5-((methylsulfonyl)methyl)pyridin-3-yl)-5-(trifluoromethyl)-1,2,4-oxadiazole

To a solution of N′-hydroxy-6-methyl-5-((methylsulfonyl)methyl)nicotinimidamide (750 mg, 3.09 mmol) in THF (10 mL) were added TFAA (2.2 mL, 15.4 mmol) in THF (2 mL) in a dropwise manner at 0° C. The mixture was stirred at rt for 16 h. The mixture was poured into sodium bicarbonate aqueous solution (100 mL) and extracted with EtOAc (2×100 mL), the combined organic layer was washed with brine, dried over sodium sulfate, filtered and the filtrate was concentrated. The residue was purified by column chromatography on silica gel to give 3-(6-methyl-5-((methylsulfonyl)methyl)pyridin-3-yl)-5-(trifluoromethyl)-1,2,4-oxadiazole (240 mg, yield: 21.4%). MS Calcd: 321.0; MS Found: 322.4 [M+H]⁺.

### Step E: 3-((methylsulfonyl)methyl)-5-(5-(trifluoromethyl)-1,2,4-oxadiazol-3-yl)picolinaldehyde

A mixture of 3-(6-methyl-5-((methylsulfonyl)methyl)pyridin-3-yl)-5-(trifluoromethyl)-1,2,4-oxadiazole (120 mg, 0.374 mmol) and SeO₂ (415 mg, 3.74 mmol) in 1,4-dioxane (5 mL) was stirred at 80° C. for 16 h. The mixture was filtered and the filtrate was concentrated to give 3-((methylsulfonyl)methyl)-5-(5-(trifluoromethyl)-1,2,4-oxadiazol-3-yl)picolinaldehyde (126 mg, crude). MS Calcd: 335.0; MS Found: 336.0 [M+H]⁺.

### Step F: (S)-3-(6-((4-(2-(4-chloro-2-fluorophenyl)-2-methylbenzo[d][1,3]dioxol-4-yl)piperidin-1-yl)methyl)-5-((methylsulfonyl)methyl)pyridin-3-yl)-5-(trifluoromethyl)-1,2,4-oxadiazole

A mixture of (S)-4-(2-(4-chloro-2-fluorophenyl)-2-methylbenzo[d][1,3]dioxol-4-yl)piperidine 4-methylbenzenesulfonic acid salt (65 mg, 0.125 mmol) and TEA (63 mg, 0.627 mmol) in DCM (5 mL) was stirred at rt for 0.5 h. 3-((methylsulfonyl)methyl)-5-(5-(trifluoromethyl)-1,2,4-oxadiazol-3-yl)picolinaldehyde (42 mg, 0.125 mmol) was added and the mixture was stirred for 2 h. NaBH(OAc)₃ (106 mg, 0.5 mmol) was added and the mixture was stirred at rt for 16 h. The mixture was poured into water (50 mL) and extracted with DCM (2×50 mL), the combined organic layer was washed with brine, dried over sodium sulfate, filtered and the filtrate was concentrated. The residue was purified by column chromatography on silica gel to give (S)-3-(6-((4-(2-(4-chloro-2-fluorophenyl)-2-methylbenzo[d][1,3]dioxol-4-yl)piperidin-1-yl)methyl)-5-((methylsulfonyl)methyl)pyridin-3-yl)-5-(trifluoromethyl)-1,2,4-oxadiazole (83 mg, crude). MS Calcd: 666.1; MS Found: 667.2 [M+H]⁺.

### Step G: 2-({4-[(2S)-2-(4-chloro-2-fluorophenyl)-2-methyl-2H-1,3-benzodioxol-4-yl]piperidin-1-yl}methyl)-3-(methanesulfonylmethyl)-5-[5-(trifluoromethyl)-4H-1,2,4-triazol-3-yl]pyridine (55)

A mixture of (S)-3-(6-((4-(2-(4-chloro-2-fluorophenyl)-2-methylbenzo[d][1,3]dioxol-4-yl)piperidin-1-yl)methyl)-5-((methylsulfonyl)methyl)pyridin-3-yl)-5-(trifluoromethyl)-1,2,4-oxadiazole (83 mg, 0.125 mmol) and NH₂NH₂·H₂O (5 drops) in EtOH (2 mL) was stirred at 70° C. for 1 h. The mixture was purified by prep-HPLC to give 2-({4-[(2S)-2-(4-chloro-2-fluorophenyl)-2-methyl-2H-1,3-benzodioxol-4-yl]piperidin-1-yl}methyl)-3-(methanesulfonylmethyl)-5-[5-(trifluoromethyl)-4H-1,2,4-triazol-3-yl]pyridine (55) (21.9 mg, yield: 26.4%). MS Calcd: 665.1; MS Found: 666.1 [M+H]⁺. ¹H NMR (400 MHz, CD₃OD): δ 9.28 (d, J₁=1.6 Hz, 1H), 8.47 (d, J₁=2.0 Hz, 1H), 7.61 (t, J₁=8.4 Hz, 1H), 7.30 (dd, J1=2.0 Hz/J₂=11.2 Hz, 1H), 7.22 (dd, J₁=1.2 Hz/J=8.4 Hz, 1H), 6.85-6.73 (m, 3H), 4.82 (s, 2H), 4.60 (s, 2H), 3.65-3.53 (m, 2H), 3.20-2.95 (m, 6H), 2.30-2.13 (m, 2H), 2.10-2.00 (m, 5H). ¹⁹F-NMR (377 MHz, CD₃OD): δ −65.21, −112.22.

### Example 56: (S)-2-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)-N,N-dimethylacetamide (56)

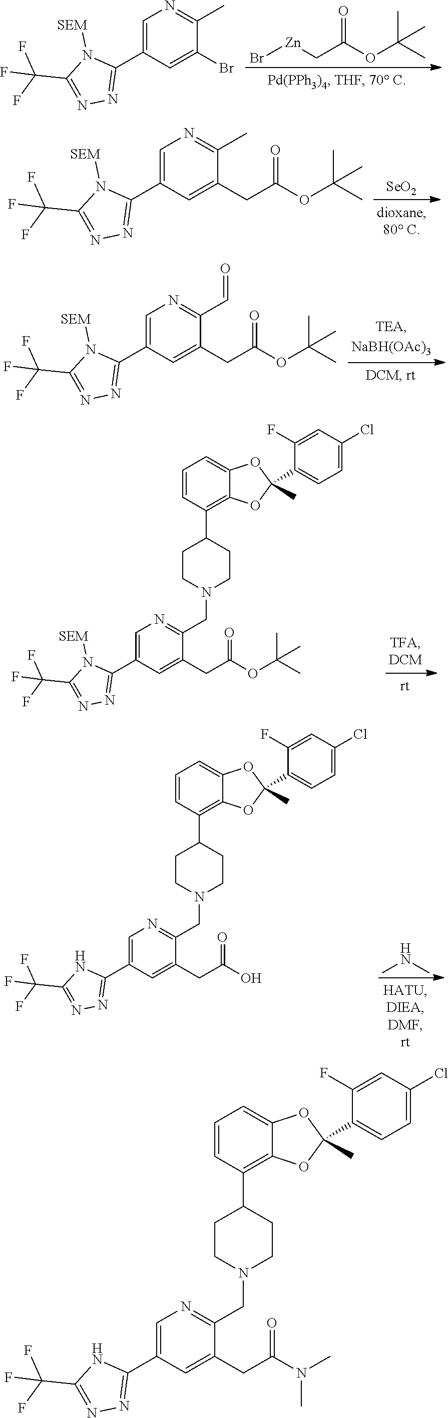

### Step B: tert-butyl 2-(2-formyl-5-(5-(trifluoromethyl)-4-((2-(trimethylsilyl)ethoxy)methyl)-4H-1,2,4-triazol-3-yl)pyridin-3-yl)acetate

A mixture of tert-butyl 2-(2-methyl-5-(5-(trifluoromethyl)-4-((2-(trimethylsilyl)ethoxy)methyl)-4H-1,2,4-triazol-3-yl)pyridin-3-yl)acetate (30 mg, 0.06 mmol) and SeO₂ (71 mg, 0.64 mmol) in 1,4-dioxane (3 mL) was stirred at 80° C. for 16 h. The mixture was filtered, and the residue was concentrated to obtain tert-butyl 2-(2-formyl-5-(5-(trifluoromethyl)-4-((2-(trimethylsilyl)ethoxy)methyl)-4H-1,2,4-triazol-3-yl)pyridin-3-yl)acetate (25 mg, yield: 86.2%). MS Calcd: 486.2; MS Found: 487.3 [M+H]⁺.

### Step C: tert-butyl (S)-2-(2-((4-(2-(4-chloro-2-fluorophenyl)-2-methylbenzo[d][1,3]dioxol-4-yl)piperidin-1-yl)methyl)-5-(5-(trifluoromethyl)-4-((2-(trimethylsilyl)ethoxy)methyl)-4H-1,2,4-triazol-3-yl)pyridin-3-yl)acetate

A mixture of (S)-4-(2-(4-chloro-2-fluorophenyl)-2-methylbenzo[d][1,3]dioxol-4-yl)piperidine 4-methylbenzenesulfonate (29 mg, 0.06 mmol) and TEA (25 mg, 0.26 mmol) in DCM (5 mL) was stirred at rt for 0.5 h. The mixture was added tert-butyl 2-(2-formyl-5-(5-(trifluoromethyl)-4-((2-(trimethylsilyl)ethoxy)methyl)-4H-1,2,4-triazol-3-yl)pyridin-3-yl)acetate (25 mg, 0.05 mmol) and the mixture was stirred for 2 h. The mixture was added NaBH(OAC)₃ (44 mg, 0.21 mmol) and the mixture was stirred at rt for 16 h. The mixture was poured into water (30 mL) and extracted with DCM (2×30 mL), the combined organic layer was washed with brine, dried over sodium sulfate, filtered and the residue was concentrated to obtain tert-butyl (S)-2-(2-((4-(2-(4-chloro-2-fluorophenyl)-2-methylbenzo[d][1,3]dioxol-4-yl)piperidin-1-yl)methyl)-5-(5-(trifluoromethyl)-4-((2-(trimethylsilyl)ethoxy)methyl)-4H-1,2,4-triazol-3-yl)pyridin-3-yl)acetate (45 mg, yield: >99.9%). MS Calcd: 817.3; MS Found: 818.4 [M+H]⁺.

### Step D: (S)-2-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)acetic acid

A mixture of tert-butyl (S)-2-(2-((4-(2-(4-chloro-2-fluorophenyl)-2-methylbenzo[d][1,3]dioxol-4-yl)piperidin-1-yl)methyl)-5-(5-(trifluoromethyl)-4-((2-(trimethylsilyl)ethoxy)methyl)-4H-1,2,4-triazol-3-yl)pyridin-3-yl)acetate (45 mg, 0.06 mmol) and TFA (1 mL) in DCM (2 mL) was stirred at rt for 1 h. The mixture was poured into saturated NaHCO₃ aq (50 mL) and extracted with DCM (2×50 mL), the combined organic layer was washed with brine, dried over sodium sulfate, filtered and the residue was concentrated to obtain (S)-2-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)acetic acid (37 mg, yield: >99.9%). MS Calcd: 631.2; MS Found: 632.3 [M+H]⁺.

### Step E: (S)-2-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)-N,N-dimethylacetamide

A mixture of obtain (S)-2-(2-((4-(2-(4-chloro-2-fluorophenyl)-2-methylbenzo[d][1,3]dioxol-4-yl)piperidin-1-yl)methyl)-5-(5-(trifluoromethyl)-4H-1,2,4-triazol-3-yl)pyridin-3-yl)acetic acid (37 mg, 0.06 mmol), DIEA (39 mg, 0.30 mmol) and dimethylamine (11 mg, 0.24 mmol) in DMF (3 mL) was stirred at rt for 5 min. HATU (45 mg, 0.12 mmol) was added, the mixture was stirred at rt for 2 h. The mixture was purified by prep-HPLC to obtain compound 56 (2.35 mg, yield: 6.03%). MS Calcd.: 658.2; MS Found: 659.4 [M+H]⁺. ¹H NMR (400 MHz, CD₃OD-d₄): δ 9.16 (s, 1H), 8.27 (s, 1H), 7.61 (t, J=8.4 Hz, 1H), 7.25 (dd, J₁=10.8 Hz/J=29.2 Hz, 2H), 6.84-6.80 (m, 1H), 6.75 (d, J=8.0 Hz, 2H), 4.39-4.24 (m, 1H), 4.06 (s, 2H), 3.64-3.61 (m, 3H), 3.48-3.45 (m, 2H), 3.43-3.39 (m, 3H), 3.03-2.99 (m, 3H), 2.99-2.95 (m, 1H), 2.20-1.97 (m, 7H). ¹⁹F NMR (377 MHz, CD₃OD-d₄): δ (−74.04), (−76.04), (−112.25).

### Example 57: 2-[(4-{6-[(4-chloro-2-fluorophenyl)methoxy]pyridin-2-yl}piperidin-1-yl)methyl]-3-(methanesulfonylmethyl)-5-[5-(trifluoromethyl)-4H-1,2,4-triazol-3-yl]pyridine (57)

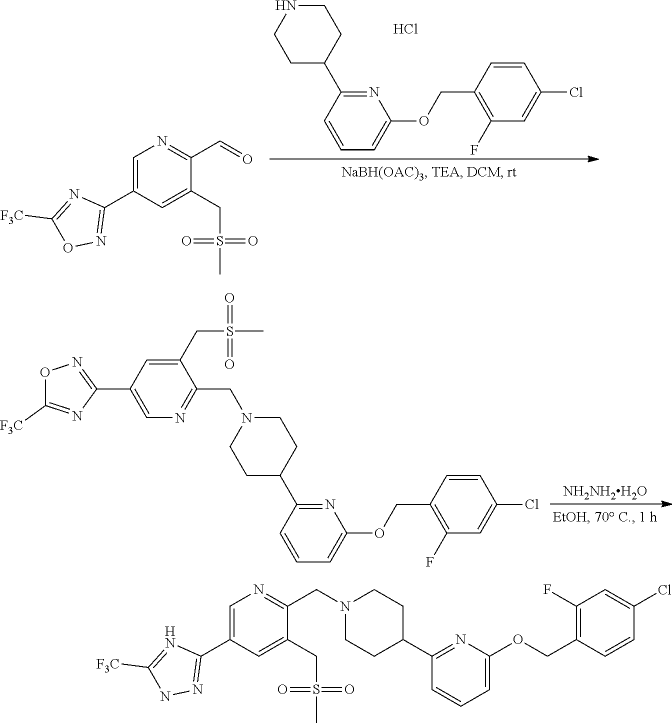

### Step A: 3-(6-((4-(6-((4-chloro-2-fluorobenzyl)oxy)pyridin-2-yl)piperidin-1-yl)methyl)-5-((methylsulfonyl)methyl)pyridin-3-yl)-5-(trifluoromethyl)-1,2,4-oxadiazole

A mixture of 2-((4-chloro-2-fluorobenzyl)oxy)-6-(piperidin-4-yl)pyridine hydrochloric acid salt (45 mg, 0.12 mmol) and TEA (63 mg, 0.63 mmol) in DCM (5 mL) was stirred at rt for 0.5 h. 3-((methylsulfonyl)methyl)-5-(5-(trifluoromethyl)-1,2,4-oxadiazol-3-yl)picolinaldehyde (42 mg, 0.12 mmol) was added and the mixture was stirred for 2 h. NaBH(OAc)₃ (106 mg, 0.5 mmol) was added and the mixture was stirred at rt for 16 h. The mixture was poured into water (50 mL) and extracted with DCM (2×50 mL), the combined organic layer was washed with brine, dried over sodium sulfate, filtered and the filtrate was concentrated. The residue was purified by column chromatography on silica gel to give 3-(6-((4-(6-((4-chloro-2-fluorobenzyl)oxy)pyridin-2-yl)piperidin-1-yl)methyl)-5-((methylsulfonyl)methyl)pyridin-3-yl)-5-(trifluoromethyl)-1,2,4-oxadiazole (20 mg, yield: 25%). MS Calcd: 639.1; MS Found: 640.0 [M+H]⁺.

### Step B: 2-[(4-{6-[(4-chloro-2-fluorophenyl)methoxy]pyridin-2-yl}piperidin-1-yl)methyl]-3-(methanesulfonylmethyl)-5-[5-(trifluoromethyl)-4H-1,2,4-triazol-3-yl]pyridine (57)

A mixture of 3-(6-((4-(6-((4-chloro-2-fluorobenzyl)oxy)pyridin-2-yl)piperidin-1-yl)methyl)-5-((methylsulfonyl)methyl)pyridin-3-yl)-5-(trifluoromethyl)-1,2,4-oxadiazole (20 mg, 0.031 mmol) and NH₂NH₂·H₂O (2 drops) in EtOH (1 mL) was stirred at 70° C. for 1 h. The mixture was purified by prep-HPLC to give 2-[(4-{6-[(4-chloro-2-fluorophenyl)methoxy]pyridin-2-yl}piperidin-1-yl)methyl]-3-(methanesulfonylmethyl)-5-[5-(trifluoromethyl)-4H-1,2,4-triazol-3-yl]pyridine (57) (4.8 mg, yield: 23.9%). MS Calcd: 638.2; MS Found: 639.1 [M+H]⁺.

¹H NMR (400 MHz, CD₃OD): δ 9.28 (d, J₁=1.2 Hz, 1H), 8.46 (s, 1H), 7.63 (t, J=8.0 Hz, 1H), 7.51 (t, J=8.4 Hz, 1H), 7.28-7.18 (m, 2H), 6.90 (d, J=7.2 Hz, 1H), 6.71 (d, J=8.4 Hz, 1H), 5.45 (s, 2H), 4.62 (s, 2H), 3.65-3.54 (m, 2H), 3.27-3.20 (m, 2H), 3.18-2.90 (m, 6H), 2.30-2.10 (m, 2H), 2.18-2.06 (m, 2H). ¹⁹F-NMR (377 MHz, CD₃OD): δ −65.14, −117.55.

### Example 58: 3-[6-({4-[(2S)-2-(4-chloro-2-fluorophenyl)-2-methyl-2H-1,3-benzodioxol-4-yl]piperidin-1-yl}methyl)-5-(methanesulfonylmethyl)pyridin-3-yl]-2,5-dihydro-1,2,4-oxadiazol-5-one (58)

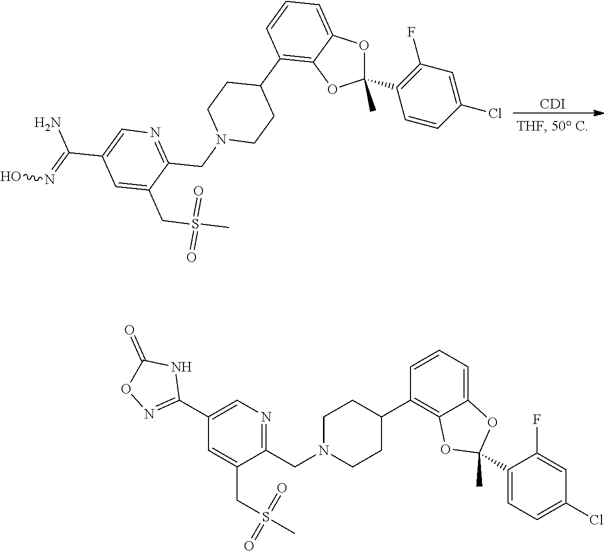

A mixture of (S)-6-((4-(2-(4-chloro-2-fluorophenyl)-2-methylbenzo[d][1,3]dioxol-4-yl)piperidin-1-yl)methyl)-N′-hydroxy-5-((methylsulfonyl)methyl)nicotinimidamide (10 mg, 0.017 mmol) and CDI (14 mg, 0.085 mmol) in THF (3 mL) was stirred at 50° C. for 16 h. The mixture was purified by prep-HPLC to give 3-[6-({4-[(2S)-2-(4-chloro-2-fluorophenyl)-2-methyl-2H-1,3-benzodioxol-4-yl]piperidin-1-yl}methyl)-5-(methanesulfonylmethyl)pyridin-3-yl]-2,5-dihydro-1,2,4-oxadiazol-5-one (58) (2.3 mg, yield: 21.9%). MS Calcd: 614.1; MS Found: 615.3 [M+H]⁺.

¹H NMR (400 MHz, CD₃OD): δ 9.08 (s, 1H), 8.31 (s, 1H), 7.61 (t, J=8.4 Hz, 1H), 7.29 (d, J=10.4 Hz, 1H), 7.22 (d, J=8.8 Hz, 1H), 6.85-6.72 (m, 3H), 4.55 (brs, 2H), 3.60-3.45 (m, 2H), 3.34 (s, 2H), 3.09 (s, 3H), 3.08-2.90 (m, 3H), 2.28-2.10 (m, 2H), 2.04 (s, 3H), 2.10-1.97 (m, 2H). ¹⁹F NMR(377 MHz, CD₃OD): δ −112.23.

### Example 59: 2-({4-[(2S)-2-(4-chloro-2-fluorophenyl)-2-methyl-2H-1,3-benzodioxol-4-yl]piperidin-1-yl}methyl)-3-[(ethanesulfonyl)methyl]-5-[5-(trifluoromethyl)-4H-1,2,4-triazol-3-yl]pyridine (59)

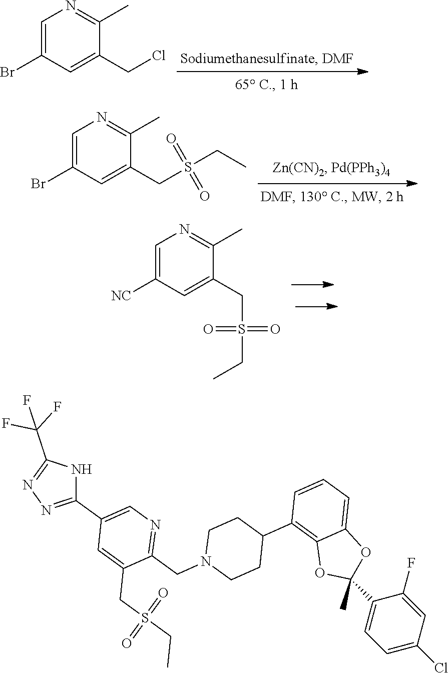

### Step A: 5-bromo-3-((ethylsulfonyl)methyl)-2-methylpyridine

To a solution of 5-bromo-3-(chloromethyl)-2-methylpyridine (200 mg, 0.91 mmol) in DMF (10 mL) was added Sodium ethanesulfinate (159 mg, 1.8 mmol) was stirred at 65° C. for 1 h. The mixture was poured into water (100 mL) and extracted with EtOAc (2×100 mL), the combined organic layer was washed with brine, dried over sodium sulfate, filtered and the filtrate was concentrated to give 5-bromo-3-((ethylsulfonyl)methyl)-2-methylpyridine (210 mg, yield: 83.0%). MS Calcd: 277.0; MS Found: 277.8 [M+H]⁺.

### Step B: 5-((ethylsulfonyl)methyl)-6-methylnicotinonitrile

A mixture of 5-bromo-3-((ethylsulfonyl)methyl)-2-methylpyridine (210 mg, 0.76 mmol), Zn(CN)₂ (266 mg, 2.3 mmol) and Pd(PPh₃)₄ (87 mg, 0.08 mmol) in DMF (5 mL) was stirred at 130° C. under Ar with microwave irradiation for 2 h. The mixture was filtered, and the residue was purified by column chromatography on silica gel to give 5-((ethylsulfonyl)methyl)-6-methylnicotinonitrile (60 mg, yield: 35.3%). MS Calcd: 224.1; MS Found: 224.8 [M+H]⁺.

### Step C: 2-({4-[(2S)-2-(4-chloro-2-fluorophenyl)-2-methyl-2H-1,3-benzodioxol-4-yl]piperidin-1-yl}methyl)-3-[(ethanesulfonyl)methyl]-5-[5-(trifluoromethyl)-4H-1,2,4-triazol-3-yl]pyridine (59)

2-({4-[(2S)-2-(4-chloro-2-fluorophenyl)-2-methyl-2H-1,3-benzodioxol-4-yl]piperidin-1-yl}methyl)-3-[(ethanesulfonyl)methyl]-5-[5-(trifluoromethyl)-4H-1,2,4-triazol-3-yl]pyridine (59) (14.3 mg) was obtained with the similar procedure of Example 55. MS Calcd: 679.2; MS Found: 680.3 [M+H]⁺.

¹H NMR (400 MHz, CD₃OD): δ 9.27 (s, 1H), 8.45 (d, J=2.0 Hz, 1H), 7.61 (t, J=8.4 Hz, 1H), 7.29 (dd, J=8.4 Hz, 1.7 Hz, 1H), 7.22 (dd, J=8 Hz, 1.6 Hz, 1H), 6.86-6.72 (m, 3H), 4.65-4.80 (s, 2H), 4.51 (m, 2H), 3.61-3.48 (m, 2H), 3.27-3.17 (m, 2H), 3.13-2.92 (m, 3H), 2.30-2.13 (m, 2H), 2.12-1.98 (m, 5H), 1.43 (t, J=7.6 Hz, 3H). ¹⁹F NMR (377 MHz, CD₃OD): δ −65.23, −112.24.

### Example 60: 2-((4-((S)-2-(4-chloro-2-fluorophenyl)-2-methylbenzo[d][1,3]dioxol-4-yl)piperidin-1-yl)methyl)-3-(1-(methylsulfonyl)ethyl)-5-(5-(trifluoromethyl)-4H-1,2,4-triazol-3-yl)pyridine (60)

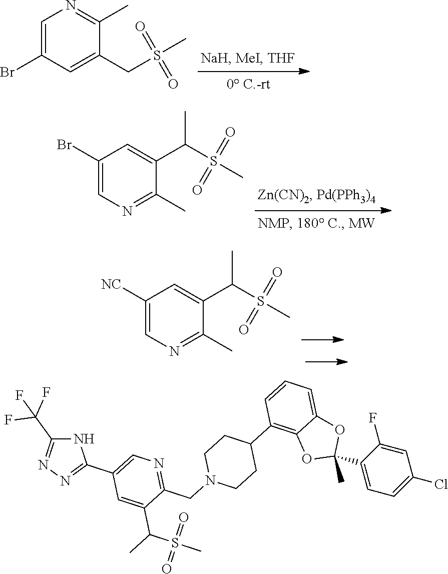

### Step A: The synthesis of 5-bromo-2-methyl-3-(1-(methylsulfonyl)ethyl)pyridine

To a solution of 5-bromo-2-methyl-3-((methylsulfonyl)methyl)pyridine (100 mg, 0.38 mmol) in THF (5 mL) was added NaH (60%) (30 mg, 0.76 mmol) at 0° C. and the mixture was stirred for 0.5 h. The mixture was added Mel (65 mg, 0.46 mmol) and stirred at room temperature for 16 h. The mixture was poured into water (50 mL) and extracted with EtOAC (2×50 mL), the combined organic layer was washed with brine, dried over sodium sulfate, filtered and the residue was concentrated. The residue was purified by column chromatography on silica gel to obtain 5-bromo-2-methyl-3-(1-(methylsulfonyl)ethyl)pyridine (90 mg, yield: 85.5%). MS Calcd: 277.0; MS Found: 278.1 [M+H]⁺.

### Step B: The synthesis of 6-methyl-5-(1-(methylsulfonyl)ethyl)nicotinonitrile

A mixture of 5-bromo-2-methyl-3-(1-(methylsulfonyl)ethyl)pyridine (105 mg, 0.38 mmol), Zn(CN)₂ (133 mg, 1.13 mmol) and Pd(PPh₃)₄(44 mg, 0.038 mmol) in NMP (2 mL) was stirred at 180° C. under Ar with M.W for 2 h. The mixture was filtered, and the residue was purified by column chromatography on silica gel to obtain 6-methyl-5-(1-(methylsulfonyl)ethyl)nicotinonitrile (45 mg, yield: 53.2%). MS Calcd: 224.1; MS Found: 225.0 [M+H]⁺.

### Step C: 2-((4-((S)-2-(4-chloro-2-fluorophenyl)-2-methylbenzo[d][1,3]dioxol-4-yl)piperidin-1-yl)methyl)-3-(1-(methylsulfonyl)ethyl)-5-(5-(trifluoromethyl)-4H-1,2,4-triazol-3-yl)pyridine

2-((4-((S)-2-(4-chloro-2-fluorophenyl)-2-methylbenzo[d][1,3]dioxol-4-yl)piperidin-1-yl)methyl)-3-(1-(methylsulfonyl)ethyl)-5-(5-(trifluoromethyl)-4H-1,2,4-triazol-3-yl)pyridine (21.0 mg) was obtained with the similar procedure of Example 55. MS Calcd: 679.2; MS Found: 680.3 [M+H]⁺.

¹H NMR (400 MHz, CD₃OD-d₄): δ 9.25 (s, 1H), 8.67 (s, 1H), 7.61 (t, J=8.4 Hz, 1H), 7.29 (dd, J₁=1.6 Hz/J=10.8 Hz, 1H), 7.22 (d, J₁=8.4 Hz, 1H), 6.84-6.75 (m, 3H), 5.06-5.00 (m, 1H), 4.50 (brs, 2H), 3.52-3.44 (m, 2H), 3.03 (s, 3H), 3.01-2.91 (m, 3H), 2.23-2.13 (m, 2H), 2.04 (s, 3H), 2.02-1.97 (m, 2H), 1.91 (d, J=7.2 Hz, 3H). ¹⁹F NMR (377 MHz, CD₃OD-d₄): δ (−65.50), (−112.23).

### Example 61: 2-[(4-{6-[(4-chloro-2-fluorophenyl)methoxy]-5-fluoropyridin-2-yl}piperidin-1-yl)methyl]-3-(methanesulfonylmethyl)-5-[5-(trifluoromethyl)-4H-1,2,4-triazol-3-yl]pyridine (61)

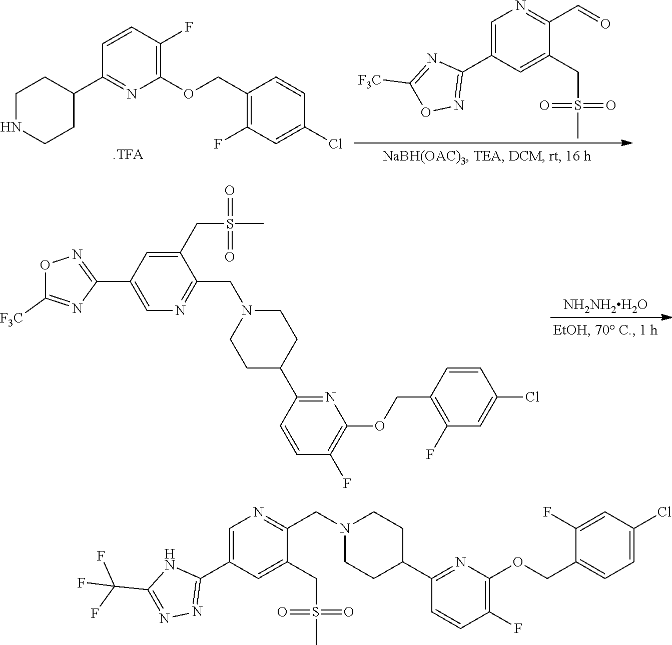

### Step A: 3-(6-((4-(6-((4-chloro-2-fluorobenzyl)oxy)-5-fluoropyridin-2-yl)piperidin-1-yl)methyl)-5-((methylsulfonyl)methyl)pyridin-3-yl)-5-(trifluoromethyl)-1,2,4-oxadiazole

A mixture of 2-((4-chloro-2-fluorobenzyl)oxy)-3-fluoro-6-(piperidin-4-yl)pyridine 2,2,2-trifluoroacetic acid salt (40 mg, 0.089 mmol) and TEA (45 mg, 0.45 mmol) in DCM (5 mL) was stirred at rt for 0.5 h. 3-((methylsulfonyl)methyl)-5-(5-(trifluoromethyl)-1,2,4-oxadiazol-3-yl)picolinaldehyde (30 mg, 0.089 mmol) was added and the mixture was stirred for 2 h. NaBH(OAc)₃ (75 mg, 0.36 mmol) was added and the mixture was stirred at rt for 16 h. The mixture was poured into water (30 mL) and extracted with DCM (2×30 mL), the combined organic layer was washed with brine, dried over sodium sulfate, filtered and the filtrate was concentrated. The residue was purified by column chromatography on silica gel to give 3-(6-((4-(6-((4-chloro-2-fluorobenzyl)oxy)-5-fluoropyridin-2-yl)piperidin-1-yl)methyl)-5-((methylsulfonyl)methyl)pyridin-3-yl)-5-(trifluoromethyl)-1,2,4-oxadiazole (10 mg, yield: 17.0%). MS Calcd: 657.1; MS Found: 658.0 [M+H]⁺.

### Step B: 2-[(4-{6-[(4-chloro-2-fluorophenyl)methoxy]-5-fluoropyridin-2-yl}piperidin-1-yl)methyl]-3-(methanesulfonylmethyl)-5-[5-(trifluoromethyl)-4H-1,2,4-triazol-3-yl]pyridine (61)

A mixture of 3-(6-((4-(6-((4-chloro-2-fluorobenzyl)oxy)-5-fluoropyridin-2-yl)piperidin-1-yl)methyl)-5-((methylsulfonyl)methyl)pyridin-3-yl)-5-(trifluoromethyl)-1,2,4-oxadiazole (10 mg, 0.015 mmol) and NH₂NH₂·H₂O (2 drops) in EtOH (1 mL) was stirred at 70° C. for 1 h. The mixture was purified by prep-HPLC to give 2-[(4-{6-[(4-chloro-2-fluorophenyl)methoxy]-5-fluoropyridin-2-yl}piperidin-1-yl)methyl]-3-(methanesulfonylmethyl)-5-[5-(trifluoromethyl)-4H-1,2,4-triazol-3-yl]pyridine (61) (3.1 mg, yield: 30.7%). MS Calcd: 656.1; MS Found: 657.3 [M+H]⁺.

¹H NMR (400 MHz, CD₃OD): δ 9.26 (d, J₁=1.2 Hz, 1H), 8.46 (s, 1H), 7.54 (t, J=8.4 Hz, 1H), 7.43 (dd, J=8.0 Hz/J=10.0 Hz, 1H), 7.29-7.21 (m, 2H), 6.89 (dd, J=2.8 Hz/J=8.0 Hz, 1H), 5.53 (s, 2H), 4.53 (s, 2H), 3.56-3.46 (m, 2H), 3.31 (s, 2H), 3.09 (s, 3H), 3.08-2.85 (m, 3H), 2.21-2.02 (m, 4H). ¹⁹F NMR (377 MHz, CD₃OD): δ −65.02, −117.28, 144.90.

### Example 62: 1-{[3-({4-[(2S)-2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]pyrazin-2-yl]methyl}cyclopropane-1-carbonitrile (62)

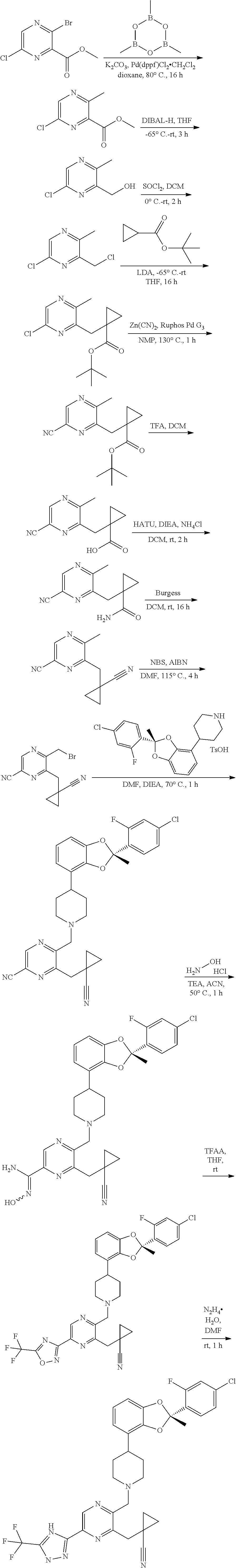

### Step A: 6-chloro-3-methylpyrazine-2-carboxylate

A mixture of methyl 3-bromo-6-chloropyrazine-2-carboxylate (5.0 g, 20.0 mmol), 2,4,6-trimethyl-1,3,5,2,4,6-trioxatriborinane (6.86 mL, 3.5 mol/L), Pd(dppf)Cl₂.DCM (1.63 g, 2.00 mmol) and K₂CO₃ (5.52 g, 40.00 mmol) in dioxane (60 mL) was stirred for 16 hours at 80° C. under nitrogen atmosphere. The mixture was allowed to cool down to room temperature. The resulting mixture was diluted with water (100 mL), extracted with EtOAc (3×100 mL). The combined organic layers were washed with brine (2×60 mL) and dried over anhydrous Na₂SO₄. After filtration, the filtrate was concentrated under reduced pressure. The residue was purified by silica gel column chromatography, eluted with PE/EA (20:1) to afford 6-chloro-3-methylpyrazine-2-carboxylate (1.6 g, yield: 44.1%). MS Calcd.: 186.0; MS Found: 187.0 [M+H]⁺.

### Step B: (6-chloro-3-methylpyrazin-2-yl)methanol

To a solution of methyl 6-chloro-3-methylpyrazine-2-carboxylate (1.64 g, 8.82 mmol) in THF (20 mL) was added DIBAL-H (35.3 mL, 1 mol/L in toluene) at −65° C. The resulting mixture was stirred at room temperature for 3 hours under N₂. The reaction mixture was quenched with aqueous NaOH (2 M, 30 mL) at 0° C., then added H₂O (100 mL), and extracted with EA (3×60 mL). The organic layer was dried over Na2SO4, filtered, and concentrated under reduced pressure. The residue was purified by silica gel column chromatography, eluted with PE/EA (10:1) to to afford (6-chloro-3-methylpyrazin-2-yl)methanol (910 mg, yield: 65.3%). MS Calcd.: 158.0; MS Found: 159.0 [M+H]⁺.

### Step C: 5-chloro-3-(chloromethyl)-2-methylpyrazine

To a solution of (6-chloro-3-methylpyrazin-2-yl)methanol (910 mg, 5.8 mmol) in DCM (12 mL) was added SOCl₂ (1.03 g, 8.7 mmol) at 0° C. The resulting mixture was stirred at room temperature for 2 hours. The reaction mixture was quenched with sat.NaHCO₃ (20 mL), and then extracted with DCM (3×40 mL). The organic layer was dried over Na₂SO₄, filtered, and concentrated under reduced pressure. The residue was purified by silica gel column chromatography, eluted with PE/EA (20:1) to afford 5-chloro-3-(chloromethyl)-2-methylpyrazine (950 mg, yield: 93.7%). MS Calcd.: 176.0; MS Found: 177.1 [M+H]⁺.

### Step D: tert-butyl 1-((6-chloro-3-methylpyrazin-2-yl)methyl)cyclopropane-1-carboxylate

To a solution of tert-butyl cyclopropanecarboxylate (1.15 g, 8.1 mmol) in THF (10 mL) was dropwise added LDA (5.4 mL, 2 mol/L in THF) at −65° C. under N₂. The mixture was stirred at −65° C. for 1 hour. The 5-chloro-3-(chloromethyl)-2-methylpyrazine (950 mg, 5.40 mmol) in THF (10 mL) was dropwise added. The reaction mixture was warmed up to rt slowly and stirred overnight. The reaction was diluted with EA (50 mL), washed with NH₄Cl (aq, 20 mL) and brine, dried over Na2SO4, filtered, and concentrated under reduced pressure. The residue was purified by prep-TLC (PE/EA=10/1) to afford tert-butyl 1-((6-chloro-3-methylpyrazin-2-yl)methyl)cyclopropane-1-carboxylate (560 mg, yield: 36.8%). MS Calcd.: 282.1; MS Found: 283.1 [M+H]⁺.

### Step E: tert-butyl 1-((6-cyano-3-methylpyrazin-2-yl)methyl)cyclopropane-1-carboxylate

To a mixture of tert-butyl 1-((6-chloro-3-methylpyrazin-2-yl)methyl)cyclopropane-1-carboxylate (460 mg, 1.6 mmol) in NMP (4.0 mL) was added Zn(CN)₂ (572 mg, 4.9 mmol) and Ruphos Pd G3 (136 mg, 0.16 mmol). The resulting mixture was stirred at 130° C. for 1h under Ar. The reaction mixture was quenched with H₂O (20 mL), and then extracted with EA (2×20 mL). The organic layers were combined, washed with brine (20 mL). The organic layer was dried over Na₂SO₄, filtered and concentrated under reduced pressure. The residue was purified by silica gel column chromatography, eluted with PE/EA (10:1) to afford tert-butyl 1-((6-cyano-3-methylpyrazin-2-yl)methyl)cyclopropane-1-carboxylate (290 mg, yield: 65.2%). MS Calcd.: 273.2; MS Found: 274.1 [M+H]⁺.

### Step F: 1-((6-cyano-3-methylpyrazin-2-yl)methyl)cyclopropane-1-carboxylic acid

To a solution of tert-butyl 1-((6-cyano-3-methylpyrazin-2-yl)methyl)cyclopropane-1-carboxylate (270 mg, 0.99 mmol) in DCM (6.0 mL) was added TFA (2.0 mL) at room temperature. The resulting mixture was stirred at room temperature for 2 hours. The reaction mixture was concentrated under reduced pressure to afford crude 1-((6-cyano-3-methylpyrazin-2-yl)methyl)cyclopropane-1-carboxylic acid (340 mg, yield: 100%). MS Calcd.: 217.1; MS Found: 218.0 [M+H]⁺.

### Step G: 1-((6-cyano-3-methylpyrazin-2-yl)methyl)cyclopropane-1-carboxamide

To a mixture of 1-((6-cyano-3-methylpyrazin-2-yl)methyl)cyclopropane-1-carboxylic acid (340 mg, 1.57 mmol) in DCM (6.0 mL) was added DIEA (608 mg, 4.71 mmol), NH₄Cl (126 mg, 2.35 mmol) and HATU (775 mg, 2.04 mmol). The resulting mixture was stirred at rt for 2 h under Ar. The reaction mixture was quenched with H₂O (20 mL), and then extracted with DCM (2×30 mL), The organic layer was dried over Na₂SO₄, filtered, and concentrated under reduced pressure. The residue was purified by silica gel column chromatography, eluted with PE/EA (3:1) to afford 1-((6-cyano-3-methylpyrazin-2-yl)methyl)cyclopropane-1-carboxamide (250 mg, yield: 73.5%). MS Calcd.: 216.1; MS Found: 217.1 [M+H]⁺.

### Step H: 6-((1-cyanocyclopropyl)methyl)-5-methylpyrazine-2-carbonitrile

To a mixture of 1-((6-cyano-3-methylpyrazin-2-yl)methyl)cyclopropane-1-carboxamide (230 mg, 1.06 mmol) in DCM (6.0 mL) was added Burgess reagent (818 mg, 3.19 mmol). The resulting mixture was stirred at rt for 16 hours under Ar. The reaction mixture was quenched with H₂O (10 mL), and then extracted with DCM (2×20 mL), The organic layer was dried over Na₂SO₄, filtered, and concentrated under reduced pressure. The residue was purified by silica gel column chromatography, eluted with PE/EA (3:1) to afford 6-((1-cyanocyclopropyl)methyl)-5-methylpyrazine-2-carbonitrile (65 mg, yield: 30.9%). MS Calcd.: 198.1; MS Found: 199.1 [M+H]⁺.

### Step I: 5-(bromomethyl)-6-((1-cyanocyclopropyl)methyl)pyrazine-2-carbonitrile

A mixture of 6-((1-cyanocyclopropyl)methyl)-5-methylpyrazine-2-carbonitrile (30 mg, 0.151 mmol), AIBN (12 mg, 0.075 mmol) and NBS (54 mg, 0.303 mmol) in DMF (1.5 mL) was stirred at 115° C. for 4 hours under N₂ atmosphere. The reaction mixture was cooled to room temperature. The mixture was quenched with H₂O (5 mL), extracted with EA (3×10 mL). The combined organic layers were washed with brine (10 mL). The organic layer was dried over Na₂SO₄, filtered, and concentrated under reduced pressure. The residue was purified by Prep-TLC (PE/EA=2/1) to give 5-(bromomethyl)-6-((1-cyanocyclopropyl)methyl)pyrazine-2-carbonitrile (13 mg, yield: 31%).

### Step J: (S)-5-((4-(2-(4-chloro-2-fluorophenyl)-2-methylbenzo[d][1,3]dioxol-4-yl)piperidin-1-yl)methyl)-6-((1-cyanocyclopropyl)methyl)pyrazine-2-carbonitrile

To a solution of (S)-4-(2-(4-chloro-2-fluorophenyl)-2-methylbenzo[d][1,3]dioxol-4-yl)piperidine (27 mg, 0.051 mmol, TsOH salt) in DMF (1.5 mL) were added DIEA (12 mg, 0.094 mmol) and 5-(bromomethyl)-6-((1-cyanocyclopropyl)methyl)pyrazine-2-carbonitrile (13 mg, 0.047 mmol). The reaction mixture was stirred at 70° C. for an hour. The reaction mixture was cooled to room temperature. The mixture was quenched with H₂O (5 mL), extracted with EA (3×10 mL). The combined organic layers were washed with brine (5 mL). The organic layer was dried over Na₂SO₄, filtered and concentrated under reduced pressure. The residue was purified by Prep-TLC (PE/EA=4/1) to give (S)-5-((4-(2-(4-chloro-2-fluorophenyl)-2-methylbenzo[d][1,3]dioxol-4-yl)piperidin-1-yl)methyl)-6-((1-cyanocyclopropyl)methyl)pyrazine-2-carbonitrile (15 mg, yield: 58.6%). MS Calcd.: 543.2; MS Found: 544.2 [M+H]⁺.

### Step K: (S)-5-((4-(2-(4-chloro-2-fluorophenyl)-2-methylbenzo[d][1,3]dioxol-4-yl)piperidin-1-yl)methyl)-6-((1-cyanocyclopropyl)methyl)-N′-hydroxypyrazine-2-carboximidamide

To a solution of (S)-5-((4-(2-(4-chloro-2-fluorophenyl)-2-methylbenzo[d][1,3]dioxol-4-yl)piperidin-1-yl)methyl)-6-((1-cyanocyclopropyl)methyl)pyrazine-2-carbonitrile (15 mg, 0.02 mmol) in ACN (1.5 mL) was added hydroxylamine hydrochloride (3.9 mg, 0.055 mmol) and TEA (8 mg, 0.081 mmol). The resulting mixture was stirred at 50° C. for an hour. The reaction mixture was diluted with EA (10 mL), washed with H₂O (5 mL). The organic layer was dried over Na₂SO₄ and filtered. The filtrate was concentrated under reduced pressure. The residue was purified by Prep-TLC (DCM/MeOH=20/1) to give (S)-5-((4-(2-(4-chloro-2-fluorophenyl)-2-methylbenzo[d][1,3]dioxol-4-yl)piperidin-1-yl)methyl)-6-((1-cyanocyclopropyl)methyl)-N′-hydroxypyrazine-2-carboximidamide (13 mg, yield: 81.7%). MS Calcd.: 576.2; MS Found: 577.2 [M+H]⁺.

### Step L: (S)-1-((3-((4-(2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)pyrazin-2-yl)methyl)cyclopropane-1-carbonitrile

To a solution of (S)-5-((4-(2-(4-chloro-2-fluorophenyl)-2-methylbenzo[d][1,3]dioxol-4-yl)piperidin-1-yl)methyl)-6-((1-cyanocyclopropyl)methyl)-N′-hydroxypyrazine-2-carboximidamide (13 mg, 0.022 mmol) in THF (1.5 mL) was added TFAA (19 mg, 0.090 mmol). The resulting mixture was stirred at room temperature for 2 hours, then the reaction mixture was heated to 60° C. for 12 hours. The reaction mixture was quenched with saturated sodium bicarbonate aqueous solution (5 mL), extracted with EA (3×10 mL). The organic layers were combined, washed with brine (5 mL). The organic layer was dried over Na₂SO₄, filtered and concentrated under reduced pressure. The residue was purified by Prep-TLC (DCM/MeOH=40/1) to afford (S)-1-((3-((4-(2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)pyrazin-2-yl)methyl)cyclopropane-1-carbonitrile (8.0 mg, yield: 54.2%). MS Calcd.: 654.2; MS Found: 655.2 [M+H]⁺.

### Step M: 1-{[3-({4-[(2S)-2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]pyrazin-2-yl]methyl}cyclopropane-1-carbonitrile (62)

To a solution of (S)-1-((3-((4-(2-(4-chloro-2-fluorophenyl)-2-methylbenzo[d][1,3]dioxol-4-yl)piperidin-1-yl)methyl)-6-(5-(trifluoromethyl)-1,2,4-oxadiazol-3-yl)pyrazin-2-yl)methyl)cyclopropane-1-carbonitrile (8 mg, 0.012 mmol) in DMF (1 mL) was added hydrazine hydrate (2.4 mg, 0.049 mmol). The resulting mixture was stirred at room temperature for an hour. The reaction mixture was quenched with water (5 mL), extracted with EA (3×10 mL). The organic layers were combined, washed with brine (5 mL). The organic layer was dried over Na₂SO₄, filtered, and concentrated under reduced pressure. The residue was purified by prep-TLC (DCM/MeOH=50/1) to afford 1-{[3-({4-[(2S)-2-(4-chloro-2-fluorophenyl)-2-methyl-2H-1,3-benzodioxol-4-yl]piperidin-1-yl}methyl)-6-[5-(trifluoromethyl)-4H-1,2,4-triazol-3-yl]pyrazin-2-yl]methyl}cyclopropane-1-carbonitrile (62) (4.3 mg, yield: 53%). MS Calcd.: 653.2; MS Found: 654.0 [M+H]⁺. ¹H NMR (400 MHz, DMSO-d6): δ 9.15 (s, 1H), 7.51-7.60 (m, 2H), 7.33 (dd, J=8.4 Hz, 1.6 Hz, 1H), 6.70-6.81 (m, 3H), 3.85 (s, 2H), 3.37 (s, 2H), 2.89-3.00 (m, 2H), 2.62-2.72 (m, 1H), 2.20-2.33 (m, 2H), 2.02 (s, 3H), 1.67-1.85 (m, 4H), 1.35-1.42 (m, 2H), 1.21-1.29 (m, 2H). ¹⁹F NMR (377 MHz, DMSO-d6): δ −63.52, −110.75.

### Example 63: 1-((2-((4-(6-((4-chlorobenzofuran-7-yl)methoxy)-5-fluoropyridin-2-yl)piperidin-1-yl)methyl)-5-(5-(trifluoromethyl)-4H-1,2,4-triazol-3-yl)pyridin-3-yl)methyl)cyclopropane-1-carbonitrile (63)

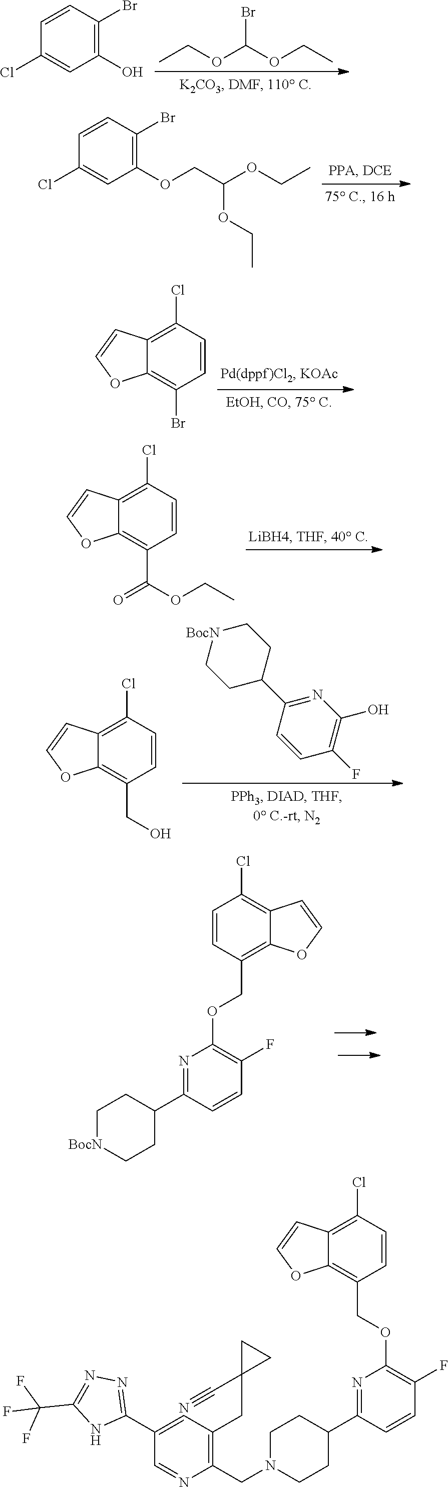

### Step A: 1-bromo-4-chloro-2-(2,2-diethoxyethoxy)benzene

To a solution of 2-bromo-5-chlorophenol (3.2 g, 15.5 mmol) in DMF (50 mL) was added K₂CO₃ (6.43 g, 46.6 mmol) and 2-bromo-1,1-diethoxyethane (6.1 g, 31.1 mmol). The resulting mixture was stirred at 110° C. for 16 hours. The reaction mixture was quenched with H₂O (100 mL), extracted with EA (2×200 mL). The combined organic layers were washed with brine (100 mL). The organic layer was dried over Na₂SO₄, filtered, and concentrated under reduced pressure. The residue was purified by silica gel column chromatography, eluted with (PE/EA=10/1) to give 1-bromo-4-chloro-2-(2,2-diethoxyethoxy)benzene (4.8 g, yield: 97.0%).

### Step B: 7-bromo-4-chlorobenzofuran

To a solution of 1-bromo-4-chloro-2-(2,2-diethoxyethoxy)benzene (4.6 g, 14.4 mmol) in DCE (50 mL) was added PPA (7.3 g, 21.7 mmol). The resulting mixture was stirred at 75° C. for 16 hours under N₂. The reaction mixture was quenched with H₂O (100 mL), extracted with EA (2×200 mL). The organic layer was dried over Na₂SO₄, filtered, and concentrated under reduced pressure. The residue was purified by silica gel column chromatography, eluted with (PE/EA=10/1) to give 7-bromo-4-chlorobenzofuran (2.3 g, yield: 68.0%). ¹H NMR: (400 MHz, DMSO-d6): δ 8.26 (d, J=2.0 Hz, 1H), 7.59 (m, 1H), 7.33 (dd, J₁=1.2 Hz, 8.4 Hz, 1H), 7.16 (m, 1H).

### Step C: ethyl 4-chlorobenzofuran-7-carboxylate

To a solution of 7-bromo-4-chlorobenzofuran (2.3 g, 9.8 mmol) in EtOH (40 mL) was added Pd(dppf)Cl₂ (1.4 g, 2.0 mmol) and KOAc (2.9 g, 29.5 mmol). The resulting mixture was stirred at 75° C. for 16 hours under CO. The reaction mixture was quenched with H₂O (50 mL), extracted with EA (2×100 mL). The organic layer was dried over Na₂SO₄, filtered, and concentrated under reduced pressure. The residue was purified by silica gel column chromatography, eluted with (PE/EA=20/1) to give ethyl 4-chlorobenzofuran-7-carboxylate (840 mg, yield: 38.2%). MS Calcd.: 224.0; MS Found: 224.9 [M+H]⁺.

### Step D: (4-chlorobenzofuran-7-yl)methanol

To a solution of ethyl 4-chlorobenzofuran-7-carboxylate (840 mg, 3.75 mmol) in THF (9 mL) was added LiBH₄ (330 mg, 15.0 mmol). The resulting mixture was stirred at 40° C. for 3 hours under N₂. The reaction mixture was quenched with H₂O (20 mL), extracted with EA (2×40 mL). The organic layer was dried over Na₂SO₄, filtered, and concentrated under reduced pressure. The residue was purified by silica gel column chromatography, eluted with (PE/EA=10/1) to give (4-chlorobenzofuran-7-yl)methanol (606 mg, yield: 88.8%).

### Step E: tert-butyl 4-(6-((4-chlorobenzofuran-7-yl)methoxy)-5-fluoropyridin-2-yl)piperidine-1-carboxylate

To a solution of (4-chlorobenzofuran-7-yl)methanol (250 mg, 1.37 mmol) in THF (8 mL) was added tert-butyl 4-(5-fluoro-6-hydroxypyridin-2-yl)piperidine-1-carboxylate (488 mg, 1.65 mmol) and PPh₃ (720 mg, 2.75 mmol). To the mixture was added DIAD (555 mg, 2.75 mmol) at 0° C. under N₂. The resulting mixture was stirred at room temperature for 3 hours under N₂. The reaction mixture was quenched with H₂O (10 mL), extracted with EA (2×20 mL). The combined organic layers were washed with brine (10 mL). The organic layer was dried over Na₂SO₄, filtered and concentrated under reduced pressure. The residue was purified by silica gel column chromatography, eluted with (PE/EA=6/1) to give tert-butyl 4-(6-((4-chlorobenzofuran-7-yl)methoxy)-5-fluoropyridin-2-yl)piperidine-1-carboxylate (410 mg, yield: 65%). MS Calcd.: 460.1; MS Found: 460.9 [M+H]⁺.

### Step F: 1-((2-((4-(6-((4-chlorobenzofuran-7-yl)methoxy)-5-fluoropyridin-2-yl)piperidin-1-yl)methyl)-5-(5-(trifluoromethyl)-4H-1,2,4-triazol-3-yl)pyridin-3-yl)methyl)cyclopropane-1-carbonitrile

1-((2-((4-(6-((4-chlorobenzofuran-7-yl)methoxy)-5-fluoropyridin-2-yl)piperidin-1-yl)methyl)-5-(5-(trifluoromethyl)-4H-1,2,4-triazol-3-yl)pyridin-3-yl)methyl)cyclopropane-1-carbonitrile (21.9 mg) was obtained with the similar method of Example 1. MS Calcd.: 665.2; MS Found: 666.0 [M+H]⁺. ¹H NMR (400 MHz, DMSO-d₆): δ 9.05 (d, J=1.6 Hz, 1H), 8.43 (d, J=2.0 Hz, 1H), 8.17 (d, J=1.6 Hz, 1H), 7.56-7.61 (m, 1H), 7.45 (d, J=8.0 Hz, 1H), 7.36 (d, J=8.0 Hz, 1H), 7.05 (d, J=2.4 Hz, 1H), 6.89 (dd, J=8.0 Hz, 2.8 Hz, 1H), 5.70 (s, 2H), 3.82 (s, 2H), 3.20 (s, 2H), 2.87-3.00 (m, 2H), 2.61-2.70 (m, 1H), 2.19-2.37 (m, 2H), 1.63-1.83 (m, 4H), 1.32-1.40 (m, 2H), 1.18-1.27 (m, 2H). ¹⁹F NMR (377 MHz, DMSO-d6): δ −62.76, −143.95.

### Example 64: 1-((2-((4-(6-((4-chlorobenzofuran-7-yl)methoxy)pyridin-2-yl)piperidin-1-yl)methyl)-5-(5-(trifluoromethyl)-4H-1,2,4-triazol-3-yl)pyridin-3-yl)methyl)cyclopropane-1-carbonitrile (64)

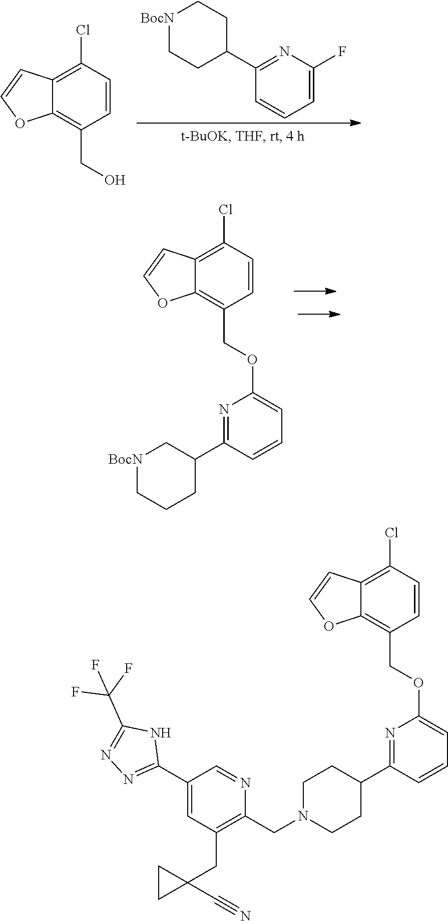 [SMILES: OCc1ccc(Cl)c2ccoc12 ; CC(C)(C)OC(=O)N1CCC(c2cccc(F)n2)CC1 ; CC(C)(C)OC(=O)N1CCCC(c2cccc(OCc3ccc(Cl)c4ccoc34)n2)C1 ; N#CC1(Cc2cc(-c3nnc(C(F)(F)F)[nH]3)cnc2CN2CCC(c3cccc(OCc4ccc(Cl)c5ccoc45)n3)CC2)CC1]

### Step A: tert-butyl 4-(6-((4-chlorobenzofuran-7-yl)methoxy)pyridin-2-yl)piperidine-1-carboxylate

To a solution of (4-chlorobenzofuran-7-yl)methanol (195 mg, 1.07 mmol) in THF (5 mL) was added tert-butyl 4-(6-fluoropyridin-2-yl)piperidine-1-carboxylate (300 mg, 1.07 mmol) and t-BuOK (2.1 mL, 2.14 mmol, 1M solution in THF). The resulting mixture was stirred at room temperature for 4 hours. The reaction mixture was quenched with H₂O (10 mL), extracted with EA (2×20 mL). The combined organic layers were washed with brine (10 mL). The organic layer was dried over Na₂SO₄, filtered, and concentrated under reduced pressure. The residue was purified by silica gel column chromatography, eluted with (PE/EA=6/1) to give tert-butyl 4-(6-((4-chlorobenzofuran-7-yl)methoxy)pyridin-2-yl)piperidine-1-carboxylate (380 mg, yield: 80%). MS Calcd.: 442.2; MS Found: 443.0 [M+H]⁺.

### Step B: 1-((2-((4-(6-((4-chlorobenzofuran-7-yl)methoxy)pyridin-2-yl)piperidin-1-yl)methyl)-5-(5-(trifluoromethyl)-4H-1,2,4-triazol-3-yl)pyridin-3-yl)methyl)cyclopropane-1-carbonitrile

1-((2-((4-(6-((4-chlorobenzofuran-7-yl)methoxy)pyridin-2-yl)piperidin-1-yl)methyl)-5-(5-(trifluoromethyl)-4H-1,2,4-triazol-3-yl)pyridin-3-yl)methyl)cyclopropane-1-carbonitrile (26.4 mg) was obtained with the similar method of Example 1. MS Calcd.: 647.20; MS Found: 648.0 [M+H]⁺. ¹H NMR (400 MHz, DMSO-d₆): δ 9.07 (d, J=2.0 Hz, 1H), 8.44 (d, J=2.0 Hz, 1H), 8.15-8.16 (m, 1H), 7.63 (t, J=7.6 Hz, 1H), 7.44 (d, J=8.0 Hz, 1H), 7.34 (d, J=8.0 Hz, 1H), 7.04 (d, J=2.4 Hz, 1H), 6.86 (d, J=7.2 Hz, 1H), 6.68 (d, J=8.0 Hz, 1H), 5.62 (s, 2H), 3.87 (s, 2H), 3.20 (s, 2H), 2.91-3.03 (m, 2H), 2.60-2.69 (m, 1H), 2.25-2.39 (m, 2H), 1.71-1.86 (m, 4H), 1.35-1.38 (m, 2H), 1.21-1.24 (m, 2H). ¹⁹F NMR (377 MHz, DMSO-d₆): δ −62.87.

### Example 65: 1-((2-((4-(2-(2-chloro-4-methylphenyl)-2-methylbenzo[d][1,3]dioxol-4-yl)-3,6-dihydropyridin-1(2H)-yl)methyl)-5-(5-(trifluoromethyl)-4H-1,2,4-triazol-3-yl)pyridin-3-yl)methyl)cyclopropane-1-carbonitrile (65)

### Step A: tert-butyl 4-(2-(2-chloro-4-methylphenyl)-2-methylbenzo[d][1,3]dioxol-4-yl)-3,6-dihydropyridine-1(2H)-carboxylate

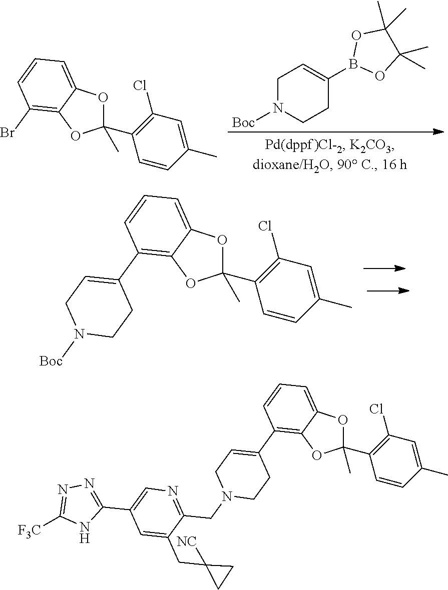

A mixture of 4-bromo-2-(2-chloro-4-methylphenyl)-2-methylbenzo[d][1,3]dioxole (360 mg, 1.07 mmol), tert-butyl 4-(4,4,5,5-tetramethyl-1,3,2-dioxaborolan-2-yl)-3,6-dihydropyridine-1(2H)-carboxylate (494 mg, 1.60 mmol), Pd(dppf)Cl₂ (78 mg, 0.11 mmol) and K₂CO₃ (441 mg, 3.2 mmol) in 1,4-dioxane/H₂O (10 mL/1 mL) was stirred at 90° C. under Ar for 16 h. The mixture was poured into water (200 mL) and extracted with EtOAC (2×200 mL), the combined organic layer was washed with brine, dried over sodium sulfate, filtered and the residue was concentrated. The residue was purified by column chromatography (PE:EA=15: 1) on silica gel to obtain tert-butyl 4-(2-(2-chloro-4-methylphenyl)-2-methylbenzo[d][1,3]dioxol-4-yl)-3,6-dihydropyridine-1(2H)-carboxylate (250 mg, yield: 53.2%) as a brown oil. MS Calcd: 441.2; MS Found: 464.2 [M+23]*.

### Step B:1-((2-((4-(2-(2-chloro-4-methylphenyl)-2-methylbenzo[d][1,3]dioxol-4-yl)-3,6-dihydropyridin-1(2H)-yl)methyl)-5-(5-(trifluoromethyl)-4H-1,2,4-triazol-3-yl)pyridin-3-yl)methyl)cyclopropane-1-carbonitrile

1-((2-((4-(2-(2-chloro-4-methylphenyl)-2-methylbenzo[d][1,3]dioxol-4-yl)-3,6-dihydropyridin-1(2H)-yl)methyl)-5-(5-(trifluoromethyl)-4H-1,2,4-triazol-3-yl)pyridin-3-yl)methyl)cyclopropane-1-carbonitrile (2.5 mg) was obtained with the similar method to Example 1. MS Calcd: 646.2; MS Found: 647.4.

¹H NMR (400 MHz, CD₃OD): δ 9.11 (d, J=2.0 Hz, 1H), 8.52 (d, J=2.0 Hz, 1H), 7.58 (d, J=8.0 Hz, 1H), 7.27 (s, 1H), 7.09 (d, J=8.0 Hz, 1H), 6.83-6.70 (m, 3H), 6.45 (s, 1H), 3.93 (s, 2H), 3.28-3.24 (m, 4H), 2.82 (t, J=5.6 Hz, 2H), 2.68-2.52 (m, 2H), 2.30 (s, 3H), 2.08 (s, 3H), 1.39 (dd, J₁=7.6 Hz/J₂=5.2 Hz, 2H), 1.24 (dd, J₁=7.2 Hz/J₂=4.8 Hz, 2H). ¹⁹F NMR (377 MHz, CD₃OD): δ −64.58.

### Example 66: 1-((2-((4-(2-(4-chloro-2-fluorophenyl)-2-methylbenzo[d][1,3]dioxol-4-yl)-3,6-dihydropyridin-1(2H)-yl)methyl)-5-(5-(trifluoromethyl)-4H-1,2,4-triazol-3-yl)pyridin-3-yl)methyl)cyclopropane-1-carbonitrile (66)

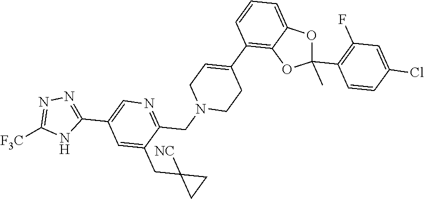 [SMILES: CC1(c2ccc(Cl)cc2F)Oc2cccc(C3=CCN(Cc4ncc(-c5nnc(C(F)(F)F)[nH]5)cc4CC4(C#N)CC4)CC3)c2O1]

Starting from 4-(2-(4-chloro-2-fluorophenyl)-2-methylbenzo[d][1,3]dioxol-4-yl)-1,2,3,6-tetrahydropyridine TFA salt, 1-((2-((4-(2-(4-chloro-2-fluorophenyl)-2-methylbenzo[d][1,3]dioxol-4-yl)-3,6-dihydropyridin-1(2H)-yl)methyl)-5-(5-(trifluoromethyl)-4H-1,2,4-triazol-3-yl)pyridin-3-yl)methyl)cyclopropane-1-carbonitrile (3.7 mg) was obtained with the similar method to Example 1. MS Calcd: 650.2; MS Found: 651.2 [M+H]⁺. ¹H NMR (400 MHz, CD₃OD): δ 9.17 (s, 1H), 8.52 (s, 1H), 7.58 (t, J=8.4 Hz, 1H), 7.31-7.18 (m, 2H), 6.92-6.78 (m, 3H), 6.43 (s, 1H), 4.41 (s, 2H), 3.74 (s, 2H), 3.25-3.13 (m, 4H), 2.82 (s, 2H), 2.05 (s, 3H), 1.44-1.39 (m, 2H), 1.28-1.24 (m, 2H). ¹⁹F NMR (377 MHz, CD₃OD): δ −66.56, −112.46.

### Example 67: 2-(6-((4-((S)-2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)cyclopropane-1-carboxylic acid (67)

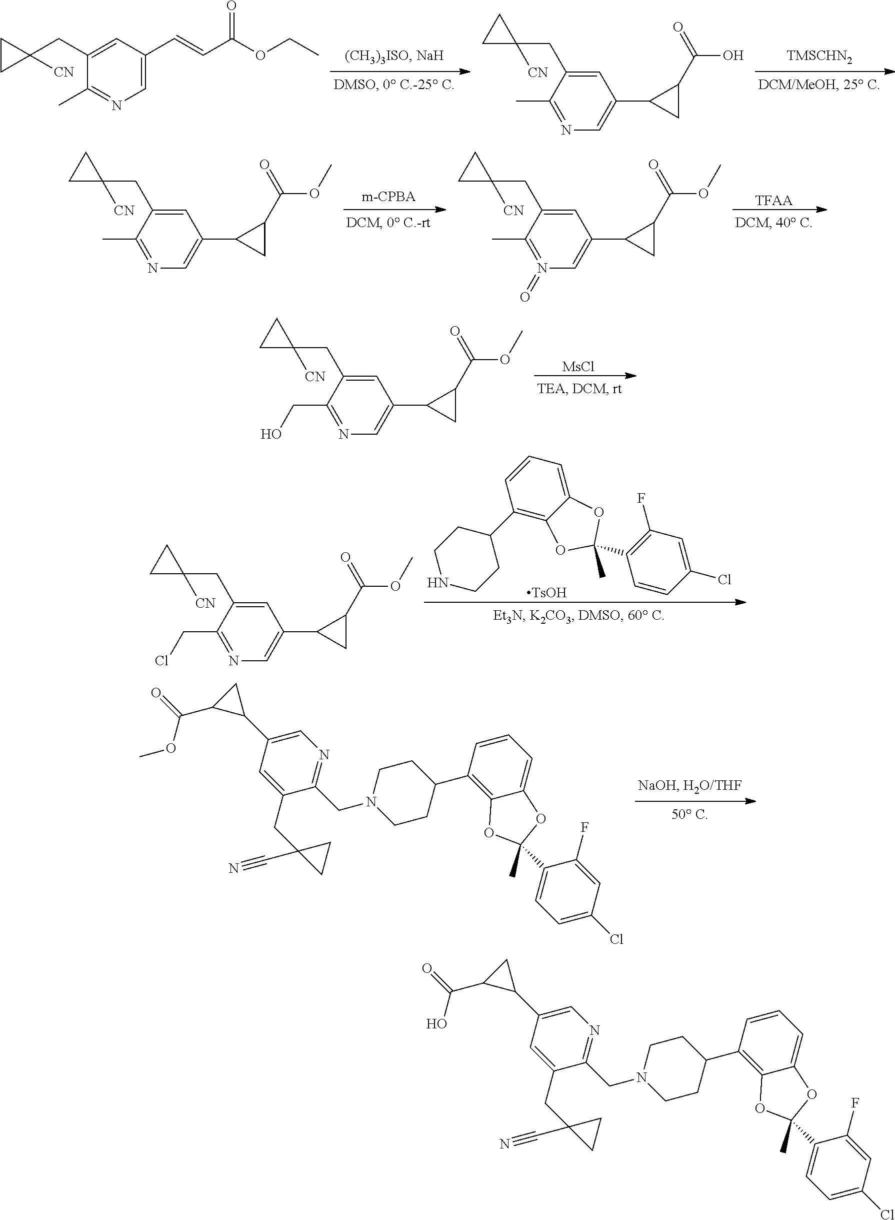

### Step A: 2-(5-((1-cyanocyclopropyl)methyl)-6-methylpyridin-3-yl)cyclopropane-1-carboxylic acid

To the mixture of NaH (60%, 89 mg, 2.2 mmol) in DMSO (4 mL) was added (CH₃)₃ISO (489 mg, 2.2 mmol) at room temperature. The mixture was stirred at 25° C. for 1 h and added dropwise ethyl (E)-3-(5-((1-cyanocyclopropyl)methyl)-6-methylpyridin-3-yl)acrylate (500 mg, 1.8 mmol) in DMSO (3 mL). The mixture was stirred at 25° C. for 16 h. The mixture was purified by prep-HPLC to obtain 2-(5-((1-cyanocyclopropyl)methyl)-6-methylpyridin-3-yl)cyclopropane-1-carboxylic acid (60 mg, yield: 12.6%). MS Calcd: 256.1; MS Found: 257.3 [M+H]⁺.

### Step B: methyl 2-(5-((1-cyanocyclopropyl)methyl)-6-methylpyridin-3-yl)cyclopropane-1-carboxylate

A mixture of 2-(5-((1-cyanocyclopropyl)methyl)-6-methylpyridin-3-yl)cyclopropane-1-carboxylic acid (60 mg, 0.23 mmol) and tetrahydrofuran solution of TMSCHN₂ (2 M, 0.23 mL, 0.46 mmol) in MeOH/DCM (5 mL/5 mL) was stirred at 25° C. for 16 h. The mixture was poured into water (30 mL) and extracted with DCM (2×30 mL), the combined organic layer was washed with brine, dried over sodium sulfate, filtered and the residue was concentrated to obtain methyl 2-(5-((1-cyanocyclopropyl)methyl)-6-methylpyridin-3-yl)cyclopropane-1-carboxylate (40 mg, yield: 63.2%). MS Calcd: 270.1; MS Found: 271.1 [M+H]⁺.

### Step C: 3-((1-cyanocyclopropyl)methyl)-5-(2-(methoxycarbonyl)cyclopropyl)-2-methylpyridine 1-oxide

To a solution of methyl 2-(5-((1-cyanocyclopropyl)methyl)-6-methylpyridin-3-yl)cyclopropane-1-carboxylate (40 mg, 0.15 mmol) in DCM (5 mL) was added m-CPBA (153 mg, 0.89 mmol) at 0° C. The mixture was stirred at room temperature for 16 h. The mixture was added aqueous sodium bicarbonate solution (2 mL) and concentrated. The residue was purified by prep-HPLC to obtain 3-((1-cyanocyclopropyl)methyl)-5-(2-(methoxycarbonyl)cyclopropyl)-2-methylpyridine 1-oxide (45 mg). MS Calcd: 286.1; MS Found: 287.2 [M+H]⁺.

### Step D: methyl 2-(5-((1-cyanocyclopropyl)methyl)-6-(hydroxymethyl)pyridin-3-yl)cyclopropane-1-carboxylate

A mixture of 3-((1-cyanocyclopropyl)methyl)-5-(2-(methoxycarbonyl)cyclopropyl)-2-methylpyridine 1-oxide (45 mg, 0.16 mmol) and TFAA (1 mL) in DCM (5 mL) was stirred at 40° C. for 48 h. The mixture was concentrated, and the residue was purified by prep-HPLC to obtain methyl 2-(5-((1-cyanocyclopropyl)methyl)-6-(hydroxymethyl)pyridin-3-yl)cyclopropane-1-carboxylate (20 mg, yield: 44%). MS Calcd: 286.1; MS Found: 287.0 [M+H]⁺.

### Step E: methyl 2-(6-(chloromethyl)-5-((1-cyanocyclopropyl)methyl)pyridin-3-yl)cyclopropane-1-carboxylate

A mixture of methyl 2-(5-((1-cyanocyclopropyl)methyl)-6-(hydroxymethyl)pyridin-3-yl)cyclopropane-1-carboxylate (20 mg, 0.07 mmol), MsCl (16 mg, 0.14 mmol) and TEA (21 mg, 0.21 mmol) in DCM (3 mL) was stirred at room temperature for 16 h. The mixture was poured into water (20 mL) and extracted with DCM (2×20 mL), the combined organic layer was washed with brine, dried over sodium sulfate, filtered and the residue was concentrated to obtain methyl 2-(6-(chloromethyl)-5-((1-cyanocyclopropyl)methyl)pyridin-3-yl)cyclopropane-1-carboxylate (21 mg). MS Calcd: 304.1; MS Found: 305.1 [M+H]⁺.

### Step F: methyl 2-(6-((4-((S)-2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)cyclopropane-1-carboxylate

A mixture of methyl 2-(6-(chloromethyl)-5-((1-cyanocyclopropyl)methyl)pyridin-3-yl)cyclopropane-1-carboxylate (21 mg, 0.069 mmol), (S)-4-(2-(4-chloro-2-fluorophenyl)-2-methylbenzo[d][1,3]dioxol-4-yl)piperidine 4-methylbenzenesulfonic acid salt (36 mg, 0.069 mmol), TEA (21 mg, 0.21 mmol) and K₂CO₃ in DMSO (2 mL) was stirred at 60° C. for 3 h. The mixture was poured into water (30 mL) and extracted with DCM (2×30 mL), the combined organic layer was washed with brine, dried over sodium sulfate, filtered and the residue was concentrated to obtain methyl 2-(6-((4-((S)-2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)cyclopropane-1-carboxylate (42 mg). MS Calcd: 615.2; MS Found: 616.2 [M+H]⁺.

### Step G: 2-(6-((4-((S)-2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)cyclopropane-1-carboxylic acid

A mixture of methyl 2-(6-((4-((S)-2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)cyclopropane-1-carboxylate (42 mg, 0.068 mmol) and NaOH (27 mg, 0.68 mmol) in THF/H₂O (2 mL/0.4 mL) was stirred at 50° C. for 4 h. The mixture was purified by prep-HPLC to obtain 2-(6-((4-((S)-2-(4-chloro-2-fluorophenyl)-2-methylbenzo[d][1,3]dioxol-4-yl)piperidin-1-yl)methyl)-5-((1-cyanocyclopropyl)methyl)pyridin-3-yl)cyclopropane-1-carboxylic acid (5.2 mg, yield: 12.6%). MS Calcd: 601.2; MS Found: 602.4 [M+H]⁺.

¹H NMR (400 MHz, CD₃OD): δ 8.41 (s, 1H), 7.64-7.60 (m, 2H), 7.31 (d, J=10.8 Hz, 1H), 7.24 (d, J=8.8 Hz, 1H), 6.85-6.76 (m, 3H), 4.28 (brs, 2H), 3.43-3.38 (m, 2H), 3.03 (s, 2H), 2.98-2.88 (m, 2H), 2.51-2.44 (m, 1H), 2.21-2.12 (m, 3H), 2.09 (s, 3H), 2.06-1.87 (m, 3H), 1.63-1.56 (m, 1H), 1.39-1.36 (m, 2H), 1.34-1.28 (m, 1H), 1.23-1.20 (m, 2H). ¹⁹F NMR (377 MHz, CD₃OD): δ −112.27.

The remaining compounds in Table 1 were synthesized using a similar procedure described in the Examples above using the appropriate starting materials.

| No. | Structure | LC-MS (m/z) |
|---|---|---|
| 71 | 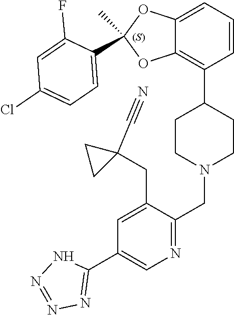 | Calcd: 585.2; MS Found: 586.2 |
| 73 | 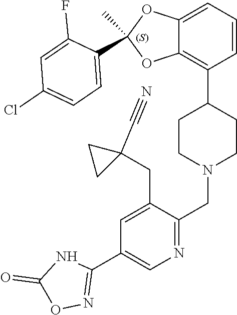 | Calcd: 601.2; MS Found: 602.2 |
| 75 | 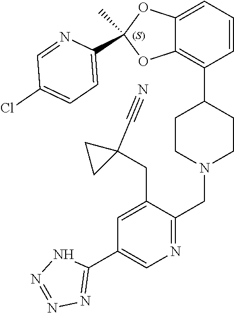 | Calcd: 568.2; MS Found: 569.2 |
| 77 | 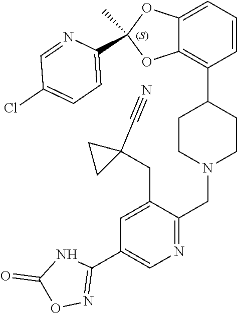 | Calcd: 584.2; MS Found: 585.2 |
| 78 | 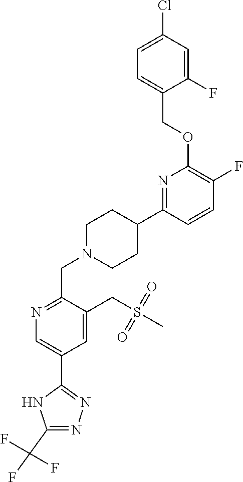 [SMILES: CS(=O)(=O)Cc1cc(-c2nnc(C(F)(F)F)[nH]2)cnc1CN1CCC(c2ccc(F)c(OCc3ccc(Cl)cc3F)n2)CC1] | Calcd: 656.1; MS Found: 657.3 |
| 79 | 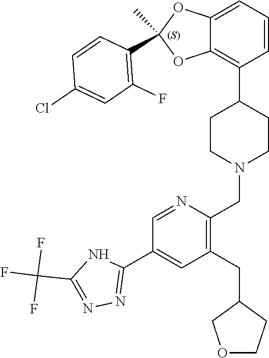 | Calcd: 657.2; MS Found: 658.1 |
| 80 | 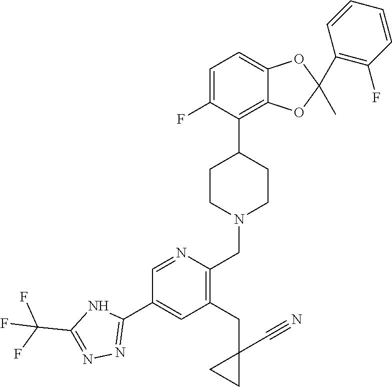 [SMILES: CC1(c2ccccc2F)Oc2ccc(F)c(C3CCN(Cc4ncc(-c5nnc(C(F)(F)F)[nH]5)cc4CC4(C#N)CC4)CC3)c2O1] | Calcd: 636.2; MS Found: 637.3 |
| 81 | 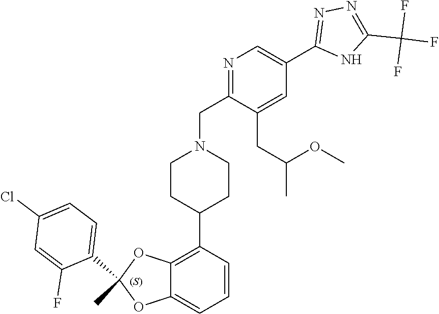 | Calcd: 645.2; MS Found: 646.2 |
| 82 | 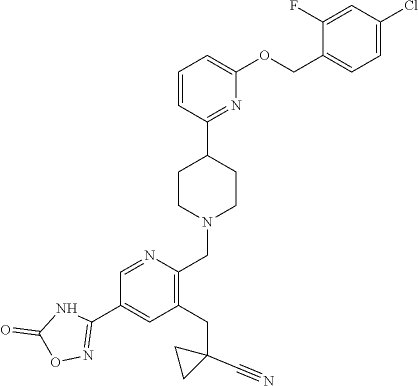 [SMILES: N#CC1(Cc2cc(-c3noc(=O)[nH]3)cnc2CN2CCC(c3cccc(OCc4ccc(Cl)cc4F)n3)CC2)CC1] | Calcd: 574.2; MS Found: 575.2 |
| 83 | 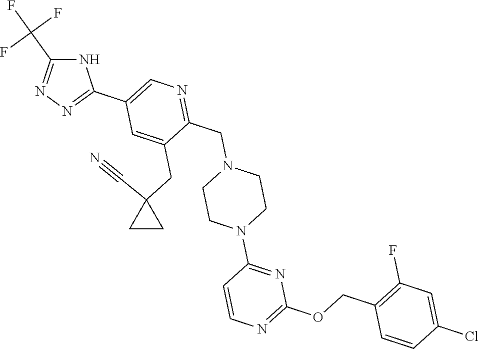 [SMILES: N#CC1(Cc2cc(-c3nnc(C(F)(F)F)[nH]3)cnc2CN2CCN(c3ccnc(OCc4ccc(Cl)cc4F)n3)CC2)CC1] | Calcd: 627.2; MS Found: 628.1 |
| 85 | 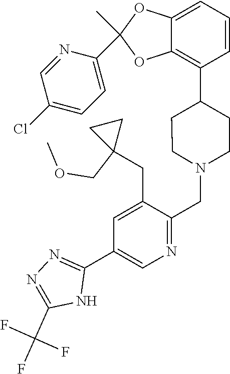 [SMILES: COCC1(Cc2cc(-c3nnc(C(F)(F)F)[nH]3)cnc2CN2CCC(c3cccc4c3OC(C)(c3ccc(Cl)cn3)O4)CC2)CC1] | Calcd: 654.2; MS Found: 655.2 |
| 86 | 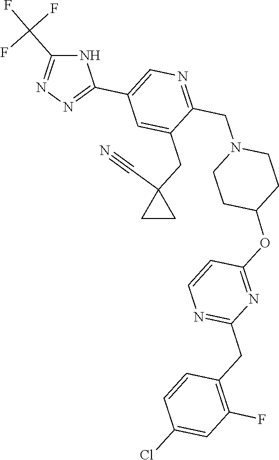 [SMILES: N#CC1(Cc2cc(-c3nnc(C(F)(F)F)[nH]3)cnc2CN2CCC(Oc3ccnc(Cc4ccc(Cl)cc4F)n3)CC2)CC1] | Calcd: 626.2; MS Found: 627.2 |
| 87 | 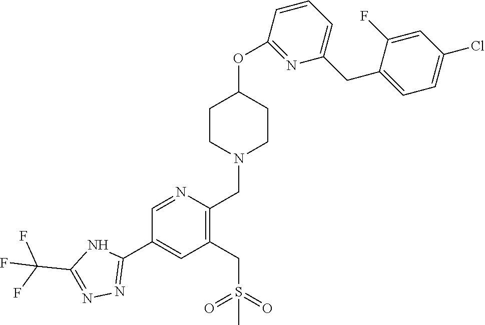 [SMILES: CS(=O)(=O)Cc1cc(-c2nnc(C(F)(F)F)[nH]2)cnc1CN1CCC(Oc2cccc(Cc3ccc(Cl)cc3F)n2)CC1] | Calcd: 638.1; MS Found: 639.2 |
| 89 | 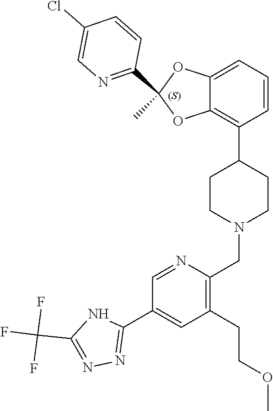 | Calcd: 614.2; MS Found: 615.2 |
| 90 | 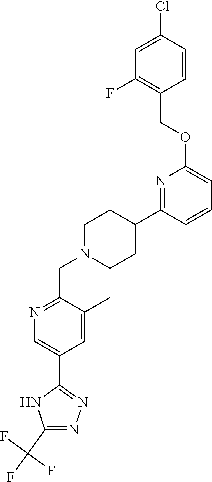 [SMILES: Cc1cc(-c2nnc(C(F)(F)F)[nH]2)cnc1CN1CCC(c2cccc(OCc3ccc(Cl)cc3F)n2)CC1] | Calcd: 560.2; MS Found: 561.3 |
| 91 | 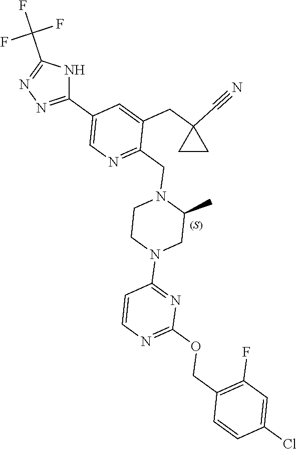 | Calcd: 641.2; MS Found: 642.1 |
| 92 | 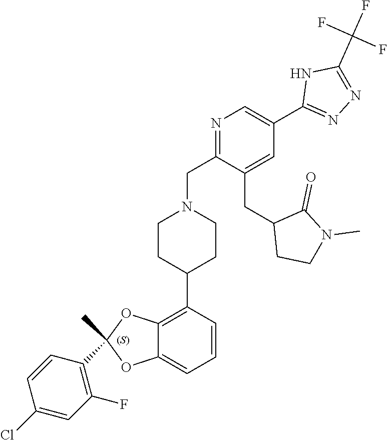 | Calcd: 684.2; MS Found: 685.3 |
| 93 | 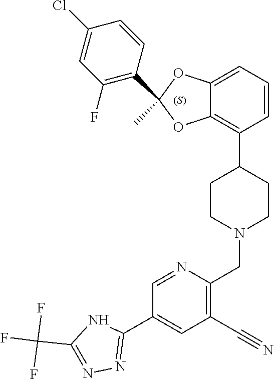 | Calcd: 598.1; MS Found: 599.1 |
| 94 | 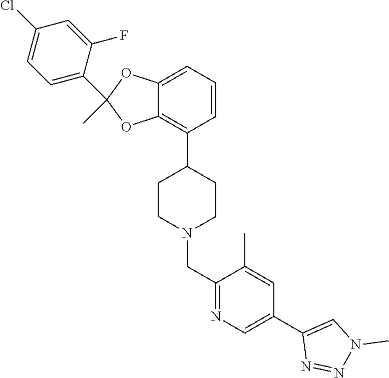 [SMILES: Cc1cc(-c2cn(C)nn2)cnc1CN1CCC(c2cccc3c2OC(C)(c2ccc(Cl)cc2F)O3)CC1] | Calcd: 533.2; MS Found: 534.2 |
| 95 | 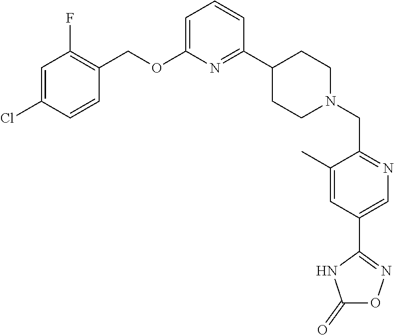 [SMILES: Cc1cc(-c2noc(=O)[nH]2)cnc1CN1CCC(c2cccc(OCc3ccc(Cl)cc3F)n2)CC1] | Calcd: 509.1; MS Found: 510.2 |
| 96 | 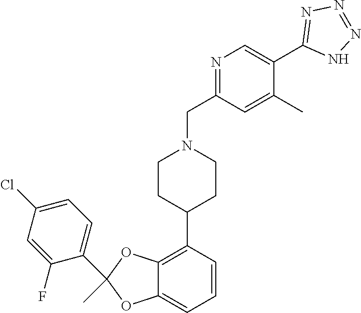 [SMILES: Cc1cc(CN2CCC(c3cccc4c3OC(C)(c3ccc(Cl)cc3F)O4)CC2)ncc1-c1nnn[nH]1] | Calcd: 520.2; MS Found: 521.3 |
| 97 | 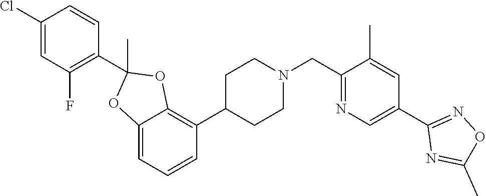 [SMILES: Cc1nc(-c2cnc(CN3CCC(c4cccc5c4OC(C)(c4ccc(Cl)cc4F)O5)CC3)c(C)c2)no1] | Calcd: 534.2; MS Found: 535.2 |
| 98 | 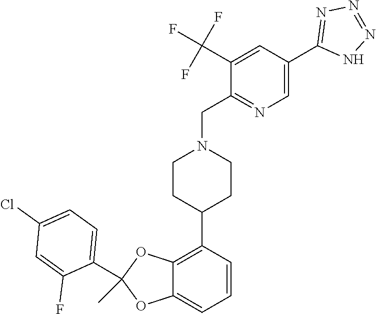 [SMILES: CC1(c2ccc(Cl)cc2F)Oc2cccc(C3CCN(Cc4ncc(-c5nnn[nH]5)cc4C(F)(F)F)CC3)c2O1] | Calcd: 574.2; MS Found: 575.2 |
| 99 | 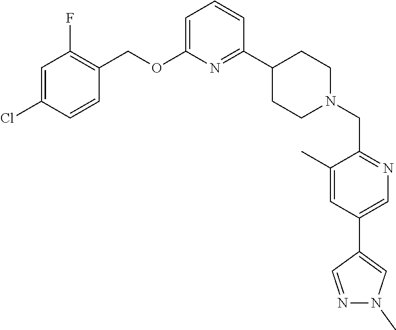 [SMILES: Cc1cc(-c2cnn(C)c2)cnc1CN1CCC(c2cccc(OCc3ccc(Cl)cc3F)n2)CC1] | Calcd: 505.2; MS Found: 506.4 |
| 100 | 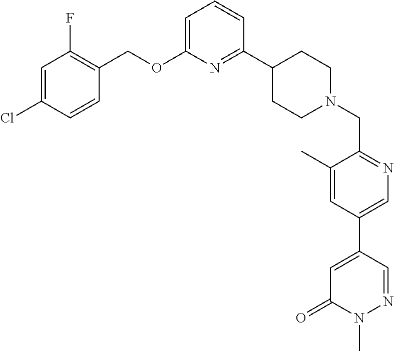 [SMILES: Cc1cc(-c2cnn(C)c(=O)c2)cnc1CN1CCC(c2cccc(OCc3ccc(Cl)cc3F)n2)CC1] | Calcd: 533.2; MS Found: 534.4 |

### BIOLOGICAL EXAMPLES

### Biological Example 1: cAMP Assays

Activation of GLP-1 receptor is known to stimulate cyclic AMP (cAMP) production in cells which indicates primary coupling to the Gas subunit of the G protein heterotrimeric complex. Evidence suggests signaling through Gas induced cAMP stimulation elicits the desired pharmacological response regarding insulin release from pancreatic β-cells.

To optimize functional activity directed toward Gas coupling, a HEK293/CRE-Luc cell line developed by HDB stably expressing the GLP-1 Receptor was used. 200× concentration of compound working solutions were prepared (Agilent Technologies Bravo) with ½ log serial dilution in 384-well Echo LDV plate (Labcyte, Cat #LP-0200). 50 nL/well 200× concentration of compound working solutions were moved to 384-well white low volume plate (Greiner, Cat #784075) using Labcyte ECHO550. 1×105 cells/mL HEK293/GLPlR/CRE-LUC (HD Biosciences) cell suspensions prepared with assay buffer [DPBS containing 0.5 mM IBMX (Sigma, Cat #15879) and 0.1% BSA (GENVIEW, Cat #FA016-100g)], 10 μL cell suspensions were added to each well of previous generated assay plate which already contains 50 nL compound at 200× concentration using ThermoFisher Multidrop Combi (1000 cells/well). Seal the plate and incubate at 37° C. with 5% CO₂ for 30 min.

After incubation the cAMP assay signal was generated using cAMP dynamic 2 Kit (Cisbio). 5 μL cAMP-d2 working solution was added to each well, followed with 5 μL Anti-cAMP antibody-cryptate working solution added to each well using ThermoFisher Multidrop Combi. Incubate at room temperature for 1 hour protected from light. Read the fluorescence at 665 and 615 nm with Reader PerkinElmer EnVision.

% Activity=100%×(mean RLU of test sample mean RLU of vehicle control)/(mean RLU of MAX control−mean RLU of vehicle control))

Table 3 shows the biological activity of compounds in GLP-1R agonist cAMP stimulation assay (EC₅₀). Activity of the tested compounds is provided in Table 3 below as follows: +++=EC₅₀<1 nM; ++=EC₅₀ 1-100 nM; +=EC₅₀>100 nM.

TABLE 3

| Ex. | Activity |
|---|---|
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | ++ |
| 20 | +++ |
| 21 | ++ |
| 22 | +++ |
| 23 | ++ |
| 24 | +++ |
| 25 | +++ |
| 26 | +++ |
| 27 | +++ |
| 28 | +++ |
| 29 | +++ |
| 30 | +++ |
| 31 | +++ |
| 32 | +++ |
| 33 | +++ |
| 34 | +++ |
| 35 | ++ |
| 36 | +++ |
| 37 | +++ |
| 38 | +++ |
| 39 | ++ |
| 40 | +++ |
| 41 | +++ |
| 42 | ++ |
| 43 | +++ |
| 44 | +++ |
| 45 | ++ |
| 46 | ++ |
| 47 | +++ |
| 48 | ++ |
| 49 | ++ |
| 50 | ++ |
| 51 | ++ |
| 52 | ++ |
| 53 | ++ |
| 54 | ++ |
| 55 | +++ |
| 56 | ++ |
| 57 | ++ |
| 58 | +++ |
| 59 | +++ |
| 60 | ++ |
| 61 | +++ |
| 62 | +++ |
| 63 | +++ |
| 64 | +++ |
| 65 | +++ |
| 66 | +++ |
| 67 | ++ |
| 71 | +++ |
| 73 | +++ |
| 75 | +++ |
| 77 | +++ |
| 78 | +++ |
| 79 | ++ |
| 80 | ++ |
| 81 | ++ |
| 82 | ++ |
| 83 | ++ |
| 85 | ++ |
| 86 | ++ |
| 87 | ++ |
| 89 | ++ |
| 90 | ++ |
| 91 | ++ |
| 92 | ++ |
| 93 | ++ |
| 94 | ++ |
| 95 | + |
| 96 | + |
| 97 | + |
| 98 | + |
| 99 | + |
| 100 | + |

1. A compound selected from: 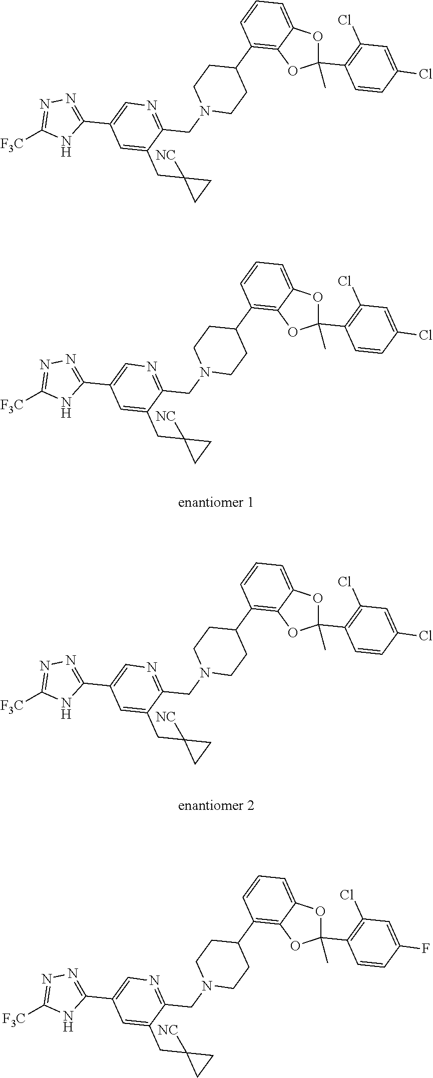 [SMILES: CC1(c2ccc(Cl)cc2Cl)Oc2cccc(C3CCN(Cc4ncc(-c5nnc(C(F)(F)F)[nH]5)cc4CC4(C#N)CC4)CC3)c2O1 ; CC1(c2ccc(Cl)cc2Cl)Oc2cccc(C3CCN(Cc4ncc(-c5nnc(C(F)(F)F)[nH]5)cc4CC4(C#N)CC4)CC3)c2O1 ; CC1(c2ccc(Cl)cc2Cl)Oc2cccc(C3CCN(Cc4ncc(-c5nnc(C(F)(F)F)[nH]5)cc4CC4(C#N)CC4)CC3)c2O1 ; CC1(c2ccc(F)cc2Cl)Oc2cccc(C3CCN(Cc4ncc(-c5nnc(C(F)(F)F)[nH]5)cc4CC4(C#N)CC4)CC3)c2O1] 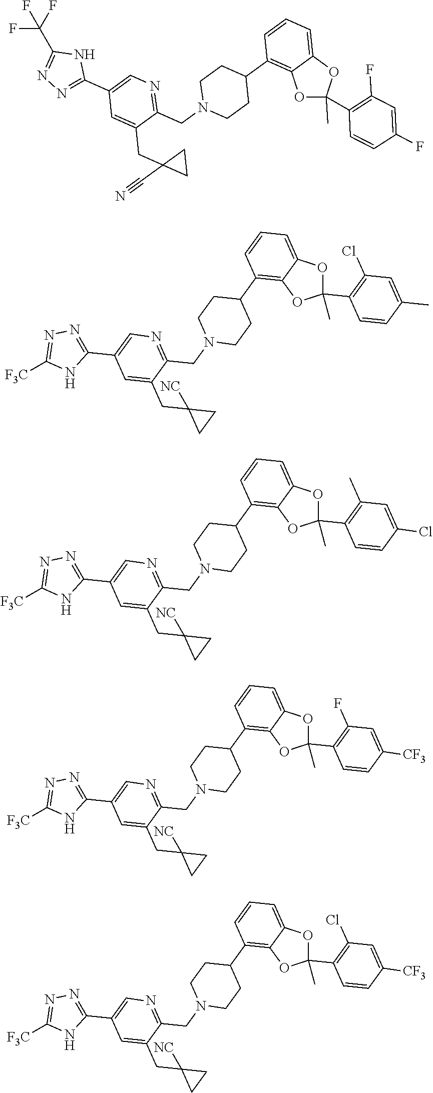 [SMILES: CC1(c2ccc(F)cc2F)Oc2cccc(C3CCN(Cc4ncc(-c5nnc(C(F)(F)F)[nH]5)cc4CC4(C#N)CC4)CC3)c2O1 ; Cc1ccc(C2(C)Oc3cccc(C4CCN(Cc5ncc(-c6nnc(C(F)(F)F)[nH]6)cc5CC5(C#N)CC5)CC4)c3O2)c(Cl)c1 ; Cc1cc(Cl)ccc1C1(C)Oc2cccc(C3CCN(Cc4ncc(-c5nnc(C(F)(F)F)[nH]5)cc4CC4(C#N)CC4)CC3)c2O1 ; CC1(c2ccc(C(F)(F)F)cc2F)Oc2cccc(C3CCN(Cc4ncc(-c5nnc(C(F)(F)F)[nH]5)cc4CC4(C#N)CC4)CC3)c2O1 ; CC1(c2ccc(C(F)(F)F)cc2Cl)Oc2cccc(C3CCN(Cc4ncc(-c5nnc(C(F)(F)F)[nH]5)cc4CC4(C#N)CC4)CC3)c2O1] 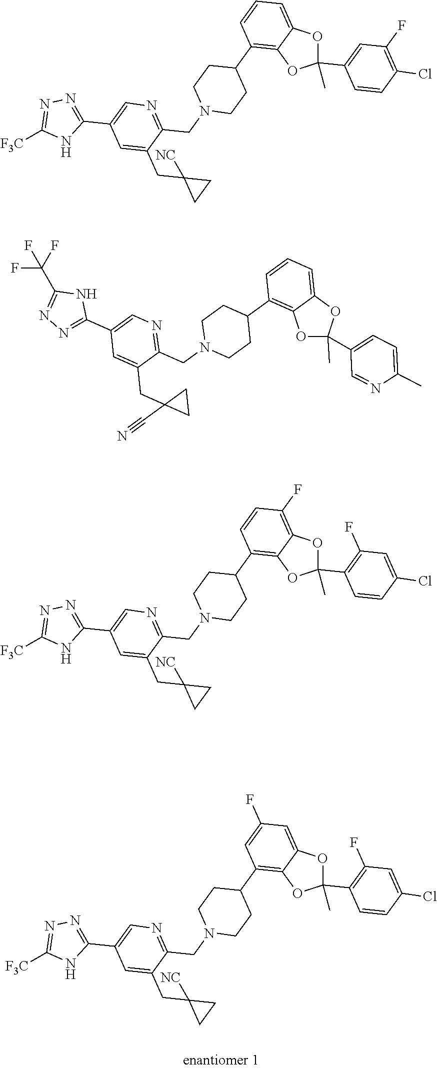 [SMILES: CC1(c2ccc(Cl)c(F)c2)Oc2cccc(C3CCN(Cc4ncc(-c5nnc(C(F)(F)F)[nH]5)cc4CC4(C#N)CC4)CC3)c2O1 ; Cc1ccc(C2(C)Oc3cccc(C4CCN(Cc5ncc(-c6nnc(C(F)(F)F)[nH]6)cc5CC5(C#N)CC5)CC4)c3O2)cn1 ; CC1(c2ccc(Cl)cc2F)Oc2c(F)ccc(C3CCN(Cc4ncc(-c5nnc(C(F)(F)F)[nH]5)cc4CC4(C#N)CC4)CC3)c2O1 ; CC1(c2ccc(Cl)cc2F)Oc2cc(F)cc(C3CCN(Cc4ncc(-c5nnc(C(F)(F)F)[nH]5)cc4CC4(C#N)CC4)CC3)c2O1] 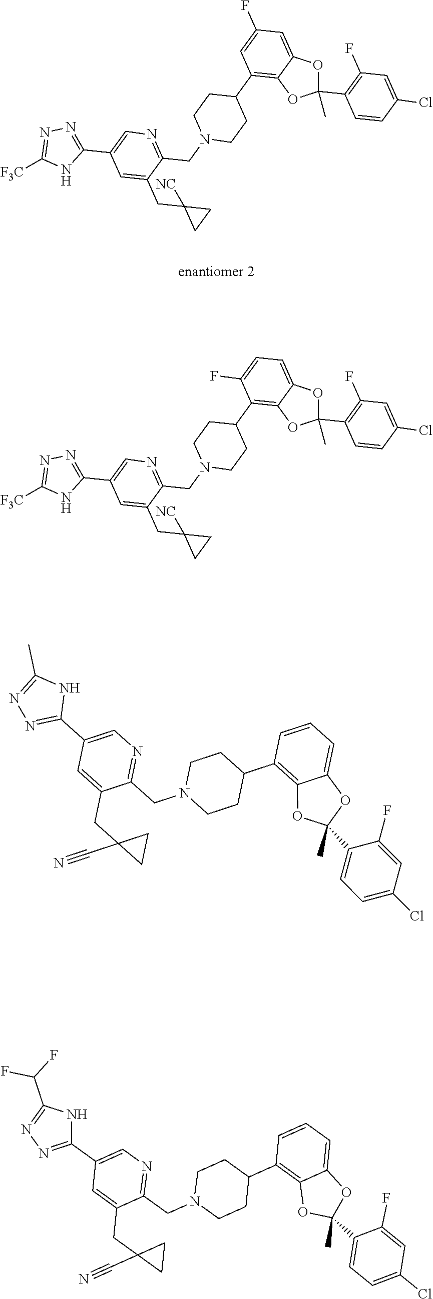 [SMILES: CC1(c2ccc(Cl)cc2F)Oc2cc(F)cc(C3CCN(Cc4ncc(-c5nnc(C(F)(F)F)[nH]5)cc4CC4(C#N)CC4)CC3)c2O1 ; CC1(c2ccc(Cl)cc2F)Oc2ccc(F)c(C3CCN(Cc4ncc(-c5nnc(C(F)(F)F)[nH]5)cc4CC4(C#N)CC4)CC3)c2O1 ; Cc1nnc(-c2cnc(CN3CCC(c4cccc5c4O[C@@](C)(c4ccc(Cl)cc4F)O5)CC3)c(CC3(C#N)CC3)c2)[nH]1 ; C[C@]1(c2ccc(Cl)cc2F)Oc2cccc(C3CCN(Cc4ncc(-c5nnc(C(F)F)[nH]5)cc4CC4(C#N)CC4)CC3)c2O1] 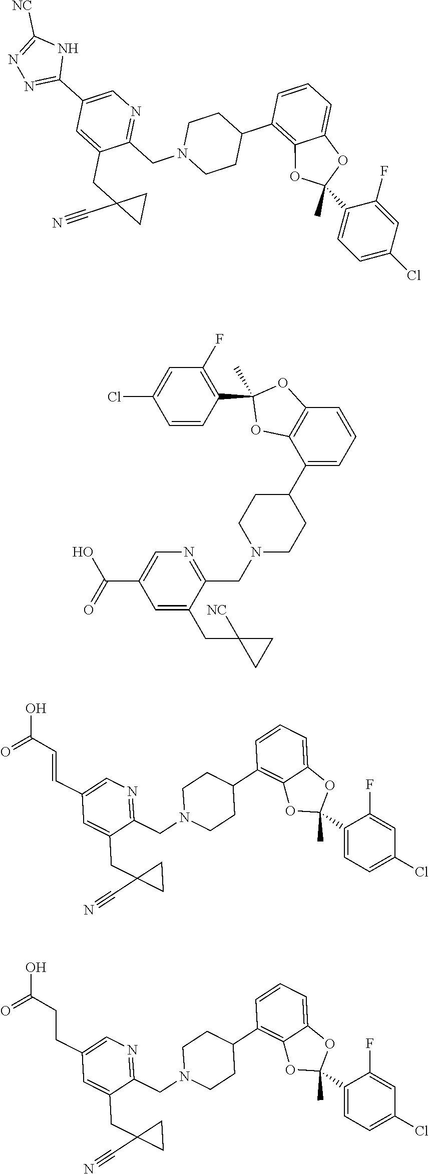 [SMILES: C[C@]1(c2ccc(Cl)cc2F)Oc2cccc(C3CCN(Cc4ncc(-c5nnc(C#N)[nH]5)cc4CC4(C#N)CC4)CC3)c2O1 ; C[C@]1(c2ccc(Cl)cc2F)Oc2cccc(C3CCN(Cc4ncc(C(=O)O)cc4CC4(C#N)CC4)CC3)c2O1 ; C[C@]1(c2ccc(Cl)cc2F)Oc2cccc(C3CCN(Cc4ncc(/C=C/C(=O)O)cc4CC4(C#N)CC4)CC3)c2O1 ; C[C@]1(c2ccc(Cl)cc2F)Oc2cccc(C3CCN(Cc4ncc(CCC(=O)O)cc4CC4(C#N)CC4)CC3)c2O1] 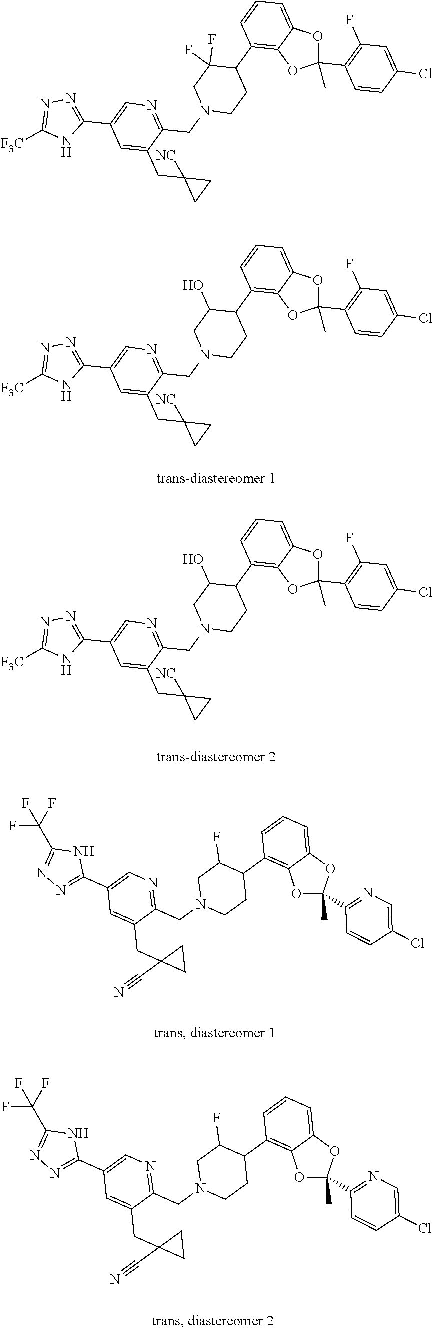 [SMILES: CC1(c2ccc(Cl)cc2F)Oc2cccc(C3CCN(Cc4ncc(-c5nnc(C(F)(F)F)[nH]5)cc4CC4(C#N)CC4)CC3(F)F)c2O1 ; CC1(c2ccc(Cl)cc2F)Oc2cccc(C3CCN(Cc4ncc(-c5nnc(C(F)(F)F)[nH]5)cc4CC4(C#N)CC4)CC3O)c2O1 ; CC1(c2ccc(Cl)cc2F)Oc2cccc(C3CCN(Cc4ncc(-c5nnc(C(F)(F)F)[nH]5)cc4CC4(C#N)CC4)CC3O)c2O1 ; C[C@]1(c2ccc(Cl)cn2)Oc2cccc(C3CCN(Cc4ncc(-c5nnc(C(F)(F)F)[nH]5)cc4CC4(C#N)CC4)CC3F)c2O1 ; C[C@]1(c2ccc(Cl)cn2)Oc2cccc(C3CCN(Cc4ncc(-c5nnc(C(F)(F)F)[nH]5)cc4CC4(C#N)CC4)CC3F)c2O1] 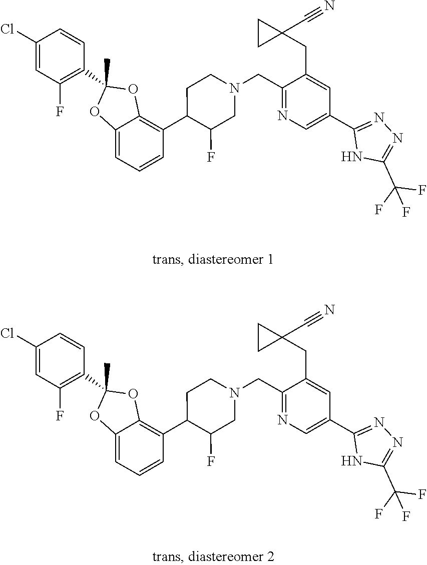 [SMILES: C[C@]1(c2ccc(Cl)cc2F)Oc2cccc(C3CCN(Cc4ncc(-c5nnc(C(F)(F)F)[nH]5)cc4CC4(C#N)CC4)CC3F)c2O1 ; C[C@]1(c2ccc(Cl)cc2F)Oc2cccc(C3CCN(Cc4ncc(-c5nnc(C(F)(F)F)[nH]5)cc4CC4(C#N)CC4)CC3F)c2O1] 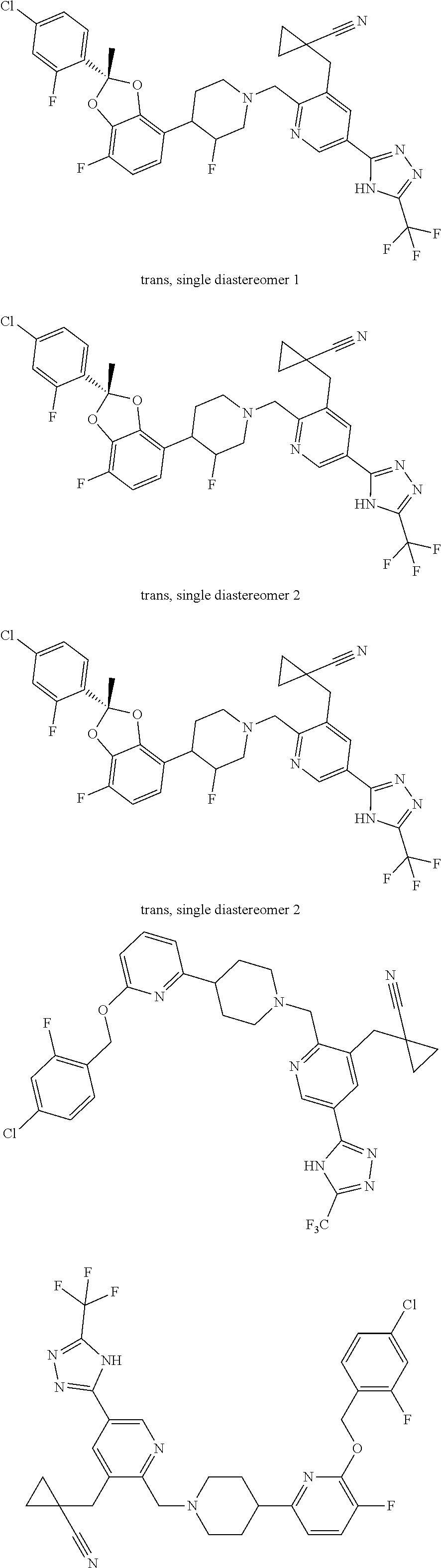 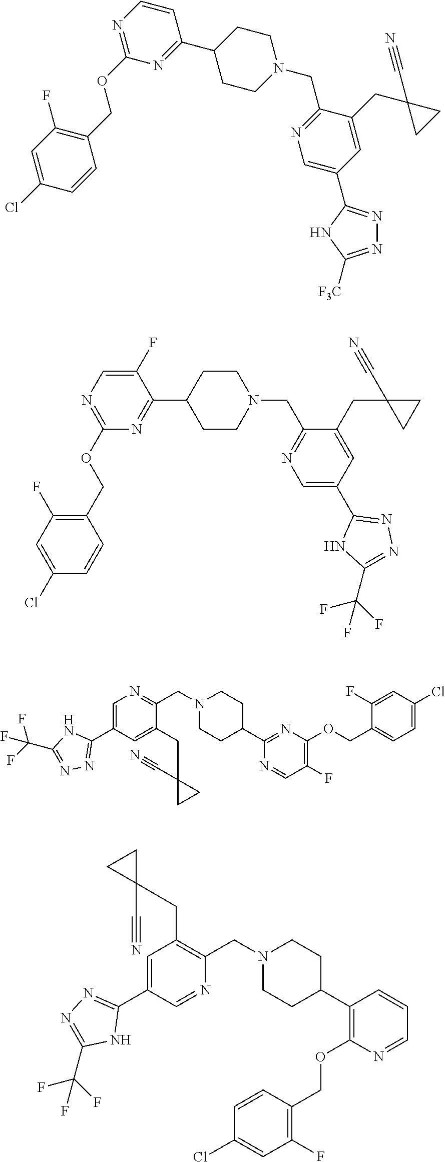 [SMILES: N#CC1(Cc2cc(-c3nnc(C(F)(F)F)[nH]3)cnc2CN2CCC(c3ccnc(OCc4ccc(Cl)cc4F)n3)CC2)CC1 ; N#CC1(Cc2cc(-c3nnc(C(F)(F)F)[nH]3)cnc2CN2CCC(c3nc(OCc4ccc(Cl)cc4F)ncc3F)CC2)CC1 ; N#CC1(Cc2cc(-c3nnc(C(F)(F)F)[nH]3)cnc2CN2CCC(c3ncc(F)c(OCc4ccc(Cl)cc4F)n3)CC2)CC1 ; N#CC1(Cc2cc(-c3nnc(C(F)(F)F)[nH]3)cnc2CN2CCC(c3cccnc3OCc3ccc(Cl)cc3F)CC2)CC1] 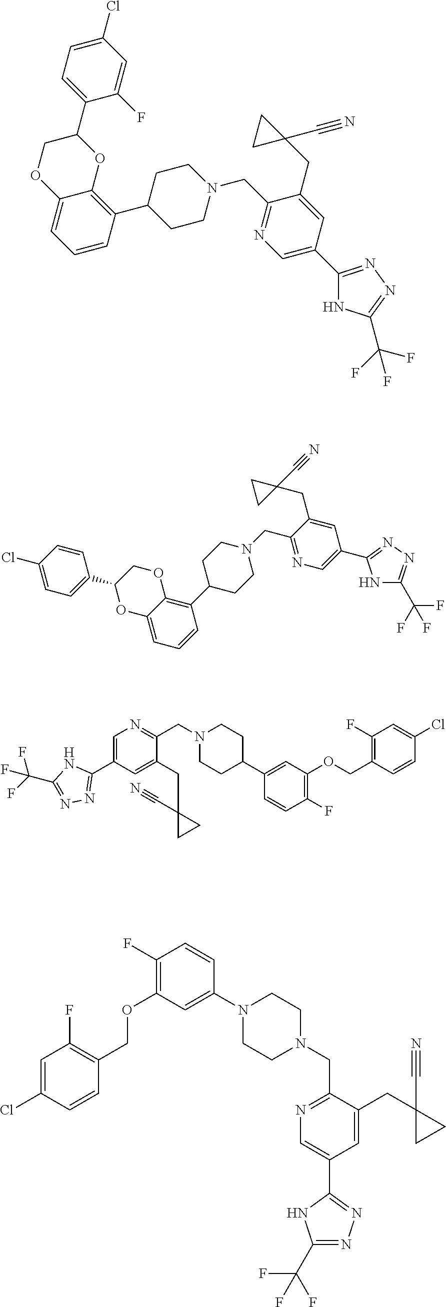 [SMILES: N#CC1(Cc2cc(-c3nnc(C(F)(F)F)[nH]3)cnc2CN2CCC(c3cccc4c3OC(c3ccc(Cl)cc3F)CO4)CC2)CC1 ; N#CC1(Cc2cc(-c3nnc(C(F)(F)F)[nH]3)cnc2CN2CCC(c3cccc4c3OC[C@@H](c3ccc(Cl)cc3)O4)CC2)CC1 ; N#CC1(Cc2cc(-c3nnc(C(F)(F)F)[nH]3)cnc2CN2CCC(c3ccc(F)c(OCc4ccc(Cl)cc4F)c3)CC2)CC1 ; N#CC1(Cc2cc(-c3nnc(C(F)(F)F)[nH]3)cnc2CN2CCN(c3ccc(F)c(OCc4ccc(Cl)cc4F)c3)CC2)CC1] 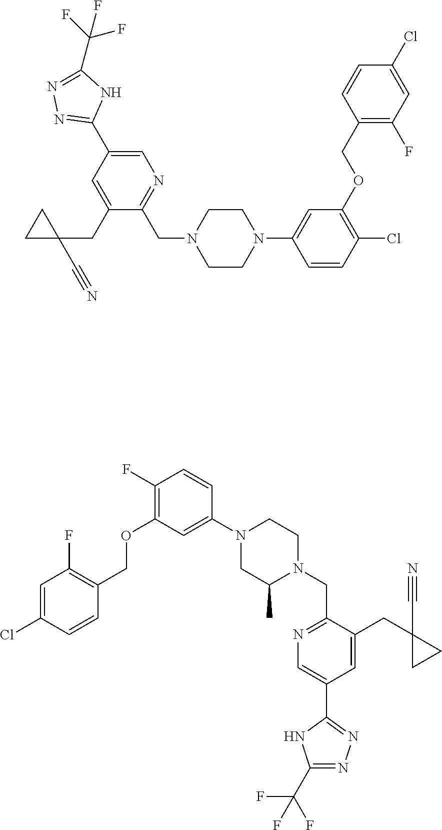 [SMILES: N#CC1(Cc2cc(-c3nnc(C(F)(F)F)[nH]3)cnc2CN2CCN(c3ccc(Cl)c(OCc4ccc(Cl)cc4F)c3)CC2)CC1 ; C[C@H]1CN(c2ccc(F)c(OCc3ccc(Cl)cc3F)c2)CCN1Cc1ncc(-c2nnc(C(F)(F)F)[nH]2)cc1CC1(C#N)CC1] 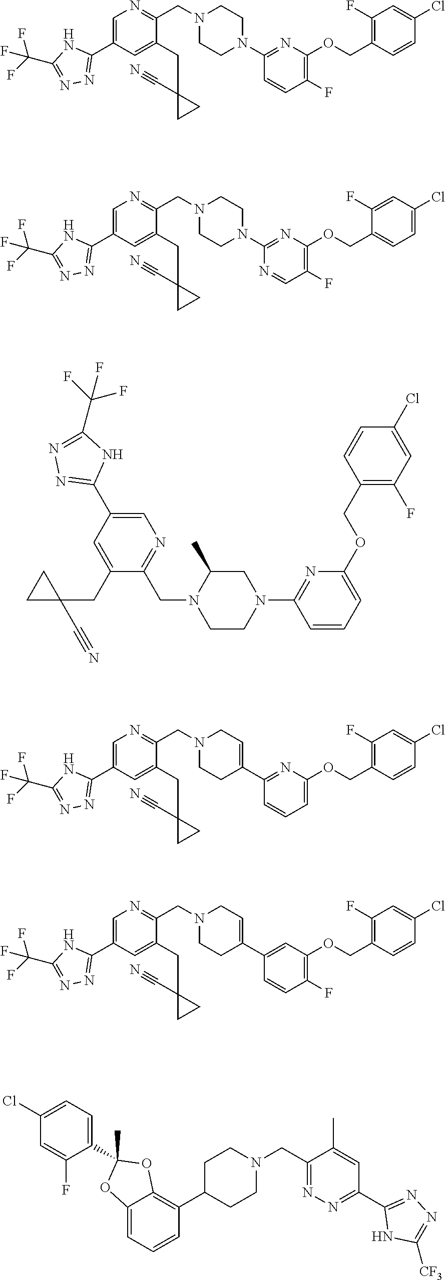 [SMILES: N#CC1(Cc2cc(-c3nnc(C(F)(F)F)[nH]3)cnc2CN2CCN(c3ccc(F)c(OCc4ccc(Cl)cc4F)n3)CC2)CC1 ; N#CC1(Cc2cc(-c3nnc(C(F)(F)F)[nH]3)cnc2CN2CCN(c3ncc(F)c(OCc4ccc(Cl)cc4F)n3)CC2)CC1 ; C[C@H]1CN(c2cccc(OCc3ccc(Cl)cc3F)n2)CCN1Cc1ncc(-c2nnc(C(F)(F)F)[nH]2)cc1CC1(C#N)CC1 ; N#CC1(Cc2cc(-c3nnc(C(F)(F)F)[nH]3)cnc2CN2CC=C(c3cccc(OCc4ccc(Cl)cc4F)n3)CC2)CC1 ; N#CC1(Cc2cc(-c3nnc(C(F)(F)F)[nH]3)cnc2CN2CC=C(c3ccc(F)c(OCc4ccc(Cl)cc4F)c3)CC2)CC1 ; Cc1cc(-c2nnc(C(F)(F)F)[nH]2)nnc1CN1CCC(c2cccc3c2O[C@@](C)(c2ccc(Cl)cc2F)O3)CC1] 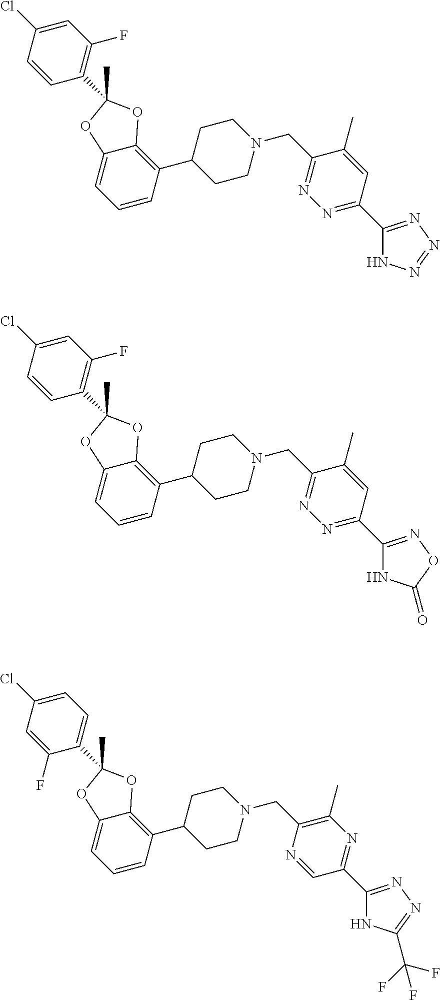 [SMILES: Cc1cc(-c2nnn[nH]2)nnc1CN1CCC(c2cccc3c2O[C@@](C)(c2ccc(Cl)cc2F)O3)CC1 ; Cc1cc(-c2noc(=O)[nH]2)nnc1CN1CCC(c2cccc3c2O[C@@](C)(c2ccc(Cl)cc2F)O3)CC1 ; Cc1nc(-c2nnc(C(F)(F)F)[nH]2)cnc1CN1CCC(c2cccc3c2O[C@@](C)(c2ccc(Cl)cc2F)O3)CC1] 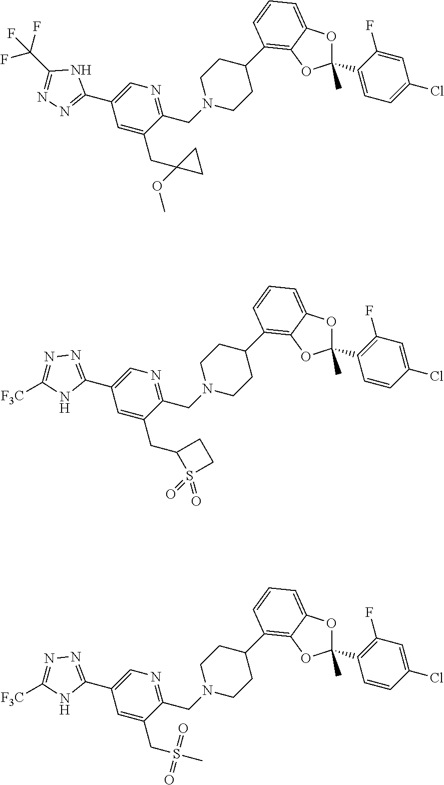 [SMILES: COC1(Cc2cc(-c3nnc(C(F)(F)F)[nH]3)cnc2CN2CCC(c3cccc4c3O[C@@](C)(c3ccc(Cl)cc3F)O4)CC2)CC1 ; C[C@]1(c2ccc(Cl)cc2F)Oc2cccc(C3CCN(Cc4ncc(-c5nnc(C(F)(F)F)[nH]5)cc4CC4CCS4(=O)=O)CC3)c2O1 ; C[C@]1(c2ccc(Cl)cc2F)Oc2cccc(C3CCN(Cc4ncc(-c5nnc(C(F)(F)F)[nH]5)cc4CS(C)(=O)=O)CC3)c2O1] 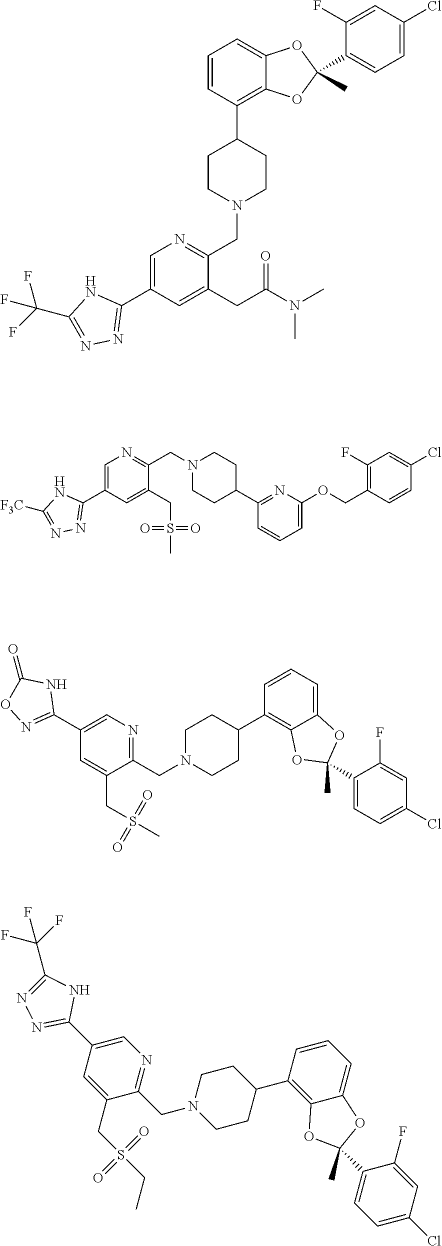 [SMILES: CN(C)C(=O)Cc1cc(-c2nnc(C(F)(F)F)[nH]2)cnc1CN1CCC(c2cccc3c2O[C@@](C)(c2ccc(Cl)cc2F)O3)CC1 ; CS(=O)(=O)Cc1cc(-c2nnc(C(F)(F)F)[nH]2)cnc1CN1CCC(c2cccc(OCc3ccc(Cl)cc3F)n2)CC1 ; C[C@]1(c2ccc(Cl)cc2F)Oc2cccc(C3CCN(Cc4ncc(-c5noc(=O)[nH]5)cc4CS(C)(=O)=O)CC3)c2O1 ; CCS(=O)(=O)Cc1cc(-c2nnc(C(F)(F)F)[nH]2)cnc1CN1CCC(c2cccc3c2O[C@@](C)(c2ccc(Cl)cc2F)O3)CC1] 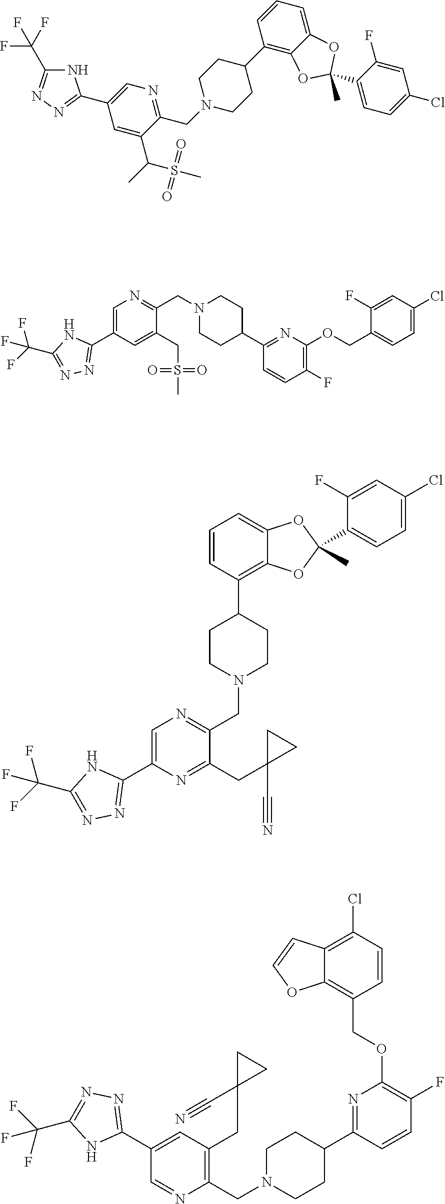 [SMILES: CC(c1cc(-c2nnc(C(F)(F)F)[nH]2)cnc1CN1CCC(c2cccc3c2O[C@@](C)(c2ccc(Cl)cc2F)O3)CC1)S(C)(=O)=O ; CS(=O)(=O)Cc1cc(-c2nnc(C(F)(F)F)[nH]2)cnc1CN1CCC(c2ccc(F)c(OCc3ccc(Cl)cc3F)n2)CC1 ; C[C@]1(c2ccc(Cl)cc2F)Oc2cccc(C3CCN(Cc4ncc(-c5nnc(C(F)(F)F)[nH]5)nc4CC4(C#N)CC4)CC3)c2O1 ; N#CC1(Cc2cc(-c3nnc(C(F)(F)F)[nH]3)cnc2CN2CCC(c3ccc(F)c(OCc4ccc(Cl)c5ccoc45)n3)CC2)CC1] 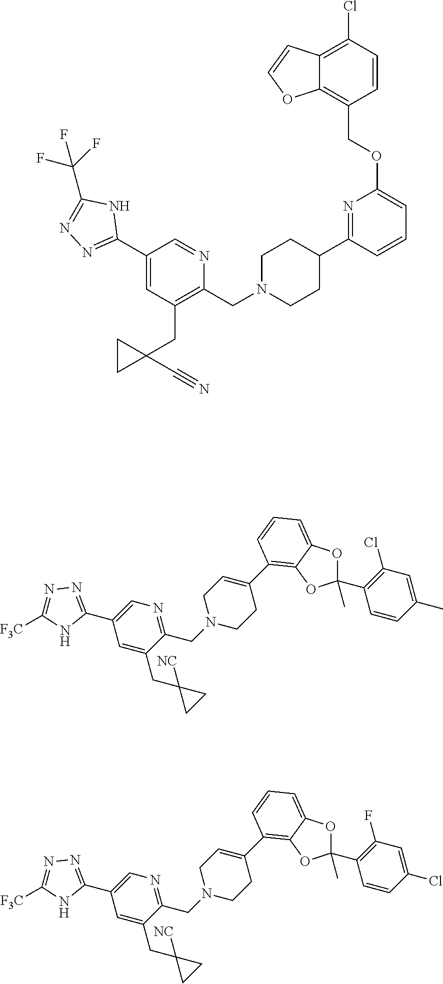 [SMILES: N#CC1(Cc2cc(-c3nnc(C(F)(F)F)[nH]3)cnc2CN2CCC(c3cccc(OCc4ccc(Cl)c5ccoc45)n3)CC2)CC1 ; Cc1ccc(C2(C)Oc3cccc(C4=CCN(Cc5ncc(-c6nnc(C(F)(F)F)[nH]6)cc5CC5(C#N)CC5)CC4)c3O2)c(Cl)c1 ; CC1(c2ccc(Cl)cc2F)Oc2cccc(C3=CCN(Cc4ncc(-c5nnc(C(F)(F)F)[nH]5)cc4CC4(C#N)CC4)CC3)c2O1] 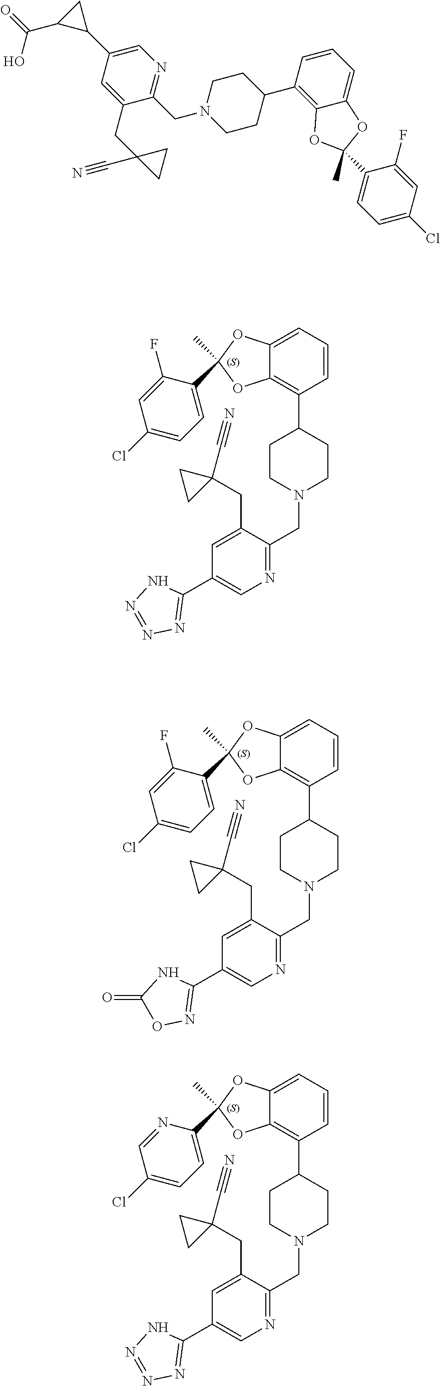 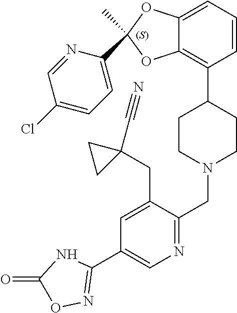 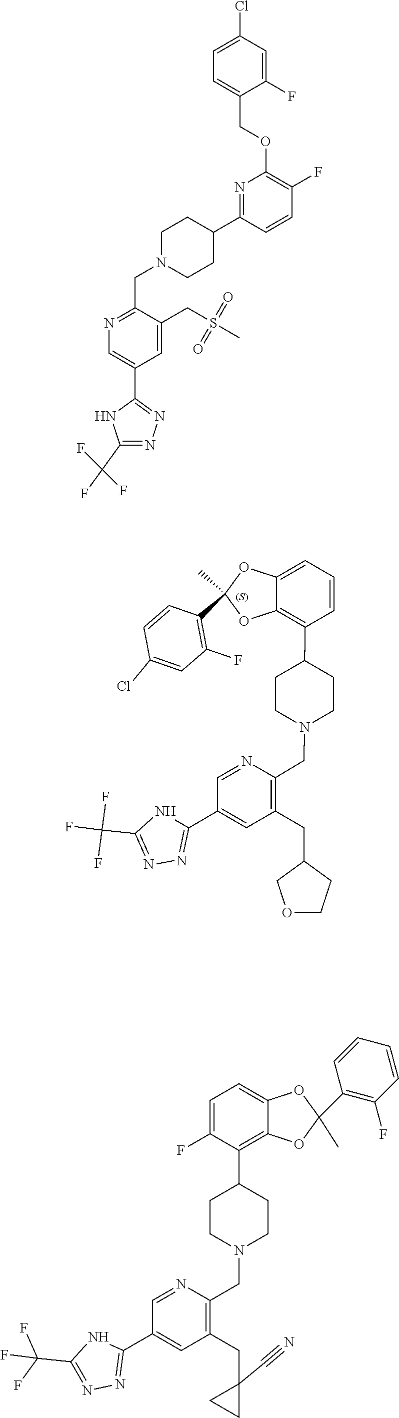 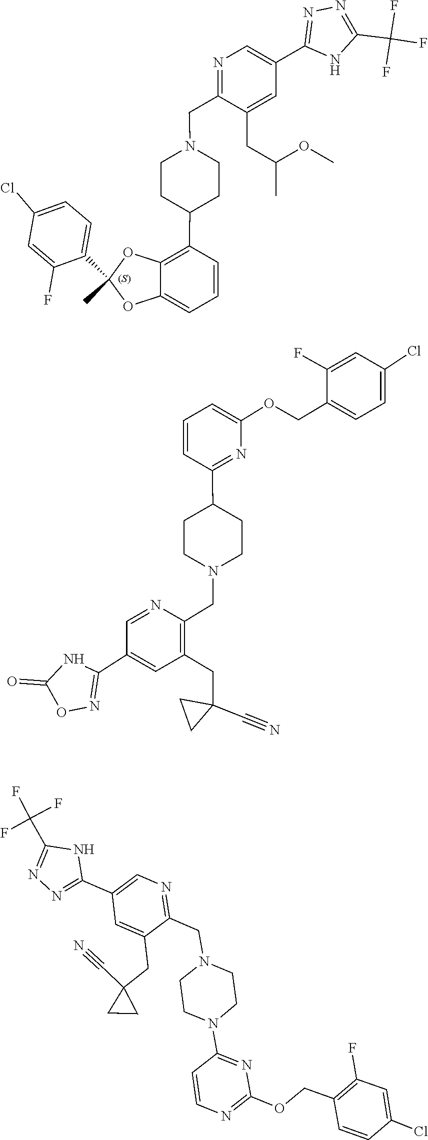 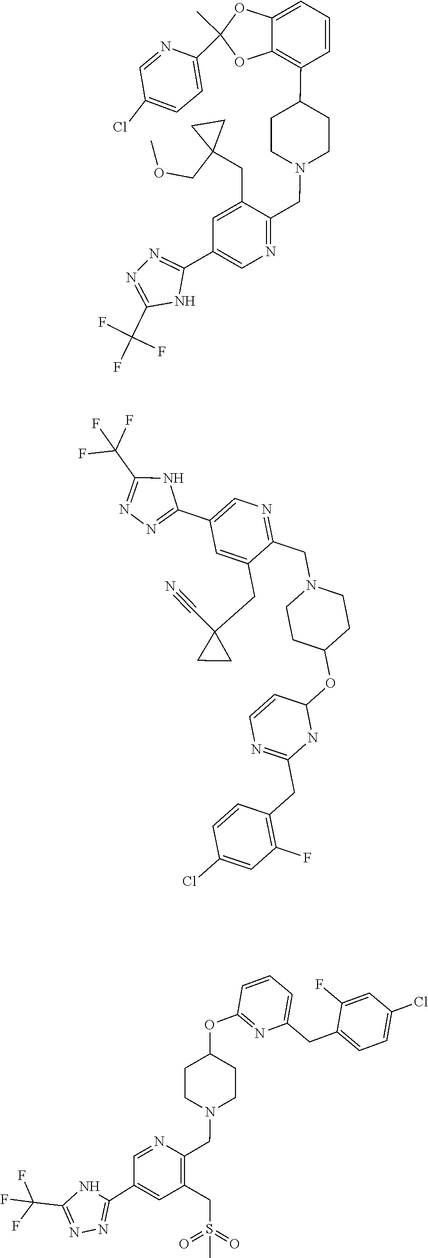 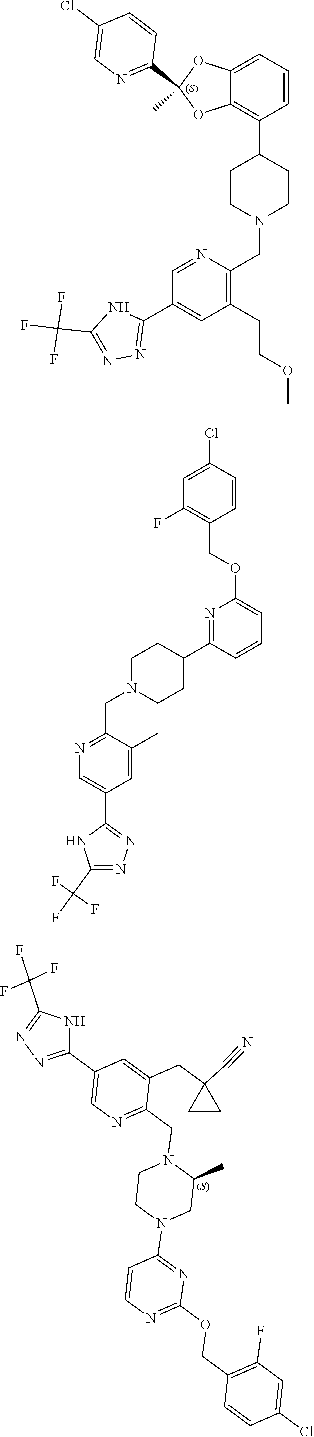 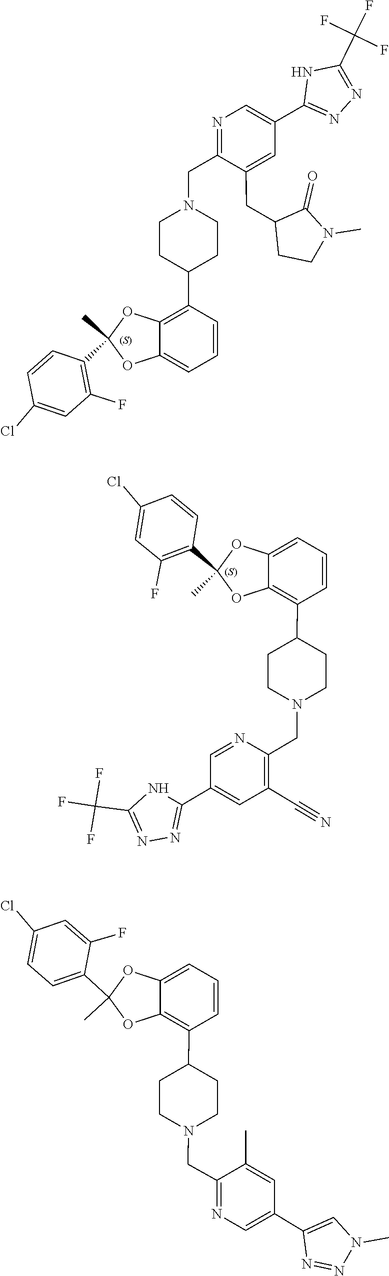 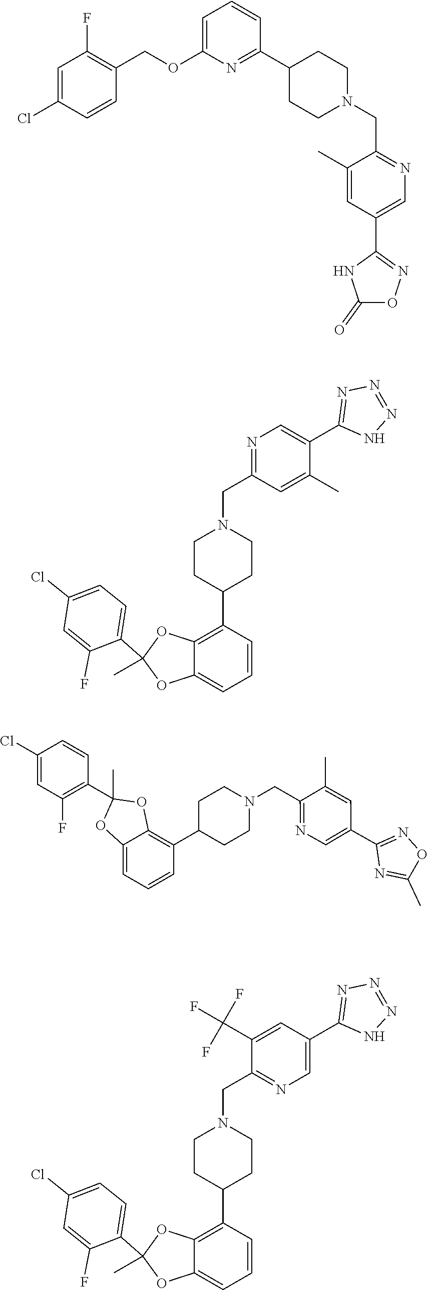 [SMILES: Cc1cc(-c2noc(=O)[nH]2)cnc1CN1CCC(c2cccc(OCc3ccc(Cl)cc3F)n2)CC1 ; Cc1cc(CN2CCC(c3cccc4c3OC(C)(c3ccc(Cl)cc3F)O4)CC2)ncc1-c1nnn[nH]1 ; Cc1nc(-c2cnc(CN3CCC(c4cccc5c4OC(C)(c4ccc(Cl)cc4F)O5)CC3)c(C)c2)no1 ; CC1(c2ccc(Cl)cc2F)Oc2cccc(C3CCN(Cc4ncc(-c5nnn[nH]5)cc4C(F)(F)F)CC3)c2O1] 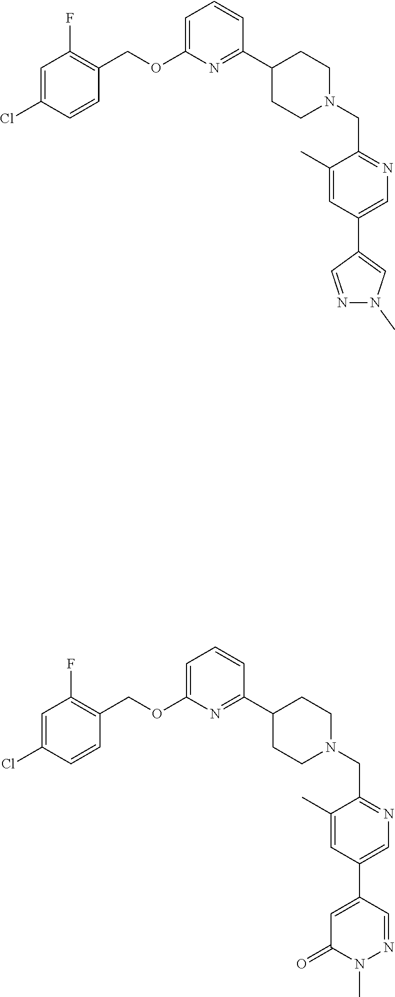 [SMILES: Cc1cc(-c2cnn(C)c2)cnc1CN1CCC(c2cccc(OCc3ccc(Cl)cc3F)n2)CC1 ; Cc1cc(-c2cnn(C)c(=O)c2)cnc1CN1CCC(c2cccc(OCc3ccc(Cl)cc3F)n2)CC1] 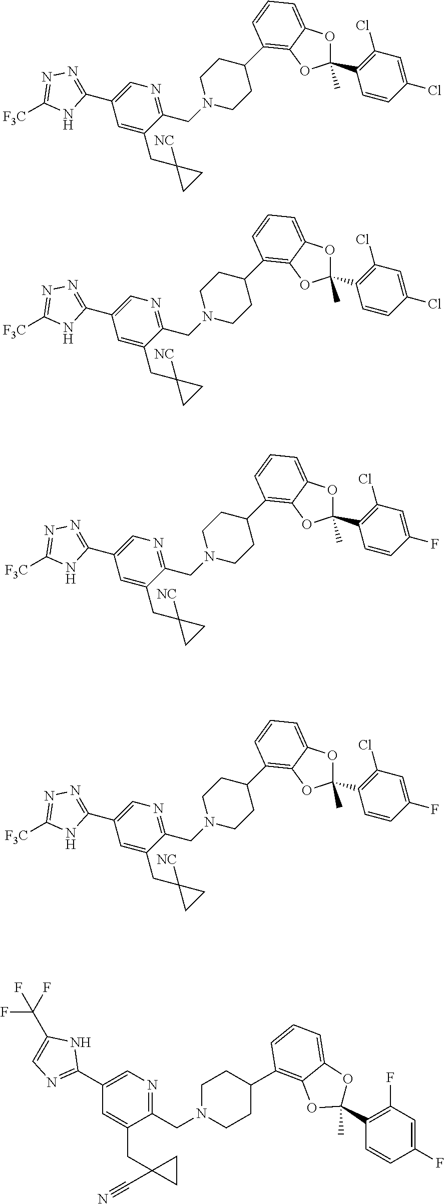 [SMILES: C[C@@]1(c2ccc(Cl)cc2Cl)Oc2cccc(C3CCN(Cc4ncc(-c5nnc(C(F)(F)F)[nH]5)cc4CC4(C#N)CC4)CC3)c2O1 ; C[C@]1(c2ccc(Cl)cc2Cl)Oc2cccc(C3CCN(Cc4ncc(-c5nnc(C(F)(F)F)[nH]5)cc4CC4(C#N)CC4)CC3)c2O1 ; C[C@@]1(c2ccc(F)cc2Cl)Oc2cccc(C3CCN(Cc4ncc(-c5nnc(C(F)(F)F)[nH]5)cc4CC4(C#N)CC4)CC3)c2O1 ; C[C@]1(c2ccc(F)cc2Cl)Oc2cccc(C3CCN(Cc4ncc(-c5nnc(C(F)(F)F)[nH]5)cc4CC4(C#N)CC4)CC3)c2O1 ; C[C@@]1(c2ccc(F)cc2F)Oc2cccc(C3CCN(Cc4ncc(-c5ncc(C(F)(F)F)[nH]5)cc4CC4(C#N)CC4)CC3)c2O1] 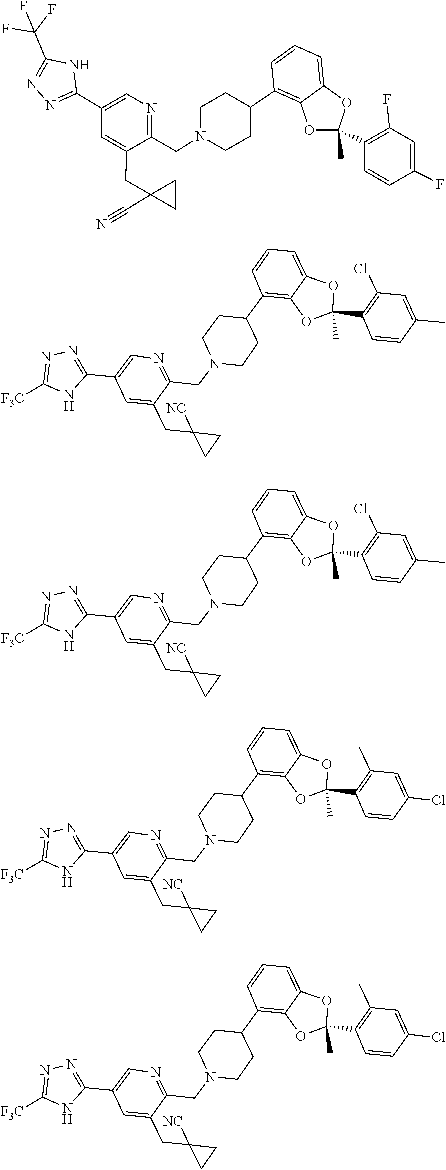 [SMILES: C[C@]1(c2ccc(F)cc2F)Oc2cccc(C3CCN(Cc4ncc(-c5nnc(C(F)(F)F)[nH]5)cc4CC4(C#N)CC4)CC3)c2O1 ; Cc1ccc([C@]2(C)Oc3cccc(C4CCN(Cc5ncc(-c6nnc(C(F)(F)F)[nH]6)cc5CC5(C#N)CC5)CC4)c3O2)c(Cl)c1 ; Cc1ccc([C@@]2(C)Oc3cccc(C4CCN(Cc5ncc(-c6nnc(C(F)(F)F)[nH]6)cc5CC5(C#N)CC5)CC4)c3O2)c(Cl)c1 ; Cc1cc(Cl)ccc1[C@]1(C)Oc2cccc(C3CCN(Cc4ncc(-c5nnc(C(F)(F)F)[nH]5)cc4CC4(C#N)CC4)CC3)c2O1 ; Cc1cc(Cl)ccc1[C@@]1(C)Oc2cccc(C3CCN(Cc4ncc(-c5nnc(C(F)(F)F)[nH]5)cc4CC4(C#N)CC4)CC3)c2O1] 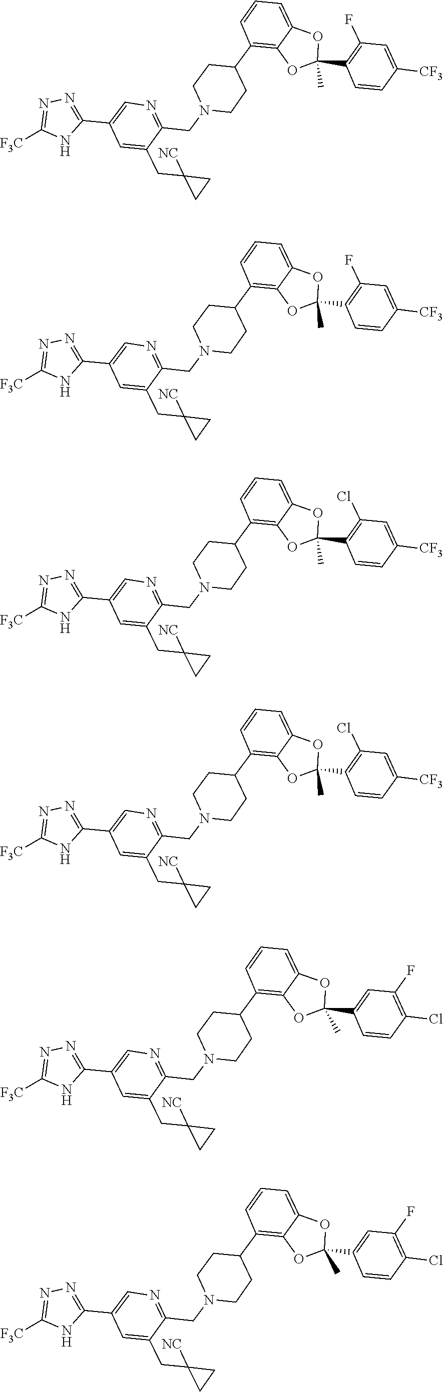 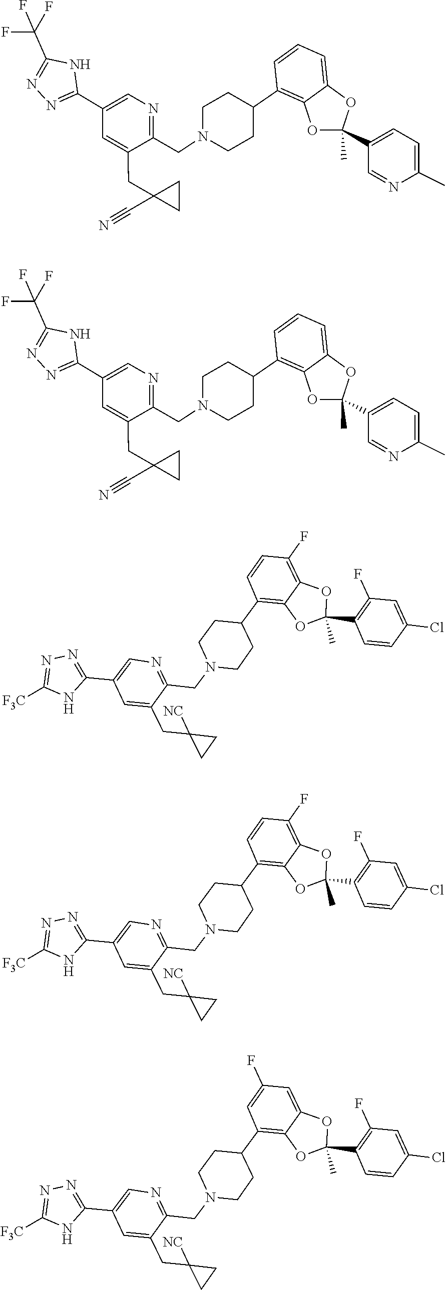 [SMILES: Cc1ccc([C@]2(C)Oc3cccc(C4CCN(Cc5ncc(-c6nnc(C(F)(F)F)[nH]6)cc5CC5(C#N)CC5)CC4)c3O2)cn1 ; Cc1ccc([C@@]2(C)Oc3cccc(C4CCN(Cc5ncc(-c6nnc(C(F)(F)F)[nH]6)cc5CC5(C#N)CC5)CC4)c3O2)cn1 ; C[C@@]1(c2ccc(Cl)cc2F)Oc2c(F)ccc(C3CCN(Cc4ncc(-c5nnc(C(F)(F)F)[nH]5)cc4CC4(C#N)CC4)CC3)c2O1 ; C[C@]1(c2ccc(Cl)cc2F)Oc2c(F)ccc(C3CCN(Cc4ncc(-c5nnc(C(F)(F)F)[nH]5)cc4CC4(C#N)CC4)CC3)c2O1 ; C[C@@]1(c2ccc(Cl)cc2F)Oc2cc(F)cc(C3CCN(Cc4ncc(-c5nnc(C(F)(F)F)[nH]5)cc4CC4(C#N)CC4)CC3)c2O1] 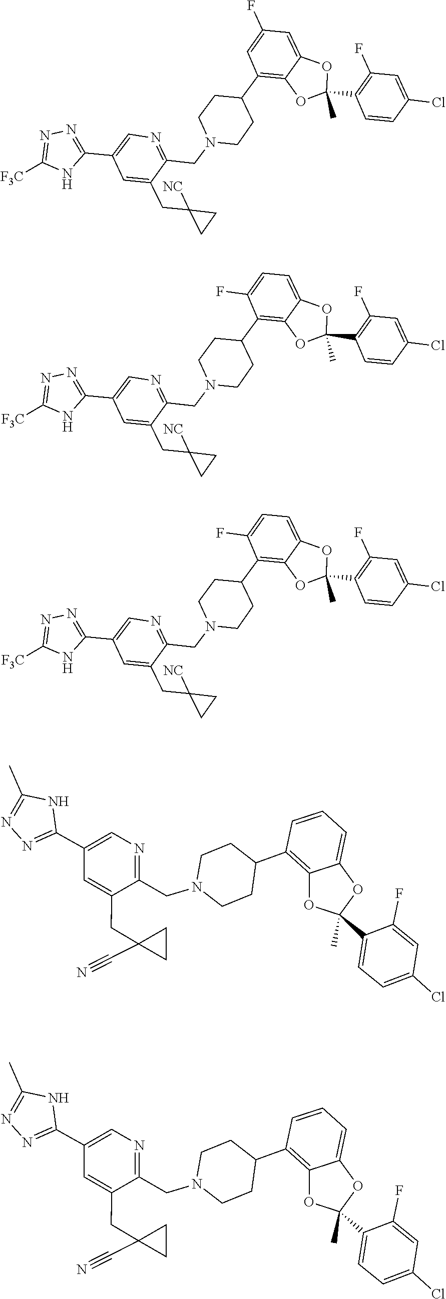 [SMILES: C[C@]1(c2ccc(Cl)cc2F)Oc2cc(F)cc(C3CCN(Cc4ncc(-c5nnc(C(F)(F)F)[nH]5)cc4CC4(C#N)CC4)CC3)c2O1 ; C[C@@]1(c2ccc(Cl)cc2F)Oc2ccc(F)c(C3CCN(Cc4ncc(-c5nnc(C(F)(F)F)[nH]5)cc4CC4(C#N)CC4)CC3)c2O1 ; C[C@]1(c2ccc(Cl)cc2F)Oc2ccc(F)c(C3CCN(Cc4ncc(-c5nnc(C(F)(F)F)[nH]5)cc4CC4(C#N)CC4)CC3)c2O1 ; Cc1nnc(-c2cnc(CN3CCC(c4cccc5c4O[C@](C)(c4ccc(Cl)cc4F)O5)CC3)c(CC3(C#N)CC3)c2)[nH]1 ; Cc1nnc(-c2cnc(CN3CCC(c4cccc5c4O[C@@](C)(c4ccc(Cl)cc4F)O5)CC3)c(CC3(C#N)CC3)c2)[nH]1] 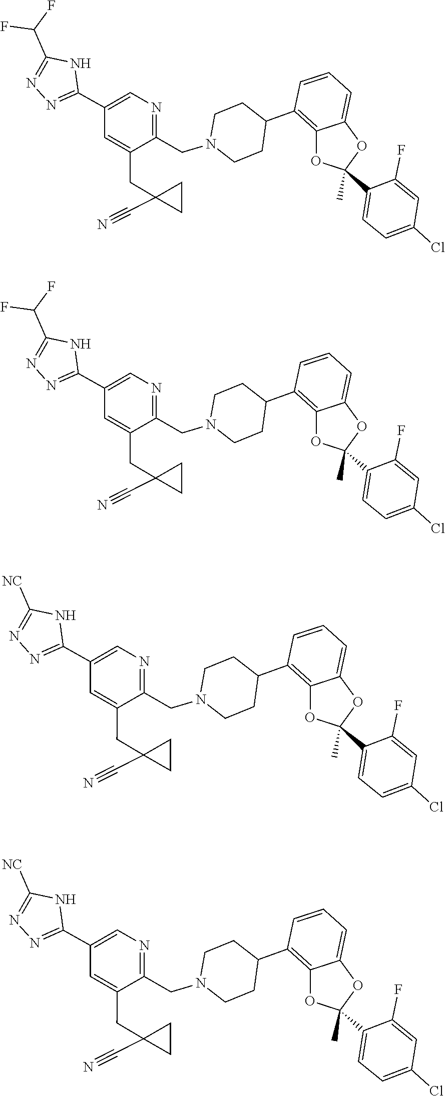 [SMILES: C[C@@]1(c2ccc(Cl)cc2F)Oc2cccc(C3CCN(Cc4ncc(-c5nnc(C(F)F)[nH]5)cc4CC4(C#N)CC4)CC3)c2O1 ; C[C@]1(c2ccc(Cl)cc2F)Oc2cccc(C3CCN(Cc4ncc(-c5nnc(C(F)F)[nH]5)cc4CC4(C#N)CC4)CC3)c2O1 ; C[C@@]1(c2ccc(Cl)cc2F)Oc2cccc(C3CCN(Cc4ncc(-c5nnc(C#N)[nH]5)cc4CC4(C#N)CC4)CC3)c2O1 ; C[C@]1(c2ccc(Cl)cc2F)Oc2cccc(C3CCN(Cc4ncc(-c5nnc(C#N)[nH]5)cc4CC4(C#N)CC4)CC3)c2O1] 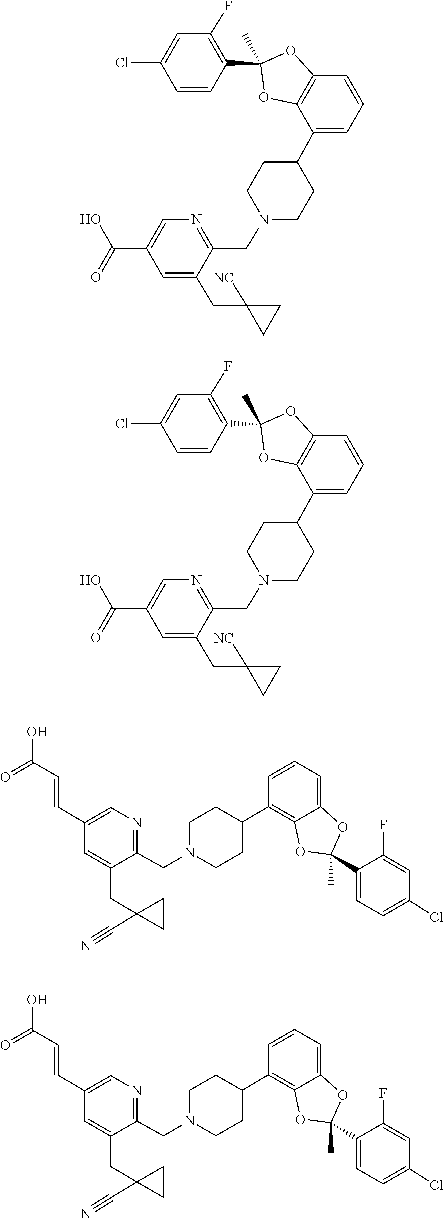 [SMILES: C[C@]1(c2ccc(Cl)cc2F)Oc2cccc(C3CCN(Cc4ncc(C(=O)O)cc4CC4(C#N)CC4)CC3)c2O1 ; C[C@@]1(c2ccc(Cl)cc2F)Oc2cccc(C3CCN(Cc4ncc(C(=O)O)cc4CC4(C#N)CC4)CC3)c2O1 ; C[C@@]1(c2ccc(Cl)cc2F)Oc2cccc(C3CCN(Cc4ncc(/C=C/C(=O)O)cc4CC4(C#N)CC4)CC3)c2O1 ; C[C@]1(c2ccc(Cl)cc2F)Oc2cccc(C3CCN(Cc4ncc(/C=C/C(=O)O)cc4CC4(C#N)CC4)CC3)c2O1] 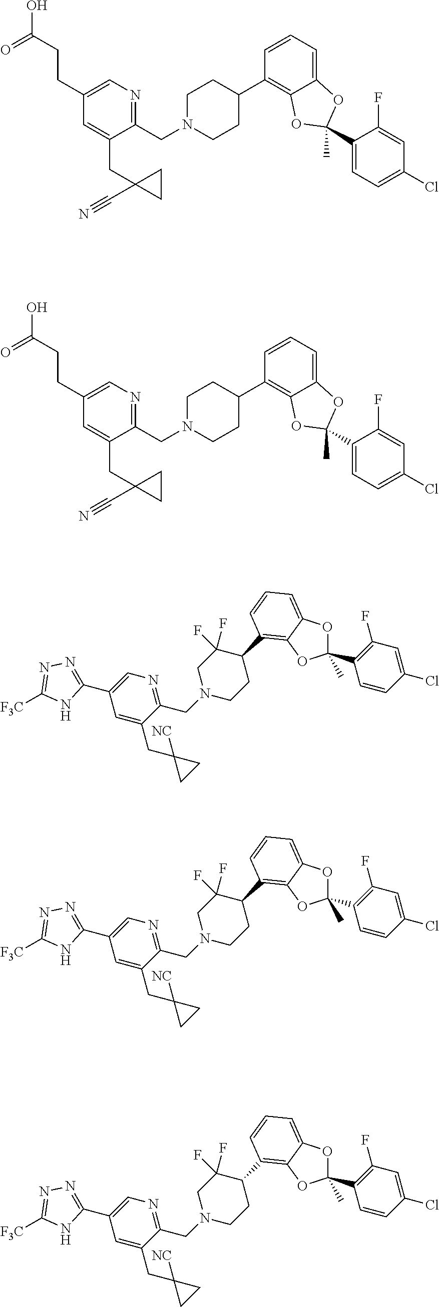 [SMILES: C[C@@]1(c2ccc(Cl)cc2F)Oc2cccc(C3CCN(Cc4ncc(CCC(=O)O)cc4CC4(C#N)CC4)CC3)c2O1 ; C[C@]1(c2ccc(Cl)cc2F)Oc2cccc(C3CCN(Cc4ncc(CCC(=O)O)cc4CC4(C#N)CC4)CC3)c2O1 ; C[C@@]1(c2ccc(Cl)cc2F)Oc2cccc([C@H]3CCN(Cc4ncc(-c5nnc(C(F)(F)F)[nH]5)cc4CC4(C#N)CC4)CC3(F)F)c2O1 ; C[C@]1(c2ccc(Cl)cc2F)Oc2cccc([C@H]3CCN(Cc4ncc(-c5nnc(C(F)(F)F)[nH]5)cc4CC4(C#N)CC4)CC3(F)F)c2O1 ; C[C@@]1(c2ccc(Cl)cc2F)Oc2cccc([C@@H]3CCN(Cc4ncc(-c5nnc(C(F)(F)F)[nH]5)cc4CC4(C#N)CC4)CC3(F)F)c2O1] 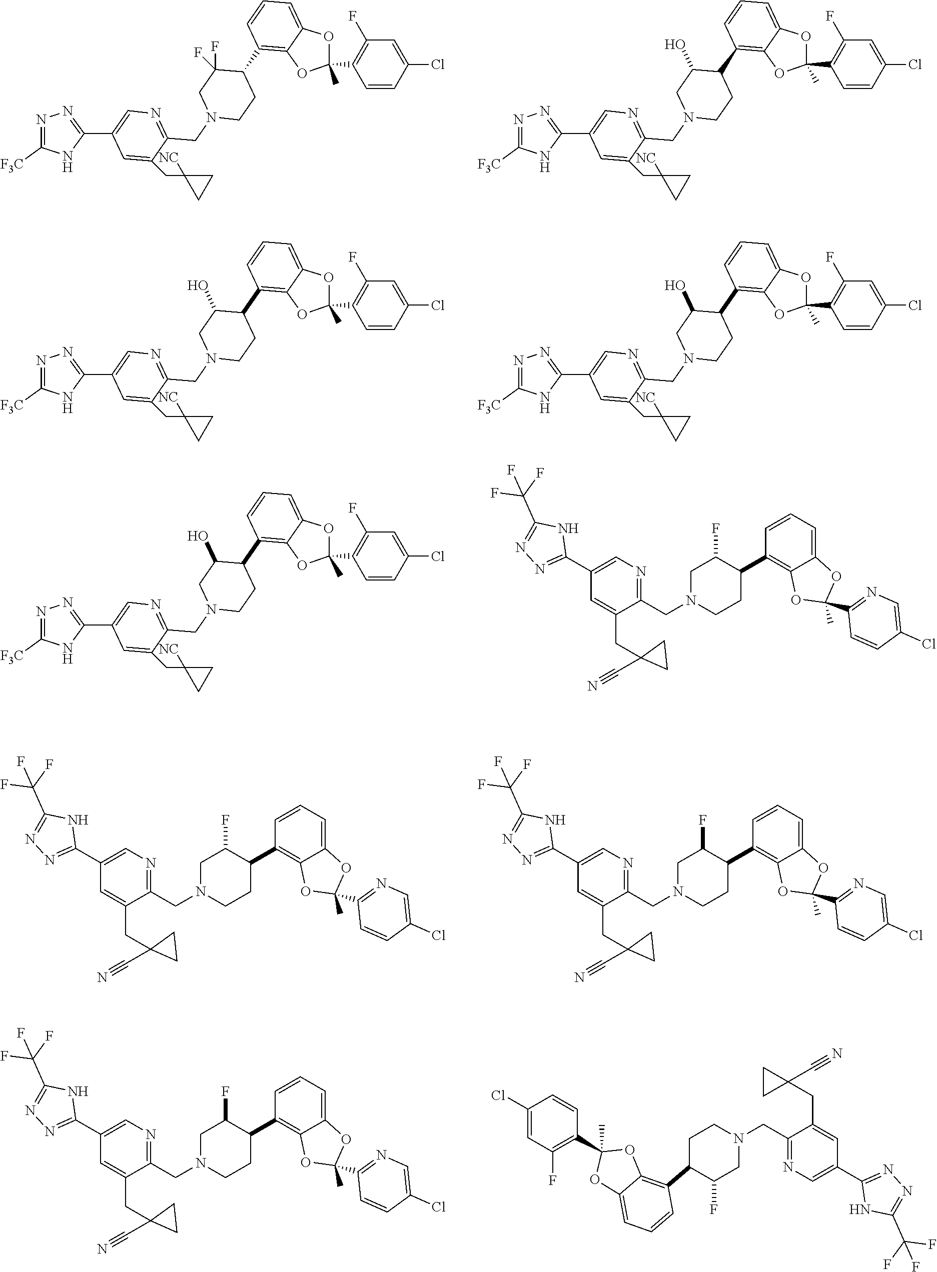 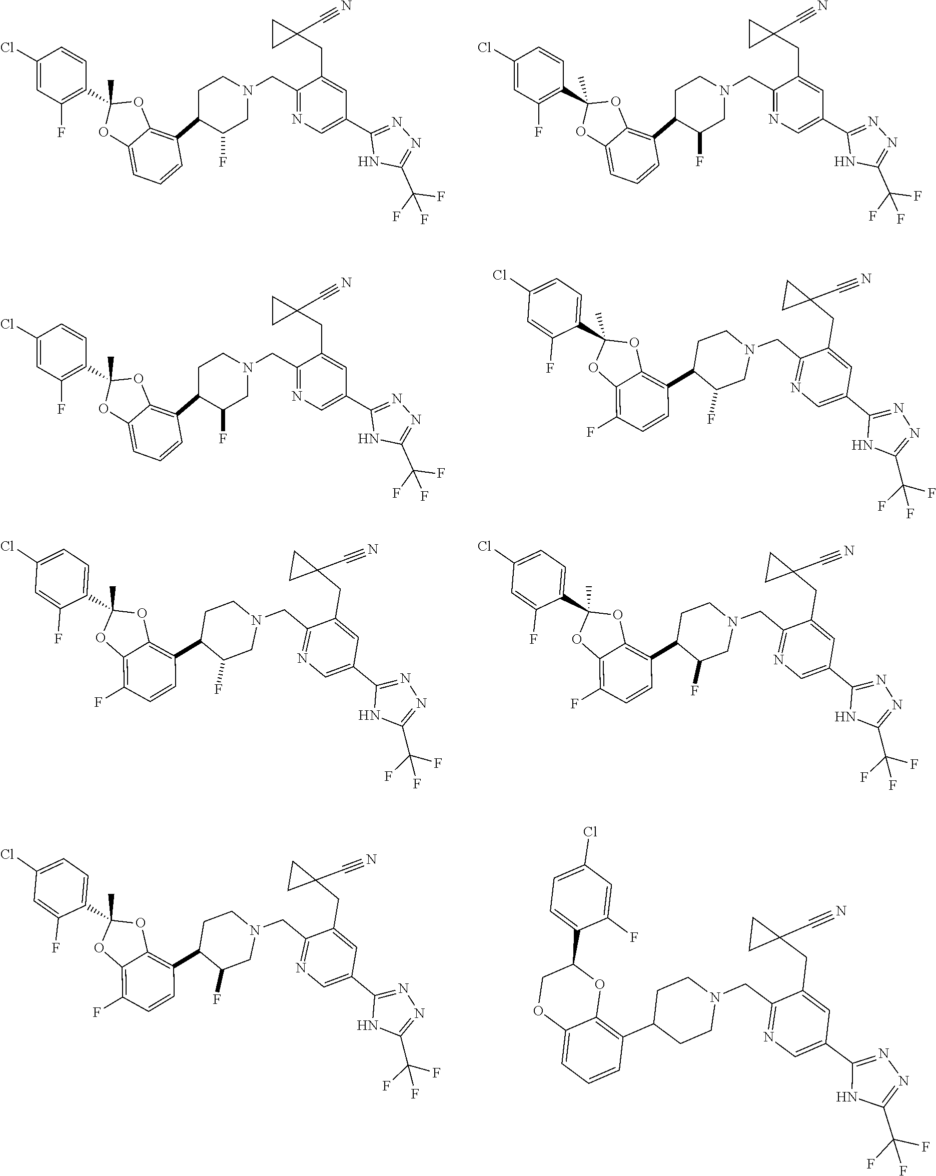 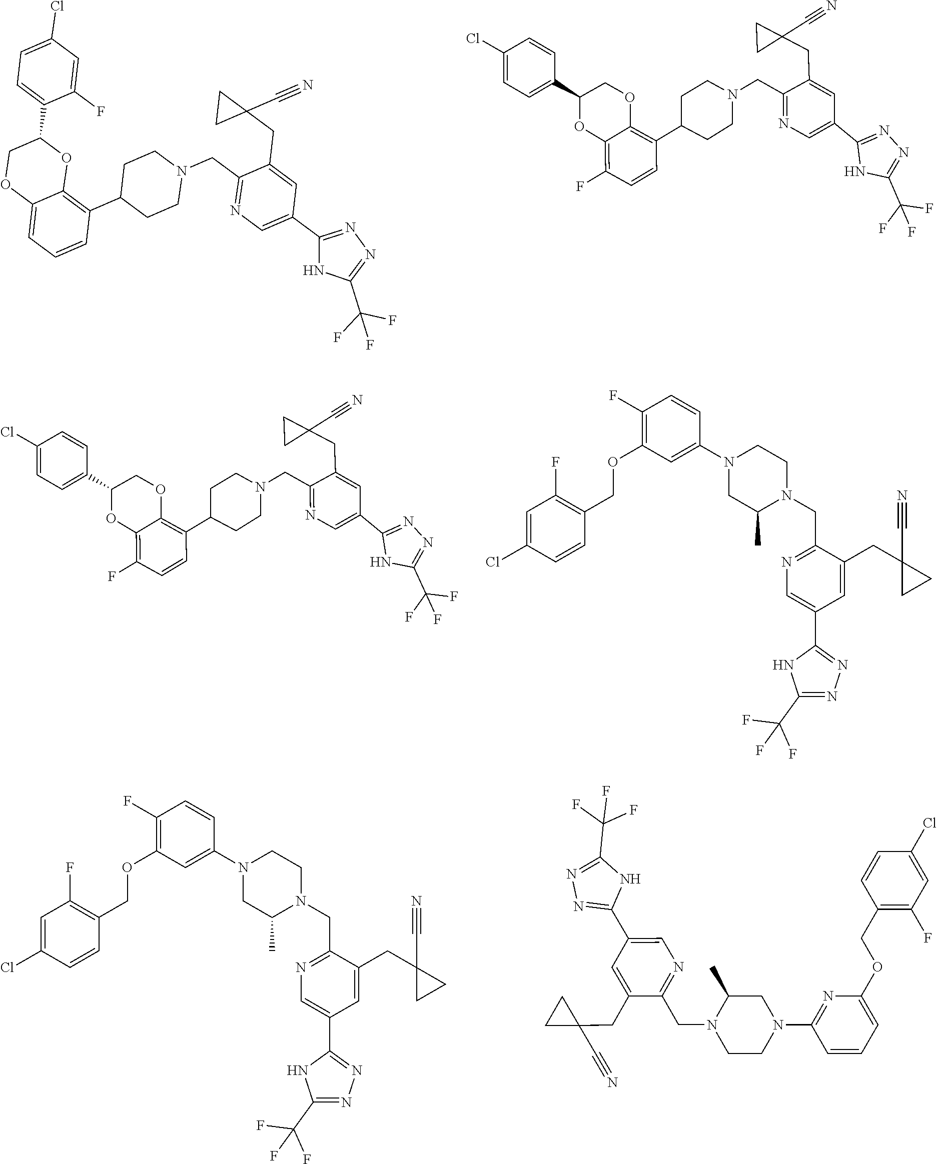 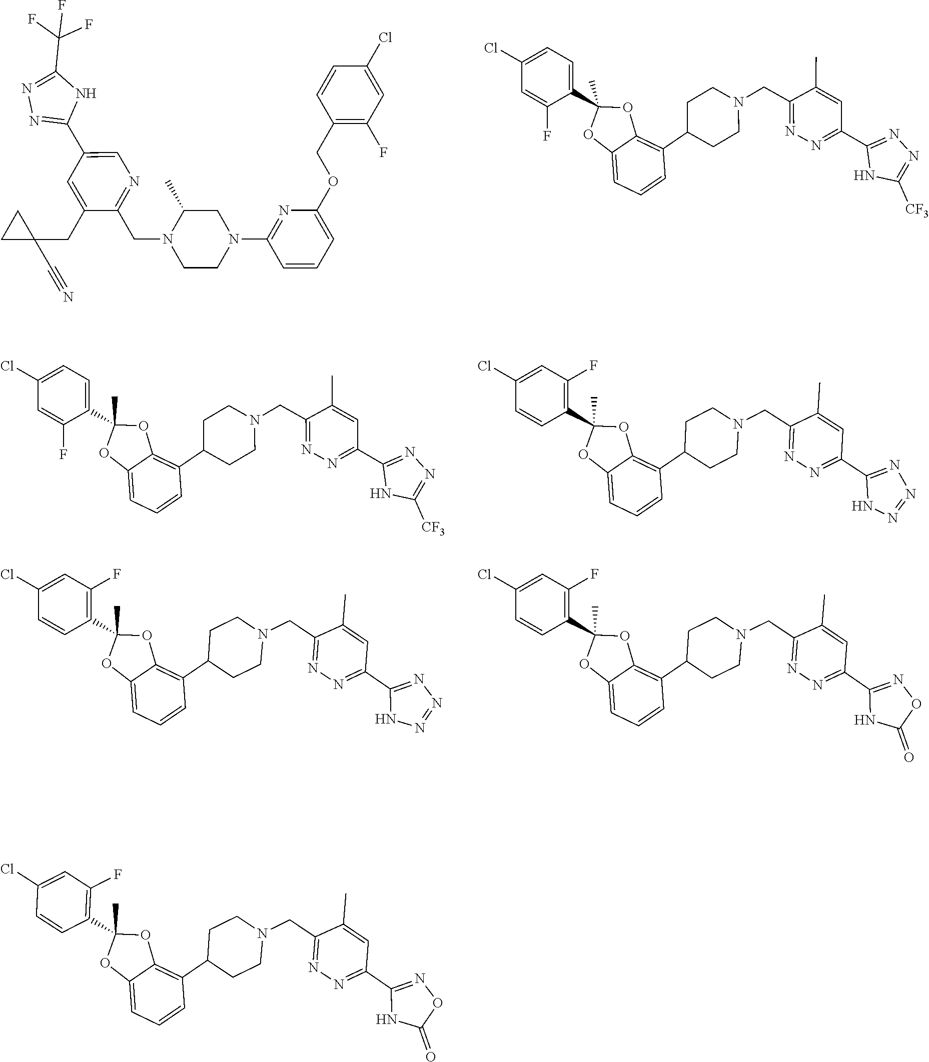 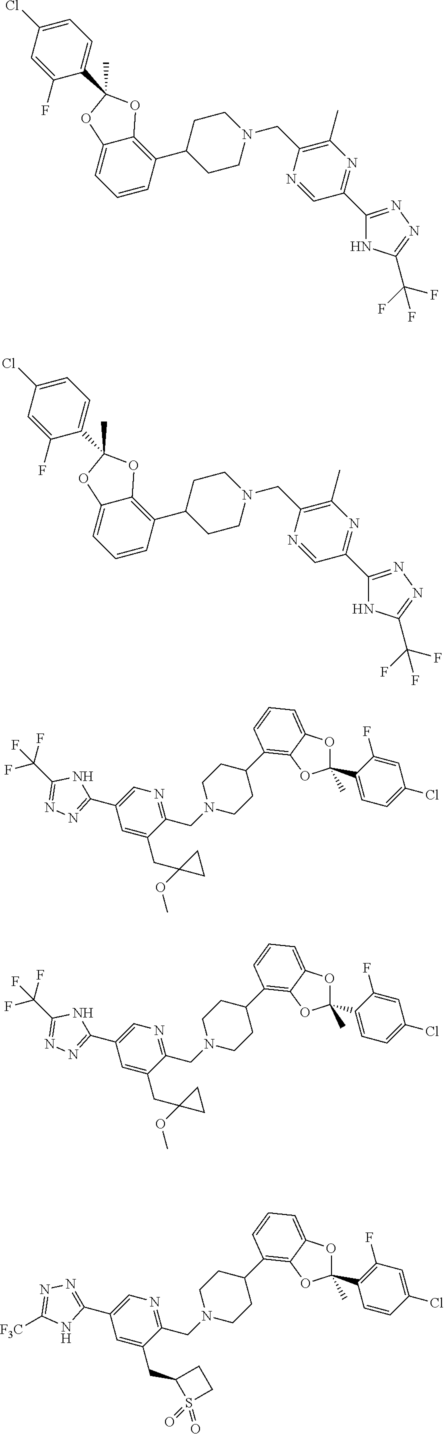 [SMILES: Cc1nc(-c2nnc(C(F)(F)F)[nH]2)cnc1CN1CCC(c2cccc3c2O[C@](C)(c2ccc(Cl)cc2F)O3)CC1 ; Cc1nc(-c2nnc(C(F)(F)F)[nH]2)cnc1CN1CCC(c2cccc3c2O[C@@](C)(c2ccc(Cl)cc2F)O3)CC1 ; COC1(Cc2cc(-c3nnc(C(F)(F)F)[nH]3)cnc2CN2CCC(c3cccc4c3O[C@](C)(c3ccc(Cl)cc3F)O4)CC2)CC1 ; COC1(Cc2cc(-c3nnc(C(F)(F)F)[nH]3)cnc2CN2CCC(c3cccc4c3O[C@@](C)(c3ccc(Cl)cc3F)O4)CC2)CC1 ; C[C@@]1(c2ccc(Cl)cc2F)Oc2cccc(C3CCN(Cc4ncc(-c5nnc(C(F)(F)F)[nH]5)cc4C[C@H]4CCS4(=O)=O)CC3)c2O1] 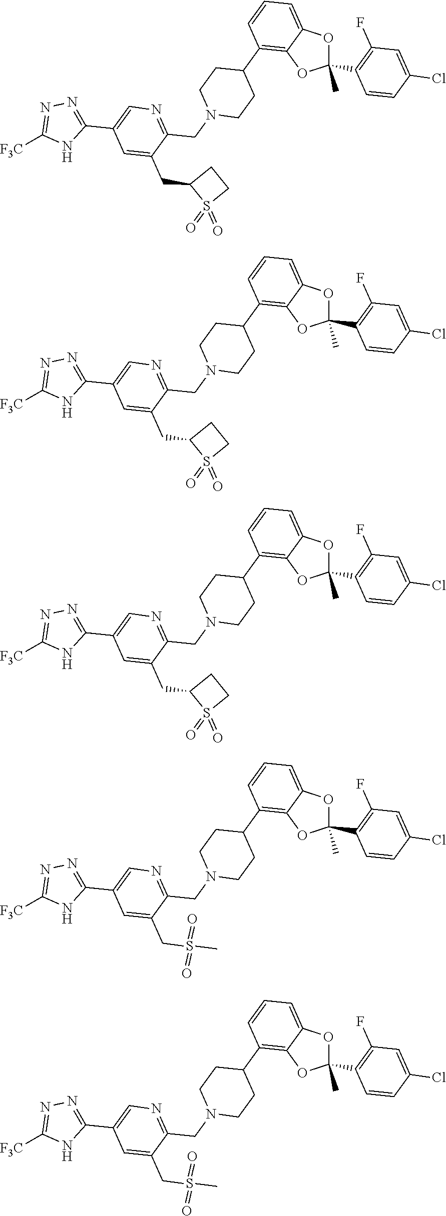 [SMILES: C[C@]1(c2ccc(Cl)cc2F)Oc2cccc(C3CCN(Cc4ncc(-c5nnc(C(F)(F)F)[nH]5)cc4C[C@H]4CCS4(=O)=O)CC3)c2O1 ; C[C@@]1(c2ccc(Cl)cc2F)Oc2cccc(C3CCN(Cc4ncc(-c5nnc(C(F)(F)F)[nH]5)cc4C[C@@H]4CCS4(=O)=O)CC3)c2O1 ; C[C@]1(c2ccc(Cl)cc2F)Oc2cccc(C3CCN(Cc4ncc(-c5nnc(C(F)(F)F)[nH]5)cc4C[C@@H]4CCS4(=O)=O)CC3)c2O1 ; C[C@@]1(c2ccc(Cl)cc2F)Oc2cccc(C3CCN(Cc4ncc(-c5nnc(C(F)(F)F)[nH]5)cc4CS(C)(=O)=O)CC3)c2O1 ; C[C@]1(c2ccc(Cl)cc2F)Oc2cccc(C3CCN(Cc4ncc(-c5nnc(C(F)(F)F)[nH]5)cc4CS(C)(=O)=O)CC3)c2O1] 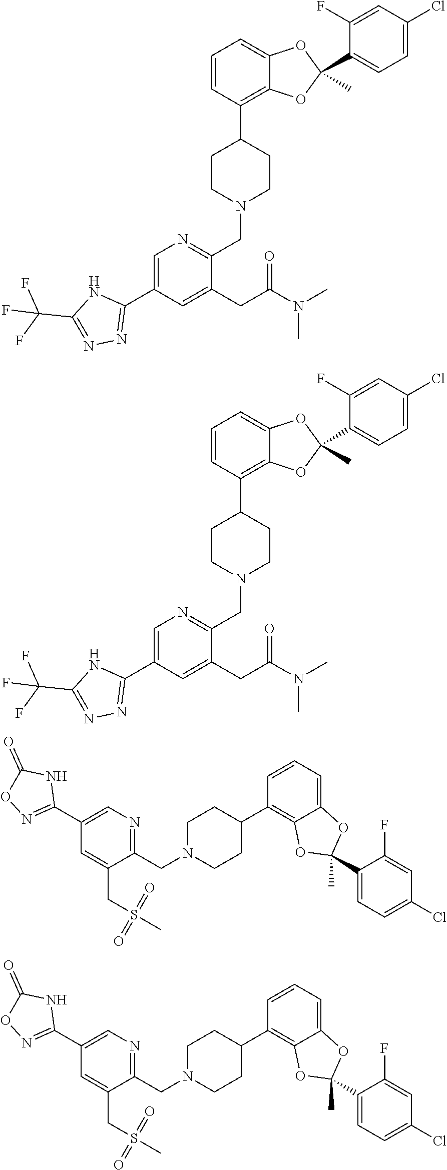 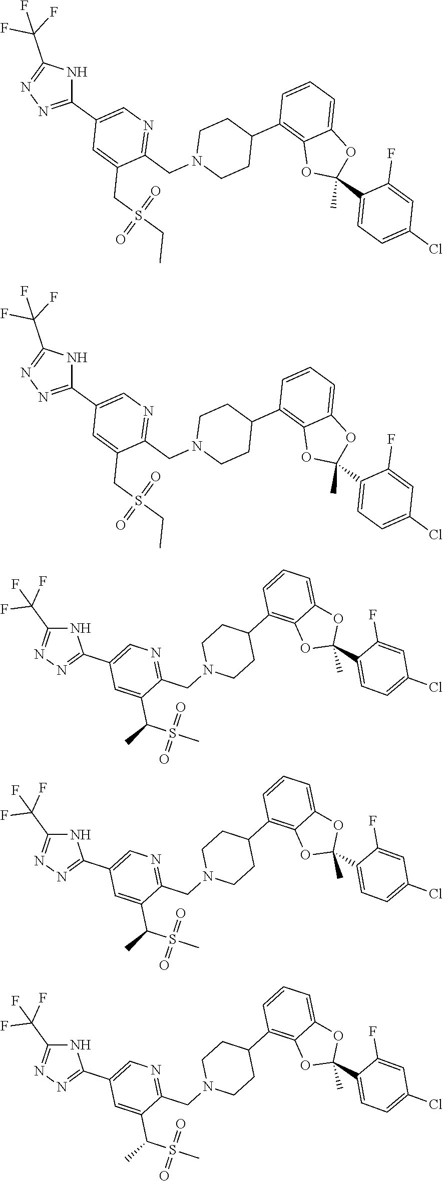 [SMILES: CCS(=O)(=O)Cc1cc(-c2nnc(C(F)(F)F)[nH]2)cnc1CN1CCC(c2cccc3c2O[C@](C)(c2ccc(Cl)cc2F)O3)CC1 ; CCS(=O)(=O)Cc1cc(-c2nnc(C(F)(F)F)[nH]2)cnc1CN1CCC(c2cccc3c2O[C@@](C)(c2ccc(Cl)cc2F)O3)CC1 ; C[C@@H](c1cc(-c2nnc(C(F)(F)F)[nH]2)cnc1CN1CCC(c2cccc3c2O[C@](C)(c2ccc(Cl)cc2F)O3)CC1)S(C)(=O)=O ; C[C@@H](c1cc(-c2nnc(C(F)(F)F)[nH]2)cnc1CN1CCC(c2cccc3c2O[C@@](C)(c2ccc(Cl)cc2F)O3)CC1)S(C)(=O)=O ; C[C@H](c1cc(-c2nnc(C(F)(F)F)[nH]2)cnc1CN1CCC(c2cccc3c2O[C@](C)(c2ccc(Cl)cc2F)O3)CC1)S(C)(=O)=O] 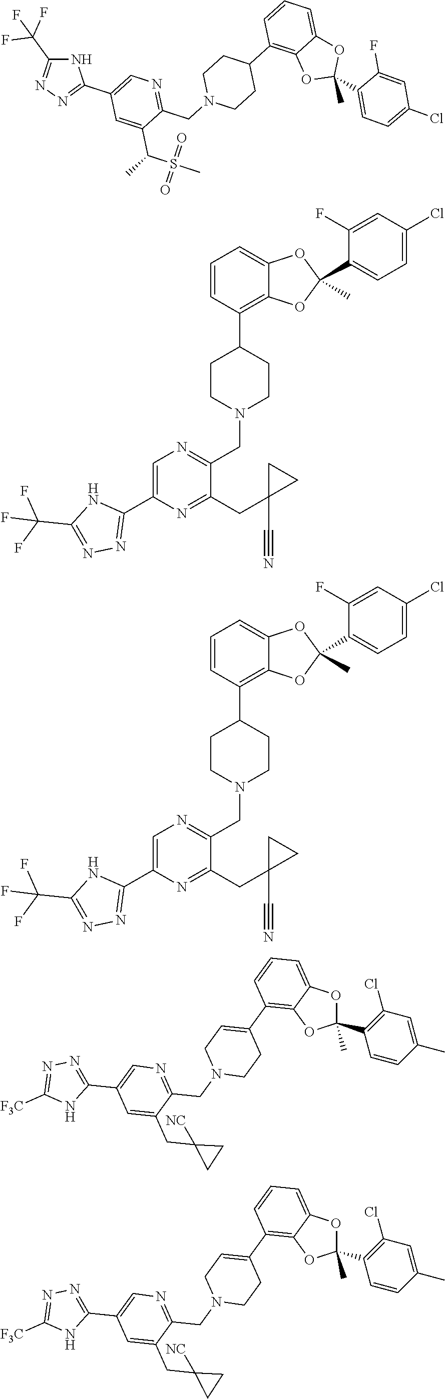 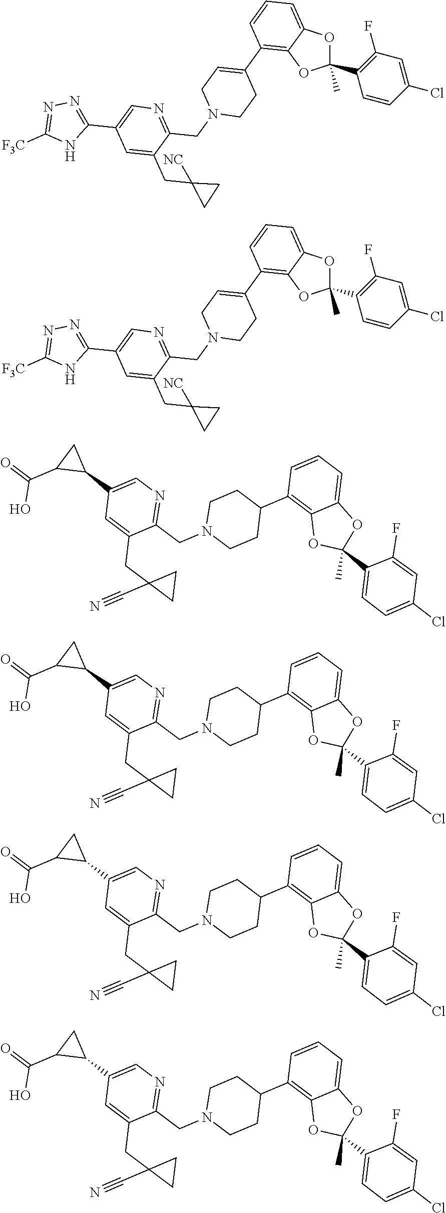 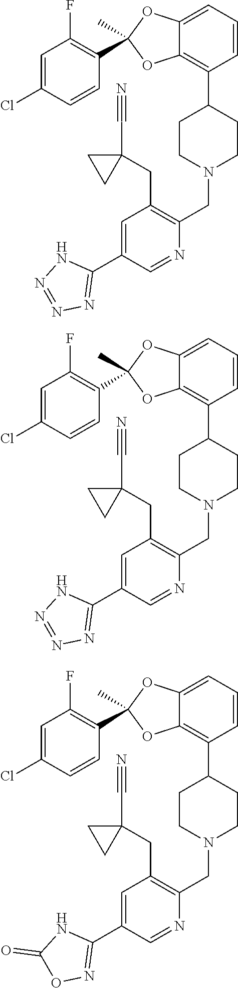 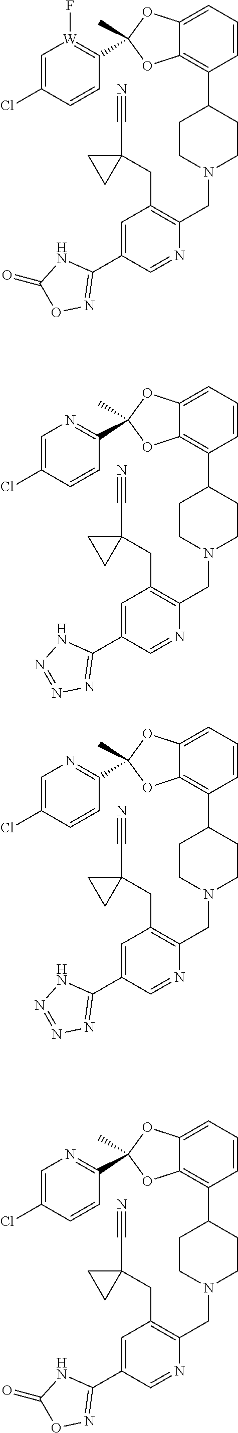 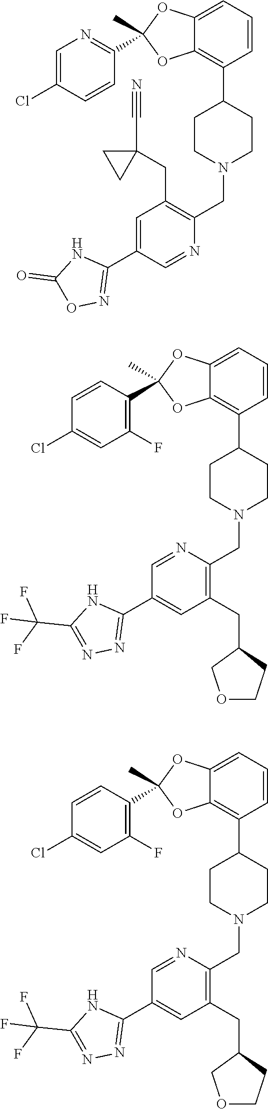 [SMILES: C[C@@]1(c2ccc(Cl)cn2)Oc2cccc(C3CCN(Cc4ncc(-c5noc(=O)[nH]5)cc4CC4(C#N)CC4)CC3)c2O1 ; C[C@]1(c2ccc(Cl)cc2F)Oc2cccc(C3CCN(Cc4ncc(-c5nnc(C(F)(F)F)[nH]5)cc4C[C@H]4CCOC4)CC3)c2O1 ; C[C@@]1(c2ccc(Cl)cc2F)Oc2cccc(C3CCN(Cc4ncc(-c5nnc(C(F)(F)F)[nH]5)cc4C[C@H]4CCOC4)CC3)c2O1] 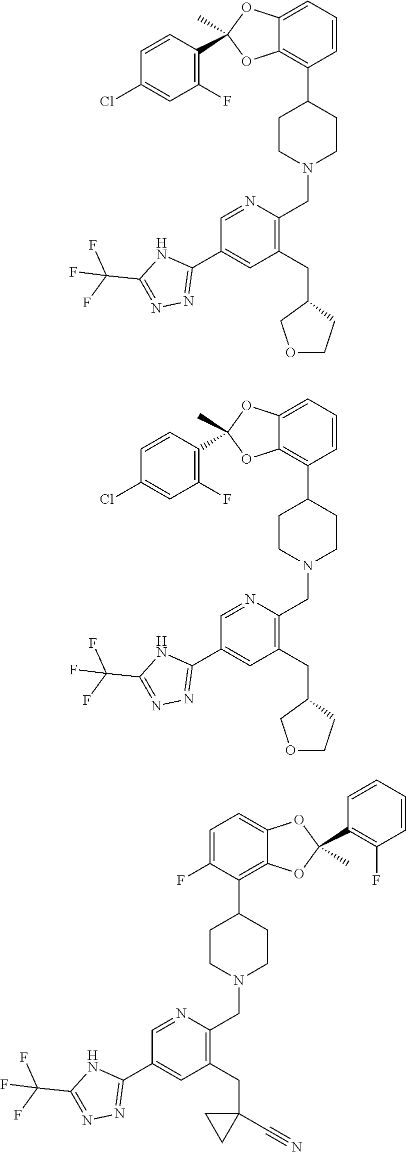 [SMILES: C[C@]1(c2ccc(Cl)cc2F)Oc2cccc(C3CCN(Cc4ncc(-c5nnc(C(F)(F)F)[nH]5)cc4C[C@@H]4CCOC4)CC3)c2O1 ; C[C@@]1(c2ccc(Cl)cc2F)Oc2cccc(C3CCN(Cc4ncc(-c5nnc(C(F)(F)F)[nH]5)cc4C[C@@H]4CCOC4)CC3)c2O1 ; C[C@@]1(c2ccccc2F)Oc2ccc(F)c(C3CCN(Cc4ncc(-c5nnc(C(F)(F)F)[nH]5)cc4CC4(C#N)CC4)CC3)c2O1] 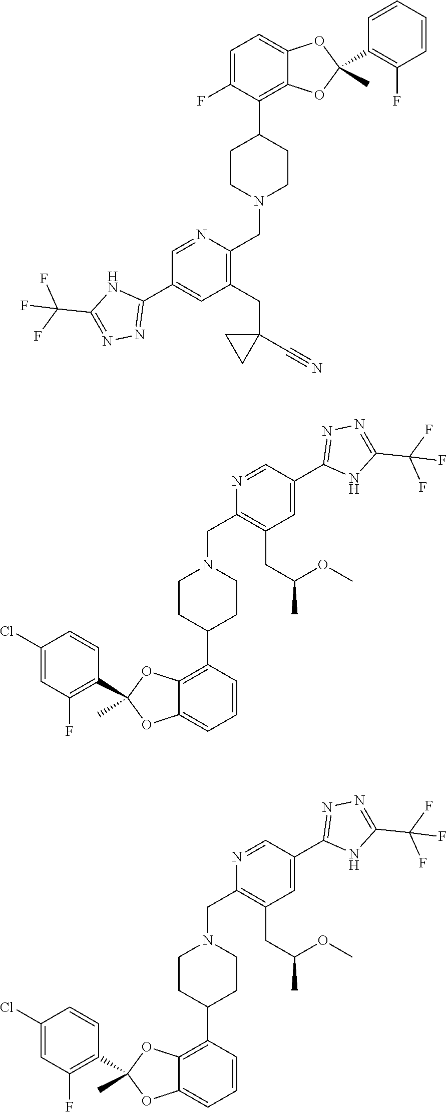 [SMILES: C[C@]1(c2ccccc2F)Oc2ccc(F)c(C3CCN(Cc4ncc(-c5nnc(C(F)(F)F)[nH]5)cc4CC4(C#N)CC4)CC3)c2O1 ; CO[C@@H](C)Cc1cc(-c2nnc(C(F)(F)F)[nH]2)cnc1CN1CCC(c2cccc3c2O[C@](C)(c2ccc(Cl)cc2F)O3)CC1 ; CO[C@@H](C)Cc1cc(-c2nnc(C(F)(F)F)[nH]2)cnc1CN1CCC(c2cccc3c2O[C@@](C)(c2ccc(Cl)cc2F)O3)CC1] 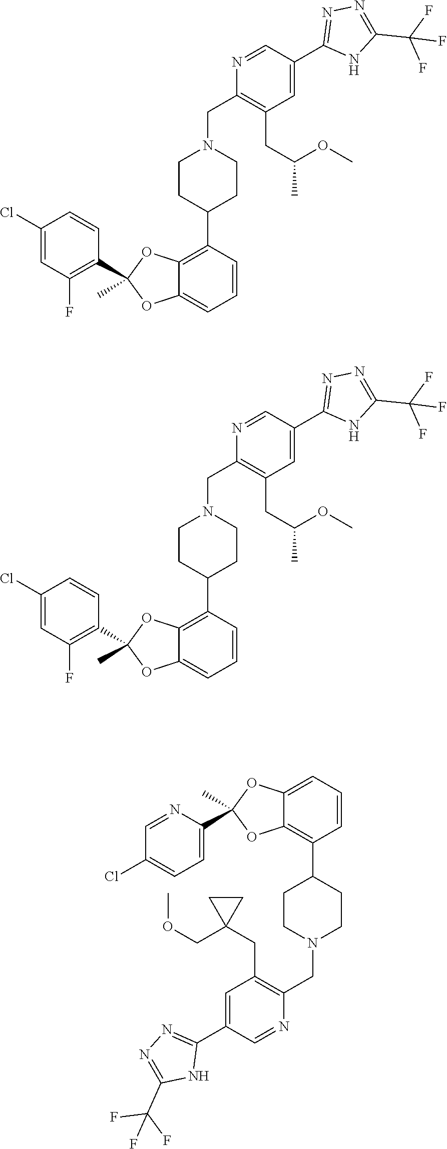 [SMILES: CO[C@H](C)Cc1cc(-c2nnc(C(F)(F)F)[nH]2)cnc1CN1CCC(c2cccc3c2O[C@](C)(c2ccc(Cl)cc2F)O3)CC1 ; CO[C@H](C)Cc1cc(-c2nnc(C(F)(F)F)[nH]2)cnc1CN1CCC(c2cccc3c2O[C@@](C)(c2ccc(Cl)cc2F)O3)CC1 ; COCC1(Cc2cc(-c3nnc(C(F)(F)F)[nH]3)cnc2CN2CCC(c3cccc4c3O[C@@](C)(c3ccc(Cl)cn3)O4)CC2)CC1] 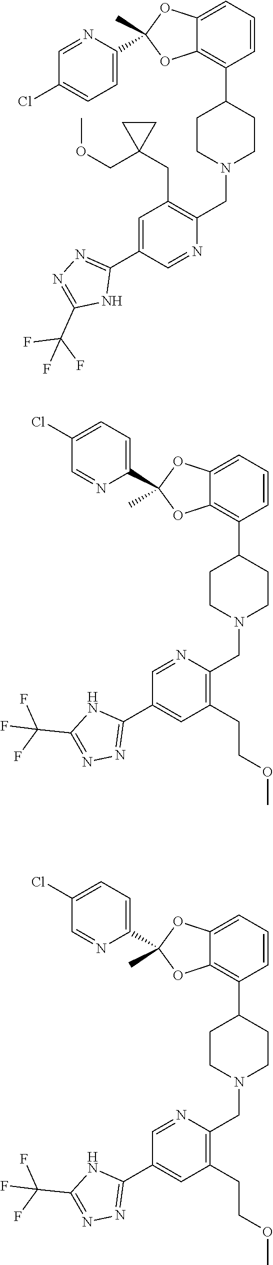 [SMILES: COCC1(Cc2cc(-c3nnc(C(F)(F)F)[nH]3)cnc2CN2CCC(c3cccc4c3O[C@](C)(c3ccc(Cl)cn3)O4)CC2)CC1 ; COCCc1cc(-c2nnc(C(F)(F)F)[nH]2)cnc1CN1CCC(c2cccc3c2O[C@@](C)(c2ccc(Cl)cn2)O3)CC1 ; COCCc1cc(-c2nnc(C(F)(F)F)[nH]2)cnc1CN1CCC(c2cccc3c2O[C@](C)(c2ccc(Cl)cn2)O3)CC1] 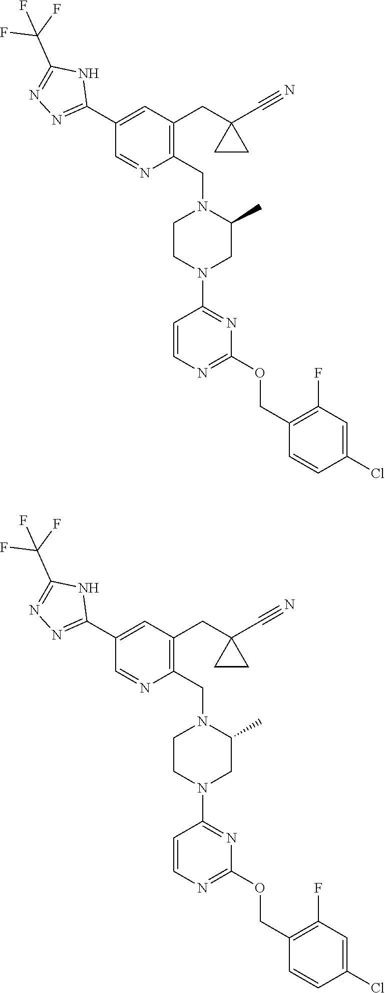 [SMILES: C[C@H]1CN(c2ccnc(OCc3ccc(Cl)cc3F)n2)CCN1Cc1ncc(-c2nnc(C(F)(F)F)[nH]2)cc1CC1(C#N)CC1 ; C[C@@H]1CN(c2ccnc(OCc3ccc(Cl)cc3F)n2)CCN1Cc1ncc(-c2nnc(C(F)(F)F)[nH]2)cc1CC1(C#N)CC1] 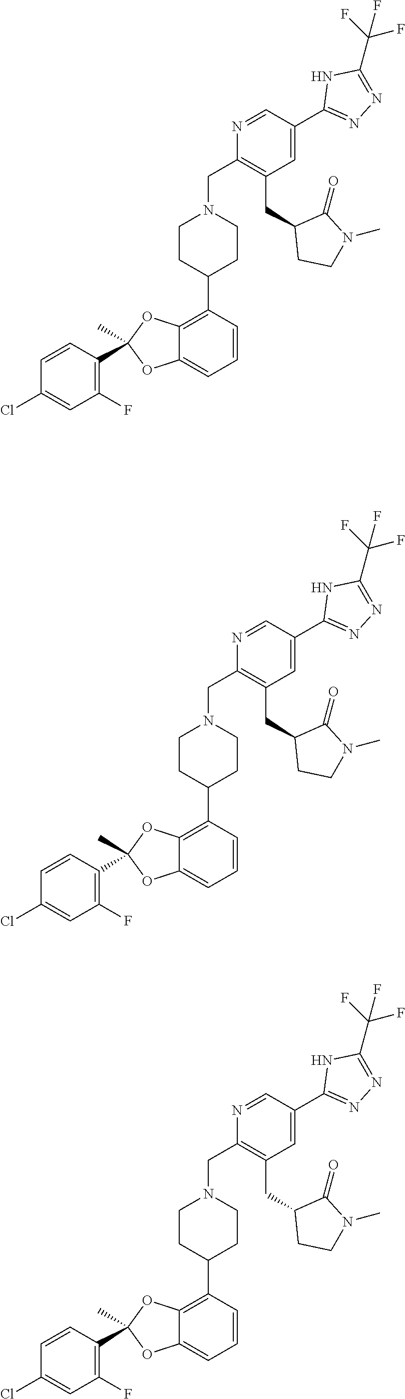 [SMILES: CN1CC[C@@H](Cc2cc(-c3nnc(C(F)(F)F)[nH]3)cnc2CN2CCC(c3cccc4c3O[C@](C)(c3ccc(Cl)cc3F)O4)CC2)C1=O ; CN1CC[C@@H](Cc2cc(-c3nnc(C(F)(F)F)[nH]3)cnc2CN2CCC(c3cccc4c3O[C@@](C)(c3ccc(Cl)cc3F)O4)CC2)C1=O ; CN1CC[C@H](Cc2cc(-c3nnc(C(F)(F)F)[nH]3)cnc2CN2CCC(c3cccc4c3O[C@](C)(c3ccc(Cl)cc3F)O4)CC2)C1=O] 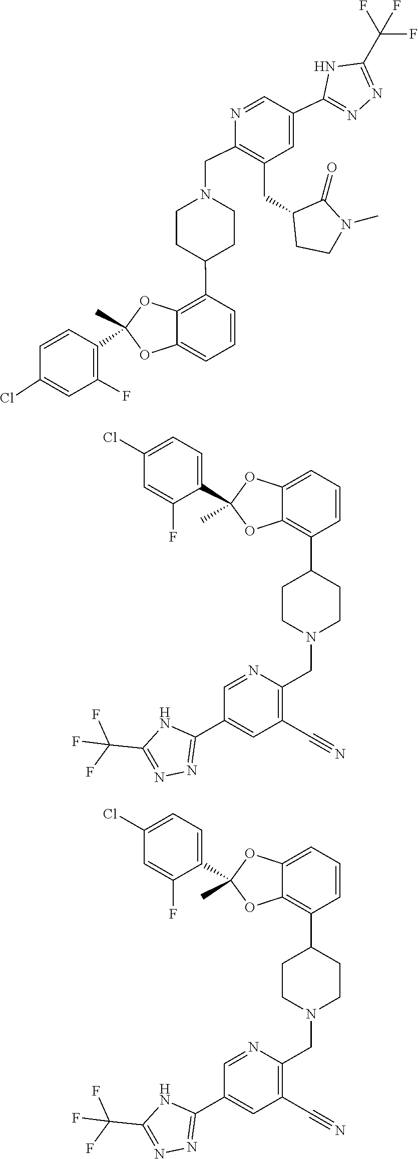 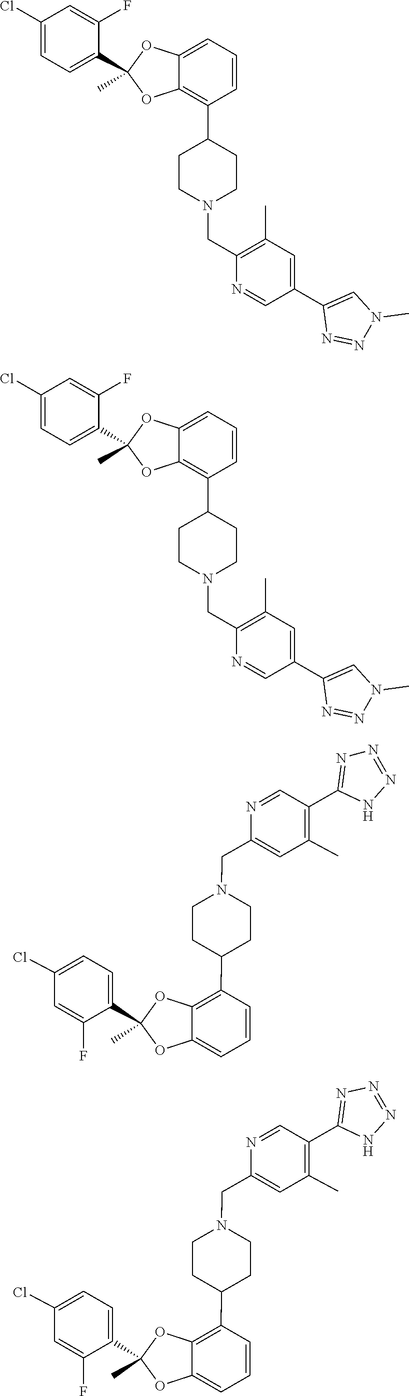 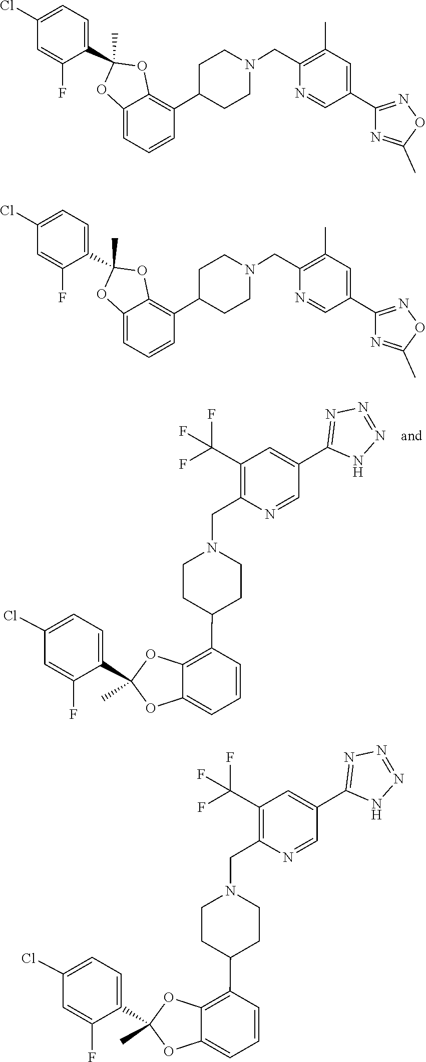 [SMILES: Cc1nc(-c2cnc(CN3CCC(c4cccc5c4O[C@](C)(c4ccc(Cl)cc4F)O5)CC3)c(C)c2)no1 ; Cc1nc(-c2cnc(CN3CCC(c4cccc5c4O[C@@](C)(c4ccc(Cl)cc4F)O5)CC3)c(C)c2)no1 ; C[C@@]1(c2ccc(Cl)cc2F)Oc2cccc(C3CCN(Cc4ncc(-c5nnn[nH]5)cc4C(F)(F)F)CC3)c2O1 ; C[C@]1(c2ccc(Cl)cc2F)Oc2cccc(C3CCN(Cc4ncc(-c5nnn[nH]5)cc4C(F)(F)F)CC3)c2O1]
or a pharmaceutically acceptable salt or solvate thereof.

2. A pharmaceutical composition comprising a compound of claim 1, or a pharmaceutically acceptable salt or solvate thereof, and a pharmaceutically acceptable excipient.

3. A method for treating a GLP-1 associated disease, disorder, or condition, the method comprising administering to a patient in need thereof an effective amount of a compound of claim 1, or a pharmaceutically acceptable salt or solvate thereof.

4. The method of claim 3, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof.

5. A method of treating type 2 diabetes mellitus in a patient in need thereof, the method comprising administering to a patient in need thereof an effective amount of a compound of claim 1, or a pharmaceutically acceptable salt or solvate thereof.

6. A method for modulating insulin levels in a patient in need of such modulating, the method comprising administering to a patient in need thereof an effective amount of a compound of claim 1, or a pharmaceutically acceptable salt or solvate thereof.

7. A method for modulating glucose levels in a patient in need of such modulating, the method comprising administering to a patient in need thereof an effective amount of a compound of claim 1, or a pharmaceutically acceptable salt or solvate thereof.

8. The method of claim 3, further comprising administering an additional therapy or therapeutic agent to the patient.

9. The method of claim 8, wherein the additional therapy or therapeutic agent is selected from the group consisting of an antidiabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.

10. The method of claim 5, further comprising administering an additional therapy or therapeutic agent to the patient.

11. The method of claim 10, wherein the additional therapy or therapeutic agent is selected from the group consisting of an antidiabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.

12. The method of claim 6, further comprising administering an additional therapy or therapeutic agent to the patient.

13. The method of claim 12, wherein the additional therapy or therapeutic agent is selected from the group consisting of an antidiabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.

14. The method of claim 7, further comprising administering an additional therapy or therapeutic agent to the patient.

15. The method of claim 14, wherein the additional therapy or therapeutic agent is selected from the group consisting of an antidiabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.
